(19) United States
(12) Patent Application Publication
(10) Pub. No.: US 2019/0016775 A1
(43) Pub. Date: Jan. 17, 2019

(54) Immunogenic Compositions

(71) Applicant: Pfizer Inc., New York, NY (US)

(72) Inventors: Joseph John Binder, San Diego, CA (US); Helen Kim Cho, San Diego, CA (US); Paul Jason Cockle, San Anselmo, CA (US); Derek John Falconer, San Diego, CA (US); Siradanahalli Guru, San Diego, CA (US); Marianne Marcela Andrea Martinic, Bottmingen (CH)

(21) Appl. No.: 16/029,878

(22) Filed: Jul. 9, 2018

Related U.S. Application Data
(60) Provisional application No. 62/531,227, filed on Jul. 11, 2017.
(60) Provisional application No. 62/682,044, filed on Jun. 7, 2018.

(51) Int. Cl. C07K 14/705; C12N 9/12

(52) U.S. Cl. CPC C07K 14/705; A61K 38/00; C12Y 207/07049; C12N 9/1276

(57) ABSTRACT

The present disclosure provides: (a) isolated immunogenic CEA polypeptides; (b) isolated nucleic acid molecules encoding (i) an immunogenic CEA polypeptide, (ii) an immunogenic CEA polypeptide and an immunogenic MUC1 polypeptide, (iii) an immunogenic CEA polypeptide and an immunogenic TERT polypeptide, or (iv) an immunogenic CEA polypeptide, an immunogenic MUC1 polypeptide, and an immunogenic TERT polypeptide; (c) compositions comprising an isolated nucleic acid molecule; and (d) methods relating to uses of the immunogenic CEA polypeptides, nucleic acid molecules, and compositions.

### REFERENCE TO RELATED APPLICATIONS

This application claims the benefit of U.S. Provisional Application No. 62/531,227 filed on Jul. 11, 2017 and U.S. Provisional Application No. 62/682,044 filed on Jun. 7, 2018. The entire content of each of the forgoing applications is incorporated herein by reference.

### REFERENCE TO SEQUENCE LISTING

This application is being filed along with a sequence listing in electronic format. The sequence listing is provided as a file in .txt format entitled “PC72354A_FF_SeqList_ST25.txt”, created on Jul. 3, 2018, and having a size of 963 KB. The sequence listing contained in the .txt file is part of the specification and is herein incorporated by reference in its entity.

### FIELD OF THE INVENTION

The present invention relates generally to immunotherapy and specifically to vaccines and methods for treating or preventing neoplastic disorders.

### BACKGROUND OF THE INVENTION

Cancers are a leading cause of mortality worldwide. They may occur in a variety of organs and tissues, such as pancreas, breasts, lungs, stomach, colon, and rectum. Pancreatic cancers are the fourth most common cause of cancer deaths in the United States. Pancreatic cancers may occur in the exocrine or endocrine component of the pancreas. Exocrine cancers include (1) pancreatic adenocarcinoma, which is by far the most common type, (2) acinar cell carcinoma, which represents 5% of exocrine pancreatic cancers, (3) cystadenocarcinomas, which account for 1% of pancreatic cancers, and (4) other rare forms of cancers, such as pancreatoblastoma, adenosquamous carcinomas, signet ring cell carcinomas, hepatoid carcinomas, colloid carcinomas, undifferentiated carcinomas, and undifferentiated carcinomas with osteoclast-like giant cells.

Breast cancer (BrC) is another common cancer among American women and the second leading cause of cancer death in women. Based on various tumor markers such as estrogen receptor (ER), progesterone receptor (PR), and human epidermal growth factor receptor 2 (HER2), breast cancers can be classified into major subtypes, such as (1) hormone receptor-positive cancers (where the cancer cells contain either estrogen receptors or progesterone receptors); (2) hormone receptor-negative cancers (where the cancer cells don't have either estrogen receptors or progesterone receptors); (3) HER2/neu positive (wherein cancers that have excessive HER2/neu protein or extra copies of the HER2/neu gene); (4) HER2/neu negative cancers (where the cancers don't have excess HER2/neu); (5) triple-negative cancers (wherein the breast cancer cells have neither estrogen receptors, nor progesterone receptors, nor excessive HER2); and (6) triple-positive cancers (where the cancers are estrogen receptor-positive, progesterone receptor-positive, and have too much HER2).

Lung cancer accounts for more than a quarter of all cancer deaths and is the leading cause of cancer-related mortality worldwide. Approximately 85% of cases are histologically classified as non-small cell lung cancers (NSCLC). NSCLC may be further classified into several subtypes, such as squamous cell (epidermoid) carcinoma, adenocarcinoma, large cell (undifferentiated) carcinoma, adenosquamous carcinoma, and sarcomatoid carcinoma. The second common type of lung cancer is small cell lung cancer (SCLC), which accounts for about 10% to 15% of all lung cancers.

Gastric cancer (GaC) is the third most common cause of cancer-related death in the world. About 90-95% of gastric cancers are adenocarcinomas; other less common types include lymphoma, GISTs, and carcinoid tumors.

Colorectal cancer (CRC) is also a leading cause of cancer-related deaths in the United States. Adenocarcinomas are the most common type of CRC, which accounts for more than 95% of colorectal cancers. Other less common types of CRC include Carcinoid tumors, gastrointestinal stromal tumors (GISTs), lymphomas, and sarcomas.

Traditional regimens of cancer management have been successful in the management of a selective group of circulating and solid cancers. However, many types of cancers are resistant to traditional approaches. In recent years, immunotherapy for cancers has been explored, particularly cancer vaccines and antibody therapies. One approach of cancer immunotherapy involves the administering an immunogen to generate an active systemic immune response towards a tumor-associated antigen (TAA) on the target cancer cell. While a large number of tumor-associated antigens have been identified and many of these antigens have been explored as viral-, bacterial-, protein-, peptide-, or DNA-based vaccines for the treatment or prevention of cancers, most clinical trials so far have failed to produce a therapeutic product. Therefore, there exists a need for an immunogen or vaccine that may be used in the treatment or prevention of cancers.

The present disclosure relates to immunogenic polypeptides derived from the tumor-associated antigens MUC1, CEA, or TERT, nucleic acid molecules encoding such immunogenic polypeptides, compositions comprising such an immunogenic polypeptide or nucleic acid molecule, such as vaccines, and uses of the polypeptides, nucleic acid molecules, and compositions.

The human mucin 1 protein (MUC1; also known as episialin, PEM, H23Ag, EMA, CA15-3, and MCA) is a polymorphic transmembrane glycoprotein expressed on the apical surfaces of simple and glandular epithelia. The MUC1 gene encodes a single polypeptide chain precursor that includes a signal peptide sequence. Immediately after translation the signal peptide sequence is removed and the remaining portion of the MUC1 precursor is further cleaved into two peptide fragments: the longer N-terminal subunit (MUC1-N or MUC1α) and the shorter C-terminal subunit (MUC1-C or MUC1β). The mature MUC1 comprises a MUC1-N and a MUC1-C associated through stable hydrogen bonds. MUC1-N, which is an extracellular domain, contains variable number tandem repeats (VNTR) of 20 amino acid residues, with the number of repeats varying from 20 to 125 in different individuals. The region of the MUC1 protein that is composed of the variable number tandem repeats is also referred to in the present disclosure as “VNTR region.” MUC1-C contains a short extracellular region (approximately 53 amino acids), a transmembrane domain (approximately 28 amino acid), and a cytoplasmic tail (approximately 72 amino acids). The cytoplasmic tail of MUC1 (MUC1-CT) contains highly conserved serine and tyrosine residues that are phosphorylated by growth factor receptors and intracellular kinases. Human MUC1 exists in multiple isoforms resulting from different types of MUC1 RNA alternative splicing. The amino acid sequence of full length human MUC1 isoform 1 protein precursor (isoform 1, Uniprot P15941-1) is provided in SEQ ID NO: 1 (“MUC1 Reference Polypeptide”). At least 16 other isoforms of human MUC-1 have been reported so far (Uniprot P15941-2 through P15941-17), which include various insertions, deletions, or substitutions as compared to the sequence of isoform 1. These isoforms are known as isoform 2, 3, 4, 5, 6, Y, 8, 9, F, Y-LSP, S2, M6, ZD, T10, E2, and J13 (Uniprot P15941-2 through P15941-17, respectively). The full length human MUC1 isoform 1 precursor protein consists of 1255 amino acids, which includes a signal peptide sequence at amino acids 1-23. The MUC1-N and MUC1-C domains of the mature MUC1 protein consist of amino acids 24-1097 and 1098-1255, respectively.

Carcinoembryonic antigen-related cell adhesion molecules (also known as CEACAMs) are a group of glycoproteins in the immunoglobulin (Ig) superfamily group. Structurally, the CEACAM group consists of a single N-terminal domain and a maximum of six disulfide-linked internal domains similar to C2-type Ig domains. The group contains 12 proteins (CEACAM1, 3-8, 16, 18-21), several of which, such as CEACAM1, CEACAM5, and CEACAM6, have been considered valid clinical markers and promising therapeutic targets in various cancers such as melanoma, lung, colorectal, and pancreatic cancers. Overexpression of CEACAM5, also referred to herein and known in the art as CEA, has been found to be in the majority of human carcinomas. CEACAM5 is expressed as a 702-amino acid precursor protein consisting of: (1) a signal peptide (amino acids 1-34); (2) the N-domain (amino acids 35-144); (3) three repeating units comprising six constant C2-like domains termed as A1 (amino acids 146-237), B1 (amino acids 238-322), A2 (amino acids 324-415), and B2 (amino acids 416-498), A3 (amino acids 502-593), and B3 (amino acids 594-677); and (4) a propeptide (amino acids 686-702). The signal peptide is cleaved off from the mature protein during transport to the cell surface. The amino acid sequence of a full length human CEA precursor protein is available at UniProt (Accession No. P06731) and is also set forth herein in SEQ ID NO:2 (“CEA Reference Polypeptide”).

Telomerase reverse transcriptase (or TERT) is the catalytic component of the telomerase, which is a ribonucleoprotein polymerase responsible for maintaining telomere ends by addition of the telomere repeat TTAGGG. In addition to TERT, telomerase also includes an RNA component which serves as a template for the telomere repeat. Human TERT gene encodes an 1132 amino acid protein. Several isoforms of human TERT exist, which result from alternative splicing. The amino acid sequences of isoform 1, isoform 2, isoform 3, and isoform 4 are available at Uniprot (<www.uniprot.org>; Uniprot identifiers O14746-1, O14746-2, O14746-3, and O14746-4, respectively). The amino acid sequence of human full length TERT isoform 1 protein (isoform 1, Genbank AAD30037, Uniprot O14746-1) is also provided herein in SEQ ID NO:3 (“TERT Reference Polypeptide”). As compared with TERT isoform 1 (O14746-1), isoform 2 (O14746-2) has replacement of amino acids 764-807 (STLTDLQPYM . . . LNEASSGLFD→LRPVPGDPAG . . . AGRAAPAFGG) and deletion of C-terminal amino acids 808-1132), isoform 3 (O14746-3) has deletion of amino acids 885-947, and isoform 4 (O14746-4) has deletions of amino acids 711-722 and 808-1132, and replacement of amino acids 764-807 (STLTDLQPYM . . . LNEASSGLFD→LRPVPGDPAG . . . AGRAAPAFGG).

### SUMMARY OF THE INVENTION

In some aspects, the present disclosure provides isolated immunogenic polypeptides derived from tumor-associated antigen (TAA) MUC1, CEA, or TERT, which is useful, for example, in eliciting an immune response in vivo (e.g. in an animal, including humans), or for use as a component in pharmaceutical compositions, including vaccines, for the treatment of cancer.

In other aspects, the present disclosure provides nucleic acid molecules (also referred to as “antigen constructs”) that each encode one or more immunogenic polypeptides provided by the present disclosure. In some embodiments, the present disclosure provides multi-antigen nucleic acid constructs that each encode two, three, or more different immunogenic TAA polypeptides, such as: (1) one immunogenic CEA polypeptide and one immunogenic MUC1 polypeptide; (2) one immunogenic CEA polypeptide and one immunogenic TERT polypeptide; and (3) one immunogenic CEA polypeptide, one immunogenic MUC1 polypeptide, and one immunogenic TERTpolypeptide.

The disclosure also provides vectors (such as plasmid vectors and virual vectors) that contain one or more antigen constructs provided by the present disclosure. The vectors are useful for cloning or expressing the encoded immunogenic TAA polypeptides, or for delivering the antigen constructs in a composition, such as a vaccine, to a host cell or to a host animal or a human.

The present disclosure further provides compositions comprising one or more of the immunogenic polypeptides, isolated antigen constructs encoding one or more immunogenic TAA polypeptides, or vectors containing an antigen construct encoding one or more immunogenic TAA polypeptides. In some embodiments, the composition is an immunogenic composition useful for eliciting an immune response against a TAA in a mammal, such as a mouse, dog, monkey, or human. In some embodiments, the composition is a vaccine composition useful for immunization of a mammal, such as a human, for inhibiting abnormal cell proliferation, for providing protection against the development of cancer (used as a prophylactic), or for treatment of disorders (used as a therapeutic) associated with TAA over-expression, such as cancer, particularly pancreatic, ovarian, lung, colorectal, gastric, and breast cancer.

In some further aspects, the present disclosure provides a method of eliciting an immune response against tumor-associated antigen (TAA) CEA, MUC1, or TERT, which comprises administering to the mammal an effective amount of (1) an immunogenic CEA polypeptide, an immunogenic MUC1 polypeptide, and/or an immunogenic TERT polypeptide, (2) an antigen construct encoding one or more immunogenic TAA polypeptides provided by the present disclosure, (3) vectors (such as viral vectors and plasmid vectors) that contain one or more antigen constructs, or (4) compositions containing one or more immunogenic TAA polypeptides, one or more antigen constructs, or one or more vectors as disclosed herein. Examples of the mammals in each of htemehtods include mouse, dog, monkey, or human.

In some further aspects, the present disclosure provides a method of inhibiting abnormal cell proliferation, a method of providing protection against the development of cancer, a method for treatment of cancer, and a method for treatment of a disorder associated with over-expression of a TAA in a mammal, each of which comprises administering to the mammal an effective amount of (1) an immunogenic CEA polypeptide, an immunogenic MUC1 polypeptide, an immunogenic TERT polypeptide, or a combination of any of these immunogenic polypeptides, (2) an antigen construct encoding one or more of these immunogenic TAA polypeptides, (3) vectors (such as viral vectors and plasmid vectors) that contain one or more antigen constructs, or (4) compositions containing one or more of these immunogenic TAA polypeptides, one or more antigen constructs, or one or more vectors as disclosed herein. Examples of the mammals in each of htemehtods include mouse, dog, monkey, or human. In the methods related to cancer management, examples of cancers include pancreatic, ovarian, lung, colorectal, gastric, or breast cancer.

In an aspect of the present invention, the following embodiments, each described by a numbered clause, are contemplated:

1. An antigen construct, comprising a nucleotide sequence encoding an immunogenic CEA polypeptide as disclosed herein.

2. The antigen construct according to clause 1, further comprising a nucleotide sequence encoding an immunogenic MUC1 polypeptide as disclosed herein.

3. The antigen construct according to clause 1 or 2, further comprising a nucleotide sequence encoding an immunogenic TERT polypeptide as disclosed herein.

4. The antigen construct according to clause 1, further comprising a nucleotide sequence encoding an immunogenic MUC1 polypeptide as disclosed herein and a nucleotide sequence encoding an immunogenic TERT polypeptide as disclosed herein.

5. The antigen construct according to any one of clauses 2, 3, or 4, further comprising a spacer nucleotide sequence as disclosed herein.

6. The antigen construct according to clause 5, wherein the spacer nucleotide sequence encodes a 2A peptide.

7. The antigen construct according to clause 5, wherein the spacer nucleotide sequence encodes a 2A peptide selected from the group consisting of EMC2A, ERA2A, ERB2A, and T2A.

8. The antigen construct according to any one of clauses 1-7, wherein the immunogenic CEA polypeptide is selected from the group consisting of:

(1) a polypeptide comprising or consisting of amino acids 2-702 of SEQ ID NO:2, amino acids 323-702 of SEQ ID NO:2, or amino acids 323-677 of SEQ ID NO:2;

(2) a polypeptide comprising or consisting of amino acid sequence of SEQ ID NO:15 or amino acids 4-704 of SEQ ID NO:15;

(3) a polypeptide comprising or consisting of the amino acid sequence of SEQ ID NO:17 or amino acids 4-526 of SEQ ID NO:17;

(4) a polypeptide comprising or consisting of the sequence of SEQ ID NO:19 or amino acids 4-468 of SEQ ID NO:19; or

(5) a polypeptide that is a functional variant of any of the polypeptides of (1)-(4) above.

9. The antigen construct according to any one of clauses 3-8, wherein the immunogenic TERT polypeptide is selected from the group consisting of:

(1) a polypeptide comprising the amino acid sequence of SEQ ID NO:9 or amino acids 2-893 of SEQ ID NO:9;

(2) a polypeptide comprising the amino acid sequence of SEQ ID NO:11 or amino acids 3-791 of SEQ ID NO:11;

(3) a polypeptide comprising the amino acid sequence of SEQ ID NO:13 or amino acids 4-594 of SEQ ID NO:13; and

(4) a polypeptide that is a functional variant of any of the polypeptides of (1)-(3) above.

10. The antigen construct according to any one of clauses 2, and 4-9, wherein the immunogenic MUC1 polypeptide is selected from the group consisting of:

(1) a polypeptide comprising the amino acid sequence of SEQ ID NO:5 or amino acids 4-537 of SEQ ID NO:5;

(2) a polypeptide comprising the amino acid sequence of SEQ ID NO:7 or amino acids 4-517 of SEQ ID NO:7; and

(3) a functional variant of the polypeptide of (1) or (2) above.

11. The antigen construct according to any one of clauses 1-10, which comprises a nucleotide sequence encoding an amino acid sequence selected from the group consisting of:

(1) the amino acid sequence of SEQ ID NO:31 or an amino acid sequence comprising amino acids 4-1088 of SEQ ID NO:31;

(2) the amino acid sequence of SEQ ID NO:33 or an amino acid sequence comprising amino acids 4-1081 of SEQ ID NO:33;

(3) the amino acid sequence of SEQ ID NO:35 or an amino acid sequence comprising amino acids 4-1085 of SEQ ID NO:35;

(4) the amino acid sequence of SEQ ID NO:37 or an amino acid sequence comprising an amino acid sequence comprising amino acids 4-1030 of SEQ ID NO:37;

(5) the amino acid sequence of SEQ ID NO:39 or an amino acid sequence comprising amino acids 4-1381 of SEQ ID NO:39; and

(6) the amino acid sequence of SEQ ID NO:41 or an amino acid sequence comprising amino acids 4-1441 of SEQ ID NO:41.

12. The antigen construct according to any one of clauses 1-11, which comprises a nucleotide sequence selected from the group consisting of:

(1) the nucleotide sequence of SEQ ID NO:30 or a nucleotide sequence comprising nucleotides 10-3264 of SEQ ID NO:30;

(2) the nucleotide sequence of SEQ ID NO:32 or a nucleotide sequence comprising nucleotides 10-3243 of SEQ ID NO:32;

(3) the nucleotide sequence of SEQ ID NO:34 or a nucleotide sequence comprising nucleotides 10-3255 of SEQ ID NO:34;

(4) the nucleotide sequence of SEQ ID NO:36 or a nucleotide sequence comprising nucleotides 10-3090 of SEQ ID NO:36;

(5) the nucleotide sequence of SEQ ID NO:38 or a nucleotide sequence comprising nucleotides 10-4143 of SEQ ID NO:38;

(6) the nucleotide sequence of SEQ ID NO:40 or a nucleotide sequence comprising nucleotides 10-4323 of SEQ ID NO:40; and

(7) a nucleotide sequences that is a degenerate variant of any of the nucleotide sequences of (1)-(6) above.

13. The antigen construct according to any one of clauses 1-12, which comprises a nucleotide sequence encoding an amino acid sequence selected from the group consisting of:

(1) the amino acid sequence of SEQ ID NO:43 or an amino acid sequence comprising amino acids 4-2003 of SEQ ID NO:43;

(2) the amino acid sequence of SEQ ID NO:45 or an amino acid sequence comprising amino acids 4-2001 of SEQ ID NO:45;

(3) the amino acid sequence of SEQ ID NO:47 or an amino acid sequence comprising amino acids 4-2008 of SEQ ID NO:47;

(4) the amino acid sequence of SEQ ID NO:49 or an amino acid sequence comprising amino acids 4-1996 of SEQ ID NO: 49;

(5) the amino acid sequence of SEQ ID NO:51 or an amino acid sequence comprising amino acids 4-1943 of SEQ ID NO:51; and

(6) the amino acid sequence of SEQ ID NO:53 or an amino acid sequence comprising amino acids 4-1943 of SEQ ID NO:53.

14. The antigen construct according to any one of clauses 1-13, which comprises a nucleotide sequence selected from the group consisting of:

(1) the nucleotide sequence of SEQ ID NO:42 or a nucleotide sequence comprising nucleotides 10-6009 of SEQ ID NO:42;

(2) the nucleotide sequence of SEQ ID NO:44 or a nucleotide sequence comprising nucleotides 10-6003 of SEQ ID NO:44;

(3) the nucleotide sequence of SEQ ID NO:46 or a nucleotide sequence comprising nucleotides 10-6024 of SEQ ID NO:46;

(4) the nucleotide sequence of SEQ ID NO:48 or a nucleotide sequence comprising nucleotides 10-5988 of SEQ ID NO:48;

(5) the nucleotide sequence of SEQ ID NO:50 or a nucleotide sequence comprising nucleotides 10-5829 of SEQ ID NO:50;

(6) the nucleotide sequence of SEQ ID NO:52 or a nucleotide sequence comprising nucleotides 10-5829 of SEQ ID NO:52; and

(7) a nucleotide sequence that is a degenerate variant of any of the nucleotide sequences of (1)-(6) above.

15. The antigen construct according to any one of clauses 1-14, which comprises:

(1) a nucleotide sequence of any of SEQ ID NOS: 87, 88, 89, 90, 91, and 92; or

(2) a degenerate variant of a nucleotide sequence of any of SEQ ID NOS: 87, 88, 89, 90, 91, and 92.

16. A pharmaceutical composition comprising: (i) an antigen construct according to any one of clauses 1-15 and (ii) a pharmaceutically acceptable carrier.

17. The pharmaceutical composition according to clause 16, which is a vaccine.

18. A method of treating cancer in a human in need of treatment, comprising administering to the human an effective amount of the pharmaceutical composition according to clause 16 or clause 17.

19. The method according to clause 18, wherein the cancer over-expresses one or more tumor-associated antigens selected from MUC1, CEA, or TERT.

20. The method according to clause 18, wherein the cancer is pancreatic cancer, ovarian cancer, breast cancer, gastric cancer, lung cancer, or colorectal cancer.

21. The method according to clause 18, wherein the cancer is triple negative breast cancer, estrogen receptor positive breast cancer, or HER2 positive breast cancer.

22. The method according to clause 18, further comprising administering to the patient an effective amount of an immune modulator.

23. The method according to clause 22, wherein the immune modulator is a CTLA-4 inhibitor, an 001 inhibitor, a PD-1 inhibitor, or a PD-L1 inhibitor.

24. The method according to clause 18, further comprising administering to the human an adjuvant.

25. A vector, comprising an antigen construct according to any one of clauses 1-15.

26. The vector according to clause 25, which is a plasmid vector.

27. The vector according to clause 26, which comprises a nucleotide sequence of any of SEQ ID NOs:57, 59, 61, 63, 65, 67, 69, 70, 71, 72, 73, and 74.

28. The vector according to clause 25, which is a viral vector.

29. The vector according to clause 28, which comprises a nucleotide sequence of any of SEQ ID NOs:58, 60, 62, 64, 66, and 68.

30. A method for the treatment of cancer in a human, comprising administering to the human an effective amount of (1) an antigen construct according to any one of clauses 1-15, (2) a pharmaceutical composition according to clause 16 or clause 17, or (3) a vector according to any one of clauses 25-29.

### BRIEF DESCRIPTION OF DRAWINGS

FIG. 1. Diagram depicting the organization of AdC68 vectors carrying a triple-antigen construct (i.e., referred to as vectors AdC68Y-1424, AdC68Y-1425, AdC68Y-1426, AdC68Y-1427, AdC68Y-1428, and AdC68Y-1429). The E1 and E3 deleted AdC68 vector backbone was designed from Genbank reference sequence AC_000011.1. Transgene open reading frames were inserted into the E1 region, between the CMV immediate early enhancer/promoter and SV40 poly A terminator. A tet operator sequence was inserted after the promoter.

### DETAILED DESCRIPTION OF THE INVENTION

### A. Definitions

The term “adjuvant” refers to a substance that, when administered to a host mammal, such as human, is capable of enhancing, accelerating, or prolonging an antigen-specific immune response elicited by a vaccine or an immunogen in the host.

The term “agonist” refers to a substance which promotes (induces, causes, enhances or increases) the activity of another molecule (such as a receptor). The term agonist encompasses substances which bind a receptor and substances which promote receptor function without binding thereto.

The term “antagonist” or “inhibitor” refers to a substance that partially or fully blocks, inhibits, or neutralizes a biological activity of another molecule or a receptor.

The term “antigen” refers to a substance that, when introduced to a host mammal (directly or upon expression as in, e.g., DNA vaccines), is capable of being recognized by the immune system of the host mammal, such as binding to an antibody or to antigen receptors on T cells. Antigens can be proteins or protein fragments, carbohydrates, gangliosides, haptens, or nucleic acids. A substance is termed “antigenic” when it is capable of specifically interacting with an antigen recognition molecule of the immune system, such as antibody or T cell antigen receptor. The term “tumor-associated antigen” or “TAA” refers to an antigen which is specifically expressed by tumor cells or expressed at a higher frequency or density by tumor cells than by non-tumor cells of the same tissue type. TAA may be molecules that are not normally expressed by the host, or mutated, truncated, misfolded, or otherwise abnormal manifestations of molecules normally expressed by the host. Examples of TAA include CEA, TERT, and MUC1.

The term “co-administration” refers to administration of two or more agents to the same subject as part of a treatment regimen. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent do not overlap in time with each other.

The term “cytosolic” or “cytoplasmic” means that after a nucleotide sequence encoding a particular polypeptide is expressed by a host cell, the expressed polypeptide is expected to be retained inside the host cell.

The term “degenerate variants” refers to nucleic acid sequences that have substitutions of bases but encode the same polypeptide or amino acid sequence.

The term “effective amount” refers to an amount administered to a mammal that is sufficient to cause a desired effect in the mammal.

The term “functional variant” of an amino acid sequence or an immunogenic TAA polypeptide (collectively “reference polypeptide”) refers to an amino acid sequence or a polypeptide that comprises from 90% to 100% of the number of amino acids of the reference polypeptide, has lower than 100% but higher than 95% identity to the amino acid sequence of the reference polypeptide, and possess the same or similar immunogenic properties of the reference polypeptide.

The term “identical” refers to two or more nucleic acids, or two or more polypeptides, that share the exact same sequence of nucleotides or amino acids, respectively. The term “percent identity” describes the level of similarity between two or more nucleic acids or polypeptides. When two sequences are aligned by bioinformatics software, “percent identity” is calculated by multiplying the number of exact nucleotide/amino acid matches between the sequences by 100, and dividing by the length of the aligned region, including gaps. For example, two 100-amino acid long polypeptides that exhibit 10 mismatches when aligned would be 90% identical.

The term “immune-effector-cell enhancer” or “IEC enhancer” refers to a substance capable of increasing and/or enhancing the number, quality, and/or function of one or more types of immune effector cells of a mammal. Examples of immune effector cells include cytolytic Dendiritic cells, CD8 T cells, CD4 T cells, NK cells, and B cells.

The term “immune modulator” refers to a substance capable of altering (e.g., inhibiting, decreasing, increasing, enhancing or stimulating) the working or function of any component of the innate, humoral, or cellular immune system of a mammal. Thus, the term “immune modulator” encompasses the “immune-effector-cell enhancer” as defined herein and the “immune-suppressive-cell inhibitor” as defined herein, as well as substance that affects any other components of the immune system of a mammal.

The term “immune response” refers to any detectable response to a particular substance (such as an antigen or immunogen) by the adaptive immune system of a host mammal, including cell-mediated immune responses (e.g., responses mediated by T cells, such as antigen-specific T cells, and non-specific cells of the immune system) and humoral immune responses (e.g., responses mediated by B cells, such as generation and secretion of antibodies into the plasma, lymph, and/or tissue fluids). Examples of immune responses include an alteration (e.g., increase) in release of cytokines (e.g., Th1, Th2 or Th17 type cytokines) or chemokine, macrophage activation, dendritic cell activation, T cell (e.g., CD4+ or CD8+ T cell) activation, induction of B cell response (e.g., antibody production), induction of a cytotoxic T lymphocyte (“CTL”) response, and expansion (e.g., growth of a population of cells) of cells of the immune system (e.g., T cells and B cells).

The term “immunogenic” or “immunogenicity” refers to the ability of a substance upon administration to a host mammal (such as a human) to cause, elicit, stimulate, or induce an immune response, or to improve, enhance, increase or prolong a pre-existing immune response, in the host mammal, whether alone or when linked to a carrier, in the presence or absence of an adjuvant. Such a substance is referred to as “immunogen.”

The term “immunogenic composition” refers to a composition that is immunogenic.

The term “immunogenic MUC1 polypeptide” refers to a polypeptide that is immunogenic against a human native MUC1 protein or against cells expressing the human native MUC1 protein. The polypeptide may have the same amino acid sequence as that of a human native MUC1 protein or display one or more mutations as compared to the amino acid sequence of a human native MUC1 protein.

The term “immunogenic CEA polypeptide” refers to a polypeptide that is immunogenic against a human native CEA protein or against cells expressing a human native CEA protein and displays one or more mutations, such as deletion of one or more amino acids, as compared to the amino acid sequence of the human native CEA protein.

The term “immunogenic TERT polypeptide” refers to a polypeptide that is immunogenic against a human native TERT protein or against cells expressing a human native TERT protein. The polypeptide may have the same amino acid sequence as that of a human native TERT protein or displays one or more mutations as compared to the amino acid sequence of a human native TERT protein.

The term “immunogenic TAA polypeptide” refers to an “immunogenic CEA polypeptide,” an “immunogenic MUC1 polypeptide, or an “immunogenic TERT polypeptide,” each as defined herein above.

The term “immune-suppressive-cell inhibitor” or “ISC inhibitor” refers to a substance capable of reducing and/or suppressing the number and/or function of immune suppressive cells of a mammal. Examples of immune suppressive cells include regulatory T cells (“Tregs”), myeloid-derived suppressor cells, and tumor-associated macrophages.

The term “mammal” refers to any animal species of the Mammalia class. Examples of mammals include: humans; non-human primates such as monkeys; laboratory animals such as rats, mice, guinea pigs; domestic animals such as cats, dogs, rabbits, cattle, sheep, goats, horses, and pigs; and captive wild animals such as lions, tigers, elephants, and the like.

The term “membrane-bound” means that after a nucleotide sequence encoding a particular polypeptide is expressed by a host cell, the expressed polypeptide is bound to, attached to, or otherwise associated with, the membrane of the cell.

The term “neoplastic disorder” refers to a condition in which cells proliferate at an abnormally high and uncontrolled rate, the rate exceeding and uncoordinated with that of the surrounding normal tissues. It usually results in a solid lesion or lump known as “tumor.” This term encompasses benign and malignant neoplastic disorders. The term “malignant neoplastic disorder”, which is used interchangeably with the term “cancer” in the present disclosure, refers to a neoplastic disorder characterized by the ability of the tumor cells to spread to other locations in the body (known as “metastasis”). The term “benign neoplastic disorder” refers to a neoplastic disorder in which the tumor cells lack the ability to metastasize.

The term “mutation” refers to deletion, addition, or substitution of amino acid residues in the amino acid sequence of a protein or polypeptide as compared to the amino acid sequence of a reference protein or polypeptide.

The term “pharmaceutical composition” refers to a solid or liquid composition suitable for administration to a subject (e.g. a human patient) for eliciting a desired physiological, pharmacological, or therapeutic effect. In addition to containing one or more active ingredients, a pharmaceutical composition may contain one or more pharmaceutically acceptable excipients.

The term “pharmaceutically acceptable excipient” refers to a substance in pharmaceutical composition, such as a vaccine, other than the active ingredients (e.g., the antigen, antigen-coding nucleic acid, immune modulator, or adjuvant) that is compatible with the active ingredients and does not cause significant untoward effect in subjects to whom it is administered.

The term “excipient” as used in the context of a pharmaceutical composition refers to a substance that generally has no medicinal properties and is included in the composition for purpose of streamlining the manufacture of the drug product and/or facilitating stabilization, delivery, and absorption of the active drug substance. The term “pharmaceutically acceptable excipient” refers to an excipient in a pharmaceutical composition, such as a vaccine composition, that is compatible with the active ingredients (e.g., the antigen or immunogen, antigen-coding nucleic acid, immune modulator, or adjuvant) in the composition and does not cause significant untoward effects in subjects to whom it is administered.

The terms “peptide,” “polypeptide,” and “protein” are used interchangeably herein, and refer to a polymeric form of amino acids linked together by peptide bonds. They may be of any length and can include coded and non-coded amino acids, chemically, or biochemically modified, or derivatized amino acids.

The term “preventing” or “prevent” refers to (a) keeping a disorder from occurring, (b) delaying the onset of a disorder or onset of symptoms of a disorder, or (c) minimizing the incidence or effects of a disorder.

The term “secreted” in the context of a polypeptide means that after a nucleotide sequence encoding the polypeptide is expressed by a host cell, the expressed polypeptide is secreted outside of the host cell.

The term “suboptimal dose” when used to describe the amount of an immune modulator, such as a protein kinase inhibitor, refers to a dose of the immune modulator that is below the minimum amount required to produce the desired therapeutic effect for the disease being treated when the immune modulator is administered alone to a patient.

The term “treating,” “treatment,” or “treat” refers to abrogating a disorder, reducing the severity of a disorder, or reducing the severity or occurrence frequency of a symptom of a disorder.

The term “vaccine” refers to an immunogenic composition for administration to a mammal (such as a human) for eliciting a protective immune response against a particular antigen or antigens. The primary active ingredient of a vaccine is the immunogen(s). A vaccine that comprises an immunogenic polypeptide as immunogen is also referred to as “peptide vaccine.” A vaccine that does not contain an immunogenic polypeptide but rather contains a nucleic acid molecule that encodes an immunogenic polypeptide is referred to as a “DNA vaccine” or “RNA vaccine” (depending on the case it may be). Upon delivery of the DNA or RNA vaccine into host cells, the immunogenic polypeptide encoded by the nucleic acid molecule will be expressed by the host cells, producing a protective immune response. The nucleic acid molecule in a DNA or RNA vaccine may be in the form of naked nucleic acid, plasmid, or virus vector, or any other form suitable for delivering the nucleic acid.

The term “vector” refers to a nucleic acid molecule, or a modified microorganism, that is capable of transporting or transferring a foreign nucleic acid molecule into a host cell. The foreign nucleic acid molecule is referred to as “insert” or “transgene.” A vector generally consists of an insert and a larger sequence that serves as the backbone of the vector. Based on the structure or origin of vectors, major types of vectors include plasmid vectors, cosmid vectors, phage vectors (such as lambda phage), viral vectors (such as adenovirus vectors), artificial chromosomes, and bacterial vectors.

### B. Immunogenic TAA Polypeptides

In some aspects, the present disclosure provides isolated immunogenic TAA polypeptides, which are useful, for example, for eliciting an immune response in vivo (e.g. in an animal, including humans) or in vitro, activating effector T cells, or generating antibodies specific for the TAA or for use as a component in a pharmaceutical composition, including a vaccine, for the treatment of a cancer, such as pancreatic, lung cancer, colorectal cancer, gastric cancer, or breast cancer.

These immunogenic TAA polypeptides can be prepared by methods known in the art in light of the present disclosure. The capability of the polypeptides to elicit an immune response can be measured in in vitro assays or in vivo assays. In vitro assays for determining the capability of a polypeptide or DNA construct to elicit immune responses are known in the art. One example of such in vitro assays is to measure the capability of the polypeptide or nucleic acid expressing a polypeptide to stimulate T cell response as described in U.S. Pat. No. 7,387,882, the disclosure of which is incorporated in this application. The assay method comprises the steps of: (1) contacting antigen presenting cells in culture with an antigen thereby the antigen can be taken up and processed by the antigen presenting cells, producing one or more processed antigens; (2) contacting the antigen presenting cells with T cells under conditions sufficient for the T cells to respond to one or more of the processed antigens; (3) determining whether the T cells respond to one or more of the processed antigens. The T cells used may be CD8⁺ T cells or CD4⁺ T cells. T cell response may be determined by measuring the release of one or more cytokines, such as interferon-gamma and interleukin-2, and lysis of the antigen presenting cells (tumor cells). B cell response may be determined by measuring the production of antibodies.

B-1. Immunogenic MUC1 Polypeptides

In one aspect, the present disclosure provides immunogenic MUC1 polypeptides derived from a human native MUC1 by introducing one or more mutations to the human native MUC1 protein. Examples of mutations include deletion of some, but not all, of the tandem repeats of 20 amino acids in the VNTR region of the MUC1 protein, deletion of the signal peptide sequence in whole or in part, and deletion of amino acids of non-consensus amino acid sequences found in the MUC1 isoforms. Thus, in some embodiments, the immunogenic MUC1 polypeptides comprise (1) the amino acid sequence of 3 to 30 tandem repeats of 20 amino acids of a human MUC1 protein and (2) the amino acid sequences of the human MUC1 protein that flank the VNTR region. In some particular embodiments, the immunogenic MUC1 polypeptides comprise (1) the amino acid sequence of 5 to 25 tandem repeats of the human MUC1 and (2) the amino acid sequences of the human MUC1 protein that flank the VNTR region. In some embodiments, the immunogenic MUC1 polypeptides consist of (1) the amino acid sequence of 3 to 30 tandem repeats of 20 amino acids of a human MUC1 protein and (2) the amino acid sequences of the human MUC1 protein that flank the VNTR region. In some particular embodiments, the immunogenic MUC1 polypeptides consist of (1) the amino acid sequence of 5 to 25 tandem repeats of the human MUC1 and (2) the amino acid sequences of the human MUC1 protein that flank the VNTR region. In some further embodiments, the immunogenic MUC1 polypeptides are in cytoplasmic form (or “cMUC1”). The term “cytoplasmic form” refers to an immunogenic MUC1 polypeptide that lacks in whole or in part the secretory sequence (amino acids 1-23; also known as “signal peptide sequence”) of the human native MUC1 protein. The deletion of amino acids of the secretory sequence is expected to prevent the polypeptide from entering the secretory pathway as it is expressed in the cells. In some other embodiments, the immunogenic MUC1 polypeptides are in membrane-bound form. The immunogenic MUC1 polypeptides can be derived, constructed, or prepared from the amino acid sequence of any of the human MUC1 isoforms known in the art or discovered in the future, including, for example, Uniprot isoforms 1, 2, 3, 4, 5, 6, Y, 8, 9, F, Y-LSP, S2, M6, ZD, T10, E2, and J13 (Uniprot P15941-1 through P15941-17, respectively). In some embodiments, the immunogenic MUC1 polypeptides comprise an amino acid sequence that is part of human MUC1 isoform 1 wherein the amino acid sequence of the human MUC1 isoform 1 is set forth in SEQ ID NO: 1. In some embodiments, the immunogenic MUC1 polypeptides consist of an amino acid sequence that is part of human MUC1 isoform 1 wherein the amino acid sequence of the human MUC1 isoform 1 is set forth in SEQ ID NO: 1. In a specific embodiment, the immunogenic MUC1 polypeptide comprises amino acids 22-225 and 946-1255 of the amino acid sequence of SEQ ID NO:1. In some other specific embodiments, the present disclosure provides an immunogenic MUC1 polypeptide selected from:

(1) a polypeptide comprising or consisting of the amino acid sequence of SEQ ID NO:5 (Plasmid 1027 polypeptide);

(2) a polypeptide comprising or consisting of amino acids 4-537 of SEQ ID NO:5;

(3) a polypeptide comprising or consisting of amino acids 24-537 of SEQ ID NO:5;

(4) a polypeptide comprising or consisting of the amino acid sequence of SEQ ID NO:7 (Plasmid 1197 polypeptide);

(5) a polypeptide comprising or consisting of amino acids 4-517 of SEQ ID NO:7;

(6) a polypeptide comprising or consisting of amino acids 4-517 of SEQ ID NO:7, wherein in SEQ ID NO:7 the amino acid at position 513 is T; and

(7) a functional variant of any of the polypeptides of (1)-(6) above.

In some specific embodiments, the immunogenic MUC1 polypeptides comprise the amino acid sequence of SEQ ID NO:5 (Plasmid 1027 polypeptide) or SEQ ID NO:7 (Plasmid 1197 polypeptide). In some specific embodiments, the immunogenic MUC1 polypeptides consists of the amino acid sequence of SEQ ID NO:5 (Plasmid 1027 polypeptide) or SEQ ID NO:7 (Plasmid 1197 polypeptide).

In one aspect, the present invention provides a functional variant of any of the immunogenic MUC1 polypeptides disclosed herein.

B-2. Immunogenic TERT Polypeptides

In another aspect, the present disclosure provides immunogenic TERT polypeptides derived from a human TERT protein by deletion of up to 600 of the N-terminal amino acids of the TERT protein. Thus, an immunogenic TERT polypeptide may comprise the C-terminal amino acid sequence starting from position 601 of any human TERT protein isoform. In some embodiments, the immunogenic TERT polypeptides comprise the amino acid sequence of TERT isoform 1 set forth in SEQ ID NO:3, wherein up to about 600 amino acids from the N-terminus (amino terminus) of the amino acid sequence of TERT isoform 1 are absent. Any number of amino acids up to 600 from the N-terminus of the TERT isoform 1 may be absent in the immunogenic TERT polypeptide. For example, the N-terminal amino acids from position 1 through position 50, 100, 50, 200, 250, 300, 350, 400, 450, 500, 550, or 600 of the TERT isoform 1 of SEQ ID NO:3 may be absent from the immunogenic TERT polypeptide. Thus, an immunogenic TERT polypeptide may comprise amino acids 51-1132, 101-1132, 151-1132, 201-1132, 251-1132, 301-1132, 351-1132, 401-1132, 451-1132, 501-1132, or 551-1132 of SEQ ID NO:3. In one embodiment, the immunogenic TERT polypeptide comprises amino acids 601-1132 of the amino acid sequence of SEQ ID NO:3. In another embodiment, the present disclosure provides an immunogenic TERT polypeptide that comprises amino acids 241-1132 of the amino acid sequence of SEQ ID NO:3.

An immunogenic TERT polypeptide may consist of amino acids 51-1132, 101-1132, 151-1132, 201-1132, 251-1132, 301-1132, 351-1132, 401-1132, 451-1132, 501-1132, or 551-1132 of SEQ ID NO:3. In one embodiment, the immunogenic TERT polypeptide consists of amino acids 601-1132 of the amino acid sequence of SEQ ID NO:3. In another embodiment, the present disclosure provides an immunogenic TERT polypeptide that consists of amino acids 241-1132 of the amino acid sequence of SEQ ID NO:3.

The immunogenic TERT polypeptides may also be constructed from other TERT isoforms. Where the immunogenic TERT polypeptides are constructed from TERT isoforms with C-terminal truncations, such as isoform 2, 3, or 4, it is preferred that fewer amino acids are deleted from the N-terminus of the protein.

In some further embodiments, the immunogenic TERT polypeptide further comprises one or more amino acid mutations that inactivate the TERT catalytic domain. Examples of such amino acid mutations include substitution of aspartic acid with alanine at position 712 of SEQ ID NO:3 (D712A) and substitution of valine with isoleucine at position 713 of SEQ ID NO:3 (V713I). In some embodiments the immunogenic TERT polypeptide comprises both mutations D712A and V713I. In an embodiment said mutations include a substitution of aspartic acid at position 712 of SEQ ID NO:3 and/or substitution of valine at position 713 of SEQ ID NO:3 (V713I) wherein said mutation(s) inactivates the TERT catalytic domain. In another embodiment said mutation consists of a substitution of aspartic acid at position 712 of SEQ ID NO:3 and/or substitution of valine at position 713 of SEQ ID NO:3 (V713I) wherein said mutation(s) inactivates the TERT catalytic domain. In still another embodiment said mutation consists of a substitution of aspartic acid with alanine at position 712 of SEQ ID NO:3 (D712A) and/or a substitution of valine with isoleucine at position 713 of SEQ ID NO:3 (V713I).

In some specific embodiments, the present disclosure provides an immunogenic TERT polypeptide selected from:

(1) a polypeptide comprising or consisting of the amino acid sequence of SEQ ID NO:9 (Plasmid 1112 Polypeptide) or amino acids 2-893 of SEQ ID NO:9;

(2) a polypeptide comprising or consisting of the amino acid sequence of SEQ ID NO:11 (Plasmid 1326 Polypeptide) or amino acids 3-791 of SEQ ID NO:11;

(3) a polypeptide comprising or consisting of the amino acid sequence of SEQ ID NO:13 (Plasmid 1330 Polypeptide) or amino acids 4-594 of SEQ ID NO:13; or

(4) a polypeptide that is a functional variant of any of the polypeptides of (1)-(3) above.

In one aspect, the present invention provides a functional variant of any of the immunogenic TERT polypeptides disclosed herein.

B-3. Immunogenic CEA Polypeptides

In another aspect, the present disclosure provides isolated immunogenic CEA polypeptides derived from a human native CEA by introducing one or more mutations to the human native CEA precursor protein. Examples of the introduced mutations include deletion of one, two, three, four, or five of the C2-like domains, deletion of the signal peptide sequence in whole or in part, and deletion of some or all of the amino acids of the propeptide. Thus, in some embodiments, the immunogenic CEA polypeptides provided by the present disclosure comprise (1) the amino acid sequence of the N-domain and (2) the amino acid sequence of 1 to 5 C2-like domains of a human CEA protein. In some particular embodiments, the immunogenic CEA polypeptides comprise (1) the amino acid sequence of at least four C2-like domains, such as A2, B2, A3, and B3, and (2) the amino acid sequence of the N-domain. In some further embodiments, the immunogenic CEA polypeptides are in cytoplasmic form (or “cCEA”). The term “cytoplasmic form” refers to an immunogenic CEA polypeptide that lacks in whole or in part the signal peptide sequence (amino acids 1-34) of the human native CEA precursor protein. The deletion of amino acids of the signal peptide is expected to prevent the polypeptide from entering the secretory pathway as it is expressed in the cells. In some other embodiments, the immunogenic CEA polypeptides are in the membrane-bound form (or “mCEA”). An immunogenic mCEA polypeptide includes amino acids of the signal peptide and, after expressed by a host cell, remains bound to, or otherwise associated with, the membrane of the host cell.

The immunogenic CEA polypeptides provided by the present disclosure can be derived, constructed, or prepared from the amino acid sequence of any of the human CEA isoforms known in the art or discovered in the future. In some embodiments, the immunogenic CEA polypeptides comprise an amino acid sequence that is part of human CEA isoform 1 precursor protein having amino acid sequence of SEQ ID NO:2.

In some specific embodiments, the present disclosure provides any of the following immunogenic CEA polypeptides:

(1) a polypeptide comprising amino acids 2-702 of SEQ ID NO:2, amino acids 323-702 of SEQ ID NO:2, or amino acids 323-677 of SEQ ID NO:2;

(2) a polypeptide consisting of amino acids 2-702 of SEQ ID NO:2, amino acids 323-702 of SEQ ID NO:2, or amino acids 323-677 of SEQ ID NO:2;

(3) a polypeptide comprising amino acids of SEQ ID NO:15 (amino acid sequence encoded by Plasmid 1361) or amino acids 4-704 of SEQ ID NO:15;

(4) a polypeptide consisting of amino acids of SEQ ID NO:15 (amino acid sequence encoded by Plasmid 1361) or amino acids 4-704 of SEQ ID NO:15;

(5) a polypeptide comprising the amino acid sequence of SEQ ID NO:17 (amino acid sequence encoded by Plasmid 1386) or amino acids 4-526 of SEQ ID NO:17;

(6) a polypeptide consisting of the amino acid sequence of SEQ ID NO:17 (amino acid sequence encoded by Plasmid 1386) or amino acids 4-526 of SEQ ID NO:17;

(7) a polypeptide comprising the sequence of SEQ ID NO:19 (amino acid sequence encoded by Plasmid 1390) or amino acids 4-468 of SEQ ID NO:19;

(8) a polypeptide consisting of the sequence of SEQ ID NO:19 (amino acid sequence encoded by Plasmid 1390) or amino acids 4-468 of SEQ ID NO:19; or

(9) a polypeptide that is a functional variant of any of the polypeptides of (1)-(8) above.

In one aspect, the present invention provides a functional variant of any of the immunogenic TERT polypeptides disclosed herein.

### C. Antigen Constructs Encoding One or More Immunogenic TAA Polypeptides

In some aspects, the present disclosure provides an isolated nucleic acid molecule that encodes one, two, three, or more separate immunogenic TAA polypeptides. Such a nucleic acid molecule is also referred to as “antigen construct” in the present disclosure. A nucleic acid molecule that encodes only one immunogenic TAA polypeptide is also referred to herein as a “single-antigen construct” and a nucleic acid molecule that encodes more than one immunogenic TAA polypeptide is also referred to as a “multi-antigen construct.” A nucleic acid molecule that encodes two different immunogenic TAA polypeptides is also referred to as a “dual-antigen construct” and a nucleic acid molecule that encodes three different immunogenic TAA polypeptides is also referred to as a “triple-antigen construct.” The nucleic acid molecules can be deoxyribonucleic acids (DNA) or ribonucleic acids (RNA). Thus, a nucleic acid molecule can comprise a nucleotide sequence disclosed herein wherein thymidine (T) can also be uracil (U), which reflects the differences between the chemical structures of DNA and RNA. In reference to a nucleotide sequence of a RNA which corresponds to a nucleotide sequence of a DNA in the present disclosure, the term “correspond” or “corresponding” refers to a nucleotide sequence of the RNA that is identical to the reference nucleotide sequence of the DNA except that thymidine (T) in the DNA nucleotide sequence is replaced with uracil (U) in the RNA nucleotide sequence. The nucleic acid molecules can be in modified forms, single or double stranded forms, or linear or circular forms.

The antigen constructs, including both DNA and RNA constructs, can be prepared using methods known in the art in light of the present disclosure. Method for making single-antigen constructs and multi-antigen constructs is further described herein below. Additionally, it's well established that the injection of mRNA into host cells leads to expression of encoded proteins and immunological responses. The in vitro transcribed mRNA can be produced stably and the encoded protein can be translated efficiently through the use of various elements/systems known in the art (such as UTR's, PolyA, capping system, and codon optimization). Further, the fusion of lysosomal or endosomal targeting signals to mRNA encoded polypeptides can enhance the T-cell immune responses. mRNA can be delivered unformulated or through EP or formulated in lipids or other vehicles.

C-1. CEA Single-Antigen Constructs

In some embodiments, the present disclosure provides antigen constructs that encode any of the immunogenic CEA polypeptides described herein above.

In some specific embodiments, the antigen construct encodes an immunogenic CEA polypeptide selected from:

(1) a polypeptide comprising amino acids 2-702 of SEQ ID NO:2, amino acids 323-702 of SEQ ID NO:2, or amino acids 323-677 of SEQ ID NO:2;

(2) a polypeptide comprising amino acids of SEQ ID NO:15 (amino acid sequence encoded by Plasmid 1361) or amino acids 4-704 of SEQ ID NO:15;

(3) a polypeptide comprising the amino acid sequence of SEQ ID NO:17 (amino acid sequence encoded by Plasmid 1386) or amino acids 4-526 of SEQ ID NO:17;

(4) a polypeptide comprising the sequence of SEQ ID NO:19 (amino acid sequence encoded by Plasmid 1390) or amino acids 4-468 of SEQ ID NO:19; or

(5) a polypeptide that is a functional variant of any of the polypeptides of (1)-(4) above.

In some specific embodiments, the antigen construct encodes an immunogenic CEA polypeptide selected from:

(1) a polypeptide consisting of amino acids 2-702 of SEQ ID NO:2, amino acids 323-702 of SEQ ID NO:2, or amino acids 323-677 of SEQ ID NO:2;

(2) a polypeptide consisting of amino acids of SEQ ID NO:15 (amino acid sequence encoded by Plasmid 1361) or amino acids 4-704 of SEQ ID NO:15;

(3) a polypeptide consisting of the amino acid sequence of SEQ ID NO:17 (amino acid sequence encoded by Plasmid 1386) or amino acids 4-526 of SEQ ID NO:17;

(4) a polypeptide consisting of the sequence of SEQ ID NO:19 (amino acid sequence encoded by Plasmid 1390) or amino acids 4-468 of SEQ ID NO:19; or

(5) a polypeptide that is a functional variant of any of the polypeptides of (1)-(4) above.

In some particular embodiments, the present disclosure provides an antigen construct that is a DNA and comprises a nucleotide sequence selected from the group consisting of:

(1) the nucleotide sequence of SEQ ID NO:14 (Plasmid 1361 open reading frame) or a nucleotide sequence comprising nucleotides 10-2112 of SEQ ID NO:14;

(2) the nucleotide sequence of SEQ ID NO:16 (Plasmid 1386 open reading frame) or a nucleotide sequence comprising nucleotides 10-1578 of SEQ ID NO:16;

(3) the nucleotide sequence of SEQ ID NO:18 (Plasmid 1390 open reading frame) or a nucleotide sequence comprising nucleotides 10-1404 of SEQ ID NO:18; and

(4) a nucleotide sequence that is a degenerate variant of the nucleotide sequences of (1)-(3).

In some other particular embodiments, the present disclosure provides an antigen construct that is a DNA and consists of a nucleotide sequence selected from the group consisting of:

(1) the nucleotide sequence of SEQ ID NO:14 (Plasmid 1361 open reading frame) or a nucleotide sequence consisting of nucleotides 10-2112 of SEQ ID NO:14;

(2) the nucleotide sequence of SEQ ID NO:16 (Plasmid 1386 open reading frame) or a nucleotide sequence consisting of nucleotides 10-1578 of SEQ ID NO:16;

(3) the nucleotide sequence of SEQ ID NO:18 (Plasmid 1390 open reading frame) or a nucleotide sequence consisting of nucleotides 10-1404 of SEQ ID NO:18; and

(4) a nucleotide sequence that is a degenerate variant of the nucleotide sequences of (1)-(3). In some other particular embodiments, the present disclosure provides an antigen construct that is a RNA and comprises a nucleotide sequence that corresponds to a nucleotide sequence selected from the group consisting of:

(1) the nucleotide sequence of SEQ ID NO:14 (Plasmid 1361 open reading frame) or a nucleotide sequence comprising nucleotides 10-2112 of SEQ ID NO:14;

(2) the nucleotide sequence of SEQ ID NO:16 (Plasmid 1386 open reading frame) or a nucleotide sequence comprising nucleotides 10-1578 of SEQ ID NO:16;

(3) the nucleotide sequence of SEQ ID NO:18 (Plasmid 1390 open reading frame) or a nucleotide sequence comprising nucleotides 10-1404 of SEQ ID NO:18; and

(4) a nucleotide sequence that is a degenerate variant of the nucleotide sequences of (1)-(3).

C-2. Multi-Antigen Constructs

In another aspect, the present disclosure provides antigen constructs that each encode two, three, or more different immunogenic TAA polypeptides.

Methods and techniques for construction of vectors for co-expression of two or more polypeptides from a single nucleic acid (also known in the art as “multicistronic vectors”) are known in the art. The multi-antigen constructs provided by the present disclosure can be prepared using such techniques in light of the disclosure. For example, a multi-antigen construct can be constructed by incorporating multiple independent promoters into a single plasmid (Huang, Y., Z. Chen, et al. (2008). “Design, construction, and characterization of a dual-promoter multigenic DNA vaccine directed against an HIV-1 subtype C/B′ recombinant.” J Acquir Immune Defic Syndr 47(4): 403-411; Xu, K., Z. Y. Ling, et al. (2011). “Broad humoral and cellular immunity elicited by a bivalent DNA vaccine encoding HA and NP genes from an H5N1 virus.” Viral Immunol 24(1): 45-56). The plasmid can be engineered to carry multiple expression cassettes, each consisting of a) a eukaryotic promoter for initiating RNA polymerase dependent transcription, with or without an enhancer element, b) a gene encoding a target antigen, and c) a transcription terminator sequence. Upon delivery of the plasmid to the transfected cell nucleus, transcription will be initiated from each promoter, resulting in the production of separate mRNAs, each encoding one of the target antigens. The mRNAs will be independently translated, thereby producing the desired antigens.

Multi-antigen constructs provided by the present disclosure can also be constructed through the use of viral 2A peptides (Szymczak, A. L. and D. A. Vignali (2005). “Development of 2A peptide-based strategies in the design of multicistronic vectors.” Expert Opin Biol Ther 5(5): 627-638; de Felipe, P., G. A. Luke, et al. (2006). “E unum pluribus: multiple proteins from a self-processing polyprotein.” Trends Biotechnol 24(2): 68-75; Luke, G. A., P. de Felipe, et al. (2008). “Occurrence, function and evolutionary origins of ‘2A-like’ sequences in virus genomes.” J Gen Virol 89 (Pt 4): 1036-1042; Ibrahimi, A., G. Vande Velde, et al. (2009). “Highly efficient multicistronic lentiviral vectors with peptide 2A sequences.” Hum Gene Ther 20(8): 845-860; Kim, J. H., S. R. Lee, et al. (2011). “High cleavage efficiency of a 2A peptide derived from porcine teschovirus-1 in human cell lines, zebrafish and mice.” PLoS One 6(4): e18556). These peptides, also called cleavage cassettes or CHYSELs (cis-acting hydrolase elements), are approximately 20 amino acids long with a highly conserved carboxy terminal D-V/I-EXNPGP motif. These peptides are rare in nature, most commonly found in viruses such as Foot-and-mouth disease virus (FMDV), Equine rhinitis A virus (ERAV), Equine rhinitis B virus (ERBV), Encephalomyocarditis virus (EMCV), Porcine teschovirus (PTV), and Thosea asigna virus (TAV) (Luke, G. A., P. de Felipe, et al. (2008). “Occurrence, function and evolutionary origins of ‘2A-like’ sequences in virus genomes.” J Gen Virol 89 (Pt 4): 1036-1042). An amino acid sequence of some of these peptides is provided in Table 17. With a 2A-based multi-antigen expression strategy, genes encoding multiple target antigens are linked together in a single open reading frame (ORF), separated by sequences encoding viral 2A peptides. The entire open reading frame can be cloned into a vector with a single promoter and terminator. Upon delivery of the constructs to a host cell, mRNA encoding the multiple antigens will be transcribed and translated as a single polyprotein. During translation of the 2A peptides, ribosomes skip the bond between the C-terminal glycine and proline. The ribosomal skipping acts like a cotranslational autocatalytic “cleavage” that releases the peptide sequences upstream of the 2A peptide from those downstream. The incorporation of a 2A peptide between two protein antigens may result in the addition of ˜20 amino acids onto the C-terminus of the upstream polypeptide and 1 amino acid (proline) to the N-terminus of downstream protein. In an adaptation of this methodology, protease cleavage sites can be incorporated at the N terminus of the 2A cassette such that ubiquitous proteases will cleave the cassette from the upstream protein (Fang, J., S. Yi, et al. (2007). “An antibody delivery system for regulated expression of therapeutic levels of monoclonal antibodies in vivo.” Mol Ther 15(6): 1153-1159). Examples of specific 2A-peptide sequences that may be used in construction of the multi-antigen constructs of the present disclosure include those that are disclosed in Andrea L. Szymczak & Darrio AA Vignali: Development of 2A peptide-based strategies in the design of multicistronic vectors. Expert Opinion Biol. Ther. (2005) 5(5) 627-638, as well as in international patent application WO2015/063674, the disclosure of which is incorporated herein by reference.

Another method that may be used for constructing the multi-antigen constructs involves the use of an internal ribosomal entry site, or IRES. Internal ribosomal entry sites are RNA elements found in the 5′ untranslated regions of certain RNA molecules (Bonnal, S., C. Boutonnet, et al. (2003). “IRESdb: the Internal Ribosome Entry Site database.” Nucleic Acids Res 31(1): 427-428). They attract eukaryotic ribosomes to the RNA to facilitate translation of downstream open reading frames. Unlike normal cellular 7-methylguanosine cap-dependent translation, IRES-mediated translation can initiate at AUG codons far within an RNA molecule. The highly efficient process can be exploited for use in multi-cistronic expression vectors (Bochkov, Y. A. and A. C. Palmenberg (2006). “Translational efficiency of EMCV IRES in bicistronic vectors is dependent upon IRES sequence and gene location.” Biotechniques 41(3): 283-284, 286, 288). Typically, two transgenes are inserted into a vector between a promoter and transcription terminator as two separate open reading frames separated by an IRES. Upon delivery of the constructs to a host cell, a single long transcript encoding both transgenes will be transcribed. The first ORF will be translated in the traditional cap-dependent manner, terminating at a stop codon upstream of the IRES. The second ORF will be translated in a cap-independent manner using the IRES. In this way, two independent proteins can be produced from a single mRNA transcribed from a vector with a single expression cassette. Examples of IRES sequences includes poliovirus (PV) IRES, encephalomyocarditis virus (EMCV) IRES, Foot-and-mouth disease virus (FMDV) IRES, Hepatitis A virus IRES, Hepatitis B virus IRES, Kaposi's sarcoma-associated herpesvirus (KSHV) IRES, and classical swine fever virus IRES. A nucleotide sequence of the EMCV IRES is disclosed in WO2013/165754 (FIG. 3) and is set forth in SEQ ID NO:93 in the present disclosure. The minimal EMCV IRES element excludes the 15 nucleotides of the 3′-end (which represent first 5 codons of the EMCV L protein) of nucleotide sequence of SEQ ID NO:93.

The nucleotide sequence that is inserted between two coding sequences or transgenes in an open reading frame (ORF) of a nucleic acid molecule and functions to allow co-expression or translation of two separate gene products from the nucleic acid molecule is referred to as “spacer nucleotide sequence” in the present disclosure. Examples of specific spacer nucleotide sequences that may be used in the multi-antigen constructs include eukaryotic promoters, nucleotide sequences encoding a 2A peptide, and internal ribosomal entry site (IRES) sequences. Examples of specific 2A peptides include 2A peptides of acute bee paralysis virus (ABP2A), cricket paralysis virus (CrP2A), equine rhinitis A virus (ERA2A), equine rhinitis B virus (ERB2A), encephalomyocarditis virus (EMC2A), foot-and-mouth disease virus (FMD2A or F2A), human rotavirus (HT2A), Infectious flacherie virus (IF2A), porcine teschovirus (PT2A or P2A), porcine rotavirus (PR2A), and Thosea asigna virus (T2A, TA2A, or TAV2A).

In some aspects, the present disclosure provides an antigen construct comprising (i) at least one coding nucleotide sequence encoding an immunogenic CEA polypeptide and (ii) one or more nucleotide sequences encoding one or more other immunogenic TAA polypeptides, such as an immunogenic TERT polypeptide, an immunogenic MUC1 polypeptide, an immunogenic MSLN polypeptide, an immunogenic PSA polypeptide, an immunogenic PSMA polypeptide, or an immunogenic PSCA polypeptide.

In some embodiments, the present disclosure provides an antigen construct comprising (i) at least one coding nucleotide sequence encoding an immunogenic CEA polypeptide and (ii) at least one coding nucleotide sequence encoding either an immunogenic TERT polypeptide or an immunogenic MUC1 polypeptide. The nucleotide sequence encoding the immunogenic CEA polypeptide may be either upstream or downstream of the other coding nucleotide sequence. The construct may further comprise a spacer nucleotide sequence between the coding nucleotide sequences. The structure of such a dual antigen construct is shown in formula (I) and formula (II):

TAA-SPACER-CEA (I)

CEA-SPACER-TAA (II)

wherein in each of formulas (I) and (II): (i) CEA represents a nucleotide sequence encoding an immunogenic CEA polypeptide; (ii) TAA represents a nucleotide sequence encoding either an immunogenic MUC1 polypeptide or an immunogenic TERT polypeptide; and (iii) SPACER is a spacer nucleotide sequence and may be absent. Examples of spacer nucleotide sequences that may be included in the dual-antigen constructs include nucleotide sequences encoding a foot-and-mouth disease virus 2A peptide (FMD2A or FMDV2A), equine rhinitis A virus 2A peptide (ERA2A), Equine rhinitis B virus 2A peptide (ERB2A), encephalomyocarditis virus 2A peptide (EMC2A or EMCV2A), porcine teschovirus 2A peptide (PT2A), and Thosea asigna virus 2A peptide (T2A, TA2A, or TAV2A). In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In some other aspects, the present disclosure provides a multi-antigen construct comprising (i) at least one coding nucleotide sequence encoding an immunogenic CEA polypeptide, (ii) at least one coding nucleotide sequence encoding an immunogenic MUC1 polypeptide, and (iii) at least one coding nucleotide sequence encoding an immunogenic TERT polypeptide. In some embodiments, the multi-antigen construct further comprises a spacer nucleotide sequence. The structure of a multi-antigen construct is shown in formula (III):

TAA1-SPACER1-TAA2-SPACER2-TAA3 (III)

wherein in formula (III): (i) TAA1, TAA2, and TAA3 each represent a nucleotide sequence encoding an immunogenic TAA polypeptide selected from the group consisting of an immunogenic MUC1 polypeptide, an immunogenic CEA polypeptide, and an immunogenic TERT polypeptide, wherein TAA1, TAA2, and TAA3 encode different immunogenic TAA polypeptides; and (ii) SPACER1 and SPACER2 each represent a spacer nucleotide sequence, wherein (a) SPACER1 and SPACER2 may be the same or different and (b) one of or both of SPACER1 and SPACER2 may be absent. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In some embodiments, the present disclosure provides a multi-antigen construct of formula (III), wherein in formula (III): (i) TAA1 is a nucleotide sequence encoding an immunogenic MUC1 polypeptide; (ii) TAA2 is a nucleotide sequence encoding an immunogenic CEA polypeptide; and (iii) TAA3 is a nucleotide sequence encoding an immunogenic TERT polypeptide. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In some other embodiments, the present disclosure provides a multi-antigen construct of formula (III), wherein in formula (III): (i) TAA1 is a nucleotide sequence encoding an immunogenic MUC1 polypeptide; (ii) TAA2 is a nucleotide sequence encoding an immunogenic TERT polypeptide; and (iii) TAA3 is a nucleotide sequence encoding an immunogenic CEA polypeptide. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In still other embodiments, the present disclosure provides a multi-antigen construct of formula (III), wherein in formula (III): (i) TAA1 is a nucleotide sequence encoding an immunogenic CEA polypeptide; (ii) TAA2 is a nucleotide sequence encoding an immunogenic TERT polypeptide; and (iii) TAA3 is a nucleotide sequence encoding an immunogenic MUC1 polypeptide. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In some further embodiments, the present disclosure provides a multi-antigen construct of formula (III), wherein in formula (III): (i) TAA1 is a nucleotide sequence encoding an immunogenic CEA polypeptide; (ii) TAA2 is a nucleotide sequence encoding an immunogenic MUC1 polypeptide; and (iii) TAA3 is a nucleotide sequence encoding an immunogenic TERT polypeptide. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In still other embodiments, the present disclosure provides a multi-antigen construct of formula (III), wherein in formula (III): (i) TAA1 is a nucleotide sequence encoding an immunogenic TERT polypeptide; (ii) TAA2 is a nucleotide sequence encoding an immunogenic MUC1 polypeptide; and (iii) TAA3 is a nucleotide sequence encoding an immunogenic CEA polypeptide. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In still other embodiments, the present disclosure provides a multi-antigen construct of formula (III), wherein in formula (III): (i) TAA1 is a nucleotide sequence encoding an immunogenic TERT polypeptide; (ii) TAA2 is a nucleotide sequence encoding an immunogenic CEA polypeptide; and (iii) TAA3 is a nucleotide sequence encoding an immunogenic MUC1 polypeptide. In some embodiments, SPACER1 and SPACER2 are, independently, a nucleotide sequence encoding a 2A peptide, or a nucleotide sequence encoding GGSGG. In some embodiments, SPACER1 and SPACER2 are a nucleotide sequence encoding a 2A peptide. In some embodiments, SPACER1 and SPACER2 are a nucleotide sequence encoding GGSGG. In some embodiments, SPACER1 is a nucleotide sequence encoding a 2A peptide and SPACER2 is a nucleotide sequence encoding GGSGG. In some embodiments, SPACER1 is a nucleotide sequence encoding GGSGG and SPACER2 is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

In some specific embodiments, the present disclosure provides a multi-antigen construct of a formula selected from the group consisting of:

(1) MUC1-2A-CEA-2A-TERT (IV)

(2) MUC1-2A-TERT-2A-CEA (V)

(3) CEA-2A-MUC1-2A-TERT (VI)

(4) CEA-2A-TERT-2A-MUC1 (VII)

(5) TERT-2A-MUC1-2A-CEA (VIII)

(6) TERT-2A-CEA-2A-MUC1 (IX)

wherein in each of formulas (IV)-(IX): (i) MUC1, CEA, and TERT represent a nucleotide sequence encoding an immunogenic MUC1 polypeptide, an immunogenic CEA polypeptide, and an immunogenic TERT polypeptide, respectively; and (ii) 2A is a nucleotide sequence encoding a 2A peptide. In some embodiments, the antigen construct encodes any of the immunogenic CEA polypeptides described herein above. In some embodiments, the antigen construct encodes any of the immunogenic TERT polypeptides described herein above. In some embodiments, the antigen construct encodes any of the immunogenic MUC1 polypeptides described herein above.

The immunogenic CEA polypeptide, immunogenic MUC1 polypeptide, and immunogenic TERT polypeptide encoded by a multi-antigen construct, including dual antigen constructs and triple-antigen constructs, may be in membrane-bound form or cytoplasmic form. In some specific embodiments, the immunogenic TAA polypeptide is in cytoplasmic form.

In some embodiments, the immunogenic CEA polypeptide encoded by a multi-antigen construct comprises (1) the amino acid sequence of the N-domain and (2) the amino acid sequence of 1, 2, 3, 4, or 5 C-like domains of a human CEA protein. In some particular embodiments, the immunogenic CEA polypeptides comprise (1) the amino acid sequence of at least four C-like domains, such as A2, B2, A3, and B3, and (2) the amino acid sequence of the N-domain. In some further embodiments, the immunogenic CEA polypeptides are in cytoplasmic form (or “cCEA”) or the membrane-bound form (or “mCEA”).

In some specific embodiments, the immunogenic CEA polypeptide encoded by a multi-antigen construct comprises an amino acid sequence selected from:

(1) an amino acid sequence comprising or consisting of (i) amino acids 323-677 of SEQ ID NO:2 or (ii) amino acids 35-144 and 323-677 of SEQ ID NO:2;

(2) an amino acid sequence comprising or consisting of (i) amino acids 323-702 of SEQ ID NO:2 or (ii) amino acids 2-144 and 323-702 of SEQ ID NO:2;

(3) the amino acid sequence of SEQ ID NO:17 (amino acid sequence encoded by Plasmid 1386 (mCEA) or amino acids 4-526 of SEQ ID NO:17;

(4) the amino acid sequence of SEQ ID NO:19 (amino acid sequence encoded Plasmid 1390 (cCEA) or amino acids 4-468 of SEQ ID NO:19; or

(5) a functional variant of any of the amino acid sequences of (1)-(4) above.

In some specific embodiments, the immunogenic CEA polypeptide encoded by a multi-antigen construct consists of an amino acid sequence selected from:

(1) an amino acid sequence comprising or consisting of (i) amino acids 323-677 of SEQ ID NO:2 or (ii) amino acids 35-144 and 323-677 of SEQ ID NO:2;

(2) an amino acid sequence comprising or consisting of (i) amino acids 323-702 of SEQ ID NO:2 or (ii) amino acids 2-144 and 323-702 of SEQ ID NO:2;

(3) the amino acid sequence of SEQ ID NO:17 (amino acid sequence encoded by Plasmid 1386 (mCEA) or amino acids 4-526 of SEQ ID NO:17;

(4) the amino acid sequence of SEQ ID NO:19 (amino acid sequence encoded Plasmid 1390 (cCEA) or amino acids 4-468 of SEQ ID NO:19; or

(5) a functional variant of any of the amino acid sequences of (1)-(4) above.

In some particular embodiments, the multi-antigen construct is a DNA and comprises (1) the nucleotide sequence of SEQ ID NO:14, (2) the nucleotide sequence of SEQ ID NO:16, (3) the nucleotide sequence of SEQ ID NO:18, or (4) a degenerate variant of the nucleotide sequence of SEQ ID NO:14, 16, or 18. In some other particular embodiments, the multi-antigen construct is a RNA and comprises a nucleotide sequence that corresponds to: (1) the nucleotide sequence of SEQ ID NO:14; (2) the nucleotide sequence of SEQ ID NO:16; (3) the nucleotide sequence of SEQ ID NO:18; or (4) a degenerate variant of the nucleotide sequence of SEQ ID NO:14, 16, or 18.

In some embodiments, the immunogenic MUC1 polypeptide encoded by a multi-antigen construct comprises (1) an amino acid sequence of 3 to 30 tandem repeats of 20 amino acids of a human MUC1 protein and (2) the amino acid sequences of the human MUC1 protein that flank the VNTR region. In some specific embodiments, the immunogenic MUC1 polypeptide encoded by a multi-antigen construct comprises an amino acid sequence selected from the group consisting of:

(1) the amino acid sequence of SEQ ID NO:5 (Plasmid 1027 polypeptide);

(2) an amino acid sequence comprising amino acids 4-537 of SEQ ID NO:5;

(3) an amino acid sequence comprising amino acids 24-537 of SEQ ID NO:5;

(4) the amino acid sequence of SEQ ID NO:7 (Plasmid 1197 polypeptide);

(5) an amino acid sequence comprising amino acids 4-517 of SEQ ID NO:7; and

(6) an amino acid sequence comprising amino acids 4-517 of SEQ ID NO:7, with the proviso that the amino acid at position 513 is T.

In some embodiments, the immunogenic MUC1 polypeptide encoded by a multi-antigen construct consists of (1) an amino acid sequence of 3 to 30 tandem repeats of 20 amino acids of a human MUC1 protein and (2) the amino acid sequences of the human MUC1 protein that flank the VNTR region. In some specific embodiments, the immunogenic MUC1 polypeptide encoded by a multi-antigen construct consists of an amino acid sequence selected from the group consisting of:

(1) the amino acid sequence of SEQ ID NO:5 (Plasmid 1027 polypeptide);

(2) an amino acid sequence comprising amino acids 4-537 of SEQ ID NO:5;

(3) an amino acid sequence comprising amino acids 24-537 of SEQ ID NO:5;

(4) the amino acid sequence of SEQ ID NO:7 (Plasmid 1197 polypeptide);

(5) an amino acid sequence comprising amino acids 4-517 of SEQ ID NO:7; and

(6) an amino acid sequence comprising amino acids 4-517 of SEQ ID NO:7, with the proviso that the amino acid at position 513 is T.

In some specific embodiments, the immunogenic MUC1 polypeptide encoded by a multi-antigen construct consists of an amino acid sequence selected from the group consisting of:

(1) an amino acid sequence consisting of amino acids 4-537 of SEQ ID NO:5;

(2) an amino acid sequence consisting of amino acids 24-537 of SEQ ID NO:5;

(3) an amino acid sequence consisting of amino acids 4-517 of SEQ ID NO:7;

and

(4) an amino acid sequence consisting of amino acids 4-517 of SEQ ID NO:7, with the proviso that the amino acid at position 513 is T.

In some particular embodiments, the multi-antigen construct is a DNA and comprises: (1) the nucleotide sequence of SEQ ID NO:4 or a nucleotide sequence comprising nucleotides 10-1611 of SEQ ID NO:4; (2) the nucleotide sequence of SEQ ID NO:6 or a nucleotide sequence comprising nucleotides 10-1551 of SEQ ID NO:6; or (3) a degenerate variant of the nucleotide sequence of SEQ ID NO:4 or SEQ ID NO:6.

In some other particular embodiments, the multi-antigen construct is a RNA and comprises a nucleotide sequence that corresponds to (1) the nucleotide sequence of SEQ ID NO:4, (2) the nucleotide sequence of SEQ ID NO:6, or (3) a degenerate variant of the nucleotide sequence of SEQ ID NO:4 or SEQ ID NO:6.

The immunogenic TERT polypeptide encoded by a multi-antigen construct may be the full length TERT protein or any truncated or mutated form of the TERT protein. The full length TERT protein is expected to generate stronger immune responses than a truncated form. However, depending on the specific vector chosen to deliver the construct, the vector may not have the capacity to carry the gene encoding the full TERT protein. Therefore, deletions of some amino acids from the protein may be made such that the transgenes would fit into a particular vector. The deletions of amino acids can be made from the N-terminus, C-terminus, or anywhere in the sequence of the TERT protein (e.g. from the TERT protein of SEQ ID NO:3). Additional deletions may be made in order to remove the nuclear localization signal, thereby rendering the polypeptides cytoplasmic, increasing access to cellular antigen processing/presentation machinery. In some embodiments, the amino acids up to position 200, 300, 400, 500, or 600 of the N-terminus of the TERT protein are absent from the immunogenic TERT polypeptides (e.g. from the TERT protein of SEQ ID NO:3).

In some specific embodiments, amino acids 1-343 (TERT343), 1-240 (TERT240), or 1-541 (TERT541) of the N-terminus of the TERT protein of SEQ ID NO:3 are absent. Thus, in an embodiment, the amino acid sequence of the immunogenic TERT polypeptide encoded by a multi-antigen construct of the invention is any of the following:

(1) an amino acid sequence comprising amino acids 51-1132 of SEQ ID NO:3 and lacking amino acids 1 to 50 of SEQ ID NO:3;

(2) an amino acid sequence comprising amino acids 101-1132 of SEQ ID NO:3 and lacking amino acids 1 to 100 of SEQ ID NO:3;

(3) an amino acid sequence comprising amino acids 151-1132 of SEQ ID NO:3 and lacking amino acids 1 to 150 of SEQ ID NO:3;

(4) an amino acid sequence comprising amino acids 201-1132 of SEQ ID NO:3 and lacking amino acids 1 to 200 of SEQ ID NO:3;

(5) an amino acid sequence comprising amino acids 241-1132 of SEQ ID NO:3 and lacking amino acids 1 to 240 of SEQ ID NO:3;

(6) an amino acid sequence comprising amino acids 301-1132 of SEQ ID NO:3 and lacking amino acids 1 to 300 of SEQ ID NO:3;

(7) an amino acid sequence comprising amino acids 351-1132 of SEQ ID NO:3 and lacking amino acids 1 to 350 of SEQ ID NO:3;

(8) an amino acid sequence comprising amino acids 401-1132 of SEQ ID NO:3 and lacking amino acids 1 to 400 of SEQ ID NO:3;

(9) an amino acid sequence comprising amino acids 451-1132 of SEQ ID NO:3 and lacking amino acids 1 to 450 of SEQ ID NO:3;

(10) an amino acid sequence comprising amino acids 501-1132 of SEQ ID NO:3 and lacking amino acids 1 to 500 of SEQ ID NO:3;

(11) an amino acid sequence comprising amino acids 551-1132 of SEQ ID NO:3 and lacking amino acids 1 to 550 of SEQ ID NO:3; or

(12) an amino acid sequence comprising amino acids 601-1132 of SEQ ID NO:3 and lacking amino acids 1-600 of SEQ ID NO:3.

In an embodiment, the amino acid sequence of the immunogenic TERT polypeptide encoded by a multi-antigen construct of the invention is any of the following:

(1) an amino acid sequence consisting of amino acids 51-1132, 101-1132, 151-1132, 201-1132, 251-1132, 301-1132, 351-1132, 401-1132, 451-1132, 501-1132, or 551-1132 of SEQ ID NO:3;

(2) an amino acid sequence consisting of amino acids 601-1132 of SEQ ID NO:3;

(3) an amino acid sequence consisting of amino acids 542-1132 of SEQ ID NO:3;

(4) an amino acid sequence consisting of amino acids 344-1132 of SEQ ID NO:3; and

(5) an amino acid sequence consisting of amino acids 241-1132 of SEQ ID NO:3.

Mutations of additional amino acids may be introduced in order to inactivate the TERT catalytic domain. Examples of such mutations include substitution of aspartic acid at position 712 of SEQ ID NO:3, such as D712A, and substitution of valine at position 713 of SEQ ID NO:3, such as V713I. Therefore, in an embodiment, the immunogenic TERT polypeptide encoded by a multi-antigen construct consists of any of the above disclosed TERT polypeptides wherein a substitution at position corresponding to aspartic acid 712 of SEQ ID NO:3 and/or substitution at position corresponding to valine 713 of SEQ ID NO:3 and wherein said mutation(s) inactivates the TERT catalytic domain. In an embodiment said mutation consists of a substitution of aspartic acid at position corresponding to position 712 of SEQ ID NO:3 and substitution of valine at position corresponding to position 713 of SEQ ID NO:3 wherein said mutation(s) inactivate the TERT catalytic domain. In an embodiment said mutation consists of a substitution of aspartic acid with alanine at position corresponding to position 712 of SEQ ID NO:3 (D712A) and a substitution of valine with isoleucine at position corresponding to position 713 of SEQ ID NO:3 (V713I).

In some specific embodiments, the immunogenic TERT polypeptide encoded by a multi-antigen construct comprises an amino acid sequence selected from the group consisting of:

(1) the amino acid sequence of SEQ ID NO:9 (Plasmid 1112 Polypeptide) or an amino acid sequence comprising amino acids 2-893 of SEQ ID NO:9;

(2) the amino acid sequence of SEQ ID NO:11 (Plasmid 1326 Polypeptide) or an amino acid sequence comprising amino acids 4-791 of SEQ ID NO:11;

(3) the amino acid sequence of SEQ ID NO:13 (Plasmid 1330 Polypeptide) or an amino acid sequence comprising amino acids 4-594 of SEQ ID NO:13; or

(4) an amino acid sequence that is a functional variant of any of the amino acid sequences (1)-(3) above.

In some specific embodiments, the immunogenic TERT polypeptide encoded by a multi-antigen construct consists of an amino acid sequence selected from the group consisting of:

(1) the amino acid sequence of SEQ ID NO:9 (Plasmid 1112 Polypeptide) or an amino acid sequence comprising amino acids 2-893 of SEQ ID NO:9;

(2) the amino acid sequence of SEQ ID NO:11 (Plasmid 1326 Polypeptide) or an amino acid sequence comprising amino acids 4-791 of SEQ ID NO:11;

(3) the amino acid sequence of SEQ ID NO:13 (Plasmid 1330 Polypeptide) or an amino acid sequence comprising amino acids 4-594 of SEQ ID NO:13; or

(4) an amino acid sequence that is a functional variant of any of the amino acid sequences (1)-(3) above.

In some specific embodiments, the immunogenic TERT polypeptide encoded by a multi-antigen construct consists of an amino acid sequence selected from the group consisting of:

(1) an amino acid sequence consisting of amino acids 2-893 of SEQ ID NO:9;

(2) an amino acid sequence consisting of amino acids 4-791 of SEQ ID NO:11;

(3) an amino acid sequence consisting of amino acids 4-594 of SEQ ID NO:13; or

(4) an amino acid sequence that is a functional variant of any of the amino acid sequences (1)-(3) above.

In some particular embodiments, the multi-antigen construct is a DNA and comprises: (1) the nucleotide sequence of SEQ ID NO:8 or a nucleotide sequence comprising nucleotides 4-2673 of SEQ ID NO:8; (2) the nucleotide sequence of SEQ ID NO:10 or a nucleotide sequence comprising nucleotides 10-2373 of SEQ ID NO:10; (3) the nucleotide sequence of SEQ ID NO:12 or a nucleotide sequence comprising nucleotides 10-1782 of SEQ ID NO:12; or (4) a degenerate variant of the nucleotide sequence of SEQ ID NO:8, SEQ ID NO:10, or SEQ ID NO:12.

In some other particular embodiments, the multi-antigen construct is a RNA and comprises a nucleotide sequence that corresponds to (1) the nucleotide sequence of SEQ ID NO:8, (2) the nucleotide sequence of SEQ ID NO:10, (3) the nucleotide sequence of SEQ ID NO:12, or (4) a degenerate variant of the nucleotide sequence of SEQ ID NO:8, 10, or 12.

In some particular embodiments, the present disclosure provides a multi-antigen construct that comprises (i) at least one nucleotide sequence encoding an immunogenic CEA polypeptide and (ii) at least one nucleotide sequence encoding either an immunogenic MUC1 polypeptide or an immunogenic TERT polypeptide, wherein the multi-antigen construct encodes an amino acid sequence comprising:

(1) the amino acid sequence of SEQ ID NO:31 or amino acids 4-1088 of SEQ ID NO:31;

(2) the amino acid sequence of SEQ ID NO:33 or amino acids 4-1081 of SEQ ID NO:33;

(3) the amino acid sequence of SEQ ID NO:35 or amino acids 4-1085 of SEQ ID NO:35;

(4) the amino acid sequence of SEQ ID NO:37 or amino acids 4-1030 of SEQ ID NO:37;

(5) the amino acid sequence of SEQ ID NO:39 or amino acids 4-1381 of SEQ ID NO:39; or

(6) the amino acid sequence of SEQ ID NO:41 or amino acids 4-1441 of SEQ ID NO:41.

In some particular embodiments, the present disclosure provides a multi-antigen construct that comprises (i) at least one nucleotide sequence encoding an immunogenic CEA polypeptide and (ii) at least one nucleotide sequence encoding either an immunogenic MUC1 polypeptide or an immunogenic TERT polypeptide, wherein the multi-antigen construct encodes an amino acid sequence consisting of:

(1) the amino acid sequence of SEQ ID NO:31 or amino acids 4-1088 of SEQ ID NO:31;

(2) the amino acid sequence of SEQ ID NO:33 or amino acids 4-1081 of SEQ ID NO:33;

(3) the amino acid sequence of SEQ ID NO:35 or amino acids 4-1085 of SEQ ID NO:35;

(4) the amino acid sequence of SEQ ID NO:37 or amino acids 4-1030 of SEQ ID NO:37;

(5) the amino acid sequence of SEQ ID NO:39 or amino acids 4-1381 of SEQ ID NO:39, or

(6) the amino acid sequence of SEQ ID NO:41 or amino acids 4-1441 of SEQ ID NO:41.

In some specific embodiments, the present disclosure provides a multi-antigen construct that is a DNA and comprises (i) at least one nucleotide sequence encoding an immunogenic CEA polypeptide and (ii) at least one nucleotide sequence encoding either an immunogenic MUC1 polypeptide or an immunogenic TERT polypeptide, wherein the a multi-antigen construct comprises a nucleotide sequence selected from the group consisting of:

(1) the nucleotide sequence of SEQ ID NO:30 or a nucleotide sequence comprising nucleotides 10-3264 of SEQ ID NO:30;

(2) the nucleotide sequence of SEQ ID NO:32 or a nucleotide sequence comprising nucleotides 10-3243 of SEQ ID NO:32;

(3) the nucleotide sequence of SEQ ID NO:34 or a nucleotide sequence comprising nucleotides 10-3255 of SEQ ID NO:34;

(4) the nucleotide sequence of SEQ ID NO:36 or a nucleotide sequence comprising nucleotides 10-3090 of SEQ ID NO:36;

(5) the nucleotide sequence of SEQ ID NO:38 or a nucleotide sequence comprising nucleotides 10-4143 of SEQ ID NO:38;

(6) the nucleotide sequence of SEQ ID NO:40 or a nucleotide sequence comprising nucleotides 10-4323 of SEQ ID NO:40; or

(7) a nucleotide sequence that is a degenerate variant of any of the nucleotide sequences of (1)-(6) above.

In some specific embodiments, the present disclosure provides a multi-antigen construct that is a DNA and comprises (i) at least one nucleotide sequence encoding an immunogenic CEA polypeptide and (ii) at least one nucleotide sequence encoding either an immunogenic MUC1 polypeptide or an immunogenic TERT polypeptide, wherein the a multi-antigen construct comprises a nucleotide sequence selected from the group consisting of:

(1) a nucleotide sequence consisting of nucleotides 10-3264 of SEQ ID NO:30;

(2) a nucleotide sequence consisting of nucleotides 10-3243 of SEQ ID NO:32;

(3) a nucleotide sequence consisting of nucleotides 10-3255 of SEQ ID NO:34;

(4) a nucleotide sequence consisting of nucleotides 10-3090 of SEQ ID NO:36;

(5) a nucleotide sequence consisting of nucleotides 10-4143 of SEQ ID NO:38;

(6) a nucleotide sequence consisting of nucleotides 10-4323 of SEQ ID NO:40; or

(7) a nucleotide sequence that is a degenerate variant of any of the nucleotide sequences (1)-(6) above.

In some other specific embodiments, the present disclosure provides a multi-antigen construct that is a RNA (e.g. mRNA) and comprises (i) at least one nucleotide sequence encoding an immunogenic CEA polypeptide and (ii) at least one nucleotide sequence encoding either an immunogenic MUC1 polypeptide or an immunogenic TERT polypeptide, wherein the a multi-antigen construct comprises a nucleotide sequence that corresponds to a nucleotide sequence selected from the group consisting of:

(1) the nucleotide sequence of SEQ ID NO:30 or a nucleotide sequence comprising nucleotides 10-3264 of SEQ ID NO:30;

(2) the nucleotide sequence of SEQ ID NO:32 or a nucleotide sequence comprising nucleotides 10-3243 of SEQ ID NO:32;

(3) the nucleotide sequence of SEQ ID NO:34 or a nucleotide sequence comprising nucleotides 10-3255 of SEQ ID NO:34;

(4) the nucleotide sequence of SEQ ID NO:36 or a nucleotide sequence comprising nucleotides 10-3090 of SEQ ID NO:36;

(5) the nucleotide sequence of SEQ ID NO:38 or a nucleotide sequence comprising nucleotides 10-4143 of SEQ ID NO:38;

(6) the nucleotide sequence of SEQ ID NO:40 or a nucleotide sequence comprising nucleotides 10-4323 of SEQ ID NO:40; or

(7) a nucleotide sequence that is a degenerate variant of any of the nucleotide sequences (1)-(6) above.

In some other specific embodiments, the present disclosure provides a multi-antigen construct that is a RNA (e.g. mRNA) and comprises (i) at least one nucleotide sequence encoding an immunogenic CEA polypeptide and (ii) at least one nucleotide sequence encoding either an immunogenic MUC1 polypeptide or an immunogenic TERT polypeptide, wherein the a multi-antigen construct comprises a nucleotide sequence that corresponds to a nucleotide sequence selected from the group consisting of:

(1) a nucleotide sequence consisting of nucleotides 10-3264 of SEQ ID NO:30;

(2) a nucleotide sequence consisting of nucleotides 10-3243 of SEQ ID NO:32;

(3) a nucleotide sequence consisting of nucleotides 10-3255 of SEQ ID NO:34;

(4) a nucleotide sequence consisting of nucleotides 10-3090 of SEQ ID NO:36;

(5) a nucleotide sequence consisting of nucleotides 10-4143 of SEQ ID NO:38;

(6) a nucleotide sequence consisting of nucleotides 10-4323 of SEQ ID NO:40; or

(7) a nucleotide sequence that is a degenerate variant of any of the nucleotide sequences (1)-(6) above.

In some other embodiments, the present disclosure provides a multi-antigen construct that comprises (1) at least one nucleotide sequence encoding an immunogenic CEA polypeptide, (2) at least one nucleotide sequence encoding an immunogenic MUC1 polypeptide, and (3) at least one nucleotide sequence encoding an immunogenic TERT polypeptide, wherein the multi-antigen construct comprises a nucleotide sequence encoding an amino acid sequence selected from the group consisting of:

(1) the amino acid sequence of SEQ ID NO:43 or an amino acid sequence comprising amino acids 4-2003 of SEQ ID NO:43;

(2) the amino acid sequence of SEQ ID NO:45 or an amino acid sequence comprising amino acids 4-2001 of SEQ ID NO:45;

(3) the amino acid sequence of SEQ ID NO:47 or an amino acid sequence comprising amino acids 4-2008 of SEQ ID NO:47;

(4) the amino acid sequence of SEQ ID NO:49 or an amino acid sequence comprising amino acids 4-1996 of SEQ ID NO: 49;

(5) the amino acid sequence of SEQ ID NO:51 or an amino acid sequence comprising amino acids 4-1943 of SEQ ID NO:51;

(6) the amino acid sequence of SEQ ID NO:53 or an amino acid sequence comprising amino acids 4-1943 of SEQ ID NO:53; or

(7) a functional variant of any of the amino acid sequences (1)-(6) above.

In some other embodiments, the present disclosure provides a multi-antigen construct that comprises (1) at least one nucleotide sequence encoding an immunogenic CEA polypeptide, (2) at least one nucleotide sequence encoding an immunogenic MUC1 polypeptide, and (3) at least one nucleotide sequence encoding an immunogenic TERT polypeptide, wherein the multi-antigen construct comprises a nucleotide sequence encoding an amino acid sequence selected from the group consisting of:

(1) an amino acid sequence consisting of amino acids 4-2003 of SEQ ID NO:43;

(2) an amino acid sequence consisting of amino acids 4-2001 of SEQ ID NO:45;

(3) an amino acid sequence consisting of amino acids 4-2008 of SEQ ID NO:47;

(4) an amino acid sequence consisting of amino acids 4-1996 of SEQ ID NO: 49;

(5) an amino acid sequence consisting of amino acids 4-1943 of SEQ ID NO:51;

or

(6) an amino acid sequence consisting of amino acids 4-1943 of SEQ ID NO:53.

In some particular embodiments, the present disclosure provides a multi-antigen construct that comprises (1) at least one nucleotide sequence encoding an immunogenic CEA polypeptide, (2) at least one nucleotide sequence encoding an immunogenic MUC1 polypeptide, and (3) at least one nucleotide sequence encoding an immunogenic TERT polypeptide, wherein the multi-antigen construct is a DNA and comprises a nucleotide sequence selected from the group consisting of:

(1) the nucleotide sequence of SEQ ID NO:42 or a nucleotide sequence comprising nucleotides 10-6009 of SEQ ID NO:42;

(2) the nucleotide sequence of SEQ ID NO:44 or a nucleotide sequence comprising nucleotides 10-6003 of SEQ ID NO:44;

(3) the nucleotide sequence of SEQ ID NO:46 or a nucleotide sequence comprising nucleotides 10-6024 of SEQ ID NO:46;

(4) the nucleotide sequence of SEQ ID NO:48 or a nucleotide sequence comprising nucleotides 10-5988 of SEQ ID NO:48;

(5) the nucleotide sequence of SEQ ID NO:50 or a nucleotide sequence comprising nucleotides 10-5829 of SEQ ID NO:50;

(6) the nucleotide sequence of SEQ ID NO:52 or a nucleotide sequence comprising nucleotides 10-5829 of SEQ ID NO:52; and

(7) a nucleotide sequence that is a degenerate variant of any of the nucleotide sequences (1)-(6) above.

In some particular embodiments, the present disclosure provides a multi-antigen construct that comprises (1) at least one nucleotide sequence encoding an immunogenic CEA polypeptide, (2) at least one nucleotide sequence encoding an immunogenic MUC1 polypeptide, and (3) at least one nucleotide sequence encoding an immunogenic TERT polypeptide, wherein the multi-antigen construct is a DNA and comprises a nucleotide sequence selected from the group consisting of:

(1) a nucleotide sequence consisting of nucleotides 10-6009 of SEQ ID NO:42;

(2) a nucleotide sequence consisting of nucleotides 10-6003 of SEQ ID NO:44;

(3) a nucleotide sequence consisting of nucleotides 10-6024 of SEQ ID NO:46;

(4) a nucleotide sequence consisting of nucleotides 10-5988 of SEQ ID NO:48;

(5) a nucleotide sequence consisting of nucleotides 10-5829 of SEQ ID NO:50;

(6) a nucleotide sequence consisting of nucleotides 10-5829 of SEQ ID NO:52; and

(7) a nucleotide sequence that is a degenerate variant of any of the nucleotide sequences (1)-(6) above.

In some other particular embodiments, the present disclosure provides a multi-antigen construct, wherein the multi-antigen construct is a RNA (e.g. mRNA) and comprises a nucleotide sequence that corresponds to a nucleotide sequence selected from the group consisting of:

(1) the nucleotide sequence of SEQ ID NO:42 or a nucleotide sequence comprising nucleotides 10-6009 of SEQ ID NO:42;

(2) the nucleotide sequence of SEQ ID NO:44 or a nucleotide sequence comprising nucleotides 10-6003 of SEQ ID NO:44;

(3) the nucleotide sequence of SEQ ID NO:46 or a nucleotide sequence comprising nucleotides 10-6024 of SEQ ID NO:46;

(4) the nucleotide sequence of SEQ ID NO:48 or a nucleotide sequence comprising nucleotides 10-5988 of SEQ ID NO:48;

(5) the nucleotide sequence of SEQ ID NO:50 or a nucleotide sequence comprising nucleotides 10-5829 of SEQ ID NO:50;

(6) the nucleotide sequence of SEQ ID NO:52 or a nucleotide sequence comprising nucleotides 10-5829 of SEQ ID NO:52; and

(7) a nucleotide sequence that is a degenerate variant of any of the nucleotide sequences (1)-(6) above.

In some other particular embodiments, the present disclosure provides a multi-antigen construct, wherein the multi-antigen construct is a RNA (e.g. mRNA) and comprises a nucleotide sequence that corresponds to a nucleotide sequence selected from the group consisting of:

(1) a nucleotide sequence consisting of nucleotides 10-6009 of SEQ ID NO:42;

(2) a nucleotide sequence consisting of nucleotides 10-6003 of SEQ ID NO:44;

(3) a nucleotide sequence consisting of nucleotides 10-6024 of SEQ ID NO:46;

(4) a nucleotide sequence consisting of nucleotides 10-5988 of SEQ ID NO:48;

(5) a nucleotide sequence consisting of nucleotides 10-5829 of SEQ ID NO:50;

(6) a nucleotide sequence consisting of nucleotides 10-5829 of SEQ ID NO:52; and

(7) a nucleotide sequence that is a degenerate variant of any of the nucleotide sequences (1)-(6) above.

In still other particular embodiments, the present disclosure provides a multi-antigen construct comprising (1) at least one nucleotide sequence encoding an immunogenic CEA polypeptide, (2) at least one nucleotide sequence encoding an immunogenic MUC1 polypeptide, and (3) at least one nucleotide sequence encoding an immunogenic TERT polypeptide, wherein the multi-antigen construct is an RNA (e.g. mRNA) and comprises a nucleotide sequence selected from the group consisting of:

(1) the nucleotide sequence of SEQ ID NO:87;

(2) the nucleotide sequence of SEQ ID NO:88;

(3) the nucleotide sequence of SEQ ID NO:89;

(4) the nucleotide sequence of SEQ ID NO:90;

(5) the nucleotide sequence of SEQ ID NO:91;

(6) the nucleotide sequence of SEQ ID NO:92; and

(7) a degenerate variant of any of the nucleotide sequence of SEQ ID NO:87, SEQ ID NO:88, SEQ ID NO:89, SEQ ID NO:90, SEQ ID NO:91, or SEQ ID NO:92.

In still other particular embodiments, the present disclosure provides a multi-antigen construct comprising (1) at least one nucleotide sequence encoding an immunogenic CEA polypeptide, (2) at least one nucleotide sequence encoding an immunogenic MUC1 polypeptide, and (3) at least one nucleotide sequence encoding an immunogenic TERT polypeptide, wherein the multi-antigen construct is an RNA (e.g. mRNA) and consists of a nucleotide sequence selected from the group consisting of:

(1) the nucleotide sequence of SEQ ID NO:87;

(2) the nucleotide sequence of SEQ ID NO:88;

(3) the nucleotide sequence of SEQ ID NO:89;

(4) the nucleotide sequence of SEQ ID NO:90;

(5) the nucleotide sequence of SEQ ID NO:91;

(6) the nucleotide sequence of SEQ ID NO:92; and

(7) a degenerate variant of any of the nucleotide sequence of SEQ ID NO:87, SEQ ID NO:88, SEQ ID NO:89, SEQ ID NO:90, SEQ ID NO:91, or SEQ ID NO:92.

### D. Vectors Containing an Antigen Construct

Another aspect of the invention relates to vectors containing one or more of any of the antigen constructs provided by the present disclosure, including single antigen constructs, dual-antigen constructs, triple-antigen constructs, and other multi-antigen constructs. The vectors are useful for cloning or expressing the immunogenic TAA polypeptides encoded by the antigen constructs, or for delivering the antigen construct in a composition, such as a vaccine, to a host cell or to a host animal, such as a human.

A wide variety of vectors may be prepared to contain and express an antigen construct provided by the present disclosure, such as plasmid vectors, cosmid vectors, phage vectors, and viral vectors. In addition to the transgene insert sequence (i.e., the single-antigen construct or multi-antigen constructs provided by the present disclosure), which is also referred to as open reading frame (ORF), the structure of a vector typically comprises other components or elements that enable or facilitate the expression, such as origin of replication, multi-cloning site, and a selectable marker.

In some embodiments, the disclosure provides a plasmid vector containing an antigen construct provided by the present disclosure. Examples of suitable plasmid vectors include pBR325, pUC18, pSKF, pET23D, and pGB-2. Other examples of plasmid vectors, as well as method of constructing such vectors, are described in U.S. Pat. Nos. 5,589,466, 5,688,688, and 5,814,482. Construction of specific exemplary plasmid vectors comprising a single-antigen construct, dual-antigen construct, or triple-antigen construct is also described in the present disclosure.

In some specific embodiments, the disclosure provides a plasmid vector comprising a nucleotide sequence of any of SEQ ID NOs:54, 55, 56, 57, 59, 61, 63, 65, 67, 69, 70, 71, 72, 73, and 74.

In other embodiments, the present invention provides vectors that are constructed from viruses (i.e., viral vectors), including DNA viruses and RNA viruses (retroviruses). Examples of DNA viruses that may be used to construct a vector include herpes simplex virus, parvovirus, vaccinia virus, and adenoviruses. Examples of RNA viruses that may be used to construct a vector include alphavirus, flavivirus, pestivirus, influenzavirus, lyssavirus, and vesiculovirus. Construction of vectors from various viruses is known in the art. Examples of retroviral vectors are described in U.S. Pat. Nos. 5,716,613, 5,716,832, and 5,817,491. Examples of vectors that can be generated from alphaviruses are described in U.S. Pat. Nos. 5,091,309, 5,843,723, and 5,789,245. Examples of other vectors include: (1) pox viruses, such as canary pox virus or vaccinia virus (U.S. Pat. Nos. 4,603,112, 4,769,330 and 5,017,487; WO 89/01973); (2) SV40 (Mulligan et al., Nature 277:108-114, 1979); (3) herpes (Kit, Adv. Exp. Med. Biol. 215:219-236, 1989; U.S. Pat. No. 5,288,641); and (4) lentivirus such as HIV (Poznansky, J. Virol. 65:532-536, 1991).

In some particular embodiments, the present disclosure provides adenoviral vectors derived from non-human primate adenoviruses, such as simian adenoviruses. Examples of such adenoviral vectors, as well as their preparation, are described in PCT application publications WO2005/071093 and WO2010/086189, and include non-replicating vectors constructed from simian adenoviruses, such as ChAd3, ChAd4, ChAdS, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, ChAd68, ChAd82, ChAd55, ChAd73, ChAd83, ChAd146, ChAd147, PanAd1, Pan Ad2, and Pan Ad3, and replication-competent vectors constructed from adenoviruses Ad4 or Ad7. It is preferred that in constructing the adenoviral vectors from the simian adenoviruses one or more of the early genes from the genomic region of the virus selected from E1A, E1B, E2A, E2B, E3, and E4 are either deleted or rendered non-functional by deletion or mutation. In a particular embodiment, the vector is constructed from ChAd68. The chimpanzee adenovirus ChAd68 is also referred to in the literature as simian adenovirus 25, C68, AdC68, Chad68, SAdV25, PanAd9, or Pan9. A method of constructing vectors from ChAd68 for expressing multi-antigen constructs is described in international patent application publication WO2015/063647. Expression vectors typically include one or more control elements that are operatively linked to the nucleic acid sequence to be expressed. The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription, and translation of a coding sequence in a recipient cell. Not all of these control elements need always be present so long as the selected coding sequence is capable of being replicated, transcribed, and translated in an appropriate host cell. The control elements are selected based on a number of factors known to those skilled in that art, such as the specific host cells and source or structures of other vector components. For enhancing the expression of an immunogenic TAA polypeptide, a Kozak sequence can be provided upstream of the sequence encoding the immunogenic TAA polypeptide. For vertebrates, a known Kozak sequence is (GCC)NCCATGG, wherein N is A or G and GCC is less conserved. Exemplary Kozak sequences that can be used include GAACATGG, ACCAUGG and ACCATGG.

In some embodiments, the vector comprises a multi-antigen construct encoding (i) at least one immunogenic CEA polypeptide and (ii) at least one immunogenic MUC1 polypeptide or at least one immunogenic TERT polypeptide. The vector may be a DNA plasmid vector, DNA virus vector, RNA plasmid vector, or RNA virus vector. In some specific embodiments, the vector comprises a multi-antigen construct which encodes an amino acid sequence comprising:

(1) the amino acid sequence of SEQ ID NO:31 or amino acids 4-1088 of SEQ ID NO:31;

(2) the amino acid sequence of SEQ ID NO:33 or amino acids 4-1081 of SEQ ID NO:33;

(3) the amino acid sequence of SEQ ID NO:35 or amino acids 4-1085 of SEQ ID NO:35;

(4) the amino acid sequence of SEQ ID NO:37 or amino acids 4-1030 of SEQ ID NO:37;

(5) the amino acid sequence of SEQ ID NO:39 or amino acids 4-1381 of SEQ ID NO:39;

(6) the amino acid sequence of SEQ ID NO:41 or amino acids 4-1441 of SEQ ID NO:41; or

(7) a functional variant of any of the amino acid sequences (1)-(6) above.

In some other specific embodiments, the vector is a DNA vector and comprises a multi-antigen construct comprising a nucleotide sequence selected from the group consisting of:

(1) the nucleotide sequence of SEQ ID NO:30 or a nucleotide sequence comprising nucleotides 10-3264 of SEQ ID NO:30;

(2) the nucleotide sequence of SEQ ID NO:32 or a nucleotide sequence comprising nucleotides 10-3243 of SEQ ID NO:32;

(3) the nucleotide sequence of SEQ ID NO:34 or a nucleotide sequence comprising nucleotides 10-3255 of SEQ ID NO:34;

(4) the nucleotide sequence of SEQ ID NO:36 or a nucleotide sequence comprising nucleotides 10-3090 of SEQ ID NO:36;

(5) the nucleotide sequence of SEQ ID NO:38 or a nucleotide sequence comprising nucleotides 10-4143 of SEQ ID NO:38;

(6) the nucleotide sequence of SEQ ID NO:40 or a nucleotide sequence comprising nucleotides 10-4323 of SEQ ID NO:40; and

(7) a nucleotide sequences that is a degenerate variant of any of the nucleotide sequences of (1)-(6) above.

In some other specific embodiments, the present disclosure provides a RNA vector, which comprises a nucleotide sequence that corresponds to a nucleotide sequence selected from the group consisting of:

(1) the nucleotide sequence of SEQ ID NO:30 or a nucleotide sequence comprising nucleotides 10-3264 of SEQ ID NO:30;

(2) the nucleotide sequence of SEQ ID NO:32 or a nucleotide sequence comprising nucleotides 10-3243 of SEQ ID NO:32;

(3) the nucleotide sequence of SEQ ID NO:34 or a nucleotide sequence comprising nucleotides 10-3255 of SEQ ID NO:34;

(4) the nucleotide sequence of SEQ ID NO:36 or a nucleotide sequence comprising nucleotides 10-3090 of SEQ ID NO:36;

(5) the nucleotide sequence of SEQ ID NO:38 or a nucleotide sequence comprising nucleotides 10-4143 of SEQ ID NO:38;

(6) the nucleotide sequence of SEQ ID NO:40 or a nucleotide sequence comprising nucleotides 10-4323 of SEQ ID NO:40; and

(7) a nucleotide sequence that is a degenerate variant of any of the nucleotide sequences of (1)-(6) above.

In some other embodiments, the vector contains a multi-antigen construct encoding (i) at least one immunogenic MUC1 polypeptide, (ii) at least one immunogenic CEA polypeptide, and (iii) at least one immunogenic TERT polypeptide. The vector may be a DNA plasmid vector, DNA virus vector, RNA plasmid vector, or RNA virus vector. In some specific embodiments, the vector comprises a multi-antigen construct which encodes an amino acid sequence selected from the group consisting of:

(1) the amino acid sequence of SEQ ID NO:43 or an amino acid sequence comprising amino acids 4-2003 of SEQ ID NO:43;

(2) the amino acid sequence of SEQ ID NO:45 or an amino acid sequence comprising amino acids 4-2001 of SEQ ID NO:45;

(3) the amino acid sequence of SEQ ID NO:47 or an amino acid sequence comprising amino acids 4-2008 of SEQ ID NO:47;

(4) the amino acid sequence of SEQ ID NO:49 or an amino acid sequence comprising amino acids 4-1996 of SEQ ID NO: 49;

(5) the amino acid sequence of SEQ ID NO:51 or an amino acid sequence comprising amino acids 4-1943 of SEQ ID NO:51;

(6) the amino acid sequence of SEQ ID NO:53 or an amino acid sequence comprising amino acids 4-1943 of SEQ ID NO:53; or

(7) a functional variant of any of the amino acid sequences of (1)-(6) above.

In some other specific embodiments, the present disclosure provides a DNA vector, which comprises a multi-antigen construct comprising a nucleotide sequence selected from the group consisting of:

(1) the nucleotide sequence of SEQ ID NO:42 or a nucleotide sequence comprising nucleotides 10-6009 of SEQ ID NO:42;

(2) the nucleotide sequence of SEQ ID NO:44 or a nucleotide sequence comprising nucleotides 10-6003 of SEQ ID NO:44;

(3) the nucleotide sequence of SEQ ID NO:46 or a nucleotide sequence comprising nucleotides 10-6024 of SEQ ID NO:46;

(4) the nucleotide sequence of SEQ ID NO:48 or a nucleotide sequence comprising nucleotides 10-5988 of SEQ ID NO:48;

(5) the nucleotide sequence of SEQ ID NO:50 or a nucleotide sequence comprising nucleotides 10-5829 of SEQ ID NO:50; or

(6) the nucleotide sequence of SEQ ID NO:52 or a nucleotide sequence comprising nucleotides 10-5829 of SEQ ID NO:52; and

(7) a nucleotide sequence that is a degenerate variant of any of the nucleotide sequences of (1)-(6) above.

In some other specific embodiments, the present disclosure provides a RNA vector, which comprises a nucleotide sequence that corresponds to a nucleotide sequence selected from the group consisting of:

(1) the nucleotide sequence of SEQ ID NO:42 or a nucleotide sequence comprising nucleotides 10-6009 of SEQ ID NO:42;

(2) the nucleotide sequence of SEQ ID NO:44 or a nucleotide sequence comprising nucleotides 10-6003 of SEQ ID NO:44;

(3) the nucleotide sequence of SEQ ID NO:46 or a nucleotide sequence comprising nucleotides 10-6024 of SEQ ID NO:46;

(4) the nucleotide sequence of SEQ ID NO:48 or a nucleotide sequence comprising nucleotides 10-5988 of SEQ ID NO:48;

(5) the nucleotide sequence of SEQ ID NO:50 or a nucleotide sequence comprising nucleotides 10-5829 of SEQ ID NO:50;

(6) the nucleotide sequence of SEQ ID NO:52 or a nucleotide sequence comprising nucleotides 10-5829 of SEQ ID NO:52; and

(7) a nucleotide sequence that is a degenerate variant of any of the nucleotide sequences of (1)-(6) above.

In some specific embodiments, the present disclosure provides a DNA viral vector comprising a nucleotide sequence of any of SEQ ID NOs:58, 60, 62, 64, 66, and 68. In some other specific embodiments, the present disclosure provides a DNA plasmid vector comprising the a nucleotide sequence of any of SEQ ID NOs:57, 59, 61, 63, 65, 67, 69, 70, 71, 72, 73, and 74.

In some specific embodiments, the vector is a DNA vector and comprises a multi-antigen construct comprising a nucleotide sequence selected from the group consisting of:

(1) a nucleotide sequence consisting of nucleotides 10-3264 of SEQ ID NO:30;

(2) a nucleotide sequence consisting of nucleotides 10-3243 of SEQ ID NO:32;

(3) a nucleotide sequence consisting of nucleotides 10-3255 of SEQ ID NO:34;

(4) a nucleotide sequence consisting of nucleotides 10-3090 of SEQ ID NO:36;

(5) a nucleotide sequence consisting of nucleotides 10-4143 of SEQ ID NO:38;

(6) a nucleotide sequence consisting of nucleotides 10-4323 of SEQ ID NO:40; and

(7) a nucleotide sequences that is a degenerate variant of any of the nucleotide sequences of (1)-(6) above.

In some other specific embodiments, the present disclosure provides a RNA vector, which comprises a nucleotide sequence that corresponds to a nucleotide sequence selected from the group consisting of:

(1) a nucleotide sequence consisting of nucleotides 10-3264 of SEQ ID NO:30;

(2) a nucleotide sequence consisting of nucleotides 10-3243 of SEQ ID NO:32;

(3) a nucleotide sequence consisting of nucleotides 10-3255 of SEQ ID NO:34;

(4) a nucleotide sequence consisting of nucleotides 10-3090 of SEQ ID NO:36;

(5) a nucleotide sequence consisting of nucleotides 10-4143 of SEQ ID NO:38;

(6) a nucleotide sequence consisting of nucleotides 10-4323 of SEQ ID NO:40; and

(7) a nucleotide sequence that is a degenerate variant of any of the nucleotide sequences of (1)-(6) above.

In some other embodiments, the vector contains a multi-antigen construct encoding (i) at least one immunogenic MUC1 polypeptide, (ii) at least one immunogenic CEA polypeptide, and (iii) at least one immunogenic TERT polypeptide. The vector may be a DNA plasmid vector, DNA virus vector, RNA plasmid vector, or RNA virus vector. In some specific embodiments, the present disclosure provides a DNA vector, which comprises a multi-antigen construct comprising a nucleotide sequence selected from the group consisting of:

(1) a nucleotide sequence consisting of nucleotides 10-6009 of SEQ ID NO:42;

(2) a nucleotide sequence consisting of nucleotides 10-6003 of SEQ ID NO:44;

(3) a nucleotide sequence consisting of nucleotides 10-6024 of SEQ ID NO:46;

(4) a nucleotide sequence consisting of nucleotides 10-5988 of SEQ ID NO:48;

(5) a nucleotide sequence consisting of nucleotides 10-5829 of SEQ ID NO:50; or

(6) a nucleotide sequence consisting of nucleotides 10-5829 of SEQ ID NO:52; and

(7) a nucleotide sequence that is a degenerate variant of any of the nucleotide sequences of (1)-(6) above.

In some specific embodiments, the present disclosure provides a DNA viral vector consisting of a nucleotide sequence of any of SEQ ID NOs:58, 60, 62, 64, 66, and 68. In some other specific embodiments, the present disclosure provides a DNA plasmid vector consisting of the a nucleotide sequence of any of SEQ ID NOs:57, 59, 61, 63, 65, 67, 69, 70, 71, 72, 73, and 74.

In some other specific embodiments, the present disclosure provides a RNA vector, which comprises a nucleotide sequence that corresponds to a nucleotide sequence selected from the group consisting of:

(1) a nucleotide sequence consisting of nucleotides 10-6009 of SEQ ID NO:42;

(2) a nucleotide sequence consisting of nucleotides 10-6003 of SEQ ID NO:44;

(3) a nucleotide sequence consisting of nucleotides 10-6024 of SEQ ID NO:46;

(4) a nucleotide sequence consisting of nucleotides 10-5988 of SEQ ID NO:48;

(5) a nucleotide sequence consisting of nucleotides 10-5829 of SEQ ID NO:50;

(6) a nucleotide sequence consisting of nucleotides 10-5829 of SEQ ID NO:52; and

(7) a nucleotide sequence that is a degenerate variant of any of the nucleotide sequences of (1)-(6) above.

### E. Compositions Comprising an Antigen Construct or Vector

The present disclosure also provides compositions, which comprise an isolated nucleic acid molecule (i.e., an antigen construct) or a vector provided by the present disclosure. A composition may comprise only one individual antigen construct, such as a dual-antigen construct or a triple-antigen construct. It may also comprise two or more different individual antigen constructs, such as a combination of a single-antigen construct and a dual-antigen construct, or a combination of three or more single-antigen constructs encoding different immunogenic TAA polypeptides. The compositions are useful for eliciting an immune response against a TAA protein in vitro or in vivo in a mammal, including a human. In some embodiments, the compositions are immunogenic compositions or pharmaceutical compositions. In some particular embodiments, the composition is a vaccine composition for administration to humans for (1) inhibiting abnormal cell proliferation, providing protection against the development of cancer (used as a prophylactic), (2) treatment of cancer (used as a therapeutic) associated with TAA over-expression, or (3) eliciting an immune response to a particular human TAA, such as CEA, MUC1, and TERT.

In some embodiments, a composition provided by the present disclosure comprises a multi-antigen construct or a vector comprising a multi-antigen construct, wherein the multi-antigen construct encodes two or more immunogenic TAA polypeptides. For example, a multi-antigen construct may encode two or more immunogenic TAA polypeptides in any of the following combinations:

(1) an immunogenic CEA polypeptide and an immunogenic MUC1 polypeptide;

(2) an immunogenic CEA polypeptide and an immunogenic TERT polypeptide; and

(3) an immunogenic CEA polypeptide, an immunogenic MUC1 polypeptide, and an immunogenic TERT polypeptide.

In some particular embodiments, the composition provided by the present disclosure comprises a dual-antigen construct or a vector comprising a dual-antigen construct, wherein the dual-antigen construct comprises a nucleotide sequence selected from the group consisting of:

(1) a nucleotide sequence encoding the amino acid sequence of SEQ ID NO:31 or amino acids 4-1088 of SEQ ID NO:31;

(2) a nucleotide sequence encoding the amino acid sequence of SEQ ID NO:33 or amino acids 4-1081 of SEQ ID NO:33;

(3) a nucleotide sequence encoding the amino acid sequence of SEQ ID NO:35 or amino acids 4-1085 of SEQ ID NO:35;

(4) a nucleotide sequence encoding the amino acid sequence of SEQ ID NO:37 or amino acids 4-1030 of SEQ ID NO:37;

(5) a nucleotide sequence encoding the amino acid sequence of SEQ ID NO:39 or amino acids 4-1381 of SEQ ID NO:39;

(6) a nucleotide sequence encoding the amino acid sequence of SEQ ID NO:41 or amino acids 4-1441 of SEQ ID NO:41;

(7) the nucleotide sequence of SEQ ID NO:30 or a nucleotide sequence comprising nucleotides 10-3264 of SEQ ID NO:30;

(8) the nucleotide sequence of SEQ ID NO:32 or a nucleotide sequence comprising nucleotides 10-3243 of SEQ ID NO:32;

(9) the nucleotide sequence of SEQ ID NO:34 or a nucleotide sequence comprising nucleotides 10-3255 of SEQ ID NO:34;

(10) the nucleotide sequence of SEQ ID NO:36 or a nucleotide sequence comprising nucleotides 10-3090 of SEQ ID NO:36;

(11) the nucleotide sequence of SEQ ID NO:38 or a nucleotide sequence comprising nucleotides 10-4143 of SEQ ID NO:38;

(12) the nucleotide sequence of SEQ ID NO:40 or a nucleotide sequence comprising nucleotides 10-4323 of SEQ ID NO:40; and

(13) a nucleotide sequences that is a degenerate variant of any of the nucleotide sequences of (1)-(12) above.

In some other particular embodiments, the compositions provided by the present disclosure comprise (1) a triple-antigen construct, or (2) a vector comprising a triple-antigen construct, wherein the triple antigen construct comprises a nucleotide sequence selected from the group consisting of:

(1) a nucleotide sequence encoding the amino acid sequence of SEQ ID NO:43 or amino acids 4-2003 of SEQ ID NO:43;

(2) a nucleotide sequence encoding the amino acid sequence of SEQ ID NO:45 or amino acids 4-2001 of SEQ ID NO:45;

(3) a nucleotide sequence encoding the amino acid sequence of SEQ ID NO:47 or amino acids 4-2008 of SEQ ID NO:47;

(4) a nucleotide sequence encoding the amino acid sequence of SEQ ID NO:49 or amino acids 4-1996 of SEQ ID NO: 49;

(5) a nucleotide sequence encoding the amino acid sequence of SEQ ID NO:51 or amino acids 4-1943 of SEQ ID NO:51;

(6) a nucleotide sequence encoding the amino acid sequence of SEQ ID NO:53 or amino acids 4-1943 of SEQ ID NO:53;

(7) the nucleotide sequence of SEQ ID NO:42 or a nucleotide sequence comprising nucleotides 10-6009 of SEQ ID NO:42;

(8) the nucleotide sequence of SEQ ID NO:44 or a nucleotide sequence comprising nucleotides 10-6003 of SEQ ID NO:44;

(9) the nucleotide sequence of SEQ ID NO:46 or a nucleotide sequence comprising nucleotides 10-6024 of SEQ ID NO:46;

(10) the nucleotide sequence of SEQ ID NO:48 or a nucleotide sequence comprising nucleotides 10-5988 of SEQ ID NO:48;

(11) the nucleotide sequence of SEQ ID NO:50 or a nucleotide sequence comprising nucleotides 10-5829 of SEQ ID NO:50;

(12) the nucleotide sequence of SEQ ID NO:52 or a nucleotide sequence comprising nucleotides 10-5829 of SEQ ID NO:52; and

(13) a nucleotide sequence that is a degenerate variant of any of the nucleotide sequences of (1)-(12) above.

In some other particular embodiments, the compositions provided by the present disclosure comprise a triple-antigen construct, or a vector comprising a triple-antigen construct, wherein the triple antigen construct comprises a nucleotide sequence selected from the group consisting of:

(1) a nucleotide sequence consisting of nucleotides 10-6009 of SEQ ID NO:42;

(2) a nucleotide sequence consisting of nucleotides 10-6003 of SEQ ID NO:44;

(3) a nucleotide sequence consisting of nucleotides 10-6024 of SEQ ID NO:46;

(4) a nucleotide sequence consisting of nucleotides 10-5988 of SEQ ID NO:48;

(5) a nucleotide sequence consisting of nucleotides 10-5829 of SEQ ID NO:50;

(6) a nucleotide sequence consisting of nucleotides 10-5829 of SEQ ID NO:52; and

(7) a nucleotide sequence that is a degenerate variant of any of the nucleotide sequences of (1)-(7 above.

In other particular embodiments, the compositions provided by the present disclosure comprises a RNA triple-antigen construct, or a RNA vector comprising a triple-antigen construct, wherein the triple antigen construct comprises a nucleotide sequence that corresponds to (1) any of the sequences of SEQ ID NOs:42, 44, 46, 48, 50, 52 or (2) a nucleotide sequence that is a degenerate variant of any of the nucleotide sequences of SEQ ID NOs:42, 44, 46, 48, 50, 52.

In other particular embodiments, the compositions provided by the present disclosure comprise a RNA triple-antigen construct, or a RNA vector comprising a triple-antigen construct, wherein the triple antigen construct consists of a nucleotide sequence that corresponds to (1) any of the sequences of SEQ ID NOs:42, 44, 46, 48, 50, 52 or (2) a nucleotide sequence that is a degenerate variant of any of the nucleotide sequences of SEQ ID NOs:42, 44, 46, 48, 50, 52.

In still other particular embodiments, the compositions provided by the present disclosure comprise a triple-antigen construct, or a vector comprising a triple-antigen construct, wherein the triple antigen construct comprises (1) a nucleotide sequence of any of SEQ ID NOS: 87, 88, 89, 90, 91, and 92 or (2) degenerate variant of a nucleotide sequence of any of SEQ ID NOS: 87, 88, 89, 90, 91, and 92. In some other particular embodiments, the present disclosure provides a composition comprising a plasmid, wherein the plasmid comprises a nucleotide sequence of any of SEQ ID Nos: 57, 59, 61, 63, 65, and 67. In still other particular embodiments, the present disclosure provides a composition comprising a vector, wherein the vector comprises a nucleotide sequence of any of SEQ ID Nos: 58, 60, 62, 64, 66, and 68.

In still other particular embodiments, the compositions provided by the present disclosure comprise a triple-antigen construct, or a vector comprising a triple-antigen construct, wherein the triple antigen construct consists of (1) a nucleotide sequence of any of SEQ ID NOS: 87, 88, 89, 90, 91, and 92 or (2) degenerate variant of a nucleotide sequence of any of SEQ ID NOS: 87, 88, 89, 90, 91, and 92. In some other particular embodiments, the present disclosure provides a composition comprising a plasmid, wherein the plasmid consists of a nucleotide sequence of any of SEQ ID Nos: 57, 59, 61, 63, 65, and 67. In still other particular embodiments, the present disclosure provides a composition comprising a vector, wherein the vector consists of a nucleotide sequence of any of SEQ ID Nos: 58, 60, 62, 64, 66, and 68.

The compositions, such as a pharmaceutical composition or a vaccine composition, may further comprise a pharmaceutically acceptable excipient. Pharmaceutically acceptable excipients for nucleic acid compositions, including DNA vaccine and RNA vaccine compositions, are well known to those skilled in the art. Such excipients may be aqueous or nonaqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides.

An immunogenic composition, pharmaceutical composition, or vaccine composition provided by the present disclosure may be used in conjunction or combination with one or more immune modulators. The composition may also be used in conjunction or combination with one or more adjuvants. Further, the composition may be used in conjunction or combination with one or more immune modulators and one or more adjuvants. The immune modulators or adjuvants may be formulated separately from the antigen construct or vector, or they may be part of the same composition formulation. Thus, in some embodiments, the present disclosure provides a pharmaceutical composition that comprises (1) an antigen construct provided by the present disclosure or vector containing such an antigen construct and (2) an immune modulator. In some further embodiments, the pharmaceutical composition further comprises an adjuvant. Examples of immune modulators and adjuvants are provided herein below.

The compositions, including vaccine compositions, can be prepared in any suitable dosage forms, such as liquid forms (e.g., solutions, suspensions, or emulsions) and solid forms (e.g., capsules, tablets, or powder), and by methods known to one skilled in the art.

### F. Uses of the Antigen Constructs, Vectors, and Compositions

In other aspects, the present disclosure provides (1) use of the antigen constructs, vectors, and compositions as medicament, (2) use of the antigen constructs, vectors, and compositions in the manufacture of a medicament for eliciting an immune response against a TAA, for inhibiting abnormal cell proliferation, or for treating a cancer, and (3) methods of using the antigen constructs, vectors, and compositions, wherein the antigen constructs, vectors, and compositions are as described herein above.

In one aspect, the present disclosure provides use of (1) an antigen construct encoding one or more immunogenic TAA polypeptides, (2) a vector containing such an antigen construct, or (3) a composition containing such as antigen-construct or vector for eliciting an immune response against a TAA in a mammal, such as a human. In some embodiments, the disclosure provides a method of eliciting an immune response against a TAA in a mammal, particularly a human, which method comprises administering to the mammal an effective amount of a composition comprising (1) an antigen construct encoding one or more immunogenic TAA polypeptides or (2) a vector containing an antigen construct encoding one or more immunogenic TAA polypeptides. In some embodiments, the disclosure provides a method of eliciting an immune response against CEA in a mammal, particularly a human, comprising administering to the mammal an effective amount of a composition comprising an antigen construct provided by the present disclosure, wherein the antigen construct comprises (1) at least one nucleotide sequence encoding an immunogenic CEA polypeptide and (2) at least one nucleotide sequence encoding an immunogenic MUC1 polypeptide or an immunogenic TERT polypeptide. In some other embodiments, the disclosure provides a method of eliciting an immune response against MUC1 in a mammal, particularly a human, comprising administering to the mammal an effective amount of a composition comprising an antigen construct provided by the present disclosure, wherein the antigen construct comprises (1) at least one nucleotide sequence encoding an immunogenic MUC1 polypeptide and (2) at least one nucleotide sequence encoding an immunogenic CEA polypeptide or an immunogenic TERT polypeptide. In some further embodiments, the disclosure provides a method of eliciting an immune response against TERT in a mammal, particularly a human, comprising administering to the mammal an effective amount of a composition comprising an antigen construct provided by the present disclosure, wherein the antigen construct comprises (1) at least one nucleotide sequence encoding an immunogenic TERT polypeptide and (2) at least one nucleotide sequence encoding an immunogenic MUC1 polypeptide or an immunogenic CEA polypeptide.

In another aspect, the present disclosure provides use of (1) an antigen construct encoding one or more immunogenic TAA polypeptides, (2) a vector containing such an antigen construct, or (3) a composition containing such as antigen-construct or vector for inhibiting abnormal cell proliferation in a mammal, such as a human. In some embodiments, the present disclosure provides a method of inhibiting abnormal cell proliferation in a mammal, particularly a human, comprising administering to the mammal an effective amount of a composition comprising (1) an antigen construct encoding one or more immunogenic TAA polypeptides or (2) a vector containing an antigen construct encoding one or more immunogenic TAA polypeptides, wherein the abnormal cell proliferation is associated with over-expression of the tumor-associated antigen CEA, MUC1, or TERT. The abnormal cell proliferation may be in any organ or tissues of a human, such as breast, stomach, ovaries, lungs, bladder, large intestine (e.g., colon and rectum), kidneys, pancreas, and prostate. In some embodiments, the method is for inhibiting abnormal cell proliferation in the breast, ovaries, pancreas, colon, lung, stomach, and rectum. The antigen construct or vector in the composition administered encodes at least one immunogenic polypeptide that is derived from, or immunogenic against, the over-expressed tumor-associated antigen. The antigen construct may be a single-antigen construct or a multi-antigen construct, such as a dual-antigen construct or a triple-antigen construct. In some specific embodiments, the composition comprises a triple-antigen construct encoding an immunogenic CEA polypeptide, an immunogenic MUC1 polypeptide, and an immunogenic TERT polypeptide.

In a further aspect, the present disclosure provides use of (1) an antigen construct encoding one or more immunogenic TAA polypeptides, (2) a vector containing such an antigen construct, or (3) a composition containing such as antigen-construct or vector as a medicament for treatment of a cancer in a mammal, particularly a human. In some embodiments, the present disclosure provides a method of treating a cancer in a human, wherein the cancer is associated with over-expression of one or more of the tumor-associated antigen CEA, MUC1, and TERT. The method comprises administering to the human an effective amount of a composition that comprises an antigen construct encoding at least one immunogenic polypeptide that is derived from, or immunogenic against, the over-expressed tumor-associated antigen in the particular cancer. The antigen construct may be a single-antigen construct or a multi-antigen construct, such as a dual-antigen construct or a triple-antigen construct. In some specific embodiments, the composition comprises a triple-antigen construct encoding an immunogenic CEA polypeptide, an immunogenic MUC1 polypeptide, and an immunogenic TERT polypeptide. Any cancer that over-expresses the tumor-associate antigen MUC1, CEA, and/or TERT may be treated by a method provided by the present disclosure. Examples of cancers include breast cancer, ovarian cancer, lung cancer (such as small cell lung cancer and non-small cell lung cancer), colorectal cancer, gastric cancer, and pancreatic cancer. In some particular embodiments, the present disclosure provide a method of treating cancer in a human, which comprises administering to the human an effective amount of a composition comprising a triple-antigen construct, wherein the cancer is (1) breast cancer, such as estrogen-receptor and/or progesterone-receptor positive breast cancer, HER2 positive breast cancer, or triple-negative breast cancer, (2) lung cancer, such as NSCLC or SCLC, (3) gastric cancer, (4) pancreatic cancer, or (5) colorectal cancer.

In some specific embodiments, the present disclosure provides a method of eliciting an immune response against a TAA, a method of inhibiting abnormal cell proliferation, or a method of treating a cancer in a mammal, particularly a human, which method comprises administering to the mammal an effective amount of a composition comprising a multi-antigen construct or vector comprising a multi-antigen construct, wherein the multi-antigen construct comprises a nucleotide sequence encoding any of the amino acid sequence of SEQ ID Nos: 43, 45, 47, 49, 51, and 53. In other specific embodiments, the present disclosure provides a method of eliciting an immune response against a TAA, a method of inhibiting abnormal cell proliferation, or a method of treating a cancer in a mammal, particularly a human, which method comprises administering to the mammal an effective amount of a composition comprising a multi-antigen construct, wherein the multi-antigen construct comprises a nucleotide sequence of any of SEQ ID Nos: 42, 44, 46, 48, 50, 52, and 87-92. In other specific embodiments, the present disclosure provides a method of eliciting an immune response against a TAA, a method of inhibiting abnormal cell proliferation, or a method of treating a cancer in a mammal, particularly a human, which method comprises administering to the mammal an effective amount of a composition comprising a vector, wherein the vector comprises a nucleotide sequence of any of SEQ ID Nos: 57-68.

The compositions can be administered to a mammal, including human, by a number of suitable methods known in the art. Examples of suitable methods include: (1) intramuscular, intradermal, intraepidermal, or subcutaneous administration, (2) oral administration, and (3) topical application (such as ocular, intranasal, and intravaginal application). One particular method of intradermal or intraepidermal administration of a nucleic acid vaccine composition, particularly composition containing a DNA plasmid, is gene gun delivery using the Particle Mediated Epidermal Delivery (PMED™) vaccine delivery device marketed by PowderMed. PMED is a needle-free method of administering vaccines to animals or humans. The PMED system involves the precipitation of DNA onto microscopic gold particles that are then propelled by helium gas into the epidermis. The DNA-coated gold particles are delivered to the APCs and keratinocytes of the epidermis, and once inside the nuclei of these cells, the DNA elutes off the gold and becomes transcriptionally active, producing encoded protein. Another particular method for intramuscular administration of a nucleic acid vaccine involves electroporation. Electroporation uses controlled electrical pulses to create temporary pores in the cell membrane, which facilitates cellular uptake of the nucleic acid vaccine injected into the muscle. Where a CpG is used in combination with a nucleic acid vaccine, the CpG and nucleic acid vaccine may be co-formulated in one formulation and the formulation is administered intramuscularly by electroporation.

The effective amount of the composition to be administered in a given method can be readily determined by a person skilled in the art and will depend on a number of factors. In a method of treating cancer, such as pancreatic cancer, ovarian cancer, lung cancer, colorectal cancer, gastric cancer, and breast cancer, factors that may be considered in determining the effective amount include the subject to be treated, including the subject's immune status and health, the severity or stage of the cancer to be treated, the specific immunogenic TAA polypeptides expressed, the degree of protection or treatment desired, the administration method and schedule, and other therapeutic agents (such as adjuvants or immune modulators) used. The method of formulation and delivery are among the key factors for determining the dose of the nucleic acid required to elicit an effective immune response. For example, the effective amounts of the nucleic acid in a vaccine may be in the range of 2 μg/dose-10 mg/dose when the vaccine is formulated as an aqueous solution and administered by hypodermic needle injection or pneumatic injection, whereas only 16 ng/dose-16 μg/dose may be required when the nucleic acid is prepared as coated gold beads and delivered using a gene gun technology. The dose range for a nucleic acid in a vaccine by electroporation is generally in the range of 0.5-10 mg/dose. In the case where the nucleic acid vaccine is administered together with a CpG by electroporation in a co-formulation, the dose of the nucleic acid vaccine may be in the range of 0.5-5 mg/dose and the dose of CpG is typically in the range of 0.05 mg-5 mg/dose, such as 0.05, 0.2, 0.6, or 1.2 mg/dose per person.

The vaccine compositions provided by the present disclosure can be used in a prime-boost strategy to induce robust and long-lasting immune response. Priming and boosting vaccination protocols based on repeated injections of the same immunogenic construct are well known. In general, the first dose of the vaccine may not be able to produce protective immunity, but only “primes” the immune system. A protective immune response develops after the second, third, or subsequent doses (the “boosts”). The boosts are performed according to conventional techniques, and can be further optimized empirically in terms of schedule of administration, route of administration, choice of adjuvant, dose, and potential sequence when administered with another vaccine. In one embodiment, the vaccine compositions are used in a conventional homologous prime-boost strategy, in which the same vaccine is administered to the animal in both the prime and boosts doses. For example, the same vaccine composition containing a plasmid vector is administered in both the initial doses (“prime’) and subsequent doses (“boost”). In another embodiment, the vaccine compositions are used in a heterologous prime-boost vaccination, in which different types of vaccines expressing the same immunogenic TAA polypeptide(s) are administered at predetermined time intervals. For example, an antigen construct is administered in the form of a plasmid vector in the prime dose and in the form of a viral vector in the boost doses, or vice versa.

The vaccine compositions may be used together with one or more adjuvants. Examples of suitable adjuvants include: (1) oil-in-water emulsion formulations, such as MF59 and AS03, (2) saponin adjuvants, such as QS21 and Iscomatrix® (Commonwealth Serum Laboratories, Australia); (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12, interferons (e.g. gamma interferon), macrophage colony stimulating factor (M-CSF), and tumor necrosis factor (TNF); (5) monophosphoryl lipid A (MPL) or 3-O-deacylated MPL (3dMPL); (6) oligonucleotides comprising CpG motifs; and (7) metal salt, including aluminum salts (alum), such as aluminum phosphate and aluminum hydroxide.

Further, for the treatment of a neoplastic disorder, including a cancer, in a mammal, including human, the compositions may be administered in combination with one or more immune modulators. The immune modulator may be an immune-suppressive-cell inhibitor (ISC inhibitor) or an immune-effector-cell enhancer (IEC enhancer). Further, one or more ISC inhibitors may be used in combination with one or more IEC enhancers. The immune modulators may be administered by any suitable methods and routes, including (1) systemic administration such as intravenous, intramuscular, or oral administration, and (2) local administration such intradermal and subcutaneous administration. Where appropriate or suitable, local administration is generally preferred over systemic administration. Local administration of any immune modulators can be carried out at any location of the body of the mammal that is suitable for local administration of pharmaceuticals; however, it is more preferable that these immune modulators are administered locally at close proximity to the vaccine draining lymph node.

The compositions, such as a vaccine, may be administered simultaneously or sequentially with any or all of the immune modulators used. Similarly, when two or more immune modulators are used, they may be administered simultaneously or sequentially with respect to each other. In some embodiments, a vaccine is administered simultaneously (e.g., in a mixture) with respect to one immune modulator, but sequentially with respect to one or more additional immune modulators. Co-administration of the vaccine and the immune modulators can include cases in which the vaccine and at least one immune modulator are administered so that each is present at the administration site, such as vaccine draining lymph node, at the same time, even though the antigen and the immune modulators are not administered simultaneously. Co-administration of the vaccine and the immune modulators also can include cases in which the vaccine or the immune modulator is cleared from the administration site, but at least one cellular effect of the cleared vaccine or immune modulator persists at the administration site, such as vaccine draining lymph node, at least until one or more additional immune modulators are administered to the administration site. In cases where a nucleic acid vaccine is administered in combination with a CpG, the vaccine and CpG may be contained in a single formulation and administered together by any suitable method. In some embodiments, the nucleic acid vaccine and CpG in a co-formulation (mixture) is administered by intramuscular injection in combination with electroporation.

In some embodiments, the immune modulator is an ISC inhibitor. Examples of ISC inhibitors include (1) protein kinase inhibitors, such as imatinib, sorafenib, lapatinib, BIRB-796, and AZD-1152, AMG706, Zactima (ZD6474), MP-412, sorafenib (BAY 43-9006), dasatinib, CEP-701 (lestaurtinib), XL647, XL999, Tykerb (lapatinib), MLN518, (formerly known as CT53518), PKC412, ST1571, AEE 788, OSI-930, OSI-817, sunitinib malate (Sutent), axitinib (AG-013736), erlotinib, gefitinib, axitinib, bosutinib, temsirolismus and nilotinib (AMN107). In some particular embodiments, the tyrosine kinase inhibitor is sunitinib, sorafenib, or a pharmaceutically acceptable salt or derivative (such as a malate or a tosylate) of sunitinib or sorafenib; (2) cyclooxygenase-2 (COX-2) inhibitors, such as celecoxib and rofecoxib; (3) phosphodiesterase type 5 (PDES) inhibitors, such as avanafil, lodenafil, mirodenafil, sildenafil, tadalafil, vardenafil, udenafil, and zaprinast, (4) DNA crosslinkers, such as cyclophosphamide, (5) PARP inhibitors, such as talazoparib, and (6) CDK inhibitors, such palbocyclib.

In some embodiments, the immune modulator that is used in combination with a nucleic acid composition is an IEC enhancer. Two or more IEC enhancers may be used together. Examples of IEC enhancers that may be used include: (1) TNFR agonists, such as agonists of OX40, 4-1BB (such as BMS-663513), GITR (such as TRX518), and CD40 (such as CD40 agonistic antibodies); (2) CTLA-4 inhibitors, such as is Ipilimumab and Tremelimumab; (3) TLR agonists, such as CpG 7909 (5′ TCGTCGTTTTGTCGTTTTGTCGTT3′), CpG 24555 (5′ TCGTCGTTTTTCGGTGCTTTT3′ (CpG 24555); and CpG 10103 (5′ TCGTCGTTTTTCGGTCGTTTT3′); (4) programmed cell death protein 1 (PD-1) inhibitors, such as nivolumab and pembrolizumab; and (5) PD-L1 inhibitors, such as atezolizumab, durvalumab, and velumab; and (6) 001 inhibitors.

In some embodiments, the IEC enhancer is CD40 agonist antibody, which may be a human, humanized or part-human chimeric anti-CD40 antibody. Examples of specific CD40 agonist antibodies include the G28-5, mAb89, EA-5 or S2C6 monoclonal antibody, and CP870,893. CP-870,893 is a fully human agonistic CD40 monoclonal antibody (mAb) that has been investigated clinically as an anti-tumor therapy. The structure and preparation of CP870,893 is disclosed in WO2003041070 (where the antibody is identified by the internal identified “21.4.1” and the amino acid sequences of the heavy chain and light chain of the antibody are set forth in SEQ ID NO: 40 and SEQ ID NO: 41, respectively). For use in combination with a composition present disclosure, CP-870,893 may be administered by any suitable route, such as intradermal, subcutaneous, or intramuscular injection. The effective amount of CP870893 is generally in the range of 0.01-0.25 mg/kg. In some embodiment, CP870893 is administered at an amount of 0.05-0.1 mg/kg.

In some other embodiments, the IEC enhancer is a CTLA-4 inhibitor, such as Ipilimumab and Tremelimumab. Ipilimumab (also known as MEX-010 or MDX-101), marketed as YERVOY, is a human anti-human CTLA-4 antibody. Ipilimumab can also be referred to by its CAS Registry No. 477202-00-9, and is disclosed as antibody 10DI in PCT Publication No. WO 01/14424. Tremelimumab (also known as CP-675,206) is a fully human IgG2 monoclonal antibody and has the CAS number 745013-59-6. Tremelimumab is disclosed in U.S. Pat. No. 6,682,736, incorporated herein by reference in its entirety, where it is identified as antibody 11.2.1 and the amino acid sequences of its heavy chain and light chain are set forth in SEQ ID NOs:42 and 43, respectively. For use in combination with a composition provided by the present disclosure, Tremelimumab may be administered locally, particularly intradermally or subcutaneously. The effective amount of Tremelimumab administered intradermally or subcutaneously is typically in the range of 5-200 mg/dose per person. In some embodiments, the effective amount of Tremelimumab is in the range of 10-150 mg/dose per person per dose. In some particular embodiments, the effective amount of Tremelimumab is about 10, 25, 50, 75, 100, 125, 150, 175, or 200 mg/dose per person.

In some other embodiments, the immune modulator is a PD-1 inhibitor or PD-L1 inhibitor. Examples of PD-1 inhibitors include nivolumab (trade name Opdivo), pembrolizumab (trade name Keytruda), RN888 (anti-PD-1 antibody), pidilizumab (Cure Tech, AMP-224 (GSK), AMP-514 (GSK), and PDR001 (Novartis). Examples of PD-L1 inhibits include atezolizumab (PD-L1-specific mAbs; trade name Tecentriq), durvalumab (PD-L1-specific mAbs; trade name Imfinzi), and avelumab (PD-L1-specific mAbs; trade name Bavencio), and BMS-936559 (BMS). See also Okazaki T et al., International Immunology (2007); 19,7:813-824 and Sunshine J et al., Curr Opin Pharmacol. 2015 August; 23:32-8). In some specific embodiment, the PD-1 inhibitor is RN888. RN888 is a monoclonal antibody that specifically binds to PD-1. RN888 is disclosed in international patent application publication WO2016/092419, in which the antibody is identified as mAb7 having a full-length heavy chain amino acid sequence of SEQ ID NO:29 and full-length light chain amino acid sequence of SEQ ID NO:39.

In other embodiments, the immune modulator is an inhibitor of indoleamine 2,3-dioxygenase 1 (also known as “IDO1”). IDO1 was found to modulate immune cell function to a suppressive phenotype and was, therefore, believed to partially account for tumor escape from host immune surveillance. The enzyme degrades the essential amino acid tryptophan into kynurenine and other metabolites. It was found that these metabolites and the paucity of tryptophan leads to suppression of effector T-cell function and augmented differentiation of regulatory T cells. The 001 inhibitors may be large molecules, such as an antibody, or a small molecule, such as a chemical compound.

In some particular embodiments, the polypeptide or nucleic acid composition provided by the present disclosure is used in combination with a 1,2,5-oxadiazole derivative 001 inhibitor disclosed in WO2010/005958. Examples of specific 1,2,5-oxadiazole derivative IDO1 inhibitors include the following compounds:
4-({2-[(aminosulfonyl)amino]ethyl}amino)-N-(3-bromo-4-fluorophenyl)-N′-hydroxy-1,2,5-oxadiazole-3-carboximidamide;
4-({2 [(aminosulfonyl)amino]ethyl} amino)-N-(3-chloro-4-fluorophenyl)-N′-hydroxy-1,2,5-oxadiazole 3-carboximidamide;
4-({2 [(aminosulfonyl)amino] ethyl} amino)-N-[4-fluoro-3-(trifluoromethyl)phenyl]-N′-hydroxy-1,2,5 oxadiazole-3-carboximidamide;
4-({2 [(aminosulfonyl)amino] ethyl} amino)-N′-hydroxy-N-[3-(trifluoromethyl)phenyl]-1,2,5 oxadiazole-3-carboximidamide;
4-({2 [(aminosulfonyl)amino]ethyl} amino)-N-(3-cyano-4-fluorophenyl)-N′-hydroxy-1,2,5-oxadiazole 3-carboximidamide;
4-({2 [(aminosulfonyl)amino] ethyl} amino)-N-[(4-bromo-2-furyl)methyl]-N′-hydroxy-1,2,5 oxadiazole-3-carboximidamide; or
4-({2 [(aminosulfonyl)amino] ethyl} amino)-N-[(4-chloro-2-furyl)methyl]-N′-hydroxy-1,2,5 oxadiazole-3-carboximidamide.

The 1,2,5-oxadiazole derivative IDO1 inhibitors are typically administered orally once or twice per day and effective amount by oral administration is generally in the range of 25 mg-1000 mg per dose per patient, such as 25 mg, 50 mg, 100 mg, 200 mg, 300 mg, 400 mg, 500 mg, 600 mg, 700 mg, 800 mg, or 1000 mg. In a particular embodiment, the polypeptide or nucleic acid composition provided by the present disclosure is used in combination with 4-({2-[(aminosulfonyl)amino]ethyl}amino)-N-(3-bromo-4-fiuorophenyl)-N′-hydroxy-1,2,5-oxadiazole-3-carboximidamide administered orally twice per day at 25 mg or 50 mg per dose. The 1,2,5-oxadiazole derivatives may be synthesized as described in U.S. Pat. No. 8,088,803, which is incorporated herein by reference in its entirety.

In some other specific embodiments, the polypeptide or nucleic acid composition provided by the present disclosure is used in combination with a pyrrolidine-2,5-dione derivative 001 inhibitor disclosed in WO2015/173764. Examples of specific pyrrolidine-2,5-dione derivative inhibitors include the following compounds:
3-(5-fluoro-1H-indol-3-yl)pyrrolidine-2,5-dione,
(3-²H)-3-(5-fluoro-1H-indol-3-yl)pyrrolidine-2,5-dione;
(−)-(R)-3-(5-fluoro-1H-indol-3-yl)pyrrolidine-2,5-dione,
3-(1H-indol-3-yl)pyrrolidine-2,5-dione,
(−)-(R)-3-(1H-indol-3-yl)pyrrolidine-2,5-dione,
3-(5-chloro-1H-indol-3-yl)pyrrolidine-2,5-dione;
(−)-(R)-3-(5-chloro-1H-indol-3-yl)pyrrolidine-2,5-dione,
3-(5-bromo-1H-indol-3-yl)pyrrolidine-2,5-dione,
3-(5,6-difluoro-1H-indol-3-yl)pyrrolidine-2,5-dione; and
3-(6-chloro-1H-indol-3-yl)pyrrolidine-2,5-dione.

The pyrrolidine-2,5-dione derivative 001 inhibitors are typically administered orally once or twice per day and the effective amount by oral administration is generally in the range of 50 mg-1000 mg per dose per patient, such as 125 mg, 250 mg, 500 mg, 750 mg, or 1000 mg. In a particular embodiment, the polypeptide or nucleic acid composition provided by the present disclosure is used in combination with 3-(5-fluoro-1H-indol-3-yl)pyrrolidine-2,5-dione administered orally once per day at 125-100 mg per dose per patient. The pyrrolidine-2,5-dione derivatives may be synthesized as described in U.S. patent application publication US2015329525, which is incorporated herein by reference in its entirety.

### G. Examples

The following examples are provided to illustrate certain embodiments of the invention. They should not be construed to limit the scope of the invention in any way. From the discussion above and these examples, one skilled in the art can ascertain the essential characteristics of the invention and, without departing from the spirit and scope thereof, can make various changes and modifications of the invention to adapt it to various usage and conditions.

### Example 1. Construction of Plasmids Containing a Single-Antigen Construct or Multi-Antigen Construct

Example 1 illustrates the construction of plasmid vectors containing a single-antigen construct, a dual-antigen construct, or a triple antigen construct. Unless as otherwise noted, reference to amino acid positions or residues of MUC1, CEA, and TERT protein refers to the amino acid sequence of human MUC1 isoform 1 precursor protein as set forth in SEQ ID NO:1, the amino acid sequence of human carcinoembryonic antigen (CEA) isoform 1 precursor protein as set forth in SEQ ID NO:2, and the amino acid sequence of human TERT isoform 1 precursor protein as set forth in SEQ ID NO:3, respectively. Structures of some of the primers used in the plasmid constructions are provided in Table 16.

1A. Plasmids Containing a Single-Antigen Construct

Plasmid 1027 (MUC1).

Plasmid 1027 was generated using the techniques of gene synthesis and restriction fragment exchange. The amino acid sequence of human MUC1 with a 5× tandem repeat VNTR region was submitted to GeneArt for gene optimization and synthesis. The gene encoding the polypeptide was optimized for expression, synthesized, and cloned. The MUC-1 open reading frame was excised from the GeneArt vector by digestion with NheI and BglII and inserted into similarly digested plasmid pPJV7563. The open reading frame (ORF) nucleotide sequence of Plasmid 1027 is set forth in SEQ ID NO:4. The amino acid sequence encoded by Plasmid 1027 is set for in SEQ ID NO:5.

Plasmid 1361 (CEA).

Plasmid 1361 was constructed using the techniques of gene synthesis, PCR and Seamless cloning. First, the gene encoding the CEA reference sequence was codon optimized for expression at DNA2.0. The sequence encoding amino acids 2-702 was amplified by PCR with primers ID1361-1362_PCRF and ID1361-1362_PCRR. The amplicon was cloned into the NheI/Bgl II sites of pPJV7563 by Seamless cloning. The open reading frame nucleotide sequence of Plasmid 1361 is set forth in SEQ ID NO:14. The amino acid sequence encoded by Plasmid 1361 is set for in SEQ ID NO:15.

Plasmid 1386 (mCEA).

Plasmid 1386, which encodes a membrane-bound immunogenic CEA polypeptide (mCEA), was constructed using the techniques of PCR and Seamless cloning. First, the gene fragment encoding CEA amino acids 2-144 was amplified by PCR from plasmid 1361 with primers f pmed CEA SS and r CEA D1. Second, the gene fragment encoding CEA amino acids 323-702 was amplified by PCR from plasmid 1361 with primers f CEA D1-D4 and r pmed CEA GPI. The amplicons were ligated and cloned into the Nhe I/Bgl II sites of pPJV7563 by Seamless cloning. The open reading frame nucleotide sequence of Plasmid 1386 is set forth in SEQ ID NO:16. The amino acid sequence encoded by Plasmid 1386 is set for in SEQ ID NO:17.

Plasmid 1390 (cCEA).

Plasmid 1390, which encodes a cytoplasmic immunogenic CEA polypeptide (cCEA), was constructed using the techniques of PCR and Seamless cloning. First, the gene fragment encoding CEA amino acids 35-144 was amplified by PCR from plasmid 1361 with primers f pmed CEA D1 and r CEA D1. Second, the gene fragment encoding CEA amino acids 323-677 was amplified by PCR from plasmid 1361 with primers f CEA D1-D4 and r pmed CEA D7. The amplicons were ligated and cloned into the Nhe I/Bgl II sites of pPJV7563 by Seamless cloning. The open reading frame nucleotide sequence of Plasmid 1390 is set forth in SEQ ID NO:18. The amino acid sequence encoded by Plasmid 1390 is set for in SEQ ID NO:19.

Plasmid 1065 (Full Length TERT D712A/V713I).

Plasmid 1065 was generated using the techniques of gene synthesis and restriction fragment exchange. The amino acid sequence of human TERT with two mutations (D712A/V713I) designed to inactivate enzymatic activity was submitted to DNA2.0 for gene optimization and synthesis. The gene encoding the polypeptide was optimized for expression, synthesized, and cloned. The TERT open reading frame was excised from the DNA2.0 vector by digestion with NheI and BglII and inserted into similarly digested plasmid pPJV7563. The amino acid sequence encoded by Plasmid 1065 is set for in SEQ ID NO:81. The open reading frame (ORF) nucleotide sequence of Plasmid 1065 is set forth in SEQ ID NO:82.

Plasmid 1112 (TERT240).

Plasmid 1112 was constructed using the techniques of PCR and Seamless cloning. First, the gene encoding TERT amino acids 241-1132 was amplified by PCR from plasmid 1065 with primers f pmed TERT 241G and r TERT co# pMed. The amplicon was cloned into the Nhe I/Bgl II sites of pPJV7563 by Seamless cloning. The open reading frame nucleotide sequence of Plasmid 1112 is set forth in SEQ ID NO:8. The amino acid sequence encoded by Plasmid 1112 is set for in SEQ ID NO:9.

Plasmid 1197 (cMUC1).

Plasmid 1197, which encodes a cytoplasmic immunogenic MUC1 polypeptide (cMUC1), was constructed using the techniques of PCR and Seamless cloning. First, the gene encoding MUC1 amino acids 22-225, 946-1255 was amplified by PCR from plasmid 1027 with primers ID1197F and ID1197R. The amplicon was cloned into the Nhe I/Bgl II sites of pPJV7563 by Seamless cloning. The open reading frame nucleotide sequence of Plasmid 1197 is set forth in SEQ ID NO:6. The amino acid sequence encoded by Plasmid 1197 is set for in SEQ ID NO:7.

Plasmid 1326 (TERT343).

Plasmid 1326 was constructed using the techniques of PCR and Seamless cloning. First, the gene encoding TERT amino acids 344-1132 was amplified by PCR from plasmid 1112 with primers TertΔ343-F and Tert-R. The amplicon was cloned into the Nhe I/Bgl II sites of pPJV7563 by Seamless cloning. The open reading frame nucleotide sequence of Plasmid 1326 is set forth in SEQ ID NO:10. The amino acid sequence encoded by Plasmid 1326 is set for in SEQ ID NO:11.

Plasmid 1330 (TERT541).

Plasmid 1330 was constructed using the techniques of PCR and Seamless cloning. First, the gene encoding TERT amino acids 542-1132 was amplified by PCR from plasmid 1112 with primers TertΔ541-F and Tert-R. The amplicon was cloned into the Nhe I/Bgl II sites of pPJV7563 by Seamless cloning. The open reading frame nucleotide sequence of Plasmid 1330 is set forth in SEQ ID NO:12. The amino acid sequence encoded by Plasmid 1330 is set for in SEQ ID NO:13.

1B. Plasmids Containing a Dual-Antigen Construct

Plasmid 1269 (Muc1-Tert240).

Plasmid 1269 was constructed using the techniques of PCR and Seamless cloning. First, the gene encoding the human telomerase amino acids 241-1132 was amplified by PCR from plasmid 1112 with primers f tg link Ter240 and r pmed Bgl Ter240. The gene encoding human Mucin-1 amino acids 2-225, 946-1255 was amplified by PCR from plasmid 1027 with primers f pmed Nhe Muc and r link muc. PCR resulted in the addition of an overlapping GGSGG linker at the 5′ end of Tert and 3′ end of Muc1. The amplicons were mixed together and cloned into the Nhe I/Bgl II sites of pPJV7563 by Seamless cloning. The open reading frame nucleotide sequence of Plasmid 1269 is set forth in SEQ ID NO:20. The amino acid sequence encoded by Plasmid 1269 is set for in SEQ ID NO:21.

Plasmid 1270 (Muc1-ERB2A-Tert240).

Plasmid 1270 was constructed using the techniques of PCR and Seamless cloning. First, the gene encoding the human telomerase amino acids 241-1132 was amplified by PCR from plasmid 1112 with primers f2 ERBV2A, f1 ERBV2A Ter240, and r pmed Bgl Ter240. The gene encoding human Mucin-1 amino acids 2-225, 946-1255 was amplified by PCR from plasmid 1027 with primers f pmed Nhe Muc and r ERB2A Bamh Muc. PCR resulted in the addition of overlapping ERBV 2A sequences at the 5′ end of Tert and 3′ end of Muc1. The amplicons were mixed together and cloned into the Nhe I/Bgl II sites of pPJV7563 by Seamless cloning. The open reading frame nucleotide sequence of Plasmid 1270 is set forth in SEQ ID NO:22. The amino acid sequence encoded by Plasmid 1270 is set for in SEQ ID NO:23.

Plasmid 1271 (Tert240-ERB2A-Muc1).

Plasmid 1271 was constructed using the techniques of PCR and Seamless cloning. First, the gene encoding the human telomerase amino acids 241-1132 was amplified by PCR from plasmid 1112 with primers f pmed Nhe Ter240 and r ERB2A Bamh Ter240. The gene encoding human Mucin-1 amino acids 2-225, 946-1255 was amplified by PCR from plasmid 1027 with primers f2 ERBV2A, f1 ERBV2A Muc, and r pmed Bgl Muc. PCR resulted in the addition of overlapping ERBV 2A sequences at the 3′ end of Tert and 5′ end of Muc1. The amplicons were mixed together and cloned into the Nhe I/Bgl II sites of pPJV7563 by Seamless cloning. The open reading frame nucleotide sequence of Plasmid 1271 is set forth in SEQ ID NO:24. The amino acid sequence encoded by Plasmid 1271 is set for in SEQ ID NO:25.

Plasmid 1286 (cMuc1-ERB2A-Tert240).

Plasmid 1286 was constructed using the techniques of PCR and Seamless cloning. First, the gene encoding the human telomerase amino acids 241-1132 was amplified by PCR from plasmid 1112 with primers f2 ERBV2A, f1 ERBV2A Ter240, and r pmed Bgl Ter240. The gene encoding human Mucin-1 amino acids 22-225, 946-1255 was amplified by PCR from plasmid 1197 with primers f pmed Nhe cytMuc and r ERB2A Bamh Muc. PCR resulted in the addition of overlapping ERBV 2A sequences at the 5′ end of Tert and 3′ end of Muc1. The amplicons were mixed together and cloned into the Nhe I/Bgl II sites of pPJV7563 by Seamless cloning. The open reading frame nucleotide sequence of Plasmid 1286 is set forth in SEQ ID NO:26. The amino acid sequence encoded by Plasmid 1286 is set for in SEQ ID NO:27.

Plasmid 1287 (Tert240-ERB2A-cMuc1).

Plasmid 1287 was constructed using the techniques of PCR and Seamless cloning. First, the gene encoding the human telomerase amino acids 241-1132 was amplified by PCR from plasmid 1112 with primers f pmed Nhe Ter240 and r ERB2A Bamh Ter240. The gene encoding human Mucin-1 amino acids 22-225, 946-1255 was amplified by PCR from plasmid 1197 with primers f2 ERBV2A, f1 ERBV2A cMuc, and r pmed Bgl Muc. PCR resulted in the addition of overlapping ERBV 2A sequences at the 3′ end of Tert and 5′ end of Muc1. The amplicons were mixed together and cloned into the Nhe I/Bgl II sites of pPJV7563 by Seamless cloning. The open reading frame nucleotide sequence of Plasmid 1287 is set forth in SEQ ID NO:28. The amino acid sequence encoded by Plasmid 1287 is set for in SEQ ID NO: 29.

Plasmid 1409 (Muc1-EMC2A-mCEA).

Plasmid 1409 was constructed using the techniques of PCR and Seamless cloning. First, the gene encoding human Mucin-1 amino acids 2-225, 946-1255 was amplified by PCR from plasmid 1027 with primers f pmed Nhe Muc and r EM2A Bamh Muc. The gene encoding CEA amino acids 2-144, 323-702 was amplified by PCR from plasmid 1386 with primers f2 EMCV2A, f1 EMC2a CEAss, and r pmed CEA GPI. PCR resulted in the addition of overlapping EMCV 2A sequences at the 5′ end of CEA and 3′ end of Muc1. The amplicons were mixed together and cloned into the Nhe I/Bgl II sites of pPJV7563 by Seamless cloning. The open reading frame nucleotide sequence of Plasmid 1409 is set forth in SEQ ID NO:30. The amino acid sequence encoded by Plasmid 1409 is set for in SEQ ID NO:31.

Plasmid 1410 (mCEA-T2A-Muc1).

Plasmid 1410 was constructed using the techniques of PCR and Seamless cloning. First, the gene encoding CEA amino acids 2-144, 323-702 was amplified by PCR from plasmid 1386 with primers f pmed CEA SS and r T2A CEA. The gene encoding human Mucin-1 amino acids 2-225, 946-1255 was amplified by PCR from plasmid 1027 with primers f2 T2A 63, f1 T2a Muc, and r pmed Bgl Muc. PCR resulted in the addition of overlapping T2A sequences at the 3′ end of CEA and 5′ end of Muc1. The amplicons were mixed together and cloned into the Nhe I/Bgl II sites of pPJV7563 by Seamless cloning. The open reading frame nucleotide sequence of Plasmid 1410 is set forth in SEQ ID NO:32. The amino acid sequence encoded by Plasmid 1410 is set for in SEQ ID NO:33.

Plasmid 1411 (mCEA-Furin-T2A-Muc1). Plasmid 1411 was constructed using the techniques of PCR and Seamless cloning. First, the gene encoding CEA amino acids 2-144, 323-702 was amplified by PCR from plasmid 1386 with primers f pmed CEA SS and r T2A furin CEA. The gene encoding human Mucin-1 amino acids 2-225, 946-1255 was amplified by PCR from plasmid 1027 with primers f2 T2A 63, f1 T2a Muc, and r pmed Bgl Muc. PCR resulted in the addition of overlapping furin cleavage site and T2A sequences at the 3′ end of CEA and 5′ end of Muc1. The amplicons were mixed together and cloned into the Nhe I/Bgl II sites of pPJV7563 by Seamless cloning. The open reading frame nucleotide sequence of Plasmid 1411 is set forth in SEQ ID NO:34. The amino acid sequence encoded by Plasmid 1411 is set for in SEQ ID NO:35.

Plasmid 1431 (Muc1-EMC2A-cCEA).

Plasmid 1431 was constructed using the techniques of PCR and Seamless cloning. First, the gene encoding human Mucin-1 amino acids 2-225, 946-1255 was amplified by PCR from plasmid 1027 with primers f pmed Nhe Muc and r EM2A Bamh Muc. The gene encoding CEA amino acids 35-144, 323-677 was amplified by PCR from plasmid 1390 with primers f2 EMCV2A, f EMC2a CEA d1, and r pmed CEA D7. PCR resulted in the addition of overlapping EMCV 2A sequences at the 5′ end of CEA and 3′ end of Muc1. The amplicons were mixed together and cloned into the Nhe I/Bgl II sites of pPJV7563 by Seamless cloning. The open reading frame nucleotide sequence of Plasmid 1431 is set forth in SEQ ID NO:36. The amino acid sequence encoded by Plasmid 1431 is set for in SEQ ID NO:37.

Plasmid 1432 (cCEA-T2A-Tert240). Plasmid 1432 was constructed using the techniques of PCR and Seamless cloning. First, the gene encoding the CEA amino acids 35-144, 323-677 was amplified by PCR from plasmid 1390 with primers f pmed CEA D1 and r T2a CEA D7. The gene encoding human telomerase amino acids 241-1132 was amplified by PCR from plasmid 1112 with primers f2 T2A 63, f1 T2A Tert240, and r pmed Bgl Ter240. The PCR resulted in the addition of overlapping TAV 2A sequences at the 5′ end of Tert and 3′ end of CEA. The amplicons were mixed together and cloned into the Nhe I/Bgl II sites of pPJV7563 by Seamless cloning. The open reading frame nucleotide sequence of Plasmid 1432 is set forth in SEQ ID NO:38. The amino acid sequence encoded by Plasmid 1432 is set for in SEQ ID NO:39.

Plasmid 1440 (Tert240-ERA2A-mCEA). Plasmid 1440 was constructed using the techniques of PCR and Seamless cloning. First, the gene encoding human telomerase amino acids 241-1132 was amplified by PCR from plasmid 1112 with primers f pmed Nhe tert240 and r ERA2A Tert. The gene encoding CEA amino acids 2-144, 323-702 was amplified by PCR from plasmid 1386 with primers f2 ERAV2A, f1 ERA2A ssCEA, and r pmed CEA GPI. The PCR resulted in the addition of overlapping ERAV 2A sequences at the 3′ end of Tert and 5′ end of CEA. The amplicons were mixed together and cloned into the Nhe I/Bgl II sites of pPJV7563 by Seamless cloning. The open reading frame nucleotide sequence of Plasmid 1440 is set forth in SEQ ID NO:40. The amino acid sequence encoded by Plasmid 1440 is set for in SEQ ID NO:41.

1C. Plasmids Containing a Triple-Antigen Construct

Plasmid 1424 (Muc1-ERB2A-Tert240-ERA2A-mCEA). Plasmid 1424 was constructed using the techniques of PCR and Seamless cloning. First, the genes encoding human Mucin-1 amino acids 2-225, 946-1255, an ERBV 2A peptide, and the amino terminal half of human Tert240 were amplified by PCR from plasmid 1270 with primers f pmed Nhe Muc and r tert 1602-1579. The genes encoding the carboxy terminal half of Tert240, an ERAV 2A peptide, and human CEA amino acids 2-144, 323-702 were amplified by PCR from plasmid 1440 with primers f tert 1584-1607 and r pmed CEA GPI. The partially overlapping amplicons were digested with Dpn I, mixed together, and cloned into the Nhe I/Bgl II sites of pPJV7563 by Seamless cloning. The open reading frame nucleotide sequence of Plasmid 1424 is set forth in SEQ ID NO:42. The amino acid sequence encoded by Plasmid 1424 is set for in SEQ ID NO:43.

Plasmid 1425 (mCEA-T2A-Muc1-ERB2A-Tert240). Plasmid 1425 was constructed using the techniques of PCR and Seamless cloning. First, the genes encoding human CEA amino acids 2-144, 323-702, a TAV 2A peptide, and the amino terminal half of human Mucin-1 were amplified by PCR from plasmid 1410 with primers f pmed CEA SS and r muc 986-963. The genes encoding the carboxy terminal half of human Mucin-1, an ERBV 2A peptide, and human telomerase amino acids 241-1132 were amplified by PCR from plasmid 1270 with primers f Muc 960-983 and r pmed Bgl Ter240. The partially overlapping amplicons were digested with Dpn I, mixed together, and cloned into the Nhe I/Bgl II sites of pPJV7563 by Seamless cloning. The open reading frame nucleotide sequence of Plasmid 1425 is set forth in SEQ ID NO:44. The amino acid sequence encoded by Plasmid 1425 is set for in SEQ ID NO:45.

Plasmid 1426 (Tert240-ERB2A-Muc1-EMC2A-mCEA). Plasmid 1426 was constructed using the techniques of PCR and Seamless cloning. First, the genes encoding human telomerase amino acids 241-1132, an ERBV 2A peptide, and the amino terminal half of human Mucin-1 were amplified by PCR from plasmid 1271 with primers f pmed Nhe Ter240 and r muc 986-963. The genes encoding the carboxy terminal half of human Mucin-1, an EMCV 2A peptide, and CEA amino acids 2-144, 323-702 were amplified by PCR from plasmid 1409 with primers f Muc 960-983 and r pmed CEA GPI. The partially overlapping amplicons were digested with Dpn I, mixed together, and cloned into the Nhe I/Bgl II sites of pPJV7563 by Seamless cloning. The open reading frame nucleotide sequence of Plasmid 1426 is set forth in SEQ ID NO:46. The amino acid sequence encoded by Plasmid 1426 is set for in SEQ ID NO:47.

Plasmid 1427 (Tert240-ERA2A-mCEA-T2A-Muc1). Plasmid 1427 was constructed using the techniques of PCR and Seamless cloning. First, the genes encoding human telomerase amino acids 241-1132, an ERAV 2A peptide, and the amino terminal half of mCEA were amplified by PCR from plasmid 1440 with primers f pmed Nhe Ter240 and R CEA SR2. The genes encoding the carboxy terminal half of mCEA, a TAV 2A peptide, and human Mucin-1 amino acids 2-225, 946-1255 were amplified by PCR from plasmid 1410 with primers f cCEA 562-592 and r pmed Bgl Muc. The partially overlapping amplicons were digested with Dpn I, mixed together, and cloned into the Nhe I/Bgl II sites of pPJV7563 by Seamless cloning. The open reading frame nucleotide sequence of Plasmid 1427 is set forth in SEQ ID NO:48. The amino acid sequence encoded by Plasmid 1427 is set for in SEQ ID NO:49.

Plasmid 1428 (Muc1-EMC2A-cCEA-T2A-Tert240). Plasmid 1428 was constructed using the techniques of PCR and Seamless cloning. First, the genes encoding human Mucin-1 amino acids 2-225, 946-1255, an EMCV 2A peptide, and the amino terminal half of cCEA were amplified by PCR from plasmid 1431 with primers f pmed Nhe Muc and r cCEA 849-820. The genes encoding the carboxy terminal half of cCEA, a TAV 2A peptide, and human telomerase amino acids 241-1132 were amplified by PCR from plasmid 1432 with primers f CEA 833-855 and r pmed Bgl Ter240. The partially overlapping amplicons were digested with Dpn I, mixed together, and cloned into the Nhe I/Bgl II sites of pPJV7563 by Seamless cloning. The open reading frame nucleotide sequence of Plasmid 1428 is set forth in SEQ ID NO:50. The amino acid sequence encoded by Plasmid 1428 is set for in SEQ ID NO:51.

Plasmid 1429 (cCEA-T2A-Tert240-ERB2A-Muc1). Plasmid 1429 was constructed using the techniques of PCR and Seamless cloning. First, the genes encoding human CEA amino acids 35-144, 323-677, a TAV 2A peptide, and the amino terminal half of human Tert240 were amplified by PCR from plasmid 1432 with primers f pmed CEA D1 and r tert 1602-1579. The genes encoding the carboxy terminal half of human Tert240, an ERBV 2A peptide, and human Mucin-1 amino acids 2-225, 946-1255 were amplified by PCR from plasmid 1271 with primers f tert 1584-1607 and r pmed Bgl Muc. The partially overlapping amplicons were digested with Dpn I, mixed together, and cloned into the Nhe I/Bgl II sites of pPJV7563 by Seamless cloning. The open reading frame nucleotide sequence of Plasmid 1429 is set forth in SEQ ID NO:52. The amino acid sequence encoded by Plasmid 1429 is set for in SEQ ID NO:53.

1D. Vector Construction

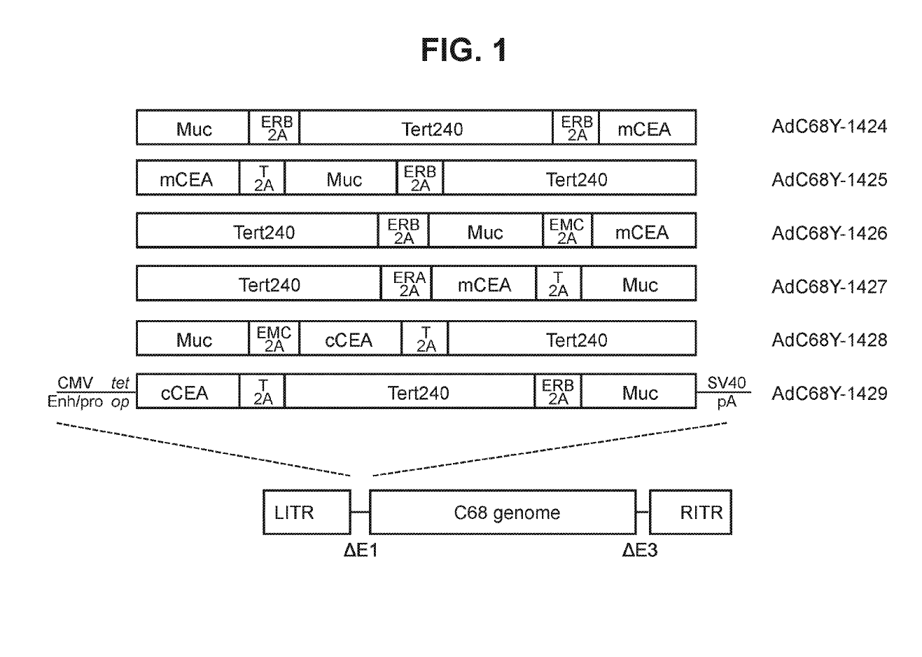

This example illustrates the construction of vectors carrying a multi-antigen construct. Vectors carrying the same triple-antigen construct (open reading frame) as that carried by each of plasmids 1424, 1425, 1426, 1427, 1428, and 1429 were constructed from chimpanzee adenovirus AdC68 genomic sequences as described in international patent application publication WO2015/063647. These vectors are referred to as AdC68Y-1424, AdC68Y-1425, AdC68Y-1426, AdC68Y-1427, AdC68Y-1428, and AdC68Y-1429, respectively. The organizations of these vectors are provided in FIG. 1.

The full length genomic sequence of AdC68 is available from Genbank having Accession Number AC_000011.1 and is also provided in WO2015/063647. The AdC68 backbone without transgenes (the “empty vector”) was designed in silico with E1 and E3 deletions engineered into the virus to render it replication incompetent and create space for transgene insertion. Vector AdC68Y, having deletions of bases 456-3256 and 27476-31831, was engineered to have improved growth properties over previous AdC68 vectors. The empty vector was biochemically synthesized in a multi-stage process utilizing in vitro oligo synthesis and subsequent recombination-mediated intermediate assembly as artificial chromosomes in Escherichia coli (E. coli) and/or yeast. Open reading frames encoding the various immunogenic TAA polypeptides were amplified by PCR from plasmids 1424, 1425, 1426, 1427, 1428, and 1429 using primer sets Muc1-20 bp-F-98/mCEA-20 bp-R-100, Y-mCEA-S2/Y-Tert-A2, Y-Tert-S/Y-CEA-A, Y-Tert-S/Y-MUC-A, Y-MUC-S2/Y-Tert-A2, and cCEA-20 bp-F-106/Muck 20BP-R-108, respectively. The amplicons were then inserted into the empty vector backbone. Recombinant viral genomes were released from the bacterial artificial chromosomes by digestion with Pacl and the linearized nucleic acids were transfected into an E1 complimenting adherent HEK293 cell line. Upon visible cytopathic effects and adenovirus foci formation, cultures were harvested by multiple rounds of freezing/thawing to release virus from the cells. Viruses were amplified and purified by standard techniques.

### Example 2. Immunogenicity of Muc1 Single-Antigen Constructs

Study in HLA-A2/DR1 Mice

Study Design.

Twelve mixed gender HLA-A2/DR1 mice were primed on day 0 and boosted on day 14 with DNA construct Plasmid 1027 (which encodes the membrane-bound immunogenic MUC1 polypeptide of SEQ ID NO:5) or Plasmid 1197 (which encodes the cytosolic immunogenic MUC1 polypeptide of SEQ ID NO:7) using the PMED method. On day 21, mice were sacrificed and splenocytes assessed for MUC1-specific cellular immunogenicity in an interferon-gamma (IFN-γ) ELISpot and intracellular cytokine staining (ICS) assay.

Particle Mediated Epidermal Delivery (PMED).

PMED is a needle-free method of administering vaccines to animals or to patients. The PMED system involves the precipitation of DNA onto microscopic gold particles that are then propelled by helium gas into the epidermis. The ND10, a single use device, uses pressurized helium from an internal cylinder to deliver gold particles and the X15, a repeater delivery device, uses an external helium tank which is connected to the X15 via high pressure hose to deliver the gold particles. Both of these devices were used in studies to deliver the MUC1 DNA plasmids. The gold particle was usually 1-3 μm in diameter and the particles were formulated to contain 2 μg of antigen DNA plasmids per 1 mg of gold particles. (Sharpe, M. et al.: P. Protection of mice from H5N1 influenza challenge by prophylactic DNA vaccination using particle mediated epidermal delivery. Vaccine, 2007, 25(34): 6392-98: Roberts L K, et al.: Clinical safety and efficacy of a powdered Hepatitis B nucleic acid vaccine delivered to the epidermis by a commercial prototype device. Vaccine, 2005; 23(40):4867-78).

IFN-γ ELISpot Assay.

Splenocytes from individual animals were co-incubated in triplicate with individual Ag-specific peptides (each peptide at 2-10 ug/ml, 2.5-5e5 cells per well) or pools of 15mer Ag-specific peptides (overlapping by 11 amino acids, covering the entire Ag-specific amino acid sequence; see Table 15; each peptide at 2-5 ug/ml, 1.25-5e5 cells per well) in IFN-γ ELISpot plates. The plates were incubated for ˜16 hours at 37° C., 5% CO₂, then washed and developed, as per manufacturer's instruction. The number of IFN-γ spot forming cells (SFC) was counted with a CTL reader. The average of the triplicates was calculated and the response of the negative control wells, which contained no peptides, subtracted. The SFC counts were then normalized to describe the response per 1e6 splenocytes. The antigen-specific responses in the tables represent the sum of the responses to the Ag-specific peptides or peptide pools.

ICS Assay.

Splenocytes from individual animals were co-incubated with H-2b-, HLA-A2-, or HLA-A24-restricted Ag-specific peptides (each peptide at 5-10 ug/ml, 1-2e6 splenocytes per well) or pools of 15mer Ag-specific peptides (overlapping by 11 amino acids, covering the entire Ag-specific amino acid sequence; see Table 15; each peptide at 2-5 ug/ml, 1-2e6 splenocytes per well) in U-bottom 96-well-plate tissue culture plates. The plates were incubated ˜16 hrs at 37° C., 5% CO₂. The cells were then stained to detect intracellular IFN-γ expression from CD8⁺ T cells and fixed. Cells were acquired on a flow cytometer. The data was presented per animal as frequency of peptide(s) Ag- or peptide pool Ag-specific IFN-γ⁺ CD8⁺ T cells after subtraction of the responses obtained in the negative control wells, which contained no peptide.

Sandwich ELISA Assay.

The standard sandwich ELISA assay was done using the Tecan Evo, Biomek Fx^{P}, and BioTek 405 Select TS automation instruments. The 384 well microplates (flat-well, high binding) were coated at 25 μl/well with 1.0 μg/mL human MUC1 or human CEA protein (antigen) in 1×PBS, and incubated overnight at 4° C. The next morning, plates were blocked for one hour at RT with 5% FBS in PBS with 0.05% Tween 20 (PBS-T). Mouse serum was prepared at a 1/100 starting dilution in PBS-T in 96 U-bottom well plates. The Tecan Evo performed ½ log serial dilutions in PBS-T over 9 dilution increment points, followed by stamping of 25 μl/well of diluted serum from the 96 well plates to 384 well plates. The 384 well plates were incubated for 1 hour at RT on a shaker at 600 RPM, then, using the BioTek EL 405 Select TS plate washer, the plates were washed 4 times in PBS-T. Secondary mouse anti-IgG-HRP antibody was diluted to an appropriate dilution and stamped by Biomek Fx^{P} at 25 μl/well into 384 well plates, and incubated for 1 hour at RT on a shaker at 600 RPM, followed by 5 repeated washes. Using the Biomek Fx^{P}, plates were stamped at 25 μl/well of RT TMB substrate and incubated in the dark at RT for 30 minutes, followed by 25 μl/well stamping of 1N H₂SO₄ acid to stop the enzymatic reaction. Plates were read on the Molecular Devices, Spectramax 340PC/384 Plus at 450 nm wavelength. Data were reported as calculated titers at OD of 1.0 with a limit of detection of 99.0. The antigen-specific commercial monoclonal antibody was used in each plate as a positive control to track plate-to-plate variation performance; irrelevant vaccinated mouse serum was used as a negative control, and PBS-T only wells were used to monitor non-specific binding background. Titers in the tables represent antigen-specific IgG titers elicited from individual animals.

Results.

Table 1 shows ELISpot and ICS data from HLA-A2/DR1 splenocytes cultured with peptide pools derived from the MUC1 peptide library (see Table 15) or MUC1 peptide aa516-530, respectively. Numbers in column 3 represent # IFN-γ spots/10⁶ splenocytes after restimulation with MUC1 peptide pools, and background subtraction. Numbers in column 4 represent the frequency of CD8⁺ T cells being IFN-γ⁺ after restimulation with MUC1 peptide aa516-530 and background subtraction. A positive response is defined as having SFC>100 and a frequency of IFN-γ⁺ CD8⁺ T cells>0.1%. As shown in Table 1, the immunogenic MUC1 polypeptides made with the full-length membrane-bound (Plasmid 1027) and cytosolic (Plasmid 1197) MUC1 constructs were capable of inducing MUC1-specific T cell responses including HLA-A2-restricted MUC1 peptide aa516-530-specific CD8⁺ T cell responses. The cytosolic MUC1 antigen format induced the highest magnitude of T cell responses. Importantly, T cell responses derived from cancer patients against the MUC1 peptide aa516-530 have been shown to correlate with anti-tumor efficacy in vitro (Jochems C et al., Cancer Immunol Immunother (2014) 63:161-174) demonstrating the importance of raising cellular responses against this specific epitope.

TABLE 1
T cell response induced by the single-antigen MUC1 DNA constructs
(Plasmid 1027 and Plasmid 1197) in HLA-A2/DR1 mice

| | | # IFN-γ spots/ | % CD8⁺ T cells |
|---|---|---|---|
| Construct ID | Animal # | 10⁶ splenocytes | being IFN-γ⁺ |
| Plasmid 1027 | 31 | 494 | 2.25 |
| | 32 | 277 | 1.44 |
| | 33 | 475 | 0.10 |
| | 34 | 1096 | 0.84 |
| | 35 | 282 | 1.45 |
| | 36 | 649 | 1.36 |
| Plasmid 1197 | 43 | 569 | 4.69 |
| | 44 | 1131 | 2.15 |
| | 45 | 122 | 2.81 |
| | 46 | 373 | 1.73 |
| | 47 | 503 | 1.80 |
| | 48 | 2114 | 5.52 |

Study in HLA-A24 Mice

Study design. Mixed gender HLA-A24 mice were primed on day 0 and boosted on days 14, 28 and 42 with DNA construct Plasmid 1027 by PMED administration. On day 21, mice were sacrificed and splenocytes assessed for MUC1-specific cellular immunogenicity (ELISpot).

Results.

Table 2 shows ELISpot data from HLA-A24 splenocytes cultured with peptide pools derived from the MUC1 peptide library (see Table 15). Numbers in column 3 represent # IFN-γ spots/10⁶ splenocytes after restimulation with MUC1 peptide pools and background subtraction. The number in bold font indicates that at least 1 peptide pool tested was too numerous to count, therefore the true figure is at least the value stated. A positive response is defined as having SFC>100. As shown in Table 2, membrane-bound MUC1 construct was capable of inducing MUC1-specific cellular responses.

TABLE 2
T cell response induced by the single-antigen DNA
construct Plasmid 1027 encoding human native full-
length membrane-bound MUC1 antigen in HLA-A24 mice

| | | # IFN-γ spots/ |
|---|---|---|
| Construct ID | Animal # | 10⁶ splenocytes |
| Plasmid 1027 | 8 | 3341 |
| | 9 | 3181 |
| | 10 | 6207 |
| | 11 | 3112 |
| | 12 | 3346 |
| | 13 | 3699 |

Study in Monkeys

Study Design.

14 Chinese-sourced cynomolgus macaques were primed with an adenovirus vector AdC68W encoding the cytosolic MUC1 polypeptide (same polypeptide as encoded by Plasmid 1197) or full-length membrane-bound MUC1 polypeptide (same polypeptide as encoded by Plasmid 1027) at 2e11 viral particles by bilateral intramuscular injection (1 mL total). 29 days later, animals were boosted with Plasmid 1197 or Plasmid 1027 delivered intramuscularly bilaterally via electroporation (2 mL total). Anti-CTLA-4 was administered subcutaneously on days 1 (32 mg) and 29 (50 mg). 14 days after the last immunization, animals were bled and PBMCs and sera isolated to assess MUC1-specific cellular (ELISpot, ICS) and humoral (ELISA) responses, respectively. The adenovirus vector AdC68W used in this and other examples of the present disclosure was constructed from the chimpanzee adenovirus AdC68 according to the method described in international patent application WO2015/063647. NHP-specific immune assays.

EL/Spot Assay.

PBMCs from individual animals were co-incubated in duplicate with pools of 15mer Ag-specific peptides (overlapping by 11 amino acids, covering the entire Ag-specific amino acid sequence), each peptide at 2 ug/ml, 4e5 cells per well, in IFN-γ ELISpot plates (see Table 15). The plates were incubated for ˜16 hrs at 37° C., 5% CO₂, then washed and developed, as per manufacturers instruction. The number of IFN-γ spot forming cells (SFC) was counted with a CTL reader. The average of the duplicates was calculated and the response of the negative control wells, which contained no peptides, subtracted. The SFC counts were then normalized to describe the response per 1e6 PBMCs. The antigen-specific responses in the tables represent the sum of the responses to the Ag-specific peptide pools.

ICS Assay.

PBMCs from individual animals were co-incubated with pools of 15mer MUC1 peptides (overlapping by 11 amino acids, covering the entire native full-length MUC1 amino acid sequence; see Table 15), each peptide at 2 ug/mL, 1.5-2e6 PBMCs per well, in U-bottom 96-well-plate tissue culture plates. The plates were incubated for ˜16 hours at 37° C., 5% CO₂, and then stained to detect intracellular IFN-γ expression from CD8 T cells. After fixation, the cells were acquired on a flow cytometer. The results are presented per individual animal as number of MUC1, CEA, or TERT-specific IFN-γ⁺ CD8⁺ T cells after subtraction of the responses obtained in the negative control wells, which contained no peptide, and normalized to 1e6 CD8⁺ T cells.

Sandwich ELISA Assay.

The standard sandwich ELISA assay was done using the Tecan Evo, Biomek Fx^{P}, and BioTek 405 Select TS automation instruments. The 384 well microplates (flat-well, high binding) were coated at 25 μl/well with 1.0 μg/mL human MUC1 or human CEA protein (antigen) in 1×PBS, and incubated overnight at 4° C. The next morning, plates were blocked for one hour at RT with 5% FBS in PBS with 0.05% Tween 20 (PBS-T). Sera from Chinese-sourced cynomolgus macaques were prepared at a 1/100 starting dilution in PBS-T in 96 U-bottom well plates. The Tecan Evo performed ½ log serial dilutions in PBS-T over 9 dilution increment points, followed by stamping of 25 μl/well of diluted serum from the 96 well plates to 384 well plates. The 384 well plates were incubated for 1 hour at RT on a shaker at 600 RPM, then, using the BioTek EL 405 Select TS plate washer, the plates were washed 4 times in PBS-T. Secondary rhesus anti-IgG-HRP antibody, which cross-reacts with cynomolgus IgG, was diluted to an appropriate dilution and stamped by Biomek Fx^{P} at 25 μl/well into 384 well plates, and incubated for 1 hour at RT on a shaker at 600 RPM, followed by 5 repeated washes. Using the Biomek Fx^{P}, plates were stamped at 25 μl/well of RT TMB substrate and incubated in the dark at RT for 30 minutes, followed by 25 μl/well stamping of 1N H₂SO₄ acid to stop the enzymatic reaction. Plates were read on the Molecular Devices, Spectramax 340PC/384 Plus at 450 nm wavelength. Data were reported as calculated titers at OD of 1.0 with a limit of detection of 99.0. The antigen-specific commercial monoclonal antibody was used in each plate as a positive control to track plate-to-plate variation performance; irrelevant vaccinated mouse serum was used as a negative control, and PBS-T only wells were used to monitor non-specific binding background. Titers in the tables represent antigen-specific IgG titers elicited from individual animals.

Results.

Table 3 shows the ELISpot and ICS data from Chinese-sourced cynomolgus macaque PBMCs cultured with peptide pools derived from the MUC1 peptide library (Table 15), and the ELISA data from Chinese-sourced cynomolgus macaque sera. Numbers in column 3 represent # IFN-γ spots/10⁶ PBMCs after restimulation with MUC1 peptide pools and background subtraction. Numbers in column 4 represent # IFN-γ⁺ CD8⁺ T cells/10⁶ CD8⁺ T cells after restimulation with MUC1 peptide pools and background subtraction. Numbers in column 5 represent the anti-MUC1 IgG titer (Optical Density (O.D)=1, Limit of Detection (L.O.D)=99.0). A positive response is defined as having SFC>50, IFN-γ⁺ CD8⁺ T cells/1e6 CD8⁺ T cells>50, and IgG titers>99. As shown in Table 3, the immunogenic MUC1 polypeptides made with the cytosolic (Plasmid 1197) and native full-length membrane-bound (Plasmid 1027) MUC1 constructs were capable of inducing MUC1-specific T and B cell responses. The native full-length membrane-bound MUC1 construct (Plasmid 1027) was shown to induce the overall best MUC1-specific cellular and humoral response.

TABLE 3
T and B cell responses induced by the single-antigen adenoviral
AdC68W vector and single-antigen DNA constructs (Plasmid 1197;
Plasmid 1027) in Chinese-sourced cynomolgus macaques

| | | # IFN-γ | # IFN-γ⁺ CD8⁺ | |
|---|---|---|---|---|
| Construct ID | | spots/10⁶ | T cells/1e6 | |
| # | Animal # | splenocytes | CD8⁺ T cells | IgG titer |
| Plasmid 1197 | 4001 | 0 | 0.0 | 8589.7 |
| | 4002 | 38 | 1549.0 | 4245.9 |
| | 4003 | 17 | 0.0 | 2631.9 |
| | 4501 | 165 | 4792.3 | 614.6 |
| | 4502 | 1703 | 47727.4 | 1882.8 |
| | 4503 | 0 | 802.8 | 4366.4 |
| | 4504 | 373 | 1857.0 | 4419.3 |
| Plasmid 1027 | 5001 | 797 | 813.5 | 5332.2 |
| | 5002 | 1013 | 312.9 | 16233.5 |
| | 5003 | 1011 | 9496.9 | 6885.8 |
| | 5004 | 175 | 170.2 | 48759.0 |
| | 5501 | 214 | 4803.3 | 13010.4 |
| | 5502 | 306 | 8367.6 | 13115.3 |
| | 5503 | 405 | 0.0 | 89423.0 |

### Example 3. Immunogenicity of CEA Single-Antigen Constructs

Immune Response Study in Pasteur (HLA-A2/DR 1) Mice

Study Design.

Mixed gender HLA-A2/DR1 mice were primed on day 0 and boosted on day 14 with a plasmid carrying a single-antigen construct encoding the human membrane-bound (Plasmid 1386) or cytosolic CEA polypeptide (Plasmid 1390) by electroporation. The antigen-specific T cell response was measured seven days later in an IFN-γ ELISpot and ICS assay.

Results.

Table 4 shows ELISpot and ICS data from HLA-A2/DR1 splenocytes cultured with peptide pools derived from the CEA peptide library composed of aa1-699 for mice immunized with construct 1386, and aa37-679 (removal of signal sequence and GPI sequence) for mice immunized with Plasmid 1390 (see also Table 15). Numbers in columns 3 and 4 represent # IFN-γ⁺ spots/1e6 splenocytes and the frequency of IFN-γ⁺ CD8⁺ T cells respectively, elicited after restimulation with relevant CEA peptides pools and background subtraction Table 5 shows ELISpot data from HLA-A2/DR1 splenocytes cultured with the CEA peptide aa693-701. A positive response is defined as having SFC>100 and a frequency of IFN-γ⁺ CD8⁺ T cells>0.1%. As shown in Table 4, the immunogenic CEA polypeptides made with the membrane-bound (Plasmid 1386) and cytosolic (Plasmid 1390) CEA constructs described in Example 1A above were capable of inducing CEA-specific T cell responses. Comparable magnitudes of CEA-specific T cell responses were induced by both membrane-bound and cytosolic CEA antigen formats. As shown in Table 5, immunization with the membrane-bound construct 1386 induced an HLA-A2 restricted T cell response against CEA peptide aa693-701, which has been shown in the literature to be processed and presented by HLA-A2 (Conforti A et al., J Immunother (2009) 32:744-754).

TABLE 4
T cell response induced by the single-antigen DNA constructs
(Plasmids 1386 and 1390) encoding human membrane-bound
or human cytosolic CEA polypeptide in HLA-A2/DR1 mice

| | | # IFN-γ spots/ | % CD8⁺ T cells |
|---|---|---|---|
| Construct ID | Animal # | 10⁶ splenocytes | being IFN-γ⁺ |
| Plasmid 1386 | 25 | 332 | 0.57 |
| | 26 | 359 | 0.50 |
| | 27 | 579 | 2.78 |
| | 28 | 1525 | 6.80 |
| | 29 | 2435 | 0.31 |
| | 30 | 321 | 0.12 |
| | 31 | 609 | Not determined |
| | 32 | 229 | Not determined |
| Plasmid 1390 | 17 | 381 | 0.85 |
| | 18 | 1035 | 0.75 |
| | 19 | 631 | 1.01 |
| | 20 | 1811 | 10.11 |
| | 21 | 289 | 1.03 |
| | 22 | 157 | 0.56 |
| | 23 | 267 | Not determined |
| | 24 | 1329 | Not determined |

TABLE 5
HLA-A2-restricted CEA peptide aa693-701-specific
T cell responses induced by single antigen DNA
construct encoding human membrane-bound CEA polypeptide
(Plasmid 1386; mCEA) in HLA-A2 mice

| | | # IFN-γ spots/ |
|---|---|---|
| Construct ID | Animal # | 10⁶ splenocytes |
| Plasmid 1386 | 25 | 397 |
| | 26 | 1279 |
| | 27 | 1658 |
| | 28 | 695 |
| | 29 | 677 |
| | 30 | 239 |
| | 31 | 593 |
| | 32 | 578 |

Immune Response Study in HLA A24 Mice

Study Designs.

Sixteen mixed-gender HLA-A24 mice were primed on day 0 and boosted on day 14 with human membrane-bound (Plasmid 1386) or cytosolic CEA (Plasmid 1390) DNA constructs via DNA electroporation. CEA-specific T cell responses were measured 7 days after the last immunization in an IFN-γ ELISpot and ICS assay.

Results.

Table 6 shows ELISpot and ICS data from HLA-A24 splenocytes cultured with peptide pools derived from the CEA peptide library (see also Table 15). Numbers in column 3 represent # IFN-γ spots/10⁶ splenocytes after restimulation with CEA peptide pools encompassing aa1-699 and background subtraction. Numbers in column 4 represent the frequency of CD8⁺ T cells being IFN-γ⁺ after restimulation with CEA peptide pools encompassing aa37-679, and background subtraction. A positive response is defined as having SFC>100 and a frequency of IFN-γ⁺ CD8⁺ T cells>0.1%. The number in bold font indicates that at least 1 peptide pool tested was too numerous to count, therefore the true figure is at least the value stated. As shown in Table 6, the immunogenic CEA polypeptides made with the membrane-bound (Plasmid 1386) and cytosolic CEA (Plasmid 1390) constructs were capable of inducing comparable CEA-specific cellular responses as measured via ELISpot. Vaccination with the cytosolic CEA construct (Plasmid 1390), however, induced higher CEA-specific IFN-γ⁺ CD8⁺ T cell responses measured via ICS.

TABLE 6
T cell response induced by the single-
antigen DNA constructs in HLA-A24 mice

| | | # IFN-γ spots/ | % CD8⁺ T cells |
|---|---|---|---|
| Construct ID | Animal # | 10⁶ splenocytes | being IFN-γ⁺ |
| Plasmid 1386 | 25 | 3037 | 0.19 |
| | 26 | 3412 | −0.26 |
| | 27 | 2385 | 1.48 |
| | 28 | 2293 | 0.62 |
| | 29 | 1845 | −0.27 |
| | 30 | 2611 | 0.07 |
| | 31 | 3534 | Not determined |
| | 32 | 1985 | Not determined |
| Plasmid 1390 | 17 | 2345 | 7.67 |
| | 18 | 1357 | 17.48 |
| | 19 | 3723 | 16.41 |
| | 20 | 3081 | 41.30 |
| | 21 | 3031 | 18.24 |
| | 22 | 1531 | 0.73 |
| | 23 | 2786 | Not determined |
| | 24 | 1419 | Not determined |

### Example 4. Immunogenicity of Tert Single-Antigen Constructs

Immune Responses Study in HLA-A2/DR1 Mice

Study Design.

Six mixed gender HLA-A2/DR1 mice were primed with an AdC68W adenovirus vector encoding the truncated (Δ240) cytosolic immunogenic TERT polypeptide (Plasmid 1112) at 1e10 viral particles by intramuscular injection (50 ul). 28 days later, animals were boosted intramuscularly with 50 ug DNA delivered bilaterally via electroporation (2×20 ul) encoding the truncated (Δ240) cytosolic TERT antigen (Plasmid 1112). The antigen-specific T cell response was measured seven days later in an IFN-γ ELISpot and ICS assay.

Results.

Table 7 shows ELISpot and ICS data from HLA-A2/DR1 splenocytes cultured with peptide pools derived from the TERT peptide library (see also Table 15) or TERT peptide aa861-875, respectively. Numbers in column 3 represent # IFN-γ spots/10⁶ splenocytes after restimulation with TERT peptide pools and background subtraction. Numbers in column 4 represent the frequency of CD8⁺ T cells being IFN-γ⁺ after restimulation with TERT peptide aa861-875 and background subtraction. A positive response is defined as having SFC>100 and a frequency of IFN-γ⁺ CD8⁺ T cells>0.1%. As shown in Table 7, the immunogenic TERT polypeptide made with the truncated (Δ240) cytosolic TERT construct was capable of inducing HLA-A2-restricted TERT-specific CD8 T cell responses.

TABLE 7
T cell response induced by the single-antigen adenoviral AdC68W
and single-antigen DNA constructs (Plasmid 1112) encoding human
truncated (Δ240) cytosolic TERT antigen in HLA-A2/DR1 mice

| | | # IFN-γ spots/ | % CD8⁺ T cells |
|---|---|---|---|
| Construct ID | Animal # | 10⁶ splenocytes | being IFN-γ⁺ |
| Plasmid 1112 | 13 | 2851 | 32.79 |
| | 14 | 2691 | 13.60 |
| | 15 | 3697 | 7.87 |
| | 16 | 2984 | 21.30 |
| | 17 | 1832 | 26.40 |
| | 18 | 1385 | 3.16 |

Immune Responses Study in HLA-A24 Mice

Study Designs.

Eight mixed gender HLA-A24 mice were primed with an AdC68W adenovirus vector encoding the truncated (Δ240) cytosolic TERT polypeptide (same polypeptide as encoded by Plasmid 1112) at 1e10 viral particles total by bilateral intramuscular injection (50 ul into each tibialis anterior muscle). 14 days later, animals were boosted intramuscularly with 50 ug DNA (Plasmid 1112) delivered bilaterally via electroporation (2×20 ul) encoding the truncated (Δ240) cytosolic TERT polypeptide. The antigen-specific T cell response was measured seven days later in an IFN-γ ELISpot and ICS assay.

Results.

Table 8 shows IFN-γ ELISpot and ICS data from HLA-A24 splenocytes cultured with peptide pools derived from the TERT peptide library (see also Table 15) or TERT peptide aa841-855), respectively. Numbers in column 3 represent # IFN-γ spots/10⁶ splenocytes after restimulation with TERT peptide pools and background subtraction. Numbers in column 4 represent the frequency of CD8⁺ T cells being IFN-γ⁺ after restimulation with TERT peptides aa841-855, and background subtraction. Numbers in bold font indicate that at least 1 peptide pool tested was too numerous to count, therefore the true figure is at least the value stated. A positive response is defined as having SFC>100 and a frequency of IFN-γ⁺ CD8⁺ T cells>0.1%. As shown in Table 8, the immunogenic TERT polypeptide made with the truncated (Δ240) cytosolic TERT (Plasmid 1112) construct is capable of inducing HLA-A24-restricted TERT-specific CD8⁺ T cell responses.

TABLE 8
T cell response induced by the single-antigen adenoviral AdC68W
vector and single-antigen DNA constructs (Plasmid 1112) encoding
human truncated (Δ240) cytosolic TERT antigen in HLA-A24 mice

| | | # IFN-γ spots/ | % CD8⁺ T cells |
|---|---|---|---|
| Construct ID | Animal # | 10⁶ splenocytes | being IFN-γ⁺ |
| Plasmid 1112 | 17 | 4233 | 41.5 |
| | 18 | 2643 | 3.34 |
| | 19 | 1741 | 31.5 |
| | 20 | 3407 | 3.05 |
| | 21 | 3213 | 0.0903 |
| | 22 | 596 | 0 |
| | 23 | 1875 | 13.8 |
| | 24 | 2011 | 19.8 |

Immune Responses Study in Monkeys

Study Design.

Eight Chinese-sourced cynomolgus macaques were primed with an AdC68W adenovirus vector encoding the truncated (A240) cytosolic TERT antigen (Plasmid 1112) at 2e11 viral particles by bilateral intramuscular injection (1 mL total). 30 and 64 days later, animals were boosted with DNA (Plasmid 1112) encoding truncated (Δ240) cytosolic TERT antigen delivered intramuscularly bilaterally via electroporation (2 mL total). Anti-CTLA-4 was administered subcutaneously on days 1 (32 mg), 31 (50 mg) and 65 (75 mg). 14 days after the last immunization, animals were bled and PBMCs isolated to assess TERT-specific cellular (ELISpot, ICS) responses.

Results.

Table 9 shows the ELISpot and ICS data from Chinese-sourced cynomolgus macaques' PBMCs cultured with peptide pools derived from the TERT peptide library (see also Table 15). Numbers in column 3 represent # IFN-γ spots/10⁶ splenocytes after restimulation with TERT peptide pools and background subtraction. Numbers in column 4 represent # IFN-γ⁺ CD8⁺ T cells/10⁶ CD8⁺ T cells after restimulation with TERT peptide pools and background subtraction. A positive response is defined as having SFC>50 and IFN-γ⁺ CD8⁺ T cells/1e6 CD8⁺ T cells>50. As shown in Table 9, the immunogenic TERT polypeptide made with the truncated (A240) cytosolic (Plasmid 1112) TERT construct was capable of inducing TERT-specific T cell responses.

TABLE 9
T cell response induced by the TERT single-antigen adenoviral
AdC68W and TERT single-antigen DNA constructs (Plasmid
1112) in Chinese-sourced cynomolgus macaques

| Construct ID | | # IFN-γ spots/ | # IFN-γ⁺ CD8⁺ T cells/ |
|---|---|---|---|
| # | Animal # | 10⁶ splenocytes | 1e6 CD8⁺ T cells |
| Plasmid 1112 | 1001 | 3487 | 29472.2 |
| | 1002 | 1130 | 4906.6 |
| | 1003 | 2077 | 2984.2 |
| | 1004 | 133 | 337.8 |
| | 1501 | 3157 | 5325.1 |
| | 1502 | 2037 | 653.2 |
| | 1503 | 2697 | 16953.4 |
| | 1504 | 1208 | 1178.9 |

### Example 5. Immunogenicity of Dual-Antigen Constructs

Immune Response Study in Monkeys

Study Design.

24 Chinese-sourced cynomolgus macaques were primed with dual-antigen adenoviral AdC68W vectors encoding human native full-length membrane-bound MUC1 (MUC1) and human truncated (Δ240) cytosolic TERT (TERT_{Δ240}) polypeptides (Plasmids 1270, 1271, and 1269) at 2e11 viral particles by bilateral intramuscular injection (1 mL total). 30 and 64 days later, animals were boosted with dual-antigen DNA constructs (Plasmids 1270, 1271, and 1269) encoding the same two antigens delivered intramuscularly bilaterally via electroporation (2 mL total). Anti-CTLA-4 was administered subcutaneously on days 1 (32 mg), 31 (50 mg) and 65 (75 mg). 14 days after the last immunization, animals were bled and PBMCs and serum isolated to assess MUC1- and TERT-specific cellular (ELISpot, ICS) and MUC1-specific humoral (ELISA) responses, respectively. In total, three different dual-antigen vaccine constructs, which co-expressed both antigens, were evaluated: a) MUC1-2A-TERT_{Δ240} (Plasmid 1270), an AdC68W vector and DNA plasmid encoding MUC1 and TERT linked by a 2A peptide; b) TERT_{Δ240}-2A-MUC1 (Plasmid 1271), an AdC68W vector and DNA plasmid encoding TERT and MUC1 linked by a 2A peptide; c) MUC1-TERT_{Δ240} (Plasmid 1269), an AdC68W vector and DNA plasmid encoding the MUC1-TERT fusion protein.

Results.

Table 10 shows the ELISpot and ICS data from Chinese-sourced cynomolgus macaque PBMCs cultured with peptide pools derived from the MUC1 and TERT peptide libraries (see also Table 15), and the ELISA data from Chinese-sourced cynomolgus macaque sera. A positive response is defined as having SFC>50, IFN-γ⁺ CD8⁺ T cells/1e6 CD8⁺ T cells>50, and IgG titers>99. Numbers in columns 3 and 6 represent # IFN-γ spots/10⁶ splenocytes after restimulation with MUC1 and TERT peptide pools and background subtraction, respectively. Numbers in bold font indicate that at least 1 peptide pool tested was too numerous to count, therefore the true figure is at least the value stated. Numbers in columns 4 and 7 represent # IFN-γ⁺ CD8⁺ T cells/10⁶ CD8⁺ T cells after restimulation with MUC1 peptide pools and TERT peptide pools, respectively, and background subtraction. Numbers in column 5 represent the anti-MUC1 IgG titer (Optical Density (O.D)=1, Limit of Detection (L.O.D)=99.0). As shown in Table 10, the immunogenic MUC1 and TERT polypeptides made with the MUC1- and TERT-expressing dual-antigen constructs (Plasmids 1270, 1271, and 1269) were capable of inducing MUC1- and TERT-specific T cell responses, and MUC1-specific B cell responses. The dual-antigen construct 1269 encoding a MUC1-TERT fusion protein was shown to induce the strongest overall MUC1-specific cellular response; in contrast, dual-antigen construct Plasmid 1271 (TERT-2A-MUC1) was shown to induce the strongest overall TERT-specific cellular response. All three dual-antigen constructs were shown to induce a comparable MUC1-specific humoral response.

TABLE 10
T and B cell responses induced by the dual-antigen adenoviral AdC68W and
single-antigen DNA constructs (Plasmid 1270, 1271, and 1269) encoding an
immunogenic MUC1 and TERT polypeptide in Chinese-sourced cynomolgus macaques

| | | MUC1 | | | TERT | |
|---|---|---|---|---|---|---|
| | | | # IFN-γ⁺ | | | # IFN-γ⁺ |
| | | | CD8⁺ T | | | CD8⁺ T |
| | | # IFN-γ | cells/1e6 | | # IFN-γ | cells/ |
| Construct | | spots/10⁶ | CD8⁺ T | | spots/10⁶ | 1e6 CD8⁺ |
| ID | Animal # | splenocytes | cells | IgG titer | splenocytes | T cells |
| Plasmid | 5001 | 813 | 1024.4 | 10725.8 | 307 | 436.9 |
| 1270 | 5002 | 2778 | 14740.6 | 27090.7 | 1573 | 423.0 |
| | 5003 | 217 | 1198.7 | 19339.6 | 1687 | 40680.3 |
| | 5004 | 298 | Excluded | 3980.3 | 252 | 805.3 |
| | 5501 | 2287 | 6255.7 | 16278.9 | 692 | 0.0 |
| | 5502 | 760 | 0.0 | 6496.2 | 3010 | 13302.0 |
| | 5503 | 1315 | 199.8 | 6446.4 | 3702 | 7259.3 |
| | 5504 | 500 | 281.8 | 39868.0 | 2005 | 13727.8 |
| Plasmid | 6001 | 1037 | 0.0 | 11770.3 | 2937 | 63106.1 |
| 1271 | 6002 | 185 | 0.0 | 13925.4 | 1295 | 194.8 |
| | 6003 | 372 | 267.4 | 15439.7 | 2138 | 46023.2 |
| | 6004 | 203 | 97.1 | 10530.7 | 1562 | 8424.0 |
| | 6501 | 1315 | 2137.3 | 43487.3 | 3794 | 20358.2 |
| | 6502 | 1008 | 179.2 | 8742.0 | 2955 | 1503.5 |
| | 6503 | 552 | 226.4 | 35183.4 | 1797 | 50008.6 |
| | 6504 | 2200 | 162.8 | 35539.9 | 4402 | 24058.6 |
| Plasmid | 7001 | 193 | 0.0 | 14868.3 | 3320 | 7321.5 |
| 1269 | 7002 | 1353 | 2153.2 | 7546.6 | 870 | 736.2 |
| | 7003 | 1253 | 133.5 | 21277.4 | 2750 | 25827.7 |
| | 7004 | 1858 | 20846.7 | 10359.9 | 3230 | 19664.0 |
| | 7501 | 2138 | 773.6 | 31272.8 | 927 | 332.0 |
| | 7502 | 2177 | 10547.7 | 16635.5 | 2640 | 7527.3 |
| | 7503 | 1460 | 5086.2 | 5465.1 | 2362 | 938.6 |
| | 7504 | 922 | 0.0 | 38530.4 | 2875 | 2949.3 |

### Example 6. Immunogenicity of Triple-Antigen Constructs

Example 6 illustrates the capability of plasmid and adenoviral vectors that carry a triple-antigen construct expressing the human native full-length membrane-bound MUC1 polypeptide (MUC1), human membrane-bound or cytosolic CEA polypeptide (mCEA or cCEA), and human truncated (Δ240) cytosolic TERT polypeptide (TERT_{Δ240}) to elicit Ag-specific T and B cell responses to all three encoded cancer antigens.

Immune Response Study in C57BL/6J Mice Using DNA Electroporation

Study Design.

48 female C57BL/6J mice were immunized with triple-antigen DNA constructs encoding human MUC1, mCEA or cCEA, and TERT_{Δ240}. The triple-antigen DNA vaccine (50 ug) was delivered intramuscularly bilaterally (20 ul total into each tibialis anterior muscle) with concomitant electroporation in a prime/boost regimen, two weeks apart between each vaccination. MUC1-, CEA-, and TERT-specific cellular responses, and MUC1- and CEA-specific humoral responses were measured 7 days after the last immunization in an IFN-γ ELISpot assay and ELISA assay, respectively. In total, six different plasmids carrying triple-antigen DNA constructs each encoding three TAA polypeptides linked by 2A peptides were used as follows: MUC1-2A-TERT_{Δ240}-2A-mCEA (Plasmid 1424), mCEA-2A-MUC1-2A-TERT_{Δ240} (Plasmid 1425), TERT_{Δ240}-2A-MUC1-2A-mCEA (Plasmid 1426), TERT_{Δ240}-2A-mCEA-2A-MUC1 (Plasmid 1427), MUC1-2A-cCEA-2A-TERT_{Δ240} (Plasmid 1428), cCEA-2A-TERT_{Δ240}-2A-MUC1 (Plasmid 1429).

Results.

Tables 11A-C show the ELISpot data from C57BL/6J splenocytes cultured with peptide pools derived from the MUC1, CEA, and TERT peptide libraries (see also Table 15), the ICS data from C57BL/6J splenocytes cultured with TERT peptide aa1025-1039, and the ELISA data from C57BL/6J mouse sera. A positive response is defined as having SFC>100, a frequency of IFN-γ⁺ CD8⁺ T cells>0.1%, and IgG titers>99. Numbers in column 3 of Tables 11A-C represent # IFN-γ spots/10⁶ splenocytes after restimulation with MUC1, CEA, or TERT peptide pools and background subtraction, respectively. Numbers in bold font indicate that at least 1 peptide pool tested was too numerous to count, therefore the true figure is at least the value stated. Numbers in column 4 of Tables 11A-B represent the anti-MUC1 and CEA IgG titer, respectively (Optical Density (O.D)=1, Limit of Detection (L.O.D)=99.0). Numbers in column 4 of Table 110 represent the frequency of CD8⁺ T cells being IFN-γ⁺ after restimulation with TERT-specific peptide TERT aa1025-1039, and background subtraction. As shown in Tables 11A-C, the immunogenic MUC1, CEA, and TERT polypeptides made with the MUC1-, CEA-, and TERT-expressing triple-antigen constructs were capable of inducing T cell responses against all three antigens, and B cell responses against MUC1. In contrast, while mCEA containing triple-antigen constructs (Plasmids 1424-1427) were capable of inducing B cell responses against CEA, cCEA containing triple-antigen constructs (Plasmids 1428-1429) induced either weaker or no CEA-specific B cell responses.

TABLE 11A
MUC1-specific T and B cell responses induced by the triple-
antigen DNA constructs (Plasmids 1424-1429) encoding human
native full-length membrane-bound MUC1, human membrane-
bound or cytosolic CEA, and human truncated (Δ240)
cytosolic TERT polypeptides in C57BL/6J mice

MUC1

| | | # IFN-γ spots/ | |
|---|---|---|---|
| Construct ID | Animal | 10⁶ splenocytes | IgG titer |
| Plasmid 1424 | 33 | 1077 | 4110 |
| | 34 | 1043 | 2280 |
| | 35 | 331 | 1120 |
| | 36 | 245 | 2060 |
| | 37 | 1133 | 3400 |
| | 38 | 660 | 4770 |
| | 39 | 547 | 3460 |
| | 40 | 332 | 838 |
| Plasmid 1425 | 41 | 660 | 3110 |
| | 42 | 603 | 1550 |
| | 43 | 501 | 3880 |
| | 44 | 357 | 884 |
| | 45 | 449 | 2040 |
| | 46 | 740 | 4070 |
| | 47 | 701 | 4740 |
| | 48 | 853 | 2460 |
| Plasmid 1426 | 49 | 732 | 2930 |
| | 50 | 189 | 2190 |
| | 51 | 589 | 2380 |
| | 52 | 1608 | 4050 |
| | 53 | 439 | 1360 |
| | 54 | 1512 | 1090 |
| | 55 | 1332 | 2650 |
| | 56 | 1909 | 2030 |
| Plasmid 1427 | 57 | 727 | 1540 |
| | 58 | 799 | 3550 |
| | 59 | 928 | 590 |
| | 60 | 459 | 1010 |
| | 61 | 455 | 2630 |
| | 62 | 1333 | 2300 |
| | 63 | 1200 | 2920 |
| | 64 | 476 | 4210 |
| Plasmid 1428 | 65 | 1483 | 5170 |
| | 66 | 501 | 573 |
| | 67 | 1435 | 3480 |
| | 68 | 1421 | 6870 |
| | 69 | 813 | 6970 |
| | 70 | 601 | 4770 |
| | 71 | 1913 | 7570 |
| | 72 | 2179 | 6510 |
| Plasmid 1429 | 73 | 951 | 1940 |
| | 74 | 765 | 2700 |
| | 75 | 1085 | 7390 |
| | 76 | 1561 | 5120 |
| | 77 | 600 | 2870 |
| | 78 | 1044 | 6430 |
| | 79 | 1777 | 12500 |
| | 80 | 991 | 5890 |

TABLE 11B
CEA-specific T and B cell responses induced by the triple-
antigen DNA constructs (1424-1429) encoding human native
full-length membrane-bound MUC1, human membrane-bound
or cytosolic CEA, and human truncated (Δ240) cytosolic
TERT polypeptides in C57BL/6J mice

CEA

| | | # IFN-γ spots/ | |
|---|---|---|---|
| Construct ID | Animal | 10⁶ splenocytes | IgG titer |
| Plasmid 1424 | 33 | 1147 | 5530 |
| | 34 | 1147 | 10700 |
| | 35 | 374 | 8880 |
| | 36 | 208 | 18500 |
| | 37 | 491 | 4620 |
| | 38 | 702 | 9190 |
| | 39 | 495 | 12200 |
| | 40 | 455 | 5960 |
| Plasmid 1425 | 41 | 735 | 18500 |
| | 42 | 495 | 5670 |
| | 43 | 1031 | 15400 |
| | 44 | 399 | 13100 |
| | 45 | 353 | 11500 |
| | 46 | 861 | 11100 |
| | 47 | 699 | 14400 |
| | 48 | 932 | 12400 |
| Plasmid 1426 | 49 | 861 | 13200 |
| | 50 | 701 | 15400 |
| | 51 | 859 | 18000 |
| | 52 | 2309 | 13500 |
| | 53 | 673 | 12600 |
| | 54 | 2169 | 10000 |
| | 55 | 1760 | 15700 |
| | 56 | 1751 | 21800 |
| Plasmid 1427 | 57 | 1070 | 8590 |
| | 58 | 803 | 11000 |
| | 59 | 561 | 8740 |
| | 60 | 343 | 7780 |
| | 61 | 191 | 6150 |
| | 62 | 819 | 10000 |
| | 63 | 839 | 7010 |
| | 64 | 484 | 7900 |
| Plasmid 1428 | 65 | 437 | 4250 |
| | 66 | 839 | 1720 |
| | 67 | 843 | 2080 |
| | 68 | 629 | 2090 |
| | 69 | 229 | 1060 |
| | 70 | 665 | 1690 |
| | 71 | 941 | 2530 |
| | 72 | 1181 | 2520 |
| Plasmid 1429 | 73 | 993 | 99 |
| | 74 | 453 | 99 |
| | 75 | 1173 | 99 |
| | 76 | 253 | 99 |
| | 77 | 843 | 99 |
| | 78 | 419 | 178 |
| | 79 | 989 | 2640 |
| | 80 | 981 | 99 |

TABLE 11C
TERT-specific T cell responses induced by the triple-
antigen DNA constructs (1424-1429) encoding human
native full-length membrane-bound MUC1, human membrane-
bound or cytosolic CEA, and human truncated (Δ240)
cytosolic TERT polypeptides in C57BL/6J mice

TERT

| | | # IFN-γ spots/ | % CD8⁺ T cells |
|---|---|---|---|
| Construct ID | Animal | 10⁶ splenocytes | being IFN-γ⁺ |
| Plasmid 1424 | 33 | 1305 | 0.98 |
| | 34 | 1619 | 0.76 |
| | 35 | 2503 | 0.64 |
| | 36 | 509 | 0.65 |
| | 37 | 709 | 0.35 |
| | 38 | 363 | 0.43 |
| | 39 | 499 | Not determined |
| | 40 | 140 | Not determined |
| Plasmid 1425 | 41 | 647 | 0.30 |
| | 42 | 252 | 0.13 |
| | 43 | 392 | 0.36 |
| | 44 | 629 | 0.43 |
| | 45 | 208 | 0.20 |
| | 46 | 515 | 0.41 |
| | 47 | 635 | Not determined |
| | 48 | 2145 | Not determined |
| Plasmid 1426 | 49 | 1742 | 0.52 |
| | 50 | 2262 | 0.65 |
| | 51 | 1959 | 1.21 |
| | 52 | 4739 | 1.14 |
| | 53 | 1123 | 1.01 |
| | 54 | 3987 | 0.77 |
| | 55 | 2925 | Not determined |
| | 56 | 5477 | Not determined |
| Plasmid 1427 | 57 | 548 | 0.17 |
| | 58 | 645 | 0.29 |
| | 59 | 956 | 0.31 |
| | 60 | 910 | 0.45 |
| | 61 | 1916 | 0.92 |
| | 62 | 1273 | 0.21 |
| | 63 | 3115 | Not determined |
| | 64 | 576 | Not determined |
| Plasmid 1428 | 65 | 2475 | 0.84 |
| | 66 | 301 | 0.07 |
| | 67 | 4977 | 2.10 |
| | 68 | 1618 | 1.40 |
| | 69 | 1995 | 1.15 |
| | 70 | 2755 | 1.17 |
| | 71 | 4469 | Not determined |
| | 72 | 3409 | Not determined |
| Plasmid 1429 | 73 | 2324 | 0.86 |
| | 74 | 2866 | 1.73 |
| | 75 | 3795 | 1.92 |
| | 76 | 4142 | 0.97 |
| | 77 | 2760 | 1.60 |
| | 78 | 2655 | 1.06 |
| | 79 | 3103 | Not determined |
| | 80 | 1913 | Not determined |

Immune Response Study in C57BL/6J Mice Using Adenoviral Vectors

Study Design.

48 female C57BL/6J mice were primed with triple-antigen adenoviral vectors encoding human MUC1, mCEA or cCEA, and TERT_{Δ240}, at 1e10 viral particles by intramuscular injection (50 ul into each tibialis anterior muscle). 14 days later, animals were boosted with triple-antigen DNA constructs (50 ug) delivered intramuscularly bilaterally (20 ul into each tibialis anterior muscle) with concomitant electroporation. MUC1-, CEA-, and TERT-specific cellular responses, and MUC1- and CEA-specific humoral responses were measured 7 days after the last immunization in an IFN-γ ELISpot and ICS assay, and an ELISA assay, respectively. In total, six triple-antigen adenoviral and DNA constructs encoding MUC1, mCEA or cCEA, and TERT_{Δ240} linked by 2A peptides were used as follows: MUC1-2A-TERT_{Δ240}-2A-mCEA (Plasmid 1424), mCEA-2A-MUC1-2A-TERT_{Δ240} (Plasmid 1425), TERT_{Δ240}-2A-MUC1-2A-mCEA (Plasmid 1426), TERT_{Δ240}-2A-mCEA-2A-MUC1 (Plasmid 1427), MUC1-2A-cCEA-2A-TERT_{Δ240} (Plasmid 1428), cCEA-2A-TERT_{Δ240}-2A-MUC1 (Plasmid 1429).

Results.

Tables 12A-C shows the ELISpot data from C57BL/6J splenocytes cultured with peptide pools derived from the MUC1, CEA, and TERT peptide libraries (see also Table 15), the ICS data from C57BL/6J splenocytes cultured with TERT peptide aa1025-1039, and the ELISA data from C57BL/6J mouse sera. A positive response is defined as having SFC>100, a frequency of IFN-γ⁺ CD8⁺ T cells>0.1%, and IgG titers>99. Numbers in column 3 in Tables 12A-C represent # IFN-γ spots/10⁶ splenocytes after restimulation with MUC1, CEA, or TERT peptide pools, and background subtraction, respectively. Numbers in bold font indicate that at least 1 peptide pool tested was too numerous to count, therefore the true figure is at least the value stated. Numbers in column 4 in Table 12C represent # IFN-γ⁺ CD8⁺ T cells/10⁶ CD8⁺ T cells after restimulation with TERT-specific peptide TERT aa1025-1039, and background subtraction. Numbers in column 4 in Tables 12A-B represent the anti-MUC1 and anti-CEA IgG titer, respectively (Optical Density (O.D)=1, Limit of Detection (L.O.D)=99.0). As shown in Tables 12A-C, the immunogenic MUC1, CEA, and TERT polypeptides made with MUC1-, CEA-, and TERT-expressing triple-antigen constructs were capable of inducing T cell responses against all three antigens, and B cell responses against MUC1. In contrast, while mCEA containing triple-antigen constructs (Plasmids 1424-1427) were capable of inducing B cell responses against CEA, cCEA containing triple-antigen constructs (Plasmids 1428-1429) induced either weaker or no CEA-specific B cell responses.

TABLE 12A
MUC1-specific T and B cell responses induced by the triple-
antigen adenoviral AdC68Y and DNA constructs (Plasmids 1424-
1429) encoding human native full-length membrane-bound MUC1,
human membrane-bound or cytosolic CEA, and human truncated
(Δ240) cytosolic TERT polypeptides in C57BL/6J mice

MUC1

| | | # IFN-γ spots/ | |
|---|---|---|---|
| Construct ID | Animal # | 10⁶ splenocytes | IgG titer |
| Plasmid 1424 | 33 | 568 | Not determined |
| | 34 | 421 | 34100 |
| | 35 | 929 | 23600 |
| | 36 | 531 | 12900 |
| | 37 | 359 | 7190 |
| | 38 | 769 | 29500 |
| | 39 | 668 | 12300 |
| | 40 | 340 | 30400 |
| Plasmid 1425 | 41 | 251 | 21200 |
| | 42 | 233 | 12400 |
| | 43 | 248 | 10700 |
| | 44 | 137 | 13400 |
| | 45 | 244 | 8400 |
| | 46 | 603 | 15000 |
| | 47 | 469 | 19800 |
| | 48 | 745 | 8370 |
| Plasmid 1426 | 49 | 239 | 6000 |
| | 50 | 291 | 4390 |
| | 51 | 381 | 12700 |
| | 52 | 677 | 5460 |
| | 53 | 548 | 8940 |
| | 54 | 232 | 8170 |
| | 55 | 468 | 8240 |
| | 56 | 224 | 5590 |
| Plasmid 1427 | 57 | 675 | 7650 |
| | 58 | 223 | 3930 |
| | 59 | 605 | 7710 |
| | 60 | 163 | 4190 |
| | 61 | Not determined | 12100 |
| | 62 | 244 | 4230 |
| | 63 | 131 | 4990 |
| | 64 | 429 | 6580 |
| Plasmid 1428 | 65 | 331 | 12700 |
| | 66 | 181 | 4550 |
| | 67 | 360 | 21700 |
| | 68 | 1252 | 25100 |
| | 69 | 345 | 10200 |
| | 70 | 304 | 9670 |
| | 71 | 261 | 11800 |
| | 72 | 485 | 16700 |
| Plasmid 1429 | 73 | 284 | 13300 |
| | 74 | 399 | 12800 |
| | 75 | 391 | 41500 |
| | 76 | 191 | 19900 |
| | 77 | 304 | 8700 |
| | 78 | 651 | 13400 |
| | 79 | 288 | 13800 |
| | 80 | 839 | 2580 |

TABLE 12B
CEA-specific T and B cell responses induced by the triple-
antigen adenoviral AdC68Y and DNA constructs (Plasmids 1424-
1429) encoding human native full-length membrane-bound MUC1,
human membrane-bound or cytosolic CEA, and human truncated
(Δ240) cytosolic TERT polypeptides in C57BL/6J mice

CEA

| | | # IFN-γ spots/ | |
|---|---|---|---|
| Construct ID | Animal # | 10⁶ splenocytes | IgG titer |
| Plasmid 1424 | 33 | 352 | 61600 |
| | 34 | 505 | 45500 |
| | 35 | 428 | 29700 |
| | 36 | 207 | 23500 |
| | 37 | 279 | 35900 |
| | 38 | 400 | 17700 |
| | 39 | 309 | 22100 |
| | 40 | 362 | 38200 |
| Plasmid 1425 | 41 | 867 | 73500 |
| | 42 | 1761 | 28900 |
| | 43 | 547 | 59400 |
| | 44 | 2123 | 44100 |
| | 45 | 359 | 39300 |
| | 46 | 2051 | 71300 |
| | 47 | 466 | 49800 |
| | 48 | 556 | 61700 |
| Plasmid 1426 | 49 | 415 | 12300 |
| | 50 | 599 | 28300 |
| | 51 | 462 | 23900 |
| | 52 | 752 | 14800 |
| | 53 | 799 | 18900 |
| | 54 | 543 | 24700 |
| | 55 | 595 | 17800 |
| | 56 | 439 | 17400 |
| Plasmid 1427 | 57 | 400 | 31000 |
| | 58 | 362 | 18400 |
| | 59 | 343 | 14800 |
| | 60 | 205 | 24000 |
| | 61 | Not determined | 44300 |
| | 62 | 207 | 17900 |
| | 63 | 227 | 29500 |
| | 64 | 329 | 28600 |
| Plasmid 1428 | 65 | 208 | 180 |
| | 66 | 362 | 2840 |
| | 67 | 224 | 7030 |
| | 68 | 583 | 5740 |
| | 69 | 303 | 2030 |
| | 70 | 239 | 2880 |
| | 71 | 489 | 1680 |
| | 72 | 234 | 1490 |
| Plasmid 1429 | 73 | 813 | 99 |
| | 74 | 421 | 619 |
| | 75 | 563 | 99 |
| | 76 | 261 | 99 |
| | 77 | 356 | 99 |
| | 78 | 600 | 99 |
| | 79 | 278 | 992 |
| | 80 | 393 | 552 |

TABLE 12C
TERT-specific T cell responses induced by the triple-antigen
adenoviral AdC68Y and DNA constructs (Plasmids 1424-1429)
encoding human native full-length membrane-bound MUC1, human
membrane-bound or cytosolic CEA, and human truncated (Δ240)
cytosolic TERT polypeptides in C57BL/6J mice

| | | TERT | |
|---|---|---|---|
| | | # IFN-γ spots/ | % CD8⁺ T cells |
| Construct ID | Animal # | 10⁶ splenocytes | being IFN-γ⁺ |
| Plasmid 1424 | 33 | 764 | 1.45 |
| | 34 | 960 | 0.96 |
| | 35 | 1675 | 1.16 |
| | 36 | 1161 | 3.24 |
| | 37 | 1037 | 1.30 |
| | 38 | 684 | 1.00 |
| | 39 | 2887 | Not determined |
| | 40 | 2019 | Not determined |
| Plasmid 1425 | 41 | 3595 | 4.15 |
| | 42 | 1839 | 2.15 |
| | 43 | 1607 | 2.06 |
| | 44 | 1283 | 2.39 |
| | 45 | 2252 | 3.15 |
| | 46 | 2080 | 2.82 |
| | 47 | 571 | Not determined |
| | 48 | 985 | Not determined |
| Plasmid 1426 | 49 | 3129 | 2.46 |
| | 50 | 2264 | 2.51 |
| | 51 | 2901 | 2.43 |
| | 52 | 4556 | 3.51 |
| | 53 | 3396 | 3.97 |
| | 54 | 4184 | 6.19 |
| | 55 | 3311 | Not determined |
| | 56 | 3464 | Not determined |
| Plasmid 1427 | 57 | 2589 | 3.13 |
| | 58 | 991 | 2.52 |
| | 59 | 1123 | 1.16 |
| | 60 | 1993 | 3.16 |
| | 61 | Not determined | 3.20 |
| | 62 | 452 | 3.03 |
| | 63 | 793 | Not determined |
| | 64 | 2100 | Not determined |
| Plasmid 1428 | 65 | 2197 | 3.10 |
| | 66 | 4530 | 7.12 |
| | 67 | 4406 | 8.91 |
| | 68 | 5134 | 7.31 |
| | 69 | 1969 | 3.15 |
| | 70 | 4199 | 5.90 |
| | 71 | 4088 | Not determined |
| | 72 | 3668 | Not determined |
| Plasmid 1429 | 73 | 3929 | 7.98 |
| | 74 | 4779 | 5.95 |
| | 75 | 5060 | 10.10 |
| | 76 | 4251 | 8.22 |
| | 77 | 3675 | 6.04 |
| | 78 | 3707 | 3.57 |
| | 79 | 4088 | Not determined |
| | 80 | 3872 | Not determined |

Immune Response Study in HLA-A24 Mice

Study Design.

Sixteen mixed gender HLA-A24 mice were primed with an adenoviral AdC68Y triple-antigen construct (Plasmid 1426: TERT_{Δ240}-2A-MUC1-2A-mCEA or Plasmid 1428: MUC1-2A-cCEA-2A-TERT_{Δ240}) encoding human MUC1, mCEA or cCEA, and TERT_{Δ240} at 1e10 viral particles by intramuscular injection (50 ul into each tibialis anterior muscle). 14 days later, animals were boosted intramuscularly with 50 ug triple-antigen DNA construct (Plasmid 1426 or 1428) encoding the same three antigens (20 ul delivered into each tibialis anterior muscle with concomitant electroporation). HLA-A24-restricted MUC1-specific cellular responses were measured 7 days after the last immunization in an IFN-γ ELISpot assay.

Results.

Table 13 shows the ELISpot data from HLA-A24 splenocytes cultured with the MUC1 peptide aa524-532. A positive response is defined as having SFC>50. Numbers in column 3 represent # IFN-γ spots/10⁶ splenocytes after restimulation with MUC1 peptide aa524-532 and background subtraction. As shown in Table 13, the immunogenic MUC1 polypeptides made with the MUC1-, CEA-, and TERT-expressing triple-antigen constructs 1426 and 1428 were capable of inducing HLA-A24-restricted MUC1 peptide aa524-532-specific CD8⁺ T cell responses. Importantly, T cell responses derived from cancer patients against this specific MUC1 peptide have been shown to correlate with anti-tumor efficacy in vitro (Jochems C et al., Cancer Immunol Immunother (2014) 63:161-174) demonstrating the importance of raising cellular responses against this specific epitope.

TABLE 13
HLA-A24-restricted MUC1 peptide aa524-532-specific T cell responses
induced by the triple-antigen adenoviral and DNA constructs Plasmid 1426
(TERT_{Δ240}-2A-MUC1-2A-mCEA) and Plasmid 1428 (MUC1-2A-cCEA-
2A-TERT_{Δ240}) encoding human native full-length membrane-bound
MUC1, human membrane-bound or cytosolic CEA, and human truncated
(Δ240) cytosolic TERT polypeptides in HLA-A24 mice

| | | # IFN-γ spots/ |
|---|---|---|
| Construct ID | Animal # | 10⁶ splenocytes |
| Plasmid 1426 | 49 | 49 |
| | 50 | 9 |
| | 51 | 109 |
| | 52 | 57 |
| | 53 | 75 |
| | 54 | 85 |
| | 55 | 69 |
| | 56 | 138 |
| Plasmid 1428 | 65 | 143 |
| | 66 | 81 |
| | 67 | 400 |
| | 68 | 205 |
| | 69 | 63 |
| | 70 | 74 |
| | 71 | 93 |
| | 72 | 233 |

Immune Response Study in Monkeys

Study Design.

42 Chinese-sourced cynomolgus macaques were primed on day 1 with AdC68Y adenoviral vectors encoding human native full-length membrane-bound MUC1 (MUC1), human membrane-bound or cytoplasmic CEA (mCEA or cCEA), and human truncated (Δ240) cytosolic TERT (TERT_{Δ240} antigens at 2e11 viral particles by bilateral intramuscular injection (1 mL total). On day 30 and day 57 animals were boosted with DNA encoding the same three antigens delivered intramuscularly bilaterally via electroporation (2 mL total). Anti-CTLA-4 was administered subcutaneously on days 1 (32 mg), 30 (50 mg) and 57 (75 mg). 15 days after the last immunization, animals were bled and PBMCs and serum isolated to assess MUC1-, CEA-, and TERT-specific cellular (ELISpot, ICS) and MUC1- and CEA-specific humoral (ELISA) responses, respectively. In total, six triple-antigen adenoviral and DNA constructs encoding MUC1, mCEA or cCEA, and TERT_{Δ240} linked by 2A peptides were evaluated: MUC1-2A-TERT_{Δ240}-2A-mCEA (Plasmid 1424), mCEA-2A-MUC1-2A-TERT_{Δ240} (Plasmid 1425), TERT_{Δ240}-2A-MUC1-2A-mCEA (Plasmid 1426), TERT_{Δ240}-2A-mCEA-2A-MUC1 (Plasmid 1427), MUC1-2A-cCEA-2A-TERT_{Δ240} (Plasmid 1428), cCEA-2A-TERT_{Δ240}-2A-MUC1 (Plasmid 1429).

Results.

Tables 14A, 14B, and 14C show the ELISpot and ICS data from Chinese-sourced cynomolgus macaque PBMCs cultured with peptide pools derived from the MUC1, CEA, and TERT peptide libraries (see also Table 15), and the ELISA data from Chinese-sourced cynomolgus macaque sera. A positive response is defined as having SFC>50, IFN-γ⁺ CD8⁺ T cells/1e6 CD8⁺ T cells>50, and IgG titers>99. Numbers in column 3 in Tables 14A-C represent # IFN-γ spots/10⁶ splenocytes after restimulation with MUC1, CEA, or TERT peptide pools, and background subtraction, respectively. Numbers in bold font indicate that at least 1 peptide pool tested was too numerous to count, therefore the true figure is at least the value stated. Numbers in column 4 in Tables 14A-C represent # IFN-γ⁺ CD8⁺ T cells/10⁶ CD8⁺ T cells after restimulation with MUC1, CEA, or TERT peptide pools, respectively, and background subtraction. Numbers in column 5 in Tables 14A-B represent the anti-MUC1 and anti-CEA IgG titer (Optical Density (O.D)=1, Limit of Detection (L.O.D)=99.0), respectively. As shown in Tables 14A-C, the immunogenic MUC1, CEA, and TERT polypeptides made with MUC1-, CEA-, and TERT-expressing triple-Ag constructs were capable of inducing cellular responses against all three antigens, and humoral responses against MUC1. However, triple-antigen constructs containing mCEA induced greater CEA-specific B cell responses than those containing cCEA.

TABLE 14A
MUC1-specific T and B cell responses induced by the
triple-antigen adenoviral AdC68Y and DNA constructs
(Plasmids 1424-1429) encoding human native full-length
membrane-bound MUC1, human membrane-bound or cytoplasmic
CEA, and human truncated (Δ240) cytosolic TERT polypeptides
in Chinese-sourced cynomolgus macaques

| | | MUC1 | | |
|---|---|---|---|---|
| | | # IFN-γ | # IFN-γ⁺ CD8⁺ | |
| | | spots/10⁶ | T cells/1e6 | |
| Construct ID | Animal # | splenocytes | CD8⁺ T cells | IgG titer |
| Plasmid 1424 | 1001 | 152 | 0 | 8100 |
| | 1002 | 687 | 2211.9 | 7230 |
| | 1003 | 313 | 0 | 7630 |
| | 1004 | 1610 | 12216.5 | 21500 |
| | 1501 | 93 | 0 | 13700 |
| | 1502 | 128 | 0 | 22700 |
| | 1503 | 637 | 1839.2 | 13800 |
| Plasmid 1425 | 2001 | 533 | 0 | 10700 |
| | 2002 | 1445 | 0 | 17200 |
| | 2003 | 833 | 4585.5 | 9640 |
| | 2004 | 143 | 0 | 20200 |
| | 2501 | 297 | 0 | 8130 |
| | 2502 | 223 | 0 | 13800 |
| | 2503 | 540 | 2096.4 | 4130 |
| Plasmid 1426 | 3001 | 135 | 0 | 14100 |
| | 3002 | 87 | 0 | 22100 |
| | 3003 | 70 | 0 | 15700 |
| | 3004 | 677 | 0 | 25900 |
| | 3501 | 1965 | 7438.9 | 17500 |
| | 3502 | 1667 | 899.2 | 34700 |
| | 3503 | 540 | 0 | 31100 |
| Plasmid 1427 | 4001 | 187 | 0 | 15800 |
| | 4002 | 37 | 0 | 4870 |
| | 4003 | 568 | 1290.0 | 11300 |
| | 4004 | 1558 | 23074.2 | 8490 |
| | 4501 | 22 | 0 | 8520 |
| | 4502 | 1267 | 561.4 | 13300 |
| | 4503 | 572 | 4615.6 | 5390 |
| Plasmid 1428 | 5001 | 40 | 1019.3 | 9960 |
| | 5002 | 973 | 6178.6 | 11100 |
| | 5003 | 47 | 0 | 18400 |
| | 5004 | 1175 | 3574.1 | 28200 |
| | 5501 | 245 | 0 | 12000 |
| | 5502 | 1663 | 1145.4 | 57100 |
| | 5503 | 1825 | 10680.4 | 15300 |
| Plasmid 1429 | 6001 | 320 | 300.1 | 19600 |
| | 6002 | 787 | 305.6 | 20900 |
| | 6003 | 233 | 0 | 11200 |
| | 6004 | 443 | 0 | 12700 |
| | 6501 | 80 | 0 | 12600 |
| | 6502 | 688 | 0 | 35600 |
| | 6503 | 1755 | 0 | 16900 |

TABLE 14B
CEA-specific T and B cell responses induced by the triple-
antigen adenoviral AdC68Y and DNA constructs (Plasmids
1424-1429) encoding human native full-length membrane-
bound MUC1, human membrane-bound or cytoplasmic CEA,
and human truncated (Δ240) cytosolic TERT polypeptides
in Chinese-sourced cynomolgus macaques

| | | CEA | | |
|---|---|---|---|---|
| | | # IFN-γ | # IFN-γ⁺ CD8⁺ | |
| | | spots/10⁶ | T cells/1e6 | |
| Construct ID | Animal # | splenocytes | CD8⁺ T cells | IgG titer |
| Plasmid 1424 | 1001 | 130 | 0 | 26300 |
| | 1002 | 268 | 0 | 31100 |
| | 1003 | 882 | 2002.6 | 16700 |
| | 1004 | 1248 | 0 | 115000 |
| | 1501 | 133 | 0 | 39600 |
| | 1502 | 350 | 0 | 97500 |
| | 1503 | 323 | 947.1 | 27200 |
| Plasmid 1425 | 2001 | 650 | 1191.4 | 37700 |
| | 2002 | 2003 | 5608.2 | 38700 |
| | 2003 | 342 | 0 | 35600 |
| | 2004 | 343 | 0 | 74900 |
| | 2501 | 1770 | 1507.0 | 41700 |
| | 2502 | 1547 | 20858.5 | 54000 |
| | 2503 | 957 | 1549.3 | 36200 |
| Plasmid 1426 | 3001 | 448 | 0 | 94400 |
| | 3002 | 158 | 0 | 88300 |
| | 3003 | 92 | 0 | 104000 |
| | 3004 | 342 | 0 | 119000 |
| | 3501 | 2383 | 0 | 64400 |
| | 3502 | 2112 | 0 | 49300 |
| | 3503 | 1673 | 0 | 177000 |
| Plasmid 1427 | 4001 | 2065 | 46600.1 | 48200 |
| | 4002 | 195 | 0 | 44300 |
| | 4003 | 657 | 0 | 35500 |
| | 4004 | 2238 | 7032.7 | 97500 |
| | 4501 | 1370 | 9081.2 | 62100 |
| | 4502 | 2065 | 1704.7 | 48500 |
| | 4503 | 1020 | 3005.5 | 23500 |
| Plasmid 1428 | 5001 | 430 | 2597.8 | 29300 |
| | 5002 | 1212 | 5461.8 | 8730 |
| | 5003 | 297 | 1150.6 | 15800 |
| | 5004 | 245 | 2367.2 | 52200 |
| | 5501 | 397 | 564.7 | 12900 |
| | 5502 | 525 | 0 | 16100 |
| | 5503 | 710 | 4371.4 | 5330 |
| Plasmid 1429 | 6001 | 263 | 248.1 | 9650 |
| | 6002 | 342 | 0 | 5320 |
| | 6003 | 367 | 1111.6 | 1960 |
| | 6004 | 1343 | 7055.3 | 1500 |
| | 6501 | 1015 | 6290.7 | 16700 |
| | 6502 | 1827 | 796.4 | 28000 |
| | 6503 | 1740 | 11848.4 | 13200 |

TABLE 14C
TERT-specific T cell responses induced by the triple-antigen adenoviral
AdC68Y and DNA constructs (Plasmids 1424-1429) encoding human
native full-length membrane-bound MUC1, human membrane-bound or
cytoplasmic CEA, and human truncated (Δ240) cytosolic TERT
polypeptides in Chinese-sourced cynomolgus macaques

| | | TERT | |
|---|---|---|---|
| | | # IFN-γ spots/ | # IFN-γ⁺ CD8⁺ T cells/ |
| Construct ID | Animal # | 10⁶ splenocytes | 1e6 CD8⁺ T cells |
| Plasmid 1424 | 1001 | 988 | 1854.9 |
| | 1002 | 512 | 609.1 |
| | 1003 | 320 | 0 |
| | 1004 | 1762 | 9865.4 |
| | 1501 | 1413 | 11844.9 |
| | 1502 | 1890 | 15520.3 |
| | 1503 | 978 | 2592.6 |
| Plasmid 1425 | 2001 | 223 | 3278.8 |
| | 2002 | 1512 | 263.9 |
| | 2003 | 318 | 6939.2 |
| | 2004 | 55 | 0 |
| | 2501 | 118 | 0 |
| | 2502 | 1808 | 0 |
| | 2503 | 233 | 373.4 |
| Plasmid 1426 | 3001 | 2798 | 37114.7 |
| | 3002 | 853 | 0 |
| | 3003 | 1458 | 32332.1 |
| | 3004 | 653 | 207.5 |
| | 3501 | 1878 | 6543.9 |
| | 3502 | 2957 | 33410.8 |
| | 3503 | 1958 | 5418.9 |
| Plasmid 1427 | 4001 | 242 | 379.5 |
| | 4002 | 1237 | 4109.2 |
| | 4003 | 903 | 1438.5 |
| | 4004 | 185 | 1196.0 |
| | 4501 | 105 | 2526.1 |
| | 4502 | 2815 | 16330.0 |
| | 4503 | 608 | 6737.9 |
| Plasmid 1428 | 5001 | 50 | 652.8 |
| | 5002 | 468 | 504.5 |
| | 5003 | 1277 | 284.7 |
| | 5004 | 333 | 2714.2 |
| | 5501 | 1217 | 8357.3 |
| | 5502 | 1715 | 833.4 |
| | 5503 | 243 | 462.6 |
| Plasmid 1429 | 6001 | 643 | 802.5 |
| | 6002 | 2557 | 5767.4 |
| | 6003 | 1082 | 3997.1 |
| | 6004 | 1890 | 2557.6 |
| | 6501 | 1447 | 22060.9 |
| | 6502 | 2032 | 0 |
| | 6503 | 2082 | 637.7 |

TABLE 15
Peptide Pools Derived from Human Tumor-Associated
Antigen (TAA) MUC1, CEA, and TERT

| TAA | Peptide Pools |
|---|---|
| MUC1 | 116 sequential 15-mer peptides, overlapping by 11 amino acids, |
| | covering amino acids 1-224 and 945-1255 (excluding all but 1 of |
| | 20 amino acid repeats) of the MUC1 precursor protein of SEQ |
| | ID NO: 1 |
| CEA | 125 sequential 15-mer peptides, overlapping by 11 amino acids, |
| | covering the CEA protein sequence of amino acids 1-147 and |
| | amino acids 325-699 (excluding domains 2-3) of SEQ ID N0: 2 |
| TERT | 221 sequential 15-mer peptides, overlapping by 11 amino acids, |
| | coveringthe TERT_{Δ240} protein sequence of SEQ ID NO: 10 |
| | (amino acids 241-1134, total 894 amino acids), excluding the |
| | first 240 amino acids of the native full-length TERT protein |
| | of SEQ ID NO: 3 |

TABLE 16
Primers for Plasmid Construction

| Primer | SEQUENCE (5′ TO 3′) | Strand |
|---|---|---|
| EMCV_Muc1_R-35 | GTTGAAGATTCTGCCGGATCCCAGGTTGGCGG | Antisense |
| | AGGCAGCGGCCACG | |
| EMCV2A_F-34 | GCTACTTCGCCGACCTGCTGATCCACGACA | Sense |
| | TCGAGACAAACCCTGGC | |
| EMCV2A_R-36 | GGTCGGCGAAGTAGCCGGCGTAGTGGGCG | Antisense |
| | TTGAAGATTCTGCCGGAT | |
| f Muc 960-983 | CGGCGTCTCATTCTTCTTTCTGTC | Sense |
| f pmed Nhe cytMuc | ACCCTGTGACGAACATGGCTAGCACAGGCT | Sense |
| | CTGGCCACGCCAG | |
| f pmed Nhe Muc | ACCCTGTGACGAACATGGCTAGCACCCCTG | Sense |
| | GAACCCAGAGCC | |
| f pmed Nhe Ter240 | ACCCTGTGACGAACATGGCTAGCGGAGCTG | Sense |
| | CCCCGGAGCCGG | |
| f tert 1584-1607 | TCTCACCGACCTCCAGCCTTACAT | Sense |
| f tg link Ter240 | TGGGAGGCTCCGGCGGAGGAGCTGCCCCG | Sense |
| | GAGCCGG | |
| f1 EM2A Muc | CCTGCTGATCCACGACATCGAGACAAACCC | Sense |
| | TGGCCCCACCCCTGGAACCCAGAGCC | |
| f1 ERBV2A cMuc | TGGCCGGCGACGTGGAACTGAACCCTGGC | Sense |
| | CCTACAGGCTCTGGCCACGCCAG | |
| f1 ERBV2A Muc | TGGCCGGCGACGTGGAACTGAACCCTGGC | Sense |
| | CCTACCCCTGGAACCCAGAGCC | |
| f1 ERBV2A Ter d342 | TGGCCGGCGACGTGGAACTGAACCCTGGC | Sense |
| | CCTAGCTTCCTCCTGTCGTCGCTCA | |
| f1 ERBV2A Ter240 | TGGCCGGCGACGTGGAACTGAACCCTGGC | Sense |
| | CCTGGAGCTGCCCCGGAGCCGG | |
| f1 ERBV2A Tert | TGGCCGGCGACGTGGAACTGAACCCTGGC | Sense |
| d541 | CCTGCCAAATTTCTGCATTGGCTGATG | |
| f1 PTV2A Muc | TGGAAGAGAACCCTGGCCCTACCCCTGGAA | Sense |
| | CCCAGAGCC | |
| f1 T2A Tert d342 | GCGACGTGGAAGAGAACCCTGGCCCCAGCT | Sense |
| | TCCTCCTGTCGTCGCTCA | |
| f1 T2A Tert d541 | GCGACGTGGAAGAGAACCCTGGCCCCGCC | Sense |
| | AAATTTCTGCATTGGCTGATG | |
| f1 T2A Tert240 | GCGACGTGGAAGAGAACCCTGGCCCCGGA | Sense |
| | GCTGCCCCGGAGCCGG | |
| f2 EMCV2A | AGAATCTTCAACGCCCACTACGCCGGCTAC | Sense |
| | TTCGCCGACCTGCTGATCCACGACATCGA | |
| f2 ERBV2A | TGTCTGAGGGCGCCACCAACTTCAGCCTGC | Sense |
| | TGAAACTGGCCGGCGACGTGGAACTG | |
| f2 PTV2A | TTCAGCCTGCTGAAACAGGCCGGCGACGTG | Sense |
| | GAAGAGAACCCTGGCCCT | |
| f2 T2A | CCGGCGAGGGCAGAGGCAGCCTGCTGACA | Sense |
| | TGTGGCGACGTGGAAGAGAACCCTG | |
| pMED_MUC1_F-31 | ACGAACATGGCTAGCACCCCTGGAACCCAG | Sense |
| | AGCCCCTTC | |
| r ERB2A Bamh Muc | TGGTGGCGCCCTCAGACAGGATTGTGCCGG | Antisense |
| | ATCCCAGGTTGGCGGAGGCAGCG | |
| r ERB2A Bamh | TGGTGGCGCCCTCAGACAGGATTGTGCCGG | Antisense |
| Ter240 | ATCCGTCCAAGATGGTCTTGAAATCTGA | |
| r link muc | TCCGCCGGAGCCTCCCAGGTTGGCGGAGG | Antisense |
| | CAGCG | |
| r link Tert240 | TCCGCCGGAGCCTCCGTCCAAGATGGTCTT | Antisense |
| | GAAATCTGA | |
| r muc 986-963 | AAGGACAGAAAGAAGAATGAGACG | Antisense |
| r pmed Bgl Muc | TTGTTTTGTTAGGGCCCAGATCTTCACAGGT | Antisense |
| | TGGCGGAGGCAGCG | |
| r pmed Bgl Ter240 | TTGTTTTGTTAGGGCCCAGATCTTCAGTCCA | Antisense |
| | AGATGGTCTTGAAATCTGA | |
| r PTV2A Bamh Muc | CTGTTTCAGCAGGCTGAAATTGGTGGCGCC | Antisense |
| | GGATCCCAGGTTGGCGGAGGCAGCG | |
| r T2A Tert240 | TGCCTCTGCCCTCGCCGGATCCGTCCAAGA | Antisense |
| | TGGTCTTGAAATCTGA | |
| r tert 1602-1579 | AGGCTGGAGGTCGGTGAGAGTGGA | Antisense |
| r2 T2A | AGGGTTCTCTTCCACGTCGCCACATGTCAG | Antisense |
| | CAGGCTGCCTCTGCCCTCGCCGGATCC | |
| TertΔ343-F | ACGAACATGGCTAGCTTCCTCCTGTCGTCG | Sense |
| | CTCAGACCGAG | |
| Tert-R | TTGTTTTGTTAGGGCCCAGATCTTCAGTCCA | Antisense |
| | AGATGGTCTTGAAATC | |
| TertΔ541-F | ACGAACATGGCTAGCGCCAAATTTCTGCATT | Sense |
| | GGCTGATGTC | |
| r TERT co# pMed | TTGTTTTGTTAGGGCCCAGATCTTCAGTCCA | Antisense |
| | AGATGGTCTTGAAATC | |
| f pmed TERT 241G | ACCCTGTGACGAACATGGGAGCTGCCCCGG | Sense |
| | AGCCGGAGA | |
| ID1197F | ACCCTGTGACGAACATGGCTAGC | Sense |
| ID1197R | AGATCTGGGCCCTAACA | Antisense |
| f cCEA 562-592 | TCCGTGGACCACAGCGACCCTGTGATCCTGA | Sense |
| f CEA 833-855 | CTGTCAAAACTATCACTGTGTCC | Sense |
| f EMC2a CEA d1 | CTTCGCCGACCTGCTGATCCACGACATCGA | Sense |
| | GACAAACCCTGGCCCCAAGCTGACCATTGA | |
| | GAGCACTCCCTTCAAC | |
| f pmed CEA D1 | ACCCTGTGACGAACATGGCTAGCAAGCTGA | Sense |
| | CCATTGAGAGCACTCCCTTCAACGTG | |
| f pmed CEA SS | ACCCTGTGACGAACATGGCTAGCGAATCGC | Sense |
| | CAAGCGCACCCCCTCATCGGTGGTGCATCC | |
| | CTTGGCAACGC | |
| f1 EMC2a CEAss | CTTCGCCGACCTGCTGATCCACGACATCGA | Sense |
| | GACAAACCCTGGCCCCGAATCGCCAAGCGC | |
| | ACCCCCTCATCGGTGG | |
| f1 ERA2A ssCEA | AAGCTGGCCGGCGACGTGGAATCTAACCCT | Sense |
| | GGCCCTGAATCGCCAAGCGCACCCCCTCAT | |
| | CGGTGG | |
| f1 T2a Muc | TGTGGCGACGTGGAAGAGAACCCTGGCCCC | Sense |
| | ACCCCTGGAACCCAGAGCCCCTTCTTCCTT | |
| f2 ERAV2A | TCCGGCCAGTGCACCAATTACGCCCTGCTG | Sense |
| | AAGCTGGCCGGCGACGTGGA | |
| f2 T2A 63 | GGATCCGGCGAGGGCAGAGGCAGCCTGCT | Sense |
| | GACATGTGGCGACGTGGAAGAGAACCCTGG | |
| | CCCC | |
| f CEA D1-D4 | AGGGTGTACCCCGAACTCCCTAAGCCGTTC | Sense |
| | ATCACCTCGAACAACAGCAAC | |
| ID1361-1362_PCRF | ACCCTGTGACGAACATGGCTAGCGAATCGC | Sense |
| | CAAGCGCACCCCCTCATC | |
| r cCEA 849-820 | AGTGATAGTTTTGACAGTGGTGCGGGAGTG | Antisense |
| R CEA SR2 | AACACTTCCTACCGGTCCGGAG | Antisense |
| r EM2A Bamh Muc | GTGGGCGTTGAAGATTCTGCCGGATCCCAG | Antisense |
| | GTTGGCGGAGGCAGCG | |
| r ERA2A Tert | ATTGGTGCACTGGCCGGATCCGTCCAAGAT | Antisense |
| | GGTCTTGAAATCTGA | |
| r pmed CEA D7 | TTGTTTTGTTAGGGCCCAGATCTTCAGGACG | Antisense |
| | CCGACACGGTAATGGACTTCACGA | |
| r pmed CEA GPI | TTGTTTTGTTAGGGCCCAGATCTTCAGATCA | Antisense |
| | GGGCCACTCCCACGAGCAC | |
| r T2a CEA | TCTGCCCTCGCCGGATCCGATCAGGGCCAC | Antisense |
| | TCCCACGAGCACGCCGAT | |
| r T2a CEA D7 | TCTGCCCTCGCCGGATCCGGACGCCGACAC | Antisense |
| | GGTAATGGACTTCACGAT | |
| r T2A furin CEA | TCTGCCCTCGCCGGATCCTCTTCTCTTCCTG | Antisense |
| | ATCAGGGCCACTCCCACGAGCACGCCGAT | |
| r CEA D1 | GAGTTCGGGGTACACCCTGAATTGGCCGGT | Antisense |
| | GGC | |
| ID1361-1362_PCRR | TTGTTAGGGCCCAGATCTTCAGATCAGGGC | Antisense |
| | CACTCCCACGAG | |
| Muc1-20bp-F-98 | CCGCTAGGGTACCGCGATCACCATGGCTAG | Sense |
| | CACCCCTGGAACCCAGAGCCCCTTC | |
| mCEA-20bp-R-100 | TTATGATCAGCTCGAGGTGCGTCAGATCAG | Antisense |
| | GGCCACTCCCACGAGCACGCCGATC | |
| Y-mCEA-S2 | GATCCGCTAGGGTACCGCGATCACCATGGC | Sense |
| | TAGCGAATCGCCAAGCGCACCCCCTCATC | |
| Y-Tert-A2 | GATCAGCTCGAGGTGCGTCAGTCCAAGATG | Antisense |
| | GTCTTGAAATCTGACGGCAATG | |
| Y-Tert-S | GATCCGCTAGGGTACCGCGATCACCATGGC | Sense |
| | TAGCGGAGCTGCCCCGGAGCC | |
| Y-CEA-A | GATCAGCTCGAGGTGCGATTCAGATCAGGG | Antisense |
| | CCACTCCCACGAGCAC | |
| Y-MUC-A | GATCAGCTCGAGGTGCGATTCACAGGTTGG | Antisense |
| | CGGAGGCAGCGGCC | |
| Y-MUC-S2 | GATCCGCTAGGGTACCGCGATCACCATGGC | Sense |
| | TAGCACCCCTGGAACCCAGAGCCCCTTC | |
| cCEA-20bp-F-106 | GATCCGCTAGGGTACCGCGATCACCATGGC | Sense |
| | TAGCAAGCTGACCATTGAGAGCACTC | |
| Muc1-20BP-R-108 | GATTATGATCAGCTCGAGGTGCGTCACAGG | Antisense |
| | TTGGCGGAGGCAGCGGCCACGGCAGG | |

TABLE 17
2A-peptide Sequences

| Virus | 2A-peptide Sequence |
|---|---|
| Foot and mouse disease | VKQTLNFDLLKLAGDVESNPG |
| virus (FMDV) | |
| Equine rhinitis A virus | QCTNYALLKLAGDVESNPG |
| (ERAV) | |
| Porcine teschovirus-1 | ATNF-SLLKQAGDVEENPG |
| (PTV1) | |
| Encephalomyocarditis | HYAGYFADLLIHDIETNPG |
| virus (EMCV) | |
| Encephalomyocarditis B | GIFN-AHYAGYFADLLIHDIETNPG |
| variant (EMC-B) | |
| Theiler murine | KAVRGYHADYYKQRLIHDVEMNPG |
| encephalomyelitis GD7 | |
| (TME-GD7) | |
| Equine rhinitis B virus | GATNF-SLLKLAGDVELNPG |
| (ERBV) | |
| Thosea asigna virus | EGRGSLLTCGDVEENPG |
| (TAV) | |
| Drosophilia C (DrosC) | AARQMLLLLSGDVETNPG |
| Cricket paralysis virus | FLRKRTQLLMSGDVESNPG |
| (CrPV) | |
| Acute bee paralysis | GSWTDILLLLSGDVETNPG |
| virus (ABPV) | |
| Infectious flacherie | TRAEIEDELIRAGIESNPG |
| virus (IFV) | |
| Porcine rotavirus | AKFQIDKILISGDVELNPG |
| Human rotavirus | SKFQIDKILISGDIELNPG |
| T. brucei TSR1 | SSIIRTKMLVSGDVEENPG |
| T. cruzi AP endonuclease | CDAQRQKLLLSGDIEQNPG |

TABLE 18
Sequence Index

| SEQ ID NO | Description |
|---|---|
| 1 | Amino acid sequence of human MUC1 Isoform 1 precursor protein |
| | (Reference Polypeptide; Uniprot P15941-1) |
| 2 | Amino acid sequence of human CEA Isoform 1 precursor protein |
| | (Reference Polypeptide; Uniprot P06731-1 (702 aa) |
| 3 | Amino acid sequence of human TERT Isoform 1 precursor protein |
| | (Reference Polypeptide; Genbank AAD30037, Uniprot O14746-1) |
| 4 | Plasmid 1027 (MUC1) - ORF nucleotide sequence (DNA) |
| 5 | Plasmid 1027 (MUC1) - encoded amino acid sequence |
| 6 | Plasmid 1197 (cMUC1) - ORF nucleotide sequence (DNA) |
| 7 | Plasmid 1197 (cMUC1) - encoded amino acid sequence |
| 8 | Plasmid 1112 (TERT240) - ORF nucleotide sequence (DNA) |
| 9 | Plasmid 1112 (TERT240) - encoded amino acid sequence |
| 10 | Plasmid 1326 (TERT343) - ORF nucleotide sequence (DNA) |
| 11 | Plasmid 1326 (TERT343) - encoded amino acid sequence |
| 12 | Plasmid 1330 (TERT541) - ORF nucleotide sequence (DNA) |
| 13 | Plasmid 1330 (TERT541) - encoded amino acid sequence |
| 14 | Plasmid 1361 (CEA) - ORF nucleotide sequence (DNA) |
| 15 | Plasmid 1361 (CEA) - encoded amino acid sequence |
| 16 | Plasmid 1386 (mCEA) - ORF nucleotide sequence (DNA) |
| 17 | Plasmid 1386 (mCEA) - encoded amino acid sequence |
| 18 | Plasmid 1390 (cCEA) - ORF nucleotide sequence (DNA) |
| 19 | Plasmid 1390 (cCEA) - encoded amino acid sequence |
| 20 | Plasmid 1269 (Muc1 - Tert240) - ORF nucleotide sequence (DNA) |
| 21 | Plasmid 1269 (Muc1 - Tert240) - encoded amino acid sequence |
| 22 | Plasmid 1270 (Muc1 - ERB2A- Tert240) - ORF nucleotide sequence (DNA) |
| 23 | Plasmid 1270 (Muc1 - ERB2A- Tert240) - encoded amino acid sequence |
| 24 | Plasmid 1271 (Tert240 - ERB2A - Muc1) - ORF nucleotide sequence (DNA) |
| 25 | Plasmid 1271 (Tert240 - ERB2A - Muc1) - encoded amino acid sequence |
| 26 | Plasmid 1286 (cMuc1 - ERB2A - Tert240) - ORF nucleotide sequence (DNA) |
| 27 | Plasmid 1286 (cMuc1 - ERB2A - Tert240) - encoded amino acid sequence |
| 28 | Plasmid 1287 (Tert240 - ERB2A - cMuc1) - ORF nucleotide sequence (DNA) |
| 29 | Plasmid 1287 (Tert240 - ERB2A - cMuc1) - encoded amino acid sequence |
| 30 | Plasmid 1409 (Muc1 - EMC2A - mCEA) - ORF nucleotide sequence (DNA) |
| 31 | Plasmid 1409 (Muc1 - EMC2A - mCEA) - encoded amino acid sequence |
| 32 | Plasmid 1410 (mCEA - T2A - Muc1) - ORF nucleotide sequence (DNA) |
| 33 | Plasmid 1410 (mCEA - T2A - Muc1) - encoded amino acid sequence |
| 34 | Plasmid 1411 (mCEA - Furin - T2A - Muc1) - ORF nucleotide sequence (DNA) |
| 35 | Plasmid 1411 (mCEA - Furin - T2A - Muc1) - encoded amino acid sequence |
| 36 | Plasmid 1431 (Muc1 - EMC2A- cCEA) - ORF nucleotide sequence (DNA) |
| 37 | Plasmid 1431 (Muc1 - EMC2A - cCEA) - encoded amino acid sequence |
| 38 | Plasmid 1432 (cCEA - T2A - Tert240) - ORF nucleotide sequence (DNA) |
| 39 | Plasmid 1432 (cCEA - T2A - Tert240) - encoded amino acid sequence |
| 40 | Plasmid 1440 (Tert240 - ERA2A - mCEA) - ORF nucleotide sequence (DNA) |
| 41 | Plasmid 1440 (Tert240 - ERA2A - mCEA) - encoded amino acid sequence |
| 42 | Plasmid 1424 (Muc1-ERB2A -Tert240- ERA2A- mCEA) - ORF nucleotide sequence (DNA) |
| 43 | Plasmid 1424 (Muc1- ERB2A - Tert240 -ERA2A- mCEA) - encoded amino acid sequence |
| 44 | Plasmid 1425 (mCEA- T2A - Muc1 - ERB2A - Tert240) - ORF nucleotide sequence (DNA) |
| 45 | Plasmid 1425 (mCEA -T2A - Muc1 - ERB2A -Tert240) - encoded amino acid sequence |
| 46 | Plasmid 1426 (Tert240 - ERB2A- Muc1- EMC2A - mCEA) - ORF nucleotide sequence (DNA) |
| 47 | Plasmid 1426 (Tert240 - ERB2A - Muc1 - EMC2A - mCEA) - encoded amino acid sequence |
| 48 | Plasmid 1427 (Tert240 - ERA2A - mCEA - T2A - Muc1) - ORF nucleotide sequence (DNA) |
| 49 | Plasmid 1427 (Tert240 - ERA2A - mCEA - T2A - Muc1) - encoded amino acid sequence |
| 50 | Plasmid 1428 (Muc1 - EMC2A - cCEA - T2A - Tert240) - ORF nucleotide sequence (DNA) |
| 51 | Plasmid 1428 (Muc1 - EMC2A - cCEA - T2A - Tert240) - encoded amino acid sequence |
| 52 | Plasmid 1429 (cCEA - T2A - Tert240 - ERB2A - Muc1) - ORF nucleotide sequence (DNA) |
| 53 | Plasmid 1429 (cCEA - T2A - Tert240 - ERB2A - Muc1) - encoded amino acid sequence |
| 54 | Plasmid 1361 - complete vector nucleotide sequence (DNA) |
| 55 | Plasmid 1390 - complete vector nucleotide sequence (DNA) |
| 56 | Plasmid 1386 - complete vector nucleotide sequence (DNA) |
| 57 | Plasmid 1424 - complete vector nucleotide sequence (DNA) |
| 58 | AdC68-1424 - complete vector nucleotide sequence (DNA) |
| 59 | Plasmid 1425 - complete vector nucleotide sequence (DNA) |
| 60 | AdC68-1425 - complete vector nucleotide sequence (DNA) |
| 61 | Plasmid 1426 - complete vector nucleotide sequence (DNA) |
| 62 | AdC68-1426 - complete vector nucleotide sequence (DNA) |
| 63 | Plasmid 1427 complete vector nucleotide sequence (DNA) |
| 64 | AdC68-1427 - complete vector nucleotide sequence (DNA) |
| 65 | Plasmid 1428 - complete vector nucleotide sequence (DNA) |
| 66 | AdC68-1428 - complete vector nucleotide sequence (DNA) |
| 67 | Plasmid 1429 - complete vector nucleotide sequence (DNA) |
| 68 | AdC68-1429 - complete vector nucleotide sequence (DNA) |
| 69 | Plasmid 1409 - complete vector nucleotide sequence (DNA) |
| 70 | Plasmid 1410 - complete vector nucleotide sequence (DNA) |
| 71 | Plasmid 1411 - complete vector nucleotide sequence (DNA) |
| 72 | Plasmid 1431 - complete vector nucleotide sequence (DNA) |
| 73 | Plasmid 1432 - complete vector nucleotide sequence (DNA) |
| 74 | Plasmid 1440 - complete vector nucleotide sequence (DNA) |
| 75 | AdC68Y Empty vector nucleotide sequence (without the transgene insert) (DNA) |
| 76 | Encephalomyocarditis Virus 2A (EMC2A) amino acid sequence |
| 77 | Equine rhinitis A virus 2A (ERA2A) amino acid sequence |
| 78 | Equine Rhinitis B Virus 2A (ERB2A) amino acid sequence |
| 79 | Porcine Teschovirus 2A (PT2A) amino acid sequence |
| 80 | Thosea Asigna Virus 2A (TA2A or T2A) amino acid sequence |
| 81 | Plasmid 1065 (TERT D712A/V713I) - encoded amino acid sequence |
| 82 | Plasmid 1065 - ORF nucleotide sequence (DNA) |
| 83 | Plasmid 1065 - complete vector nucleotide sequence (DNA) |
| 84 | Plasmid 1027 - ORF nucleotide sequence (RNA) |
| 85 | Plasmid 1112 - ORF nucleotide sequence (RNA) |
| 86 | Plasmid 1390 - ORF nucleotide sequence (RNA) |
| 87 | Plasmid 1424 - ORF nucleotide sequence (RNA) |
| 88 | Plasmid 1425 - ORF nucleotide sequence (RNA) |
| 89 | Plasmid 1426 - ORF nucleotide sequence (RNA) |
| 90 | Plasmid 1427 - ORF nucleotide sequence (RNA) |
| 91 | Plasmid 1428 - ORF nucleotide sequence (RNA) |
| 92 | Plasmid 1429 - ORF nucleotide sequence (RNA) |
| 93 | Encephalomyocarditis virus (EMCV) IRES sequence (RNA) |

RAW SEQUENCE LISTING (PARTIAL)
Plasmid 1027 ORF (MUC1)

SEQ ID NO: 4

atggctagcacccctggaacccagagccccttcttccttctgctgctgctgaccgtgctgactgtcgtgacaggctctggccacgccagctc
tacacctggcggcgagaaagagacaagcgccacccagagaagcagcgtgccaagcagcaccgagaagaacgccgtgtccatgacca
gctccgtgctgagcagccactctcctggcagcggcagcagcacaacacagggccaggatgtgacactggcccctgccacagaacctgc
ctctggatctgccgccacctggggacaggacgtgacaagcgtgccagtgaccagacctgccctgggctctacaacaccccctgcccacg
atgtgaccagcgcccctgataacaagcctgcccctggaagcacagcccctccagctcatggcgtgacctctgccccagataccagacca
gccccaggatctacagccccacccgcacacggcgtgacaagtgcccctgacacaagacccgctccaggctctactgctcctcctgcccat
ggcgtgacaagcgctcccgatacaaggccagctcctggctccacagcaccaccagcacatggcgtgacatcagctcccgacactagacc
tgctcccggatcaaccgctccaccagctcacggcgtgaccagcgcacctgataccagacctgctctgggaagcaccgcccctcccgtgc
acaatgtgacatctgcttccggcagcgccagcggctctgcctctacactggtgcacaacggcaccagcgccagagccacaacaacccca
gccagcaagagcacccccttcagcatccctagccaccacagcgacacccctaccacactggccagccactccaccaagaccgatgcctc
tagcacccaccactccagcgtgccccctctgaccagcagcaaccacagcacaagcccccagctgtctaccggcgtctcattcttctttctgt
ccttccacatcagcaacctgcagttcaacagcagcctggaagatcccagcaccgactactaccaggaactgcagcgggatatcagcgag
atgttcctgcaaatctacaagcagggcggcttcctgggcctgagcaacatcaagttcagacccggcagcgtggtggtgcagctgaccctg
gctttccgggaaggcaccatcaacgtgcacgacgtggaaacccagttcaaccagtacaagaccgaggccgccagccggtacaacctga
ccatctccgatgtgtccgtgtccgacgtgcccttcccattctctgcccagtctggcgcaggcgtgccaggatggggaattgctctgctggtgc
tcgtgtgcgtgctggtggccctggccatcgtgtatctgattgccctggccgtgtgccagtgccggcggaagaattacggccagctggacat
cttccccgccagagacacctaccaccccatgagcgagtaccccacataccacacccacggcagatacgtgccacccagctccaccgaca
gatccccctacgagaaagtgtctgccggcaacggcggcagctccctgagctacacaaatcctgccgtggccgctgcctccgccaacctg.
Plasmid 1027 Polypeptide (MUC1)

SEQ ID NO: 5

MASTPGTQSPFFLLLLLTVLTVVTGSGHASSTPGGEKETSATQRSSVPSSTEKNAVSMTS
SVLSSHSPGSGSSTTQGQDVTLAPATEPASGSAATWGQDVTSVPVTRPALGSTTPPAHD
VTSAPDNKPAPGSTAPPAHGVTSAPDTRPAPGSTAPPAHGVTSAPDTRPAPGSTAPPAHG
VTSAPDTRPAPGSTAPPAHGVTSAPDTRPAPGSTAPPAHGVTSAPDTRPALGSTAPPVHN
VTSASGSASGSASTLVHNGTSARATTTPASKSTPFSIPSHHSDTPTTLASHSTKTDASSTH
HSSVPPLTSSNHSTSPQLSTGVSFFFLSFHISNLQFNSSLEDPSTDYYQELQRDISEMFLQI
YKQGGFLGLSNIKFRPGSVVVQLTLAFREGTINVHDVETQFNQYKTEAASRYNLTISDV
SVSDVPFPFSAQSGAGVPGWGIALLVLVCVLVALAIVYLIALAVCQCRRKNYGQLDIFP
ARDTYHPMSEYPTYHTHGRYVPPSSTDRSPYEKVSAGNGGSSLSYTNPAVAAASANL.
Plasmid 1112 ORF (TERT240)

SEQ ID NO: 8

atgggagctgccccggagccggagaggacccccgttggccagggatcgtgggcccatccgggacgcaccaggggaccatccgacag
gggattctgtgtggtgtcaccggccaggccagcagaagaggcaaccagcctcgagggagcgttgtctggaaccagacattcccacccgt
cggtgggccggcagcaccacgcgggaccaccgtccacttccagaccgccacggccatgggacaccccttgcccgcctgtgtatgccga
gactaaacacttcctgtactcatccggagacaaggaacagcttcggccgtccttcctcctgtcgtcgctcagaccgagcctgaccggagca
cgcagattggtggaaactatcttccttgggtcacgtccgtggatgccaggtaccccacggcgcctcccgcgcctcccacagagatactggc
agatgcggcctctgttcctggaattgctgggaaaccacgctcagtgcccgtacggagtcctgctcaagactcactgccctctgagggcggc
ggtcactccggcggccggagtgtgcgcacgggagaagccccagggaagcgtggcagctccggaagaggaggacaccgatccgcgc
cgcctcgtgcaacttctgcgccagcactcctcgccctggcaagtctacgggttcgtccgcgcctgcctgcgccgcctggtgccgcctggg
ctctggggttcccggcataacgagcgccgcttcctgagaaatactaagaagtttatctcacttggaaaacatgccaagttgtcgctgcaagaa
ctcacgtggaagatgtcagtccgcgattgcgcctggctgcgccgctcgccgggcgtcgggtgtgttccagctgcagaacaccgcctgaga
gaagaaattctggccaaatttctgcattggctgatgtcagtgtacgtggtcgagctgctgcgctcctttttctacgtcactgagactacctttcaa
aagaaccgcctgttcttctaccgcaaatctgtgtggagcaagctgcagtcaatcggcattcgccagcatctgaagagggtgcagctgcggg
aactttccgaggcagaagtccgccagcaccgggaggcccggccggcgcttctcacgtcgcgtctgagattcatcccaaagcccgacggg
ctgaggcctatcgtcaacatggattacgtcgtgggcgctcgcacctttcgccgtgaaaagcgggccgaacgcttgacctcacgggtgaag
gccctcttctccgtgctgaactacgagagagcaagacggcctggcctgctgggagcttcggtgctgggactggacgatatccaccgggct
tggcggacctttgttctccgggtgagagcccaagaccctccgccggaactgtacttcgtgaaggtggcgatcaccggagcctatgatactat
tccgcaagatcgactcaccgaagtcatcgcctcgatcatcaaaccgcagaacacttactgcgtcaggcggtacgccgtggtccagaaggc
cgcgcatggccacgtgagaaaggcgttcaagtcgcacgtgtccactctcaccgacctccagccttacatgaggcaattcgttgcgcatttgc
aagagacttcgcccctgagagatgcggtggtcatcgagcagagctccagcctgaacgaagcgagcagcggtctgtttgacgtgttcctcc
gcttcatgtgtcatcacgcggtgcgaatcaggggaaaatcatacgtgcagtgccagggaatcccacaaggcagcattctgtcgactctcttg
tgttccctttgctacggcgatatggaaaacaagctgttcgctgggatcagacgggacgggttgctgctcagactggtggacgacttcctgct
ggtgactccgcacctcactcacgccaaaacctttctccgcactctggtgaggggagtgccagaatacggctgtgtggtcaatctccggaaa
actgtggtgaatttccctgtcgaggatgaggcactcggaggaaccgcatttgtccaaatgccagcacatggcctgttcccatggtgcggtct
gctgctggacacccgaactcttgaagtgcagtccgactactccagctatgcccggacgagcatccgcgccagcctcactttcaatcgcggc
tttaaggccggacgaaacatgcgcagaaagcttttcggagtcctccggcttaaatgccattcgctctttctcgatctccaagtcaattcgctgc
agaccgtgtgcacgaacatctacaagatcctgctgctccaagcctaccggttccacgcttgcgtgcttcagctgccgtttcaccaacaggtgt
ggaagaacccgaccttctttctgcgggtcattagcgatactgcctccctgtgttactcaatcctcaaggcaaagaacgccggaatgtcgctg
ggtgcgaaaggagccgcgggacctcttcctagcgaagcggtgcagtggctctgccaccaggctttcctcctgaagctgaccaggcacag
agtgacctacgtcccgctgctgggctcgctgcgcactgcacagacccagctgtctagaaaactccccggcaccaccctgaccgctctgga
agccgccgccaacccagcattgccgtcagatttcaagaccatcttggac.
Plasmid 1112 Polypeptide (TERT240)

SEQ ID NO: 9

MGAAPEPERTPVGQGSWAHPGRTRGPSDRGFCVVSPARPAEEATSLEGALSGTRHSHPS
VGRQHHAGPPSTSRPPRPWDTPCPPVYAETKHFLYSSGDKEQLRPSFLLSSLRPSLTGAR
RLVETIFLGSRPWMPGTPRRLPRLPQRYWQMRPLFLELLGNHAQCPYGVLLKTHCPLRA
AVTPAAGVCAREKPQGSVAAPEEEDTDPRRLVQLLRQHSSPWQVYGFVRACLRRLVPP
GLWGSRHNERRFLRNTKKFISLGKHAKLSLQELTWKMSVRDCAWLRRSPGVGCVPAA
EHRLREEILAKFLHWLMSVYVVELLRSFFYVTETTFQKNRLFFYRKSVWSKLQSIGIRQH
LKRVQLRELSEAEVRQHREARPALLTSRLRFIPKPDGLRPIVNMDYVVGARTFRREKRA
ERLTSRVKALFSVLNYERARRPGLLGASVLGLDDIHRAWRTFVLRVRAQDPPPELYFVK
VAITGAYDTIPQDRLTEVIASIIKPQNTYCVRRYAVVQKAAHGHVRKAFKSHVSTLTDL
QPYMRQFVAHLQETSPLRDAVVIEQSSSLNEASSGLFDVFLRFMCHHAVRIRGKSYVQC
QGIPQGSILSTLLCSLCYGDMENKLFAGIRRDGLLLRLVDDFLLVTPHLTHAKTFLRTLV
RGVPEYGCVVNLRKTVVNFPVEDEALGGTAFVQMPAHGLFPWCGLLLDTRTLEVQSD
YSSYARTSIRASLTFNRGFKAGRNMRRKLFGVLRLKCHSLFLDLQVNSLQTVCTNIYKIL
LLQAYRFHACVLQLPFHQQVWKNPTFFLRVISDTASLCYSILKAKNAGMSLGAKGAAG
PLPSEAVQWLCHQAFLLKLTRHRVTYVPLLGSLRTAQTQLSRKLPGTTLTALEAAANPA
LPSDFKTILD.
Plasmid 1361 ORF (nucleotide sequence)

SEQ ID NO: 14

atggctagcgaatcgccaagcgcaccccctcatcggtggtgcatcccttggcaacgcctcctcctgaccgcctcactgctgactttctggaa
cccgccgaccaccgcaaagctgaccattgagagcactcccttcaacgtggctgaggggaaggaggtgctgctcctggtgcacaatctgc
cccagcacctgttcgggtactcctggtacaagggagaacgcgtggacgggaaccggcagatcataggctacgtcatcggaacccagca
ggccacacccggtccagcgtacagcggccgggagattatctacccgaacgcctccctgctgatccaaaacatcatccagaacgacaccg
gtttctacactctgcacgtgattaagtcagatctggtcaacgaagaggccaccggccaattcagggtgtaccccgaactcccaaagccgtcc
atttcaagcaacaactccaagccggtggaggacaaagacgccgtggccttcacttgtgaacctgaaacccaggacgccacttacctttggt
gggtgaacaaccagtcgctccccgtgtcgccgaggctgcagctcagcaacggaaacagaacgctgaccctcttcaatgtgacccgcaat
gataccgcctcctataagtgcgaaacccagaatccggtgtccgcccggcgctcggatagcgtgattctgaacgtgctctacggccctgacg
cccccactatctcccctctgaacacttcctaccggtccggagagaacctgaacctgagctgccacgcggcgtccaacccgcccgcccagt
acagctggttcgtgaatgggacgttccagcagtccacccaggagctgtttatccctaacattaccgtcaacaactctggatcgtacacatgcc
aagcgcataactcggacactgggcttaacagaaccaccgtgacaaccatcactgtgtatgcggaacctcctaagccgttcatcacctcgaa
caacagcaacccggtcgaggatgaagatgcggtggccttgacgtgcgaacctgagatccagaacaccacctacttgtggtgggtgaaca
atcagagcctgccagtctccccacgactccagctgtcgaacgacaacaggaccctgactttgctgtccgtgactcggaacgacgtgggcc
cttatgaatgcggtatccagaacaagctgtccgtggaccacagcgaccctgtgatcctgaacgtcctttacgggccggacgaccccaccat
ttccccgtcgtacacttactaccggccgggcgtgaacctgtccctgtcgtgccacgctgcctccaatccgccggcccagtactcctggctca
tcgacggaaacatccagcagcacacccaagaactgttcatctccaacattaccgagaaaaactcgggactttacacctgtcaagccaacaa
ttccgccagcggccactcccgcaccactgtcaaaactatcactgtgtccgccgaactcccgaagcccagcatcagctccaacaactcgaa
gcccgtggaggataaggacgctgtcgcgttcacctgtgaaccagaggcacagaataccacctacctttggtgggtcaacggacagtccct
gcctgtctcaccgagactgcagctgtcaaacgggaataggactctgaccttgtttaacgtcacccggaacgacgcccgggcctacgtgtgc
ggcatccagaactccgtgagcgcaaaccggtctgacccagtgaccctggatgtgctgtacggccccgacactccgatcatttcacccccc
gattcatcctacctgtccggcgctaacctcaacctctcatgccactccgcatccaaccccagcccgcaatattcgtggcgcattaacggaatt
cctcagcaacatacccaggtcctgttcattgcgaagatcacccctaacaacaacggaacctacgcctgctttgtgtcaaacctggccactgg
tagaaacaactccatcgtgaagtccattaccgtgtcggcgtccggaacttccccgggcctgagcgccggcgccaccgtgggaattatgatc
ggcgtgctcgtgggagtggccctgatc.
Plasmid 1361 Polypeptide

SEQ ID NO: 15

MASESPSAPPHRWCIPWQRLLLTASLLTFWNPPTTAKLTIESTPFNVAEGKEVLLLVHNL
PQHLFGYSWYKGERVDGNRQIIGYVIGTQQATPGPAYSGREIIYPNASLLIQNIIQNDTGF
YTLHVIKSDLVNEEATGQFRVYPELPKPSISSNNSKPVEDKDAVAFTCEPETQDATYLW
WVNNQSLPVSPRLQLSNGNRTLTLFNVTRNDTASYKCETQNPVSARRSDSVILNVLYGP
DAPTISPLNTSYRSGENLNLSCHAASNPPAQYSWFVNGTFQQSTQELFIPNITVNNSGSYT
CQAHNSDTGLNRTTVTTITVYAEPPKPFITSNNSNPVEDEDAVALTCEPEIQNTTYLWW
VNNQSLPVSPRLQLSNDNRTLTLLSVTRNDVGPYECGIQNKLSVDHSDPVILNVLYGPD
DPTISPSYTYYRPGVNLSLSCHAASNPPAQYSWLIDGNIQQHTQELFISNITEKNSGLYTC
QANNSASGHSRTTVKTITVSAELPKPSISSNNSKPVEDKDAVAFTCEPEAQNTTYLWWV
NGQSLPVSPRLQLSNGNRTLTLFNVTRNDARAYVCGIQNSVSANRSDPVTLDVLYGPDT
PIISPPDSSYLSGANLNLSCHSASNPSPQYSWRINGIPQQHTQVLFIAKITPNNNGTYACFV
SNLATGRNNSIVKSITVSASGTSPGLSAGATVGIMIGVLVGVALI.
Plasmid 1386 ORF (mCEA)

SEQ ID NO: 16

atggctagcgaatcgccaagcgcaccccctcatcggtggtgcatcccttggcaacgcctcctcctgaccgcctcactgctgactttctggaa
cccgccgaccaccgcaaagctgaccattgagagcactcccttcaacgtggctgaggggaaggaggtgctgctcctggtgcacaatctgc
cccagcacctgttcgggtactcctggtacaagggagaacgcgtggacgggaaccggcagatcataggctacgtcatcggaacccagca
ggccacacccggtccagcgtacagcggccgggagattatctacccgaacgcctccctgctgatccaaaacatcatccagaacgacaccg
gtttctacactctgcacgtgattaagtcagatctggtcaacgaagaggccaccggccaattcagggtgtaccccgaactccctaagccgttc
atcacctcgaacaacagcaacccggtcgaggatgaagatgcggtggccttgacgtgcgaacctgagatccagaacaccacctacttgtgg
tgggtgaacaatcagagcctgccagtctccccacgactccagctgtcgaacgacaacaggaccctgactttgctgtccgtgactcggaacg
acgtgggcccttatgaatgcggtatccagaacaagctgtccgtggaccacagcgaccctgtgatcctgaacgtcctttacgggccggacga
ccccaccatttccccgtcgtacacttactaccggccgggcgtgaacctgtccctgtcgtgccacgctgcctccaatccgccggcccagtact
cctggctcatcgacggaaacatccagcagcacacccaagaactgttcatctccaacattaccgagaaaaactcgggactttacacctgtcaa
gccaacaattccgccagcggccactcccgcaccactgtcaaaactatcactgtgtccgccgaactcccgaagcccagcatcagctccaac
aactcgaagcccgtggaggataaggacgctgtcgcgttcacctgtgaaccagaggcacagaataccacctacctttggtgggtcaacgga
cagtccctgcctgtctcaccgagactgcagctgtcaaacgggaataggactctgaccttgtttaacgtcacccggaacgacgcccgggcct
acgtgtgcggcatccagaactccgtgagcgcaaaccggtctgacccagtgaccctggatgtgctgtacggccccgacactccgatcatttc
accccccgattcatcctacctgtccggcgctaacctcaacctctcatgccactccgcatccaaccccagcccgcaatattcgtggcgcattaa
cggaattcctcagcaacatacccaggtcctgttcattgcgaagatcacccctaacaacaacggaacctacgcctgctttgtgtcaaacctgg
ccactggtagaaacaactccatcgtgaagtccattaccgtgtcggcgtccggaacttccccgggcctgagcgccggcgccaccgtggga
attatgatcggcgtgctcgtgggagtggccctgatc.
Plasmid 1386 Polypeptide (mCEA)

SEQ ID NO: 17

MASESPSAPPHRWCIPWQRLLLTASLLTFWNPPTTAKLTIESTPFNVAEGKEVLLLVHNL
PQHLFGYSWYKGERVDGNRQIIGYVIGTQQATPGPAYSGREIIYPNASLLIQNIIQNDTGF
YTLHVIKSDLVNEEATGQFRVYPELPKPFITSNNSNPVEDEDAVALTCEPEIQNTTYLWW
VNNQSLPVSPRLQLSNDNRTLTLLSVTRNDVGPYECGIQNKLSVDHSDPVILNVLYGPD
DPTISPSYTYYRPGVNLSLSCHAASNPPAQYSWLIDGNIQQHTQELFISNITEKNSGLYTC
QANNSASGHSRTTVKTITVSAELPKPSISSNNSKPVEDKDAVAFTCEPEAQNTTYLWWV
NGQSLPVSPRLQLSNGNRTLTLFNVTRNDARAYVCGIQNSVSANRSDPVTLDVLYGPDT
PIISPPDSSYLSGANLNLSCHSASNPSPQYSWRINGIPQQHTQVLFIAKITPNNNGTYACFV
SNLATGRNNSIVKSITVSASGTSPGLSAGATVGIMIGVLVGVALI.
Plasmid 1390 ORF (cCEA)

SEQ ID NO: 18

atggctagcaagctgaccattgagagcactcccttcaacgtggctgaggggaaggaggtgctgctcctggtgcacaatctgccccagcac
ctgttcgggtactcctggtacaagggagaacgcgtggacgggaaccggcagatcataggctacgtcatcggaacccagcaggccacac
ccggtccagcgtacagcggccgggagattatctacccgaacgcctccctgctgatccaaaacatcatccagaacgacaccggtttctacac
tctgcacgtgattaagtcagatctggtcaacgaagaggccaccggccaattcagggtgtaccccgaactccctaagccgttcatcacctcga
acaacagcaacccggtcgaggatgaagatgcggtggccttgacgtgcgaacctgagatccagaacaccacctacttgtggtgggtgaac
aatcagagcctgccagtctccccacgactccagctgtcgaacgacaacaggaccctgactttgctgtccgtgactcggaacgacgtgggc
ccttatgaatgcggtatccagaacaagctgtccgtggaccacagcgaccctgtgatcctgaacgtcctttacgggccggacgaccccacca
tttccccgtcgtacacttactaccggccgggcgtgaacctgtccctgtcgtgccacgctgcctccaatccgccggcccagtactcctggctc
atcgacggaaacatccagcagcacacccaagaactgttcatctccaacattaccgagaaaaactcgggactttacacctgtcaagccaaca
attccgccagcggccactcccgcaccactgtcaaaactatcactgtgtccgccgaactcccgaagcccagcatcagctccaacaactcga
agcccgtggaggataaggacgctgtcgcgttcacctgtgaaccagaggcacagaataccacctacctttggtgggtcaacggacagtccc
tgcctgtctcaccgagactgcagctgtcaaacgggaataggactctgaccttgtttaacgtcacccggaacgacgcccgggcctacgtgtg
cggcatccagaactccgtgagcgcaaaccggtctgacccagtgaccctggatgtgctgtacggccccgacactccgatcatttcaccccc
cgattcatcctacctgtccggcgctaacctcaacctctcatgccactccgcatccaaccccagcccgcaatattcgtggcgcattaacggaat
tcctcagcaacatacccaggtcctgttcattgcgaagatcacccctaacaacaacggaacctacgcctgctttgtgtcaaacctggccactg
gtagaaacaactccatcgtgaagtccattaccgtgtcggcgtcc.
Plasmid 1390 Polypeptide (cCEA)

SEQ ID NO: 19

MASKLTIESTPFNVAEGKEVLLLVHNLPQHLFGYSWYKGERVDGNRQIIGYVIGTQQAT
PGPAYSGREIIYPNASLLIQNIIQNDTGFYTLHVIKSDLVNEEATGQFRVYPELPKPFITSN
NSNPVEDEDAVALTCEPEIQNTTYLWWVNNQSLPVSPRLQLSNDNRTLTLLSVTRNDV
GPYECGIQNKLSVDHSDPVILNVLYGPDDPTISPSYTYYRPGVNLSLSCHAASNPPAQYS
WLIDGNIQQHTQELFISNITEKNSGLYTCQANNSASGHSRTTVKTITVSAELPKPSISSNNS
KPVEDKDAVAFTCEPEAQNTTYLWWVNGQSLPVSPRLQLSNGNRTLTLFNVTRNDAR
AYVCGIQNSVSANRSDPVTLDVLYGPDTPIISPPDSSYLSGANLNLSCHSASNPSPQYSW
RINGIPQQHTQVLFIAKITPNNNGTYACFVSNLATGRNNSIVKSITVSAS.
Plasmid 1431 ORF (nucleotide sequence)

SEQ ID NO: 36

atggctagcacccctggaacccagagccccttcttccttctgctgctgctgaccgtgctgactgtcgtgacaggctctggccacgccagctc
tacacctggcggcgagaaagagacaagcgccacccagagaagcagcgtgccaagcagcaccgagaagaacgccgtgtccatgacca
gctccgtgctgagcagccactctcctggcagcggcagcagcacaacacagggccaggatgtgacactggcccctgccacagaacctgc
ctctggatctgccgccacctggggacaggacgtgacaagcgtgccagtgaccagacctgccctgggctctacaacaccccctgcccacg
atgtgaccagcgcccctgataacaagcctgcccctggaagcacagcccctccagctcatggcgtgacctctgccccagataccagacca
gccccaggatctacagccccacccgcacacggcgtgacaagtgcccctgacacaagacccgctccaggctctactgctcctcctgcccat
ggcgtgacaagcgctcccgatacaaggccagctcctggctccacagcaccaccagcacatggcgtgacatcagctcccgacactagacc
tgctcccggatcaaccgctccaccagctcacggcgtgaccagcgcacctgataccagacctgctctgggaagcaccgcccctcccgtgc
acaatgtgacatctgcttccggcagcgccagcggctctgcctctacactggtgcacaacggcaccagcgccagagccacaacaacccca
gccagcaagagcacccccttcagcatccctagccaccacagcgacacccctaccacactggccagccactccaccaagaccgatgcctc
tagcacccaccactccagcgtgccccctctgaccagcagcaaccacagcacaagcccccagctgtctaccggcgtctcattcttctttctgt
ccttccacatcagcaacctgcagttcaacagcagcctggaagatcccagcaccgactactaccaggaactgcagcgggatatcagcgag
atgttcctgcaaatctacaagcagggcggcttcctgggcctgagcaacatcaagttcagacccggcagcgtggtggtgcagctgaccctg
gctttccgggaaggcaccatcaacgtgcacgacgtggaaacccagttcaaccagtacaagaccgaggccgccagccggtacaacctga
ccatctccgatgtgtccgtgtccgacgtgcccttcccattctctgcccagtctggcgcaggcgtgccaggatggggaattgctctgctggtgc
tcgtgtgcgtgctggtggccctggccatcgtgtatctgattgccctggccgtgtgccagtgccggcggaagaattacggccagctggacat
cttccccgccagagacacctaccaccccatgagcgagtaccccacataccacacccacggcagatacgtgccacccagctccaccgaca
gatccccctacgagaaagtgtctgccggcaacggcggcagctccctgagctacacaaatcctgccgtggccgctgcctccgccaacctg
ggatccggcagaatcttcaacgcccactacgccggctacttcgccgacctgctgatccacgacatcgagacaaaccctggccccaagctg
accattgagagcactcccttcaacgtggctgaggggaaggaggtgctgctcctggtgcacaatctgccccagcacctgttcgggtactcct
ggtacaagggagaacgcgtggacgggaaccggcagatcataggctacgtcatcggaacccagcaggccacacccggtccagcgtaca
gcggccgggagattatctacccgaacgcctccctgctgatccaaaacatcatccagaacgacaccggtttctacactctgcacgtgattaag
tcagatctggtcaacgaagaggccaccggccaattcagggtgtaccccgaactccctaagccgttcatcacctcgaacaacagcaacccg
gtcgaggatgaagatgcggtggccttgacgtgcgaacctgagatccagaacaccacctacttgtggtgggtgaacaatcagagcctgcca
gtctccccacgactccagctgtcgaacgacaacaggaccctgactttgctgtccgtgactcggaacgacgtgggcccttatgaatgcggtat
ccagaacaagctgtccgtggaccacagcgaccctgtgatcctgaacgtcctttacgggccggacgaccccaccatttccccgtcgtacact
tactaccggccgggcgtgaacctgtccctgtcgtgccacgctgcctccaatccgccggcccagtactcctggctcatcgacggaaacatcc
agcagcacacccaagaactgttcatctccaacattaccgagaaaaactcgggactttacacctgtcaagccaacaattccgccagcggcca
ctcccgcaccactgtcaaaactatcactgtgtccgccgaactcccgaagcccagcatcagctccaacaactcgaagcccgtggaggataa
ggacgctgtcgcgttcacctgtgaaccagaggcacagaataccacctacctttggtgggtcaacggacagtccctgcctgtctcaccgaga
ctgcagctgtcaaacgggaataggactctgaccttgtttaacgtcacccggaacgacgcccgggcctacgtgtgcggcatccagaactcc
gtgagcgcaaaccggtctgacccagtgaccctggatgtgctgtacggccccgacactccgatcatttcaccccccgattcatcctacctgtc
cggcgctaacctcaacctctcatgccactccgcatccaaccccagcccgcaatattcgtggcgcattaacggaattcctcagcaacataccc
aggtcctgttcattgcgaagatcacccctaacaacaacggaacctacgcctgctttgtgtcaaacctggccactggtagaaacaactccatc
gtgaagtccattaccgtgtcggcgtcc.
Plasmid 1431 Polypeptide

SEQ ID NO: 37

MASTPGTQSPFFLLLLLTVLTVVTGSGHASSTPGGEKETSATQRSSVPSSTEKNAVSMTS
SVLSSHSPGSGSSTTQGQDVTLAPATEPASGSAATWGQDVTSVPVTRPALGSTTPPAHD
VTSAPDNKPAPGSTAPPAHGVTSAPDTRPAPGSTAPPAHGVTSAPDTRPAPGSTAPPAHG
VTSAPDTRPAPGSTAPPAHGVTSAPDTRPAPGSTAPPAHGVTSAPDTRPALGSTAPPVHN
VTSASGSASGSASTLVHNGTSARATTTPASKSTPFSIPSHHSDTPTTLASHSTKTDASSTH
HSSVPPLTSSNHSTSPQLSTGVSFFFLSFHISNLQFNSSLEDPSTDYYQELQRDISEMFLQI
YKQGGFLGLSNIKFRPGSVVVQLTLAFREGTINVHDVETQFNQYKTEAASRYNLTISDV
SVSDVPFPFSAQSGAGVPGWGIALLVLVCVLVALAIVYLIALAVCQCRRKNYGQLDIFP
ARDTYHPMSEYPTYHTHGRYVPPSSTDRSPYEKVSAGNGGSSLSYTNPAVAAASANLG
SGRIFNAHYAGYFADLLIHDIETNPGPKLTIESTPFNVAEGKEVLLLVHNLPQHLFGYSW
YKGERVDGNRQIIGYVIGTQQATPGPAYSGREIIYPNASLLIQNIIQNDTGFYTLHVIKSDL
VNEEATGQFRVYPELPKPFITSNNSNPVEDEDAVALTCEPEIQNTTYLWWVNNQSLPVS
PRLQLSNDNRTLTLLSVTRNDVGPYECGIQNKLSVDHSDPVILNVLYGPDDPTISPSYTY
YRPGVNLSLSCHAASNPPAQYSWLIDGNIQQHTQELFISNITEKNSGLYTCQANNSASGH
SRTTVKTITVSAELPKPSISSNNSKPVEDKDAVAFTCEPEAQNTTYLWWVNGQSLPVSPR
LQLSNGNRTLTLFNVTRNDARAYVCGIQNSVSANRSDPVTLDVLYGPDTPIISPPDSSYL
SGANLNLSCHSASNPSPQYSWRINGIPQQHTQVLFIAKITPNNNGTYACFVSNLATGRNN
SIVKSITVSAS.
Plasmid 1432 ORF (nucleotide sequence)

SEQ ID NO: 38

atggctagcaagctgaccattgagagcactcccttcaacgtggctgaggggaaggaggtgctgctcctggtgcacaatctgccccagcac
ctgttcgggtactcctggtacaagggagaacgcgtggacgggaaccggcagatcataggctacgtcatcggaacccagcaggccacac
ccggtccagcgtacagcggccgggagattatctacccgaacgcctccctgctgatccaaaacatcatccagaacgacaccggtttctacac
tctgcacgtgattaagtcagatctggtcaacgaagaggccaccggccaattcagggtgtaccccgaactccctaagccgttcatcacctcga
acaacagcaacccggtcgaggatgaagatgcggtggccttgacgtgcgaacctgagatccagaacaccacctacttgtggtgggtgaac
aatcagagcctgccagtctccccacgactccagctgtcgaacgacaacaggaccctgactttgctgtccgtgactcggaacgacgtgggc
ccttatgaatgcggtatccagaacaagctgtccgtggaccacagcgaccctgtgatcctgaacgtcctttacgggccggacgaccccacca
tttccccgtcgtacacttactaccggccgggcgtgaacctgtccctgtcgtgccacgctgcctccaatccgccggcccagtactcctggctc
atcgacggaaacatccagcagcacacccaagaactgttcatctccaacattaccgagaaaaactcgggactttacacctgtcaagccaaca
attccgccagcggccactcccgcaccactgtcaaaactatcactgtgtccgccgaactcccgaagcccagcatcagctccaacaactcga
agcccgtggaggataaggacgctgtcgcgttcacctgtgaaccagaggcacagaataccacctacctttggtgggtcaacggacagtccc
tgcctgtctcaccgagactgcagctgtcaaacgggaataggactctgaccttgtttaacgtcacccggaacgacgcccgggcctacgtgtg
cggcatccagaactccgtgagcgcaaaccggtctgacccagtgaccctggatgtgctgtacggccccgacactccgatcatttcaccccc
cgattcatcctacctgtccggcgctaacctcaacctctcatgccactccgcatccaaccccagcccgcaatattcgtggcgcattaacggaat
tcctcagcaacatacccaggtcctgttcattgcgaagatcacccctaacaacaacggaacctacgcctgctttgtgtcaaacctggccactg
gtagaaacaactccatcgtgaagtccattaccgtgtcggcgtccggatccggcgagggcagaggcagcctgctgacatgtggcgacgtg
gaagagaaccctggccccggagctgccccggagccggagaggacccccgttggccagggatcgtgggcccatccgggacgcaccag
gggaccatccgacaggggattctgtgtggtgtcaccggccaggccagcagaagaggcaaccagcctcgagggagcgttgtctggaacc
agacattcccacccgtcggtgggccggcagcaccacgcgggaccaccgtccacttccagaccgccacggccatgggacaccccttgcc
cgcctgtgtatgccgagactaaacacttcctgtactcatccggagacaaggaacagcttcggccgtccttcctcctgtcgtcgctcagaccg
agcctgaccggagcacgcagattggtggaaactatcttccttgggtcacgtccgtggatgccaggtaccccacggcgcctcccgcgcctc
ccacagagatactggcagatgcggcctctgttcctggaattgctgggaaaccacgctcagtgcccgtacggagtcctgctcaagactcact
gccctctgagggcggcggtcactccggcggccggagtgtgcgcacgggagaagccccagggaagcgtggcagctccggaagagga
ggacaccgatccgcgccgcctcgtgcaacttctgcgccagcactcctcgccctggcaagtctacgggttcgtccgcgcctgcctgcgccg
cctggtgccgcctgggctctggggttcccggcataacgagcgccgcttcctgagaaatactaagaagtttatctcacttggaaaacatgcca
agttgtcgctgcaagaactcacgtggaagatgtcagtccgcgattgcgcctggctgcgccgctcgccgggcgtcgggtgtgttccagctgc
agaacaccgcctgagagaagaaattctggccaaatttctgcattggctgatgtcagtgtacgtggtcgagctgctgcgctcctttttctacgtc
actgagactacctttcaaaagaaccgcctgttcttctaccgcaaatctgtgtggagcaagctgcagtcaatcggcattcgccagcatctgaag
agggtgcagctgcgggaactttccgaggcagaagtccgccagcaccgggaggcccggccggcgcttctcacgtcgcgtctgagattcat
cccaaagcccgacgggctgaggcctatcgtcaacatggattacgtcgtgggcgctcgcacctttcgccgtgaaaagcgggccgaacgctt
gacctcacgggtgaaggccctcttctccgtgctgaactacgagagagcaagacggcctggcctgctgggagcttcggtgctgggactgg
acgatatccaccgggcttggcggacctttgttctccgggtgagagcccaagaccctccgccggaactgtacttcgtgaaggtggcgatcac
cggagcctatgatactattccgcaagatcgactcaccgaagtcatcgcctcgatcatcaaaccgcagaacacttactgcgtcaggcggtac
gccgtggtccagaaggccgcgcatggccacgtgagaaaggcgttcaagtcgcacgtgtccactctcaccgacctccagccttacatgag
gcaattcgttgcgcatttgcaagagacttcgcccctgagagatgcggtggtcatcgagcagagctccagcctgaacgaagcgagcagcg
gtctgtttgacgtgttcctccgcttcatgtgtcatcacgcggtgcgaatcaggggaaaatcatacgtgcagtgccagggaatcccacaaggc
agcattctgtcgactctcttgtgttccctttgctacggcgatatggaaaacaagctgttcgctgggatcagacgggacgggttgctgctcaga
ctggtggacgacttcctgctggtgactccgcacctcactcacgccaaaacctttctccgcactctggtgaggggagtgccagaatacggct
gtgtggtcaatctccggaaaactgtggtgaatttccctgtcgaggatgaggcactcggaggaaccgcatttgtccaaatgccagcacatggc
ctgttcccatggtgcggtctgctgctggacacccgaactcttgaagtgcagtccgactactccagctatgcccggacgagcatccgcgcca
gcctcactttcaatcgcggctttaaggccggacgaaacatgcgcagaaagcttttcggagtcctccggcttaaatgccattcgctctttctcga
tctccaagtcaattcgctgcagaccgtgtgcacgaacatctacaagatcctgctgctccaagcctaccggttccacgcttgcgtgcttcagct
gccgtttcaccaacaggtgtggaagaacccgaccttctttctgcgggtcattagcgatactgcctccctgtgttactcaatcctcaaggcaaa
gaacgccggaatgtcgctgggtgcgaaaggagccgcgggacctcttcctagcgaagcggtgcagtggctctgccaccaggctttcctcct
gaagctgaccaggcacagagtgacctacgtcccgctgctgggctcgctgcgcactgcacagacccagctgtctagaaaactccccggca
ccaccctgaccgctctggaagccgccgccaacccagcattgccgtcagatttcaagaccatcttggac.
Plasmid 1432 Polypeptide

SEQ ID NO: 39

MASKLTIESTPFNVAEGKEVLLLVHNLPQHLFGYSWYKGERVDGNRQIIGYVIGTQQAT
PGPAYSGREIIYPNASLLIQNIIQNDTGFYTLHVIKSDLVNEEATGQFRVYPELPKPFITSN
NSNPVEDEDAVALTCEPEIQNTTYLWWVNNQSLPVSPRLQLSNDNRTLTLLSVTRNDV
GPYECGIQNKLSVDHSDPVILNVLYGPDDPTISPSYTYYRPGVNLSLSCHAASNPPAQYS
WLIDGNIQQHTQELFISNITEKNSGLYTCQANNSASGHSRTTVKTITVSAELPKPSISSNNS
KPVEDKDAVAFTCEPEAQNTTYLWWVNGQSLPVSPRLQLSNGNRTLTLFNVTRNDAR
AYVCGIQNSVSANRSDPVTLDVLYGPDTPIISPPDSSYLSGANLNLSCHSASNPSPQYSW
RINGIPQQHTQVLFIAKITPNNNGTYACFVSNLATGRNNSIVKSITVSASGSGEGRGSLLT
CGDVEENPGPGAAPEPERTPVGQGSWAHPGRTRGPSDRGFCVVSPARPAEEATSLEGAL
SGTRHSHPSVGRQHHAGPPSTSRPPRPWDTPCPPVYAETKHFLYSSGDKEQLRPSFLLSS
LRPSLTGARRLVETIFLGSRPWMPGTPRRLPRLPQRYWQMRPLFLELLGNHAQCPYGVL
LKTHCPLRAAVTPAAGVCAREKPQGSVAAPEEEDTDPRRLVQLLRQHSSPWQVYGFVR
ACLRRLVPPGLWGSRHNERRFLRNTKKFISLGKHAKLSLQELTWKMSVRDCAWLRRSP
GVGCVPAAEHRLREEILAKFLHWLMSVYVVELLRSFFYVTETTFQKNRLFFYRKSVWS
KLQSIGIRQHLKRVQLRELSEAEVRQHREARPALLTSRLRFIPKPDGLRPIVNMDYVVGA
RTFRREKRAERLTSRVKALFSVLNYERARRPGLLGASVLGLDDIHRAWRTFVLRVRAQ
DPPPELYFVKVAITGAYDTIPQDRLTEVIASIIKPQNTYCVRRYAVVQKAAHGHVRKAF
KSHVSTLTDLQPYMRQFVAHLQETSPLRDAVVIEQSSSLNEASSGLFDVFLRFMCHHAV
RIRGKSYVQCQGIPQGSILSTLLCSLCYGDMENKLFAGIRRDGLLLRLVDDFLLVTPHLT
HAKTFLRTLVRGVPEYGCVVNLRKTVVNFPVEDEALGGTAFVQMPAHGLFPWCGLLL
DTRTLEVQSDYSSYARTSIRASLTFNRGFKAGRNMRRKLFGVLRLKCHSLFLDLQVNSL
QTVCTNIYKILLLQAYRFHACVLQLPFHQQVWKNPTFFLRVISDTASLCYSILKAKNAG
MSLGAKGAAGPLPSEAVQWLCHQAFLLKLTRHRVTYVPLLGSLRTAQTQLSRKLPGTT
LTALEAAANPALPSDFKTILD.
Plasmid 1424 ORF (nucleotide sequence)

SEQ ID NO: 42

atggctagcacccctggaacccagagccccttcttccttctgctgctgctgaccgtgctgactgtcgtgacaggctctggccacgccagctc
tacacctggcggcgagaaagagacaagcgccacccagagaagcagcgtgccaagcagcaccgagaagaacgccgtgtccatgacca
gctccgtgctgagcagccactctcctggcagcggcagcagcacaacacagggccaggatgtgacactggcccctgccacagaacctgc
ctctggatctgccgccacctggggacaggacgtgacaagcgtgccagtgaccagacctgccctgggctctacaacaccccctgcccacg
atgtgaccagcgcccctgataacaagcctgcccctggaagcacagcccctccagctcatggcgtgacctctgccccagataccagacca
gccccaggatctacagccccacccgcacacggcgtgacaagtgcccctgacacaagacccgctccaggctctactgctcctcctgcccat
ggcgtgacaagcgctcccgatacaaggccagctcctggctccacagcaccaccagcacatggcgtgacatcagctcccgacactagacc
tgctcccggatcaaccgctccaccagctcacggcgtgaccagcgcacctgataccagacctgctctgggaagcaccgcccctcccgtgc
acaatgtgacatctgcttccggcagcgccagcggctctgcctctacactggtgcacaacggcaccagcgccagagccacaacaacccca
gccagcaagagcacccccttcagcatccctagccaccacagcgacacccctaccacactggccagccactccaccaagaccgatgcctc
tagcacccaccactccagcgtgccccctctgaccagcagcaaccacagcacaagcccccagctgtctaccggcgtctcattcttctttctgt
ccttccacatcagcaacctgcagttcaacagcagcctggaagatcccagcaccgactactaccaggaactgcagcgggatatcagcgag
atgttcctgcaaatctacaagcagggcggcttcctgggcctgagcaacatcaagttcagacccggcagcgtggtggtgcagctgaccctg
gctttccgggaaggcaccatcaacgtgcacgacgtggaaacccagttcaaccagtacaagaccgaggccgccagccggtacaacctga
ccatctccgatgtgtccgtgtccgacgtgcccttcccattctctgcccagtctggcgcaggcgtgccaggatggggaattgctctgctggtgc
tcgtgtgcgtgctggtggccctggccatcgtgtatctgattgccctggccgtgtgccagtgccggcggaagaattacggccagctggacat
cttccccgccagagacacctaccaccccatgagcgagtaccccacataccacacccacggcagatacgtgccacccagctccaccgaca
gatccccctacgagaaagtgtctgccggcaacggcggcagctccctgagctacacaaatcctgccgtggccgctgcctccgccaacctg
ggatccggcacaatcctgtctgagggcgccaccaacttcagcctgctgaaactggccggcgacgtggaactgaaccctggccctggagc
tgccccggagccggagaggacccccgttggccagggatcgtgggcccatccgggacgcaccaggggaccatccgacaggggattctg
tgtggtgtcaccggccaggccagcagaagaggcaaccagcctcgagggagcgttgtctggaaccagacattcccacccgtcggtgggc
cggcagcaccacgcgggaccaccgtccacttccagaccgccacggccatgggacaccccttgcccgcctgtgtatgccgagactaaac
acttcctgtactcatccggagacaaggaacagcttcggccgtccttcctcctgtcgtcgctcagaccgagcctgaccggagcacgcagatt
ggtggaaactatcttccttgggtcacgtccgtggatgccaggtaccccacggcgcctcccgcgcctcccacagagatactggcagatgcg
gcctctgttcctggaattgctgggaaaccacgctcagtgcccgtacggagtcctgctcaagactcactgccctctgagggcggcggtcact
ccggcggccggagtgtgcgcacgggagaagccccagggaagcgtggcagctccggaagaggaggacaccgatccgcgccgcctcg
tgcaacttctgcgccagcactcctcgccctggcaagtctacgggttcgtccgcgcctgcctgcgccgcctggtgccgcctgggctctgggg
ttcccggcataacgagcgccgcttcctgagaaatactaagaagtttatctcacttggaaaacatgccaagttgtcgctgcaagaactcacgtg
gaagatgtcagtccgcgattgcgcctggctgcgccgctcgccgggcgtcgggtgtgttccagctgcagaacaccgcctgagagaagaaa
ttctggccaaatttctgcattggctgatgtcagtgtacgtggtcgagctgctgcgctcctttttctacgtcactgagactacctttcaaaagaacc
gcctgttcttctaccgcaaatctgtgtggagcaagctgcagtcaatcggcattcgccagcatctgaagagggtgcagctgcgggaactttcc
gaggcagaagtccgccagcaccgggaggcccggccggcgcttctcacgtcgcgtctgagattcatcccaaagcccgacgggctgagg
cctatcgtcaacatggattacgtcgtgggcgctcgcacctttcgccgtgaaaagcgggccgaacgcttgacctcacgggtgaaggccctct
tctccgtgctgaactacgagagagcaagacggcctggcctgctgggagcttcggtgctgggactggacgatatccaccgggcttggcgg
acctttgttctccgggtgagagcccaagaccctccgccggaactgtacttcgtgaaggtggcgatcaccggagcctatgatactattccgca
agatcgactcaccgaagtcatcgcctcgatcatcaaaccgcagaacacttactgcgtcaggcggtacgccgtggtccagaaggccgcgc
atggccacgtgagaaaggcgttcaagtcgcacgtgtccactctcaccgacctccagccttacatgaggcaattcgttgcgcatttgcaagag
acttcgcccctgagagatgcggtggtcatcgagcagagctccagcctgaacgaagcgagcagcggtctgtttgacgtgttcctccgcttcat
gtgtcatcacgcggtgcgaatcaggggaaaatcatacgtgcagtgccagggaatcccacaaggcagcattctgtcgactctcttgtgttccc
tttgctacggcgatatggaaaacaagctgttcgctgggatcagacgggacgggttgctgctcagactggtggacgacttcctgctggtgact
ccgcacctcactcacgccaaaacctttctccgcactctggtgaggggagtgccagaatacggctgtgtggtcaatctccggaaaactgtggt
gaatttccctgtcgaggatgaggcactcggaggaaccgcatttgtccaaatgccagcacatggcctgttcccatggtgcggtctgctgctgg
acacccgaactcttgaagtgcagtccgactactccagctatgcccggacgagcatccgcgccagcctcactttcaatcgcggctttaaggc
cggacgaaacatgcgcagaaagcttttcggagtcctccggcttaaatgccattcgctctttctcgatctccaagtcaattcgctgcagaccgt
gtgcacgaacatctacaagatcctgctgctccaagcctaccggttccacgcttgcgtgcttcagctgccgtttcaccaacaggtgtggaaga
acccgaccttctttctgcgggtcattagcgatactgcctccctgtgttactcaatcctcaaggcaaagaacgccggaatgtcgctgggtgcga
aaggagccgcgggacctcttcctagcgaagcggtgcagtggctctgccaccaggctttcctcctgaagctgaccaggcacagagtgacct
acgtcccgctgctgggctcgctgcgcactgcacagacccagctgtctagaaaactccccggcaccaccctgaccgctctggaagccgcc
gccaacccagcattgccgtcagatttcaagaccatcttggacggatccggccagtgcaccaattacgccctgctgaagctggccggcgac
gtggaatctaaccctggccctgaatcgccaagcgcaccccctcatcggtggtgcatcccttggcaacgcctcctcctgaccgcctcactgct
gactttctggaacccgccgaccaccgcaaagctgaccattgagagcactcccttcaacgtggctgaggggaaggaggtgctgctcctggt
gcacaatctgccccagcacctgttcgggtactcctggtacaagggagaacgcgtggacgggaaccggcagatcataggctacgtcatcg
gaacccagcaggccacacccggtccagcgtacagcggccgggagattatctacccgaacgcctccctgctgatccaaaacatcatccag
aacgacaccggtttctacactctgcacgtgattaagtcagatctggtcaacgaagaggccaccggccaattcagggtgtaccccgaactcc
ctaagccgttcatcacctcgaacaacagcaacccggtcgaggatgaagatgcggtggccttgacgtgcgaacctgagatccagaacacc
acctacttgtggtgggtgaacaatcagagcctgccagtctccccacgactccagctgtcgaacgacaacaggaccctgactttgctgtccgt
gactcggaacgacgtgggcccttatgaatgcggtatccagaacaagctgtccgtggaccacagcgaccctgtgatcctgaacgtcctttac
gggccggacgaccccaccatttccccgtcgtacacttactaccggccgggcgtgaacctgtccctgtcgtgccacgctgcctccaatccgc
cggcccagtactcctggctcatcgacggaaacatccagcagcacacccaagaactgttcatctccaacattaccgagaaaaactcgggact
ttacacctgtcaagccaacaattccgccagcggccactcccgcaccactgtcaaaactatcactgtgtccgccgaactcccgaagcccagc
atcagctccaacaactcgaagcccgtggaggataaggacgctgtcgcgttcacctgtgaaccagaggcacagaataccacctacctttggt
gggtcaacggacagtccctgcctgtctcaccgagactgcagctgtcaaacgggaataggactctgaccttgtttaacgtcacccggaacga
cgcccgggcctacgtgtgcggcatccagaactccgtgagcgcaaaccggtctgacccagtgaccctggatgtgctgtacggccccgaca
ctccgatcatttcaccccccgattcatcctacctgtccggcgctaacctcaacctctcatgccactccgcatccaaccccagcccgcaatattc
gtggcgcattaacggaattcctcagcaacatacccaggtcctgttcattgcgaagatcacccctaacaacaacggaacctacgcctgctttgt
gtcaaacctggccactggtagaaacaactccatcgtgaagtccattaccgtgtcggcgtccggaacttccccgggcctgagcgccggcgc
caccgtgggaattatgatcggcgtgctcgtgggagtggccctgatc.
Plasmid 1424 Polypeptide

SEQ ID NO: 43

MASTPGTQSPFFLLLLLTVLTVVTGSGHASSTPGGEKETSATQRSSVPSSTEKNAVSMTS
SVLSSHSPGSGSSTTQGQDVTLAPATEPASGSAATWGQDVTSVPVTRPALGSTTPPAHD
VTSAPDNKPAPGSTAPPAHGVTSAPDTRPAPGSTAPPAHGVTSAPDTRPAPGSTAPPAHG
VTSAPDTRPAPGSTAPPAHGVTSAPDTRPAPGSTAPPAHGVTSAPDTRPALGSTAPPVHN
VTSASGSASGSASTLVHNGTSARATTTPASKSTPFSIPSHHSDTPTTLASHSTKTDASSTH
HSSVPPLTSSNHSTSPQLSTGVSFFFLSFHISNLQFNSSLEDPSTDYYQELQRDISEMFLQI
YKQGGFLGLSNIKFRPGSVVVQLTLAFREGTINVHDVETQFNQYKTEAASRYNLTISDV
SVSDVPFPFSAQSGAGVPGWGIALLVLVCVLVALAIVYLIALAVCQCRRKNYGQLDIFP
ARDTYHPMSEYPTYHTHGRYVPPSSTDRSPYEKVSAGNGGSSLSYTNPAVAAASANLG
SGTILSEGATNFSLLKLAGDVELNPGPGAAPEPERTPVGQGSWAHPGRTRGPSDRGFCV
VSPARPAEEATSLEGALSGTRHSHPSVGRQHHAGPPSTSRPPRPWDTPCPPVYAETKHFL
YSSGDKEQLRPSFLLSSLRPSLTGARRLVETIFLGSRPWMPGTPRRLPRLPQRYWQMRPL
FLELLGNHAQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKHAKLSLQELT
WKMSVRDCAWLRRSPGVGCVPAAEHRLREEILAKFLHWLMSVYVVELLRSFFYVTET
TFQKNRLFFYRKSVWSKLQSIGIRQHLKRVQLRELSEAEVRQHREARPALLTSRLRFIPK
PDGLRPIVNMDYVVGARTFRREKRAERLTSRVKALFSVLNYERARRPGLLGASVLGLD
DIHRAWRTFVLRVRAQDPPPELYFVKVAITGAYDTIPQDRLTEVIASIIKPQNTYCVRRY
AVVQKAAHGHVRKAFKSHVSTLTDLQPYMRQFVAHLQETSPLRDAVVIEQSSSLNEAS
SGLFDVFLRFMCHHAVRIRGKSYVQCQGIPQGSILSTLLCSLCYGDMENKLFAGIRRDGL
LLRLVDDFLLVTPHLTHAKTFLRTLVRGVPEYGCVVNLRKTVVNFPVEDEALGGTAFV
QMPAHGLFPWCGLLLDTRTLEVQSDYSSYARTSIRASLTFNRGFKAGRNMRRKLFGVL
RLKCHSLFLDLQVNSLQTVCTNIYKILLLQAYRFHACVLQLPFHQQVWKNPTFFLRVISD
TASLCYSILKAKNAGMSLGAKGAAGPLPSEAVQWLCHQAFLLKLTRHRVTYVPLLGSL
RTAQTQLSRKLPGTTLTALEAAANPALPSDFKTILDGSGQCTNYALLKLAGDVESNPGP
ESPSAPPHRWCIPWQRLLLTASLLTFWNPPTTAKLTIESTPFNVAEGKEVLLLVHNLPQH
LFGYSWYKGERVDGNRQIIGYVIGTQQATPGPAYSGREIIYPNASLLIQNIIQNDTGFYTL
HVIKSDLVNEEATGQFRVYPELPKPFITSNNSNPVEDEDAVALTCEPEIQNTTYLWWVN
NQSLPVSPRLQLSNDNRTLTLLSVTRNDVGPYECGIQNKLSVDHSDPVILNVLYGPDDPT
ISPSYTYYRPGVNLSLSCHAASNPPAQYSWLIDGNIQQHTQELFISNITEKNSGLYTCQAN
NSASGHSRTTVKTITVSAELPKPSISSNNSKPVEDKDAVAFTCEPEAQNTTYLWWVNGQ
SLPVSPRLQLSNGNRTLTLFNVTRNDARAYVCGIQNSVSANRSDPVTLDVLYGPDTPIIS
PPDSSYLSGANLNLSCHSASNPSPQYSWRINGIPQQHTQVLFIAKITPNNNGTYACFVSNL
ATGRNNSIVKSITVSASGTSPGLSAGATVGIMIGVLVGVALI.
Plasmid 1425 ORF (nucleotide sequence)

SEQ ID NO: 44

atggctagcgaatcgccaagcgcaccccctcatcggtggtgcatcccttggcaacgcctcctcctgaccgcctcactgctgactttctggaa
cccgccgaccaccgcaaagctgaccattgagagcactcccttcaacgtggctgaggggaaggaggtgctgctcctggtgcacaatctgc
cccagcacctgttcgggtactcctggtacaagggagaacgcgtggacgggaaccggcagatcataggctacgtcatcggaacccagca
ggccacacccggtccagcgtacagcggccgggagattatctacccgaacgcctccctgctgatccaaaacatcatccagaacgacaccg
gtttctacactctgcacgtgattaagtcagatctggtcaacgaagaggccaccggccaattcagggtgtaccccgaactccctaagccgttc
atcacctcgaacaacagcaacccggtcgaggatgaagatgcggtggccttgacgtgcgaacctgagatccagaacaccacctacttgtgg
tgggtgaacaatcagagcctgccagtctccccacgactccagctgtcgaacgacaacaggaccctgactttgctgtccgtgactcggaacg
acgtgggcccttatgaatgcggtatccagaacaagctgtccgtggaccacagcgaccctgtgatcctgaacgtcctttacgggccggacga
ccccaccatttccccgtcgtacacttactaccggccgggcgtgaacctgtccctgtcgtgccacgctgcctccaatccgccggcccagtact
cctggctcatcgacggaaacatccagcagcacacccaagaactgttcatctccaacattaccgagaaaaactcgggactttacacctgtcaa
gccaacaattccgccagcggccactcccgcaccactgtcaaaactatcactgtgtccgccgaactcccgaagcccagcatcagctccaac
aactcgaagcccgtggaggataaggacgctgtcgcgttcacctgtgaaccagaggcacagaataccacctacctttggtgggtcaacgga
cagtccctgcctgtctcaccgagactgcagctgtcaaacgggaataggactctgaccttgtttaacgtcacccggaacgacgcccgggcct
acgtgtgcggcatccagaactccgtgagcgcaaaccggtctgacccagtgaccctggatgtgctgtacggccccgacactccgatcatttc
accccccgattcatcctacctgtccggcgctaacctcaacctctcatgccactccgcatccaaccccagcccgcaatattcgtggcgcattaa
cggaattcctcagcaacatacccaggtcctgttcattgcgaagatcacccctaacaacaacggaacctacgcctgctttgtgtcaaacctgg
ccactggtagaaacaactccatcgtgaagtccattaccgtgtcggcgtccggaacttccccgggcctgagcgccggcgccaccgtggga
attatgatcggcgtgctcgtgggagtggccctgatcggatccggcgagggcagaggcagcctgctgacatgtggcgacgtggaagagaa
ccctggccccacccctggaacccagagccccttcttccttctgctgctgctgaccgtgctgactgtcgtgacaggctctggccacgccagct
ctacacctggcggcgagaaagagacaagcgccacccagagaagcagcgtgccaagcagcaccgagaagaacgccgtgtccatgacc
agctccgtgctgagcagccactctcctggcagcggcagcagcacaacacagggccaggatgtgacactggcccctgccacagaacctg
cctctggatctgccgccacctggggacaggacgtgacaagcgtgccagtgaccagacctgccctgggctctacaacaccccctgcccac
gatgtgaccagcgcccctgataacaagcctgcccctggaagcacagcccctccagctcatggcgtgacctctgccccagataccagacc
agccccaggatctacagccccacccgcacacggcgtgacaagtgcccctgacacaagacccgctccaggctctactgctcctcctgccc
atggcgtgacaagcgctcccgatacaaggccagctcctggctccacagcaccaccagcacatggcgtgacatcagctcccgacactaga
cctgctcccggatcaaccgctccaccagctcacggcgtgaccagcgcacctgataccagacctgctctgggaagcaccgcccctcccgt
gcacaatgtgacatctgcttccggcagcgccagcggctctgcctctacactggtgcacaacggcaccagcgccagagccacaacaaccc
cagccagcaagagcacccccttcagcatccctagccaccacagcgacacccctaccacactggccagccactccaccaagaccgatgc
ctctagcacccaccactccagcgtgccccctctgaccagcagcaaccacagcacaagcccccagctgtctaccggcgtctcattcttctttc
tgtccttccacatcagcaacctgcagttcaacagcagcctggaagatcccagcaccgactactaccaggaactgcagcgggatatcagcg
agatgttcctgcaaatctacaagcagggcggcttcctgggcctgagcaacatcaagttcagacccggcagcgtggtggtgcagctgaccc
tggctttccgggaaggcaccatcaacgtgcacgacgtggaaacccagttcaaccagtacaagaccgaggccgccagccggtacaacctg
accatctccgatgtgtccgtgtccgacgtgcccttcccattctctgcccagtctggcgcaggcgtgccaggatggggaattgctctgctggtg
ctcgtgtgcgtgctggtggccctggccatcgtgtatctgattgccctggccgtgtgccagtgccggcggaagaattacggccagctggaca
tcttccccgccagagacacctaccaccccatgagcgagtaccccacataccacacccacggcagatacgtgccacccagctccaccgac
agatccccctacgagaaagtgtctgccggcaacggcggcagctccctgagctacacaaatcctgccgtggccgctgcctccgccaacct
gggatccggcacaatcctgtctgagggcgccaccaacttcagcctgctgaaactggccggcgacgtggaactgaaccctggccctggag
ctgccccggagccggagaggacccccgttggccagggatcgtgggcccatccgggacgcaccaggggaccatccgacaggggattct
gtgtggtgtcaccggccaggccagcagaagaggcaaccagcctcgagggagcgttgtctggaaccagacattcccacccgtcggtggg
ccggcagcaccacgcgggaccaccgtccacttccagaccgccacggccatgggacaccccttgcccgcctgtgtatgccgagactaaa
cacttcctgtactcatccggagacaaggaacagcttcggccgtccttcctcctgtcgtcgctcagaccgagcctgaccggagcacgcagat
tggtggaaactatcttccttgggtcacgtccgtggatgccaggtaccccacggcgcctcccgcgcctcccacagagatactggcagatgcg
gcctctgttcctggaattgctgggaaaccacgctcagtgcccgtacggagtcctgctcaagactcactgccctctgagggcggcggtcact
ccggcggccggagtgtgcgcacgggagaagccccagggaagcgtggcagctccggaagaggaggacaccgatccgcgccgcctcg
tgcaacttctgcgccagcactcctcgccctggcaagtctacgggttcgtccgcgcctgcctgcgccgcctggtgccgcctgggctctgggg
ttcccggcataacgagcgccgcttcctgagaaatactaagaagtttatctcacttggaaaacatgccaagttgtcgctgcaagaactcacgtg
gaagatgtcagtccgcgattgcgcctggctgcgccgctcgccgggcgtcgggtgtgttccagctgcagaacaccgcctgagagaagaaa
ttctggccaaatttctgcattggctgatgtcagtgtacgtggtcgagctgctgcgctcctttttctacgtcactgagactacctttcaaaagaacc
gcctgttcttctaccgcaaatctgtgtggagcaagctgcagtcaatcggcattcgccagcatctgaagagggtgcagctgcgggaactttcc
gaggcagaagtccgccagcaccgggaggcccggccggcgcttctcacgtcgcgtctgagattcatcccaaagcccgacgggctgagg
cctatcgtcaacatggattacgtcgtgggcgctcgcacctttcgccgtgaaaagcgggccgaacgcttgacctcacgggtgaaggccctct
tctccgtgctgaactacgagagagcaagacggcctggcctgctgggagcttcggtgctgggactggacgatatccaccgggcttggcgg
acctttgttctccgggtgagagcccaagaccctccgccggaactgtacttcgtgaaggtggcgatcaccggagcctatgatactattccgca
agatcgactcaccgaagtcatcgcctcgatcatcaaaccgcagaacacttactgcgtcaggcggtacgccgtggtccagaaggccgcgc
atggccacgtgagaaaggcgttcaagtcgcacgtgtccactctcaccgacctccagccttacatgaggcaattcgttgcgcatttgcaagag
acttcgcccctgagagatgcggtggtcatcgagcagagctccagcctgaacgaagcgagcagcggtctgtttgacgtgttcctccgcttcat
gtgtcatcacgcggtgcgaatcaggggaaaatcatacgtgcagtgccagggaatcccacaaggcagcattctgtcgactctcttgtgttccc
tttgctacggcgatatggaaaacaagctgttcgctgggatcagacgggacgggttgctgctcagactggtggacgacttcctgctggtgact
ccgcacctcactcacgccaaaacctttctccgcactctggtgaggggagtgccagaatacggctgtgtggtcaatctccggaaaactgtggt
gaatttccctgtcgaggatgaggcactcggaggaaccgcatttgtccaaatgccagcacatggcctgttcccatggtgcggtctgctgctgg
acacccgaactcttgaagtgcagtccgactactccagctatgcccggacgagcatccgcgccagcctcactttcaatcgcggctttaaggc
cggacgaaacatgcgcagaaagcttttcggagtcctccggcttaaatgccattcgctctttctcgatctccaagtcaattcgctgcagaccgt
gtgcacgaacatctacaagatcctgctgctccaagcctaccggttccacgcttgcgtgcttcagctgccgtttcaccaacaggtgtggaaga
acccgaccttctttctgcgggtcattagcgatactgcctccctgtgttactcaatcctcaaggcaaagaacgccggaatgtcgctgggtgcga
aaggagccgcgggacctcttcctagcgaagcggtgcagtggctctgccaccaggctttcctcctgaagctgaccaggcacagagtgacct
acgtcccgctgctgggctcgctgcgcactgcacagacccagctgtctagaaaactccccggcaccaccctgaccgctctggaagccgcc
gccaacccagcattgccgtcagatttcaagaccatcttggac.
Plasmid 1425 Polypeptide

SEQ ID NO: 45

MASESPSAPPHRWCIPWQRLLLTASLLTFWNPPTTAKLTIESTPFNVAEGKEVLLLVHNL
PQHLFGYSWYKGERVDGNRQIIGYVIGTQQATPGPAYSGREIIYPNASLLIQNIIQNDTGF
YTLHVIKSDLVNEEATGQFRVYPELPKPFITSNNSNPVEDEDAVALTCEPEIQNTTYLWW
VNNQSLPVSPRLQLSNDNRTLTLLSVTRNDVGPYECGIQNKLSVDHSDPVILNVLYGPD
DPTISPSYTYYRPGVNLSLSCHAASNPPAQYSWLIDGNIQQHTQELFISNITEKNSGLYTC
QANNSASGHSRTTVKTITVSAELPKPSISSNNSKPVEDKDAVAFTCEPEAQNTTYLWWV
NGQSLPVSPRLQLSNGNRTLTLFNVTRNDARAYVCGIQNSVSANRSDPVTLDVLYGPDT
PIISPPDSSYLSGANLNLSCHSASNPSPQYSWRINGIPQQHTQVLFIAKITPNNNGTYACFV
SNLATGRNNSIVKSITVSASGTSPGLSAGATVGIMIGVLVGVALIGSGEGRGSLLTCGDV
EENPGPTPGTQSPFFLLLLLTVLTVVTGSGHASSTPGGEKETSATQRSSVPSSTEKNAVS
MTSSVLSSHSPGSGSSTTQGQDVTLAPATEPASGSAATWGQDVTSVPVTRPALGSTTPP
AHDVTSAPDNKPAPGSTAPPAHGVTSAPDTRPAPGSTAPPAHGVTSAPDTRPAPGSTAPP
AHGVTSAPDTRPAPGSTAPPAHGVTSAPDTRPAPGSTAPPAHGVTSAPDTRPALGSTAPP
VHNVTSASGSASGSASTLVHNGTSARATTTPASKSTPFSIPSHHSDTPTTLASHSTKTDAS
STHHSSVPPLTSSNHSTSPQLSTGVSFFFLSFHISNLQFNSSLEDPSTDYYQELQRDISEMF
LQIYKQGGFLGLSNIKFRPGSVVVQLTLAFREGTINVHDVETQFNQYKTEAASRYNLTIS
DVSVSDVPFPFSAQSGAGVPGWGIALLVLVCVLVALAIVYLIALAVCQCRRKNYGQLDI
FPARDTYHPMSEYPTYHTHGRYVPPSSTDRSPYEKVSAGNGGSSLSYTNPAVAAASANL
GSGTILSEGATNFSLLKLAGDVELNPGPGAAPEPERTPVGQGSWAHPGRTRGPSDRGFC
VVSPARPAEEATSLEGALSGTRHSHPSVGRQHHAGPPSTSRPPRPWDTPCPPVYAETKHF
LYSSGDKEQLRPSFLLSSLRPSLTGARRLVETIFLGSRPWMPGTPRRLPRLPQRYWQMRP
LFLELLGNHAQCPYGVLLKTHCPLRAAVTPAAGVCAREKPQGSVAAPEEEDTDPRRLV
QLLRQHSSPWQVYGFVRACLRRLVPPGLWGSRHNERRFLRNTKKFISLGKHAKLSLQE
LTWKMSVRDCAWLRRSPGVGCVPAAEHRLREEILAKFLHWLMSVYVVELLRSFFYVT
ETTFQKNRLFFYRKSVWSKLQSIGIRQHLKRVQLRELSEAEVRQHREARPALLTSRLRFI
PKPDGLRPIVNMDYVVGARTFRREKRAERLTSRVKALFSVLNYERARRPGLLGASVLG
LDDIHRAWRTFVLRVRAQDPPPELYFVKVAITGAYDTIPQDRLTEVIASIIKPQNTYCVR
RYAVVQKAAHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDMENKLFAGIRRD
GLLLRLVDDFLLVTPHLTHAKTFLRTLVRGVPEYGCVVNLRKTVVNFPVEDEALGGTA
FVQMPAHGLFPWCGLLLDTRTLEVQSDYSSYARTSIRASLTFNRGFKAGRNMRRKLFG
VLRLKCHSLFLDLQVNSLQTVCTNIYKILLLQAYRFHACVLQLPFHQQVWKNPTFFLRV
ISDTASLCYSILKAKNAGMSLGAKGAAGPLPSEAVQWLCHQAFLLKLTRHRVTYVPLL
GSLRTAQTQLSRKLPGTTLTALEAAANPALPSDFKTILD.
Plasmid 1426 ORF (nucleotide sequence)

SEQ ID NO: 46

atggctagcggagctgccccggagccggagaggacccccgttggccagggatcgtgggcccatccgggacgcaccaggggaccatc
cgacaggggattctgtgtggtgtcaccggccaggccagcagaagaggcaaccagcctcgagggagcgttgtctggaaccagacattccc
acccgtcggtgggccggcagcaccacgcgggaccaccgtccacttccagaccgccacggccatgggacaccccttgcccgcctgtgta
tgccgagactaaacacttcctgtactcatccggagacaaggaacagcttcggccgtccttcctcctgtcgtcgctcagaccgagcctgaccg
gagcacgcagattggtggaaactatcttccttgggtcacgtccgtggatgccaggtaccccacggcgcctcccgcgcctcccacagagat
actggcagatgcggcctctgttcctggaattgctgggaaaccacgctcagtgcccgtacggagtcctgctcaagactcactgccctctgag
ggcggcggtcactccggcggccggagtgtgcgcacgggagaagccccagggaagcgtggcagctccggaagaggaggacaccgat
ccgcgccgcctcgtgcaacttctgcgccagcactcctcgccctggcaagtctacgggttcgtccgcgcctgcctgcgccgcctggtgccg
cctgggctctggggttcccggcataacgagcgccgcttcctgagaaatactaagaagtttatctcacttggaaaacatgccaagttgtcgctg
caagaactcacgtggaagatgtcagtccgcgattgcgcctggctgcgccgctcgccgggcgtcgggtgtgttccagctgcagaacaccg
cctgagagaagaaattctggccaaatttctgcattggctgatgtcagtgtacgtggtcgagctgctgcgctcctttttctacgtcactgagacta
cctttcaaaagaaccgcctgttcttctaccgcaaatctgtgtggagcaagctgcagtcaatcggcattcgccagcatctgaagagggtgcag
ctgcgggaactttccgaggcagaagtccgccagcaccgggaggcccggccggcgcttctcacgtcgcgtctgagattcatcccaaagcc
cgacgggctgaggcctatcgtcaacatggattacgtcgtgggcgctcgcacctttcgccgtgaaaagcgggccgaacgcttgacctcacg
ggtgaaggccctcttctccgtgctgaactacgagagagcaagacggcctggcctgctgggagcttcggtgctgggactggacgatatcca
ccgggcttggcggacctttgttctccgggtgagagcccaagaccctccgccggaactgtacttcgtgaaggtggcgatcaccggagcctat
gatactattccgcaagatcgactcaccgaagtcatcgcctcgatcatcaaaccgcagaacacttactgcgtcaggcggtacgccgtggtcc
agaaggccgcgcatggccacgtgagaaaggcgttcaagtcgcacgtgtccactctcaccgacctccagccttacatgaggcaattcgttg
cgcatttgcaagagacttcgcccctgagagatgcggtggtcatcgagcagagctccagcctgaacgaagcgagcagcggtctgtttgacg
tgttcctccgcttcatgtgtcatcacgcggtgcgaatcaggggaaaatcatacgtgcagtgccagggaatcccacaaggcagcattctgtcg
actctcttgtgttccctttgctacggcgatatggaaaacaagctgttcgctgggatcagacgggacgggttgctgctcagactggtggacga
cttcctgctggtgactccgcacctcactcacgccaaaacctttctccgcactctggtgaggggagtgccagaatacggctgtgtggtcaatct
ccggaaaactgtggtgaatttccctgtcgaggatgaggcactcggaggaaccgcatttgtccaaatgccagcacatggcctgttcccatggt
gcggtctgctgctggacacccgaactcttgaagtgcagtccgactactccagctatgcccggacgagcatccgcgccagcctcactttcaa
tcgcggctttaaggccggacgaaacatgcgcagaaagcttttcggagtcctccggcttaaatgccattcgctctttctcgatctccaagtcaat
tcgctgcagaccgtgtgcacgaacatctacaagatcctgctgctccaagcctaccggttccacgcttgcgtgcttcagctgccgtttcaccaa
caggtgtggaagaacccgaccttctttctgcgggtcattagcgatactgcctccctgtgttactcaatcctcaaggcaaagaacgccggaat
gtcgctgggtgcgaaaggagccgcgggacctcttcctagcgaagcggtgcagtggctctgccaccaggctttcctcctgaagctgaccag
gcacagagtgacctacgtcccgctgctgggctcgctgcgcactgcacagacccagctgtctagaaaactccccggcaccaccctgaccg
ctctggaagccgccgccaacccagcattgccgtcagatttcaagaccatcttggacggatccggcacaatcctgtctgagggcgccacca
acttcagcctgctgaaactggccggcgacgtggaactgaaccctggccctacccctggaacccagagccccttcttccttctgctgctgctg
accgtgctgactgtcgtgacaggctctggccacgccagctctacacctggcggcgagaaagagacaagcgccacccagagaagcagc
gtgccaagcagcaccgagaagaacgccgtgtccatgaccagctccgtgctgagcagccactctcctggcagcggcagcagcacaacac
agggccaggatgtgacactggcccctgccacagaacctgcctctggatctgccgccacctggggacaggacgtgacaagcgtgccagt
gaccagacctgccctgggctctacaacaccccctgcccacgatgtgaccagcgcccctgataacaagcctgcccctggaagcacagccc
ctccagctcatggcgtgacctctgccccagataccagaccagccccaggatctacagccccacccgcacacggcgtgacaagtgcccct
gacacaagacccgctccaggctctactgctcctcctgcccatggcgtgacaagcgctcccgatacaaggccagctcctggctccacagca
ccaccagcacatggcgtgacatcagctcccgacactagacctgctcccggatcaaccgctccaccagctcacggcgtgaccagcgcacc
tgataccagacctgctctgggaagcaccgcccctcccgtgcacaatgtgacatctgcttccggcagcgccagcggctctgcctctacactg
gtgcacaacggcaccagcgccagagccacaacaaccccagccagcaagagcacccccttcagcatccctagccaccacagcgacacc
cctaccacactggccagccactccaccaagaccgatgcctctagcacccaccactccagcgtgccccctctgaccagcagcaaccacag
cacaagcccccagctgtctaccggcgtctcattcttctttctgtccttccacatcagcaacctgcagttcaacagcagcctggaagatcccag
caccgactactaccaggaactgcagcgggatatcagcgagatgttcctgcaaatctacaagcagggcggcttcctgggcctgagcaacat
caagttcagacccggcagcgtggtggtgcagctgaccctggctttccgggaaggcaccatcaacgtgcacgacgtggaaacccagttca
accagtacaagaccgaggccgccagccggtacaacctgaccatctccgatgtgtccgtgtccgacgtgcccttcccattctctgcccagtct
ggcgcaggcgtgccaggatggggaattgctctgctggtgctcgtgtgcgtgctggtggccctggccatcgtgtatctgattgccctggccgt
gtgccagtgccggcggaagaattacggccagctggacatcttccccgccagagacacctaccaccccatgagcgagtaccccacatacc
acacccacggcagatacgtgccacccagctccaccgacagatccccctacgagaaagtgtctgccggcaacggcggcagctccctgag
ctacacaaatcctgccgtggccgctgcctccgccaacctgggatccggcagaatcttcaacgcccactacgccggctacttcgccgacctg
ctgatccacgacatcgagacaaaccctggccccgaatcgccaagcgcaccccctcatcggtggtgcatcccttggcaacgcctcctcctg
accgcctcactgctgactttctggaacccgccgaccaccgcaaagctgaccattgagagcactcccttcaacgtggctgaggggaaggag
gtgctgctcctggtgcacaatctgccccagcacctgttcgggtactcctggtacaagggagaacgcgtggacgggaaccggcagatcata
ggctacgtcatcggaacccagcaggccacacccggtccagcgtacagcggccgggagattatctacccgaacgcctccctgctgatcca
aaacatcatccagaacgacaccggtttctacactctgcacgtgattaagtcagatctggtcaacgaagaggccaccggccaattcagggtgt
accccgaactccctaagccgttcatcacctcgaacaacagcaacccggtcgaggatgaagatgcggtggccttgacgtgcgaacctgag
atccagaacaccacctacttgtggtgggtgaacaatcagagcctgccagtctccccacgactccagctgtcgaacgacaacaggaccctg
actttgctgtccgtgactcggaacgacgtgggcccttatgaatgcggtatccagaacaagctgtccgtggaccacagcgaccctgtgatcct
gaacgtcctttacgggccggacgaccccaccatttccccgtcgtacacttactaccggccgggcgtgaacctgtccctgtcgtgccacgct
gcctccaatccgccggcccagtactcctggctcatcgacggaaacatccagcagcacacccaagaactgttcatctccaacattaccgaga
aaaactcgggactttacacctgtcaagccaacaattccgccagcggccactcccgcaccactgtcaaaactatcactgtgtccgccgaactc
ccgaagcccagcatcagctccaacaactcgaagcccgtggaggataaggacgctgtcgcgttcacctgtgaaccagaggcacagaatac
cacctacctttggtgggtcaacggacagtccctgcctgtctcaccgagactgcagctgtcaaacgggaataggactctgaccttgtttaacgt
cacccggaacgacgcccgggcctacgtgtgcggcatccagaactccgtgagcgcaaaccggtctgacccagtgaccctggatgtgctgt
acggccccgacactccgatcatttcaccccccgattcatcctacctgtccggcgctaacctcaacctctcatgccactccgcatccaacccc
agcccgcaatattcgtggcgcattaacggaattcctcagcaacatacccaggtcctgttcattgcgaagatcacccctaacaacaacggaac
ctacgcctgctttgtgtcaaacctggccactggtagaaacaactccatcgtgaagtccattaccgtgtcggcgtccggaacttccccgggcct
gagcgccggcgccaccgtgggaattatgatcggcgtgctcgtgggagtggccctgatc.
Plasmid 1426 Polypeptide

SEQ ID NO: 47

MASGAAPEPERTPVGQGSWAHPGRTRGPSDRGFCVVSPARPAEEATSLEGALSGTRHS
HPSVGRQHHAGPPSTSRPPRPWDTPCPPVYAETKHFLYSSGDKEQLRPSFLLSSLRPSLT
GARRLVETIFLGSRPWMPGTPRRLPRLPQRYWQMRPLFLELLGNHAQCPYGVLLKTHC
PLRAAVTPAAGVCAREKPQGSVAAPEEEDTDPRRLVQLLRQHSSPWQVYGFVRACLRR
LVPPGLWGSRHNERRFLRNTKKFISLGKHAKLSLQELTWKMSVRDCAWLRRSPGVGCV
PAAEHRLREEILAKFLHWLMSVYVVELLRSFFYVTETTFQKNRLFFYRKSVWSKLQSIGI
RQHLKRVQLRELSEAEVRQHREARPALLTSRLRFIPKPDGLRPIVNMDYVVGARTFRRE
KRAERLTSRVKALFSVLNYERARRPGLLGASVLGLDDIHRAWRTFVLRVRAQDPPPELY
FVKVAITGAYDTIPQDRLTEVIASIIKPQNTYCVRRYAVVQKAAHGHVRKAFKSHVSTL
TDLQPYMRQFVAHLQETSPLRDAVVIEQSSSLNEASSGLFDVFLRFMCHHAVRIRGKSY
VQCQGIPQGSILSTLLCSLCYGDMENKLFAGIRRDGLLLRLVDDFLLVTPHLTHAKTFLR
TLVRGVPEYGCVVNLRKTVVNFPVEDEALGGTAFVQMPAHGLFPWCGLLLDTRTLEV
QSDYSSYARTSIRASLTFNRGFKAGRNMRRKLFGVLRLKCHSLFLDLQVNSLQTVCTNI
YKILLLQAYRFHACVLQLPFHQQVWKNPTFFLRVISDTASLCYSILKAKNAGMSLGAKG
AAGPLPSEAVQWLCHQAFLLKLTRHRVTYVPLLGSLRTAQTQLSRKLPGTTLTALEAA
ANPALPSDFKTILDGSGTILSEGATNFSLLKLAGDVELNPGPTPGTQSPFFLLLLLTVLTV
VTGSGHASSTPGGEKETSATQRSSVPSSTEKNAVSMTSSVLSSHSPGSGSSTTQGQDVTL
APATEPASGSAATWGQDVTSVPVTRPALGSTTPPAHDVTSAPDNKPAPGSTAPPAHGVT
SAPDTRPAPGSTAPPAHGVTSAPDTRPAPGSTAPPAHGVTSAPDTRPAPGSTAPPAHGVT
SAPDTRPAPGSTAPPAHGVTSAPDTRPALGSTAPPVHNVTSASGSASGSASTLVHNGTSA
RATTTPASKSTPFSIPSHHSDTPTTLASHSTKTDASSTHHSSVPPLTSSNHSTSPQLSTGVS
FFFLSFHISNLQFNSSLEDPSTDYYQELQRDISEMFLQIYKQGGFLGLSNIKFRPGSVVVQ
LTLAFREGTINVHDVETQFNQYKTEAASRYNLTISDVSVSDVPFPFSAQSGAGVPGWGI
ALLVLVCVLVALAIVYLIALAVCQCRRKNYGQLDIFPARDTYHPMSEYPTYHTHGRYV
PPSSTDRSPYEKVSAGNGGSSLSYTNPAVAAASANLGSGRIFNAHYAGYFADLLIHDIET
NPGPESPSAPPHRWCIPWQRLLLTASLLTFWNPPTTAKLTIESTPFNVAEGKEVLLLVHN
LPQHLFGYSWYKGERVDGNRQIIGYVIGTQQATPGPAYSGREIIYPNASLLIQNIIQNDTG
FYTLHVIKSDLVNEEATGQFRVYPELPKPFITSNNSNPVEDEDAVALTCEPEIQNTTYLW
WVNNQSLPVSPRLQLSNDNRTLTLLSVTRNDVGPYECGIQNKLSVDHSDPVILNVLYGP
DDPTISPSYTYYRPGVNLSLSCHAASNPPAQYSWLIDGNIQQHTQELFISNITEKNSGLYT
CQANNSASGHSRTTVKTITVSAELPKPSISSNNSKPVEDKDAVAFTCEPEAQNTTYLWW
VNGQSLPVSPRLQLSNGNRTLTLFNVTRNDARAYVCGIQNSVSANRSDPVTLDVLYGPD
TPIISPPDSSYLSGANLNLSCHSASNPSPQYSWRINGIPQQHTQVLFIAKITPNNNGTYACF
VSNLATGRNNSIVKSITVSASGTSPGLSAGATVGIMIGVLVGVALI.
Plasmid 1427 ORF (nucleotide sequence)

SEQ ID NO: 48

atggctagcggagctgccccggagccggagaggacccccgttggccagggatcgtgggcccatccgggacgcaccaggggaccatc
cgacaggggattctgtgtggtgtcaccggccaggccagcagaagaggcaaccagcctcgagggagcgttgtctggaaccagacattccc
acccgtcggtgggccggcagcaccacgcgggaccaccgtccacttccagaccgccacggccatgggacaccccttgcccgcctgtgta
tgccgagactaaacacttcctgtactcatccggagacaaggaacagcttcggccgtccttcctcctgtcgtcgctcagaccgagcctgaccg
gagcacgcagattggtggaaactatcttccttgggtcacgtccgtggatgccaggtaccccacggcgcctcccgcgcctcccacagagat
actggcagatgcggcctctgttcctggaattgctgggaaaccacgctcagtgcccgtacggagtcctgctcaagactcactgccctctgag
ggcggcggtcactccggcggccggagtgtgcgcacgggagaagccccagggaagcgtggcagctccggaagaggaggacaccgat
ccgcgccgcctcgtgcaacttctgcgccagcactcctcgccctggcaagtctacgggttcgtccgcgcctgcctgcgccgcctggtgccg
cctgggctctggggttcccggcataacgagcgccgcttcctgagaaatactaagaagtttatctcacttggaaaacatgccaagttgtcgctg
caagaactcacgtggaagatgtcagtccgcgattgcgcctggctgcgccgctcgccgggcgtcgggtgtgttccagctgcagaacaccg
cctgagagaagaaattctggccaaatttctgcattggctgatgtcagtgtacgtggtcgagctgctgcgctcctttttctacgtcactgagacta
cctttcaaaagaaccgcctgttcttctaccgcaaatctgtgtggagcaagctgcagtcaatcggcattcgccagcatctgaagagggtgcag
ctgcgggaactttccgaggcagaagtccgccagcaccgggaggcccggccggcgcttctcacgtcgcgtctgagattcatcccaaagcc
cgacgggctgaggcctatcgtcaacatggattacgtcgtgggcgctcgcacctttcgccgtgaaaagcgggccgaacgcttgacctcacg
ggtgaaggccctcttctccgtgctgaactacgagagagcaagacggcctggcctgctgggagcttcggtgctgggactggacgatatcca
ccgggcttggcggacctttgttctccgggtgagagcccaagaccctccgccggaactgtacttcgtgaaggtggcgatcaccggagcctat
gatactattccgcaagatcgactcaccgaagtcatcgcctcgatcatcaaaccgcagaacacttactgcgtcaggcggtacgccgtggtcc
agaaggccgcgcatggccacgtgagaaaggcgttcaagtcgcacgtgtccactctcaccgacctccagccttacatgaggcaattcgttg
cgcatttgcaagagacttcgcccctgagagatgcggtggtcatcgagcagagctccagcctgaacgaagcgagcagcggtctgtttgacg
tgttcctccgcttcatgtgtcatcacgcggtgcgaatcaggggaaaatcatacgtgcagtgccagggaatcccacaaggcagcattctgtcg
actctcttgtgttccctttgctacggcgatatggaaaacaagctgttcgctgggatcagacgggacgggttgctgctcagactggtggacga
cttcctgctggtgactccgcacctcactcacgccaaaacctttctccgcactctggtgaggggagtgccagaatacggctgtgtggtcaatct
ccggaaaactgtggtgaatttccctgtcgaggatgaggcactcggaggaaccgcatttgtccaaatgccagcacatggcctgttcccatggt
gcggtctgctgctggacacccgaactcttgaagtgcagtccgactactccagctatgcccggacgagcatccgcgccagcctcactttcaa
tcgcggctttaaggccggacgaaacatgcgcagaaagcttttcggagtcctccggcttaaatgccattcgctctttctcgatctccaagtcaat
tcgctgcagaccgtgtgcacgaacatctacaagatcctgctgctccaagcctaccggttccacgcttgcgtgcttcagctgccgtttcaccaa
caggtgtggaagaacccgaccttctttctgcgggtcattagcgatactgcctccctgtgttactcaatcctcaaggcaaagaacgccggaat
gtcgctgggtgcgaaaggagccgcgggacctcttcctagcgaagcggtgcagtggctctgccaccaggctttcctcctgaagctgaccag
gcacagagtgacctacgtcccgctgctgggctcgctgcgcactgcacagacccagctgtctagaaaactccccggcaccaccctgaccg
ctctggaagccgccgccaacccagcattgccgtcagatttcaagaccatcttggacggatccggccagtgcaccaattacgccctgctgaa
gctggccggcgacgtggaatctaaccctggccctgaatcgccaagcgcaccccctcatcggtggtgcatcccttggcaacgcctcctcct
gaccgcctcactgctgactttctggaacccgccgaccaccgcaaagctgaccattgagagcactcccttcaacgtggctgaggggaagga
ggtgctgctcctggtgcacaatctgccccagcacctgttcgggtactcctggtacaagggagaacgcgtggacgggaaccggcagatcat
aggctacgtcatcggaacccagcaggccacacccggtccagcgtacagcggccgggagattatctacccgaacgcctccctgctgatcc
aaaacatcatccagaacgacaccggtttctacactctgcacgtgattaagtcagatctggtcaacgaagaggccaccggccaattcagggt
gtaccccgaactccctaagccgttcatcacctcgaacaacagcaacccggtcgaggatgaagatgcggtggccttgacgtgcgaacctga
gatccagaacaccacctacttgtggtgggtgaacaatcagagcctgccagtctccccacgactccagctgtcgaacgacaacaggaccct
gactttgctgtccgtgactcggaacgacgtgggcccttatgaatgcggtatccagaacaagctgtccgtggaccacagcgaccctgtgatc
ctgaacgtcctttacgggccggacgaccccaccatttccccgtcgtacacttactaccggccgggcgtgaacctgtccctgtcgtgccacg
ctgcctccaatccgccggcccagtactcctggctcatcgacggaaacatccagcagcacacccaagaactgttcatctccaacattaccga
gaaaaactcgggactttacacctgtcaagccaacaattccgccagcggccactcccgcaccactgtcaaaactatcactgtgtccgccgaa
ctcccgaagcccagcatcagctccaacaactcgaagcccgtggaggataaggacgctgtcgcgttcacctgtgaaccagaggcacagaa
taccacctacctttggtgggtcaacggacagtccctgcctgtctcaccgagactgcagctgtcaaacgggaataggactctgaccttgtttaa
cgtcacccggaacgacgcccgggcctacgtgtgcggcatccagaactccgtgagcgcaaaccggtctgacccagtgaccctggatgtgc
tgtacggccccgacactccgatcatttcaccccccgattcatcctacctgtccggcgctaacctcaacctctcatgccactccgcatccaacc
ccagcccgcaatattcgtggcgcattaacggaattcctcagcaacatacccaggtcctgttcattgcgaagatcacccctaacaacaacgga
acctacgcctgctttgtgtcaaacctggccactggtagaaacaactccatcgtgaagtccattaccgtgtcggcgtccggaacttccccggg
cctgagcgccggcgccaccgtgggaattatgatcggcgtgctcgtgggagtggccctgatcggatccggcgagggcagaggcagcctg
ctgacatgtggcgacgtggaagagaaccctggccccacccctggaacccagagccccttcttccttctgctgctgctgaccgtgctgactgt
cgtgacaggctctggccacgccagctctacacctggcggcgagaaagagacaagcgccacccagagaagcagcgtgccaagcagca
ccgagaagaacgccgtgtccatgaccagctccgtgctgagcagccactctcctggcagcggcagcagcacaacacagggccaggatgt
gacactggcccctgccacagaacctgcctctggatctgccgccacctggggacaggacgtgacaagcgtgccagtgaccagacctgcc
ctgggctctacaacaccccctgcccacgatgtgaccagcgcccctgataacaagcctgcccctggaagcacagcccctccagctcatggc
gtgacctctgccccagataccagaccagccccaggatctacagccccacccgcacacggcgtgacaagtgcccctgacacaagacccg
ctccaggctctactgctcctcctgcccatggcgtgacaagcgctcccgatacaaggccagctcctggctccacagcaccaccagcacatg
gcgtgacatcagctcccgacactagacctgctcccggatcaaccgctccaccagctcacggcgtgaccagcgcacctgataccagacct
gctctgggaagcaccgcccctcccgtgcacaatgtgacatctgcttccggcagcgccagcggctctgcctctacactggtgcacaacggc
accagcgccagagccacaacaaccccagccagcaagagcacccccttcagcatccctagccaccacagcgacacccctaccacactgg
ccagccactccaccaagaccgatgcctctagcacccaccactccagcgtgccccctctgaccagcagcaaccacagcacaagccccca
gctgtctaccggcgtctcattcttctttctgtccttccacatcagcaacctgcagttcaacagcagcctggaagatcccagcaccgactactac
caggaactgcagcgggatatcagcgagatgttcctgcaaatctacaagcagggcggcttcctgggcctgagcaacatcaagttcagaccc
ggcagcgtggtggtgcagctgaccctggctttccgggaaggcaccatcaacgtgcacgacgtggaaacccagttcaaccagtacaagac
cgaggccgccagccggtacaacctgaccatctccgatgtgtccgtgtccgacgtgcccttcccattctctgcccagtctggcgcaggcgtg
ccaggatggggaattgctctgctggtgctcgtgtgcgtgctggtggccctggccatcgtgtatctgattgccctggccgtgtgccagtgccg
gcggaagaattacggccagctggacatcttccccgccagagacacctaccaccccatgagcgagtaccccacataccacacccacggca
gatacgtgccacccagctccaccgacagatccccctacgagaaagtgtctgccggcaacggcggcagctccctgagctacacaaatcct
gccgtggccgctgcctccgccaacctg.
Plasmid 1427 Polypeptide

SEQ ID NO: 49

MASGAAPEPERTPVGQGSWAHPGRTRGPSDRGFCVVSPARPAEEATSLEGALSGTRHS
HPSVGRQHHAGPPSTSRPPRPWDTPCPPVYAETKHFLYSSGDKEQLRPSFLLSSLRPSLT
GARRLVETIFLGSRPWMPGTPRRLPRLPQRYWQMRPLFLELLGNHAQCPYGVLLKTHC
PLRAAVTPAAGVCAREKPQGSVAAPEEEDTDPRRLVQLLRQHSSPWQVYGFVRACLRR
LVPPGLWGSRHNERRFLRNTKKFISLGKHAKLSLQELTWKMSVRDCAWLRRSPGVGCV
PAAEHRLREEILAKFLHWLMSVYVVELLRSFFYVTETTFQKNRLFFYRKSVWSKLQSIGI
RQHLKRVQLRELSEAEVRQHREARPALLTSRLRFIPKPDGLRPIVNMDYVVGARTFRRE
KRAERLTSRVKALFSVLNYERARRPGLLGASVLGLDDIHRAWRTFVLRVRAQDPPPELY
FVKVAITGAYDTIPQDRLTEVIASIIKPQNTYCVRRYAVVQKAAHGHVRKAFKSHVSTL
TDLQPYMRQFVAHLQETSPLRDAVVIEQSSSLNEASSGLFDVFLRFMCHHAVRIRGKSY
VQCQGIPQGSILSTLLCSLCYGDMENKLFAGIRRDGLLLRLVDDFLLVTPHLTHAKTFLR
TLVRGVPEYGCVVNLRKTVVNFPVEDEALGGTAFVQMPAHGLFPWCGLLLDTRTLEV
QSDYSSYARTSIRASLTFNRGFKAGRNMRRKLFGVLRLKCHSLFLDLQVNSLQTVCTNI
YKILLLQAYRFHACVLQLPFHQQVWKNPTFFLRVISDTASLCYSILKAKNAGMSLGAKG
AAGPLPSEAVQWLCHQAFLLKLTRHRVTYVPLLGSLRTAQTQLSRKLPGTTLTALEAA
ANPALPSDFKTILDGSGQCTNYALLKLAGDVESNPGPESPSAPPHRWCIPWQRLLLTASL
LTFWNPPTTAKLTIESTPFNVAEGKEVLLLVHNLPQHLFGYSWYKGERVDGNRQIIGYVI
GTQQATPGPAYSGREIIYPNASLLIQNIIQNDTGFYTLHVIKSDLVNEEATGQFRVYPELP
KPFITSNNSNPVEDEDAVALTCEPEIQNTTYLWWVNNQSLPVSPRLQLSNDNRTLTLLSV
TRNDVGPYECGIQNKLSVDHSDPVILNVLYGPDDPTISPSYTYYRPGVNLSLSCHAASNP
PAQYSWLIDGNIQQHTQELFISNITEKNSGLYTCQANNSASGHSRTTVKTITVSAELPKPS
ISSNNSKPVEDKDAVAFTCEPEAQNTTYLWWVNGQSLPVSPRLQLSNGNRTLTLFNVTR
NDARAYVCGIQNSVSANRSDPVTLDVLYGPDTPIISPPDSSYLSGANLNLSCHSASNPSP
QYSWRINGIPQQHTQVLFIAKITPNNNGTYACFVSNLATGRNNSIVKSITVSASGTSPGLS
AGATVGIMIGVLVGVALIGSGEGRGSLLTCGDVEENPGPTPGTQSPFFLLLLLTVLTVVT
GSGHASSTPGGEKETSATQRSSVPSSTEKNAVSMTSSVLSSHSPGSGSSTTQGQDVTLAP
ATEPASGSAATWGQDVTSVPVTRPALGSTTPPAHDVTSAPDNKPAPGSTAPPAHGVTSA
PDTRPAPGSTAPPAHGVTSAPDTRPAPGSTAPPAHGVTSAPDTRPAPGSTAPPAHGVTSA
PDTRPAPGSTAPPAHGVTSAPDTRPALGSTAPPVHNVTSASGSASGSASTLVHNGTSAR
ATTTPASKSTPFSIPSHHSDTPTTLASHSTKTDASSTHHSSVPPLTSSNHSTSPQLSTGVSFF
FLSFHISNLQFNSSLEDPSTDYYQELQRDISEMFLQIYKQGGFLGLSNIKFRPGSVVVQLT
LAFREGTINVHDVETQFNQYKTEAASRYNLTISDVSVSDVPFPFSAQSGAGVPGWGIAL
LVLVCVLVALAIVYLIALAVCQCRRKNYGQLDIFPARDTYHPMSEYPTYHTHGRYVPPS
STDRSPYEKVSAGNGGSSLSYTNPAVAAASANL.
Plasmid 1428 ORF (nucleotide sequence)

SEQ ID NO: 50

atggctagcacccctggaacccagagccccttcttccttctgctgctgctgaccgtgctgactgtcgtgacaggctctggccacgccagctc
tacacctggcggcgagaaagagacaagcgccacccagagaagcagcgtgccaagcagcaccgagaagaacgccgtgtccatgacca
gctccgtgctgagcagccactctcctggcagcggcagcagcacaacacagggccaggatgtgacactggcccctgccacagaacctgc
ctctggatctgccgccacctggggacaggacgtgacaagcgtgccagtgaccagacctgccctgggctctacaacaccccctgcccacg
atgtgaccagcgcccctgataacaagcctgcccctggaagcacagcccctccagctcatggcgtgacctctgccccagataccagacca
gccccaggatctacagccccacccgcacacggcgtgacaagtgcccctgacacaagacccgctccaggctctactgctcctcctgcccat
ggcgtgacaagcgctcccgatacaaggccagctcctggctccacagcaccaccagcacatggcgtgacatcagctcccgacactagacc
tgctcccggatcaaccgctccaccagctcacggcgtgaccagcgcacctgataccagacctgctctgggaagcaccgcccctcccgtgc
acaatgtgacatctgcttccggcagcgccagcggctctgcctctacactggtgcacaacggcaccagcgccagagccacaacaacccca
gccagcaagagcacccccttcagcatccctagccaccacagcgacacccctaccacactggccagccactccaccaagaccgatgcctc
tagcacccaccactccagcgtgccccctctgaccagcagcaaccacagcacaagcccccagctgtctaccggcgtctcattcttctttctgt
ccttccacatcagcaacctgcagttcaacagcagcctggaagatcccagcaccgactactaccaggaactgcagcgggatatcagcgag
atgttcctgcaaatctacaagcagggcggcttcctgggcctgagcaacatcaagttcagacccggcagcgtggtggtgcagctgaccctg
gctttccgggaaggcaccatcaacgtgcacgacgtggaaacccagttcaaccagtacaagaccgaggccgccagccggtacaacctga
ccatctccgatgtgtccgtgtccgacgtgcccttcccattctctgcccagtctggcgcaggcgtgccaggatggggaattgctctgctggtgc
tcgtgtgcgtgctggtggccctggccatcgtgtatctgattgccctggccgtgtgccagtgccggcggaagaattacggccagctggacat
cttccccgccagagacacctaccaccccatgagcgagtaccccacataccacacccacggcagatacgtgccacccagctccaccgaca
gatccccctacgagaaagtgtctgccggcaacggcggcagctccctgagctacacaaatcctgccgtggccgctgcctccgccaacctg
ggatccggcagaatcttcaacgcccactacgccggctacttcgccgacctgctgatccacgacatcgagacaaaccctggccccaagctg
accattgagagcactcccttcaacgtggctgaggggaaggaggtgctgctcctggtgcacaatctgccccagcacctgttcgggtactcct
ggtacaagggagaacgcgtggacgggaaccggcagatcataggctacgtcatcggaacccagcaggccacacccggtccagcgtaca
gcggccgggagattatctacccgaacgcctccctgctgatccaaaacatcatccagaacgacaccggtttctacactctgcacgtgattaag
tcagatctggtcaacgaagaggccaccggccaattcagggtgtaccccgaactccctaagccgttcatcacctcgaacaacagcaacccg
gtcgaggatgaagatgcggtggccttgacgtgcgaacctgagatccagaacaccacctacttgtggtgggtgaacaatcagagcctgcca
gtctccccacgactccagctgtcgaacgacaacaggaccctgactttgctgtccgtgactcggaacgacgtgggcccttatgaatgcggtat
ccagaacaagctgtccgtggaccacagcgaccctgtgatcctgaacgtcctttacgggccggacgaccccaccatttccccgtcgtacact
tactaccggccgggcgtgaacctgtccctgtcgtgccacgctgcctccaatccgccggcccagtactcctggctcatcgacggaaacatcc
agcagcacacccaagaactgttcatctccaacattaccgagaaaaactcgggactttacacctgtcaagccaacaattccgccagcggcca
ctcccgcaccactgtcaaaactatcactgtgtccgccgaactcccgaagcccagcatcagctccaacaactcgaagcccgtggaggataa
ggacgctgtcgcgttcacctgtgaaccagaggcacagaataccacctacctttggtgggtcaacggacagtccctgcctgtctcaccgaga
ctgcagctgtcaaacgggaataggactctgaccttgtttaacgtcacccggaacgacgcccgggcctacgtgtgcggcatccagaactcc
gtgagcgcaaaccggtctgacccagtgaccctggatgtgctgtacggccccgacactccgatcatttcaccccccgattcatcctacctgtc
cggcgctaacctcaacctctcatgccactccgcatccaaccccagcccgcaatattcgtggcgcattaacggaattcctcagcaacataccc
aggtcctgttcattgcgaagatcacccctaacaacaacggaacctacgcctgctttgtgtcaaacctggccactggtagaaacaactccatc
gtgaagtccattaccgtgtcggcgtccggatccggcgagggcagaggcagcctgctgacatgtggcgacgtggaagagaaccctggcc
ccggagctgccccggagccggagaggacccccgttggccagggatcgtgggcccatccgggacgcaccaggggaccatccgacagg
ggattctgtgtggtgtcaccggccaggccagcagaagaggcaaccagcctcgagggagcgttgtctggaaccagacattcccacccgtc
ggtgggccggcagcaccacgcgggaccaccgtccacttccagaccgccacggccatgggacaccccttgcccgcctgtgtatgccgag
actaaacacttcctgtactcatccggagacaaggaacagcttcggccgtccttcctcctgtcgtcgctcagaccgagcctgaccggagcac
gcagattggtggaaactatcttccttgggtcacgtccgtggatgccaggtaccccacggcgcctcccgcgcctcccacagagatactggca
gatgcggcctctgttcctggaattgctgggaaaccacgctcagtgcccgtacggagtcctgctcaagactcactgccctctgagggcggcg
gtcactccggcggccggagtgtgcgcacgggagaagccccagggaagcgtggcagctccggaagaggaggacaccgatccgcgcc
gcctcgtgcaacttctgcgccagcactcctcgccctggcaagtctacgggttcgtccgcgcctgcctgcgccgcctggtgccgcctgggct
ctggggttcccggcataacgagcgccgcttcctgagaaatactaagaagtttatctcacttggaaaacatgccaagttgtcgctgcaagaact
cacgtggaagatgtcagtccgcgattgcgcctggctgcgccgctcgccgggcgtcgggtgtgttccagctgcagaacaccgcctgagag
aagaaattctggccaaatttctgcattggctgatgtcagtgtacgtggtcgagctgctgcgctcctttttctacgtcactgagactacctttcaaa
agaaccgcctgttcttctaccgcaaatctgtgtggagcaagctgcagtcaatcggcattcgccagcatctgaagagggtgcagctgcggga
actttccgaggcagaagtccgccagcaccgggaggcccggccggcgcttctcacgtcgcgtctgagattcatcccaaagcccgacgggc
tgaggcctatcgtcaacatggattacgtcgtgggcgctcgcacctttcgccgtgaaaagcgggccgaacgcttgacctcacgggtgaagg
ccctcttctccgtgctgaactacgagagagcaagacggcctggcctgctgggagcttcggtgctgggactggacgatatccaccgggctt
ggcggacctttgttctccgggtgagagcccaagaccctccgccggaactgtacttcgtgaaggtggcgatcaccggagcctatgatactatt
ccgcaagatcgactcaccgaagtcatcgcctcgatcatcaaaccgcagaacacttactgcgtcaggcggtacgccgtggtccagaaggcc
gcgcatggccacgtgagaaaggcgttcaagtcgcacgtgtccactctcaccgacctccagccttacatgaggcaattcgttgcgcatttgca
agagacttcgcccctgagagatgcggtggtcatcgagcagagctccagcctgaacgaagcgagcagcggtctgtttgacgtgttcctccg
cttcatgtgtcatcacgcggtgcgaatcaggggaaaatcatacgtgcagtgccagggaatcccacaaggcagcattctgtcgactctcttgt
gttccctttgctacggcgatatggaaaacaagctgttcgctgggatcagacgggacgggttgctgctcagactggtggacgacttcctgctg
gtgactccgcacctcactcacgccaaaacctttctccgcactctggtgaggggagtgccagaatacggctgtgtggtcaatctccggaaaa
ctgtggtgaatttccctgtcgaggatgaggcactcggaggaaccgcatttgtccaaatgccagcacatggcctgttcccatggtgcggtctg
ctgctggacacccgaactcttgaagtgcagtccgactactccagctatgcccggacgagcatccgcgccagcctcactttcaatcgcggctt
taaggccggacgaaacatgcgcagaaagcttttcggagtcctccggcttaaatgccattcgctctttctcgatctccaagtcaattcgctgca
gaccgtgtgcacgaacatctacaagatcctgctgctccaagcctaccggttccacgcttgcgtgcttcagctgccgtttcaccaacaggtgtg
gaagaacccgaccttctttctgcgggtcattagcgatactgcctccctgtgttactcaatcctcaaggcaaagaacgccggaatgtcgctgg
gtgcgaaaggagccgcgggacctcttcctagcgaagcggtgcagtggctctgccaccaggctttcctcctgaagctgaccaggcacaga
gtgacctacgtcccgctgctgggctcgctgcgcactgcacagacccagctgtctagaaaactccccggcaccaccctgaccgctctggaa
gccgccgccaacccagcattgccgtcagatttcaagaccatcttggac.
Plasmid 1428 Polypeptide

SEQ ID NO: 51

MASTPGTQSPFFLLLLLTVLTVVTGSGHASSTPGGEKETSATQRSSVPSSTEKNAVSMTS
SVLSSHSPGSGSSTTQGQDVTLAPATEPASGSAATWGQDVTSVPVTRPALGSTTPPAHD
VTSAPDNKPAPGSTAPPAHGVTSAPDTRPAPGSTAPPAHGVTSAPDTRPAPGSTAPPAHG
VTSAPDTRPAPGSTAPPAHGVTSAPDTRPAPGSTAPPAHGVTSAPDTRPALGSTAPPVHN
VTSASGSASGSASTLVHNGTSARATTTPASKSTPFSIPSHHSDTPTTLASHSTKTDASSTH
HSSVPPLTSSNHSTSPQLSTGVSFFFLSFHISNLQFNSSLEDPSTDYYQELQRDISEMFLQI
YKQGGFLGLSNIKFRPGSVVVQLTLAFREGTINVHDVETQFNQYKTEAASRYNLTISDV
SVSDVPFPFSAQSGAGVPGWGIALLVLVCVLVALAIVYLIALAVCQCRRKNYGQLDIFP
ARDTYHPMSEYPTYHTHGRYVPPSSTDRSPYEKVSAGNGGSSLSYTNPAVAAASANLG
SGRIFNAHYAGYFADLLIHDIETNPGPKLTIESTPFNVAEGKEVLLLVHNLPQHLFGYSW
YKGERVDGNRQIIGYVIGTQQATPGPAYSGREIIYPNASLLIQNIIQNDTGFYTLHVIKSDL
VNEEATGQFRVYPELPKPFITSNNSNPVEDEDAVALTCEPEIQNTTYLWWVNNQSLPVS
PRLQLSNDNRTLTLLSVTRNDVGPYECGIQNKLSVDHSDPVILNVLYGPDDPTISPSYTY
YRPGVNLSLSCHAASNPPAQYSWLIDGNIQQHTQELFISNITEKNSGLYTCQANNSASGH
SRTTVKTITVSAELPKPSISSNNSKPVEDKDAVAFTCEPEAQNTTYLWWVNGQSLPVSPR
LQLSNGNRTLTLFNVTRNDARAYVCGIQNSVSANRSDPVTLDVLYGPDTPIISPPDSSYL
SGANLNLSCHSASNPSPQYSWRINGIPQQHTQVLFIAKITPNNNGTYACFVSNLATGRNN
SIVKSITVSASGSGEGRGSLLTCGDVEENPGPGAAPEPERTPVGQGSWAHPGRTRGPSDR
GFCVVSPARPAEEATSLEGALSGTRHSHPSVGRQHHAGPPSTSRPPRPWDTPCPPVYAET
KHFLYSSGDKEQLRPSFLLSSLRPSLTGARRLVETIFLGSRPWMPGTPRRLPRLPQRYWQ
MRPLFLELLGNHAQCPYGVLLKTHCPLRAAVTPAAGVCAREKPQGSVAAPEEEDTDPR
RLVQLLRQHSSPWQVYGFVRACLRRLVPPGLWGSRHNERRFLRNTKKFISLGKHAKLS
LQELTWKMSVRDCAWLRRSPGVGCVPAAEHRLREEILAKFLHWLMSVYVVELLRSFF
YVTETTFQKNRLFFYRKSVWSKLQSIGIRQHLKRVQLRELSEAEVRQHREARPALLTSR
LRFIPKPDGLRPIVNMDYVVGARTFRREKRAERLTSRVKALFSVLNYERARRPGLLGAS
VLGLDDIHRAWRTFVLRVRAQDPPPELYFVKVAITGAYDTIPQDRLTEVIASIIKPQNTY
CVRRYAVVQKAAHGHVRKAFKSHVSTLTDLQPYMRQFVAHLQETSPLRDAVVIEQSSS
LNEASSGLFDVFLRFMCHHAVRIRGKSYVQCQGIPQGSILSTLLCSLCYGDMENKLFAGI
RRDGLLLRLVDDFLLVTPHLTHAKTFLRTLVRGVPEYGCVVNLRKTVVNFPVEDEALG
GTAFVQMPAHGLFPWCGLLLDTRTLEVQSDYSSYARTSIRASLTFNRGFKAGRNMRRK
LFGVLRLKCHSLFLDLQVNSLQTVCTNIYKILLLQAYRFHACVLQLPFHQQVWKNPTFF
LRVISDTASLCYSILKAKNAGMSLGAKGAAGPLPSEAVQWLCHQAFLLKLTRHRVTYV
PLLGSLRTAQTQLSRKLPGTTLTALEAAANPALPSDFKTILD.
Plasmid 1429 ORF (nucleotide sequence)

SEQ ID NO: 52

atggctagcaagctgaccattgagagcactcccttcaacgtggctgaggggaaggaggtgctgctcctggtgcacaatctgccccagcac
ctgttcgggtactcctggtacaagggagaacgcgtggacgggaaccggcagatcataggctacgtcatcggaacccagcaggccacac
ccggtccagcgtacagcggccgggagattatctacccgaacgcctccctgctgatccaaaacatcatccagaacgacaccggtttctacac
tctgcacgtgattaagtcagatctggtcaacgaagaggccaccggccaattcagggtgtaccccgaactccctaagccgttcatcacctcga
acaacagcaacccggtcgaggatgaagatgcggtggccttgacgtgcgaacctgagatccagaacaccacctacttgtggtgggtgaac
aatcagagcctgccagtctccccacgactccagctgtcgaacgacaacaggaccctgactttgctgtccgtgactcggaacgacgtgggc
ccttatgaatgcggtatccagaacaagctgtccgtggaccacagcgaccctgtgatcctgaacgtcctttacgggccggacgaccccacca
tttccccgtcgtacacttactaccggccgggcgtgaacctgtccctgtcgtgccacgctgcctccaatccgccggcccagtactcctggctc
atcgacggaaacatccagcagcacacccaagaactgttcatctccaacattaccgagaaaaactcgggactttacacctgtcaagccaaca
attccgccagcggccactcccgcaccactgtcaaaactatcactgtgtccgccgaactcccgaagcccagcatcagctccaacaactcga
agcccgtggaggataaggacgctgtcgcgttcacctgtgaaccagaggcacagaataccacctacctttggtgggtcaacggacagtccc
tgcctgtctcaccgagactgcagctgtcaaacgggaataggactctgaccttgtttaacgtcacccggaacgacgcccgggcctacgtgtg
cggcatccagaactccgtgagcgcaaaccggtctgacccagtgaccctggatgtgctgtacggccccgacactccgatcatttcaccccc
cgattcatcctacctgtccggcgctaacctcaacctctcatgccactccgcatccaaccccagcccgcaatattcgtggcgcattaacggaat
tcctcagcaacatacccaggtcctgttcattgcgaagatcacccctaacaacaacggaacctacgcctgctttgtgtcaaacctggccactg
gtagaaacaactccatcgtgaagtccattaccgtgtcggcgtccggatccggcgagggcagaggcagcctgctgacatgtggcgacgtg
gaagagaaccctggccccggagctgccccggagccggagaggacccccgttggccagggatcgtgggcccatccgggacgcaccag
gggaccatccgacaggggattctgtgtggtgtcaccggccaggccagcagaagaggcaaccagcctcgagggagcgttgtctggaacc
agacattcccacccgtcggtgggccggcagcaccacgcgggaccaccgtccacttccagaccgccacggccatgggacaccccttgcc
cgcctgtgtatgccgagactaaacacttcctgtactcatccggagacaaggaacagcttcggccgtccttcctcctgtcgtcgctcagaccg
agcctgaccggagcacgcagattggtggaaactatcttccttgggtcacgtccgtggatgccaggtaccccacggcgcctcccgcgcctc
ccacagagatactggcagatgcggcctctgttcctggaattgctgggaaaccacgctcagtgcccgtacggagtcctgctcaagactcact
gccctctgagggcggcggtcactccggcggccggagtgtgcgcacgggagaagccccagggaagcgtggcagctccggaagagga
ggacaccgatccgcgccgcctcgtgcaacttctgcgccagcactcctcgccctggcaagtctacgggttcgtccgcgcctgcctgcgccg
cctggtgccgcctgggctctggggttcccggcataacgagcgccgcttcctgagaaatactaagaagtttatctcacttggaaaacatgcca
agttgtcgctgcaagaactcacgtggaagatgtcagtccgcgattgcgcctggctgcgccgctcgccgggcgtcgggtgtgttccagctgc
agaacaccgcctgagagaagaaattctggccaaatttctgcattggctgatgtcagtgtacgtggtcgagctgctgcgctcctttttctacgtc
actgagactacctttcaaaagaaccgcctgttcttctaccgcaaatctgtgtggagcaagctgcagtcaatcggcattcgccagcatctgaag
agggtgcagctgcgggaactttccgaggcagaagtccgccagcaccgggaggcccggccggcgcttctcacgtcgcgtctgagattcat
cccaaagcccgacgggctgaggcctatcgtcaacatggattacgtcgtgggcgctcgcacctttcgccgtgaaaagcgggccgaacgctt
gacctcacgggtgaaggccctcttctccgtgctgaactacgagagagcaagacggcctggcctgctgggagcttcggtgctgggactgg
acgatatccaccgggcttggcggacctttgttctccgggtgagagcccaagaccctccgccggaactgtacttcgtgaaggtggcgatcac
cggagcctatgatactattccgcaagatcgactcaccgaagtcatcgcctcgatcatcaaaccgcagaacacttactgcgtcaggcggtac
gccgtggtccagaaggccgcgcatggccacgtgagaaaggcgttcaagtcgcacgtgtccactctcaccgacctccagccttacatgag
gcaattcgttgcgcatttgcaagagacttcgcccctgagagatgcggtggtcatcgagcagagctccagcctgaacgaagcgagcagcg
gtctgtttgacgtgttcctccgcttcatgtgtcatcacgcggtgcgaatcaggggaaaatcatacgtgcagtgccagggaatcccacaaggc
agcattctgtcgactctcttgtgttccctttgctacggcgatatggaaaacaagctgttcgctgggatcagacgggacgggttgctgctcaga
ctggtggacgacttcctgctggtgactccgcacctcactcacgccaaaacctttctccgcactctggtgaggggagtgccagaatacggct
gtgtggtcaatctccggaaaactgtggtgaatttccctgtcgaggatgaggcactcggaggaaccgcatttgtccaaatgccagcacatggc
ctgttcccatggtgcggtctgctgctggacacccgaactcttgaagtgcagtccgactactccagctatgcccggacgagcatccgcgcca
gcctcactttcaatcgcggctttaaggccggacgaaacatgcgcagaaagcttttcggagtcctccggcttaaatgccattcgctctttctcga
tctccaagtcaattcgctgcagaccgtgtgcacgaacatctacaagatcctgctgctccaagcctaccggttccacgcttgcgtgcttcagct
gccgtttcaccaacaggtgtggaagaacccgaccttctttctgcgggtcattagcgatactgcctccctgtgttactcaatcctcaaggcaaa
gaacgccggaatgtcgctgggtgcgaaaggagccgcgggacctcttcctagcgaagcggtgcagtggctctgccaccaggctttcctcct
gaagctgaccaggcacagagtgacctacgtcccgctgctgggctcgctgcgcactgcacagacccagctgtctagaaaactccccggca
ccaccctgaccgctctggaagccgccgccaacccagcattgccgtcagatttcaagaccatcttggacggatccggcacaatcctgtctga
gggcgccaccaacttcagcctgctgaaactggccggcgacgtggaactgaaccctggccctacccctggaacccagagccccttcttcct
tctgctgctgctgaccgtgctgactgtcgtgacaggctctggccacgccagctctacacctggcggcgagaaagagacaagcgccaccc
agagaagcagcgtgccaagcagcaccgagaagaacgccgtgtccatgaccagctccgtgctgagcagccactctcctggcagcggca
gcagcacaacacagggccaggatgtgacactggcccctgccacagaacctgcctctggatctgccgccacctggggacaggacgtgac
aagcgtgccagtgaccagacctgccctgggctctacaacaccccctgcccacgatgtgaccagcgcccctgataacaagcctgcccctg
gaagcacagcccctccagctcatggcgtgacctctgccccagataccagaccagccccaggatctacagccccacccgcacacggcgt
gacaagtgcccctgacacaagacccgctccaggctctactgctcctcctgcccatggcgtgacaagcgctcccgatacaaggccagctcc
tggctccacagcaccaccagcacatggcgtgacatcagctcccgacactagacctgctcccggatcaaccgctccaccagctcacggcgt
gaccagcgcacctgataccagacctgctctgggaagcaccgcccctcccgtgcacaatgtgacatctgcttccggcagcgccagcggct
ctgcctctacactggtgcacaacggcaccagcgccagagccacaacaaccccagccagcaagagcacccccttcagcatccctagcca
ccacagcgacacccctaccacactggccagccactccaccaagaccgatgcctctagcacccaccactccagcgtgccccctctgacca
gcagcaaccacagcacaagcccccagctgtctaccggcgtctcattcttctttctgtccttccacatcagcaacctgcagttcaacagcagcc
tggaagatcccagcaccgactactaccaggaactgcagcgggatatcagcgagatgttcctgcaaatctacaagcagggcggcttcctgg
gcctgagcaacatcaagttcagacccggcagcgtggtggtgcagctgaccctggctttccgggaaggcaccatcaacgtgcacgacgtg
gaaacccagttcaaccagtacaagaccgaggccgccagccggtacaacctgaccatctccgatgtgtccgtgtccgacgtgcccttcccat
tctctgcccagtctggcgcaggcgtgccaggatggggaattgctctgctggtgctcgtgtgcgtgctggtggccctggccatcgtgtatctg
attgccctggccgtgtgccagtgccggcggaagaattacggccagctggacatcttccccgccagagacacctaccaccccatgagcga
gtaccccacataccacacccacggcagatacgtgccacccagctccaccgacagatccccctacgagaaagtgtctgccggcaacggc
ggcagctccctgagctacacaaatcctgccgtggccgctgcctccgccaacctg.
Plasmid 1429 Polypeptide

SEQ ID NO: 53

MASKLTIESTPFNVAEGKEVLLLVHNLPQHLFGYSWYKGERVDGNRQIIGYVIGTQQAT
PGPAYSGREIIYPNASLLIQNIIQNDTGFYTLHVIKSDLVNEEATGQFRVYPELPKPFITSN
NSNPVEDEDAVALTCEPEIQNTTYLWWVNNQSLPVSPRLQLSNDNRTLTLLSVTRNDV
GPYECGIQNKLSVDHSDPVILNVLYGPDDPTISPSYTYYRPGVNLSLSCHAASNPPAQYS
WLIDGNIQQHTQELFISNITEKNSGLYTCQANNSASGHSRTTVKTITVSAELPKPSISSNNS
KPVEDKDAVAFTCEPEAQNTTYLWWVNGQSLPVSPRLQLSNGNRTLTLFNVTRNDAR
AYVCGIQNSVSANRSDPVTLDVLYGPDTPIISPPDSSYLSGANLNLSCHSASNPSPQYSW
RINGIPQQHTQVLFIAKITPNNNGTYACFVSNLATGRNNSIVKSITVSASGSGEGRGSLLT
CGDVEENPGPGAAPEPERTPVGQGSWAHPGRTRGPSDRGFCVVSPARPAEEATSLEGAL
SGTRHSHPSVGRQHHAGPPSTSRPPRPWDTPCPPVYAETKHFLYSSGDKEQLRPSFLLSS
LRPSLTGARRLVETIFLGSRPWMPGTPRRLPRLPQRYWQMRPLFLELLGNHAQCPYGVL
LKTHCPLRAAVTPAAGVCAREKPQGSVAAPEEEDTDPRRLVQLLRQHSSPWQVYGFVR
ACLRRLVPPGLWGSRHNERRFLRNTKKFISLGKHAKLSLQELTWKMSVRDCAWLRRSP
GVGCVPAAEHRLREEILAKFLHWLMSVYVVELLRSFFYVTETTFQKNRLFFYRKSVWS
KLQSIGIRQHLKRVQLRELSEAEVRQHREARPALLTSRLRFIPKPDGLRPIVNMDYVVGA
RTFRREKRAERLTSRVKALFSVLNYERARRPGLLGASVLGLDDIHRAWRTFVLRVRAQ
DPPPELYFVKVAITGAYDTIPQDRLTEVIASIIKPQNTYCVRRYAVVQKAAHGHVRKAF
KSHVSTLTDLQPYMRQFVAHLQETSPLRDAVVIEQSSSLNEASSGLFDVFLRFMCHHAV
RIRGKSYVQCQGIPQGSILSTLLCSLCYGDMENKLFAGIRRDGLLLRLVDDFLLVTPHLT
HAKTFLRTLVRGVPEYGCVVNLRKTVVNFPVEDEALGGTAFVQMPAHGLFPWCGLLL
DTRTLEVQSDYSSYARTSIRASLTFNRGFKAGRNMRRKLFGVLRLKCHSLFLDLQVNSL
QTVCTNIYKILLLQAYRFHACVLQLPFHQQVWKNPTFFLRVISDTASLCYSILKAKNAG
MSLGAKGAAGPLPSEAVQWLCHQAFLLKLTRHRVTYVPLLGSLRTAQTQLSRKLPGTT
LTALEAAANPALPSDFKTILDGSGTILSEGATNFSLLKLAGDVELNPGPTPGTQSPFFLLL
LLTVLTVVTGSGHASSTPGGEKETSATQRSSVPSSTEKNAVSMTSSVLSSHSPGSGSSTT
QGQDVTLAPATEPASGSAATWGQDVTSVPVTRPALGSTTPPAHDVTSAPDNKPAPGST
APPAHGVTSAPDTRPAPGSTAPPAHGVTSAPDTRPAPGSTAPPAHGVTSAPDTRPAPGST
APPAHGVTSAPDTRPAPGSTAPPAHGVTSAPDTRPALGSTAPPVHNVTSASGSASGSAST
LVHNGTSARATTTPASKSTPFSIPSHHSDTPTTLASHSTKTDASSTHHSSVPPLTSSNHSTS
PQLSTGVSFFFLSFHISNLQFNSSLEDPSTDYYQELQRDISEMFLQIYKQGGFLGLSNIKFR
PGSVVVQLTLAFREGTINVHDVETQFNQYKTEAASRYNLTISDVSVSDVPFPFSAQSGA
GVPGWGIALLVLVCVLVALAIVYLIALAVCQCRRKNYGQLDIFPARDTYHPMSEYPTY
HTHGRYVPPSSTDRSPYEKVSAGNGGSSLSYTNPAVAAASANL.
Plasmid 1428 complete vector (nucleotide sequence)

SEQ ID NO: 65

ggcgtaatgctctgccagtgttacaaccaattaaccaattctgattagaaaaactcatcgagcatcaaatgaaactgcaatttattcatatcagg
attatcaataccatatttttgaaaaagccgtttctgtaatgaaggagaaaactcaccgaggcagttccataggatggcaagatcctggtatcgg
tctgcgattccgactcgtccaacatcaatacaacctattaatttcccctcgtcaaaaataaggttatcaagtgagaaatcaccatgagtgacga
ctgaatccggtgagaatggcaaaagcttatgcatttctttccagacttgttcaacaggccagccattacgctcgtcatcaaaatcactcgcatc
aaccaaaccgttattcattcgtgattgcgcctgagcgagacgaaatacgcgatcgctgttaaaaggacaattacaaacaggaatcaaatgca
accggcgcaggaacactgccagcgcatcaacaatattttcacctgaatcaggatattcttctaatacctggaatgctgttttcccggggatcgc
agtggtgagtaaccatgcatcatcaggagtacggataaaatgcttgatggtcggaagaggcataaattccgtcagccagtttagtctgaccat
ctcatctgtaacatcattggcaacgctacctttgccatgtttcagaaacaactctggcgcatcgggcttcccatacaatcgatagattgtcgcac
ctgattgcccgacattatcgcgagcccatttatacccatataaatcagcatccatgttggaatttaatcgcggcctcgagcaagacgtttcccgt
tgaatatggctcataacaccccttgtattactgtttatgtaagcagacaggtcgacaatattggctattggccattgcatacgttgtatctatatcat
aatatgtacatttatattggctcatgtccaatatgaccgccatgttgacattgattattgactagttattaatagtaatcaattacggggtcattagtt
catagcccatatatggagttccgcgttacataacttacggtaaatggcccgcctggctgaccgcccaacgacccccgcccattgacgtcaat
aatgacgtatgttcccatagtaacgccaatagggactttccattgacgtcaatgggtggagtatttacggtaaactgcccacttggcagtacat
caagtgtatcatatgccaagtccgccccctattgacgtcaatgacggtaaatggcccgcctggcattatgcccagtacatgaccttacggga
ctttcctacttggcagtacatctacgtattagtcatcgctattaccatggtgatgcggttttggcagtacaccaatgggcgtggatagcggtttga
ctcacggggatttccaagtctccaccccattgacgtcaatgggagtttgttttggcaccaaaatcaacgggactttccaaaatgtcgtaataac
cccgccccgttgacgcaaatgggcggtaggcgtgtacggtgggaggtctatataagcagagctcgtttagtgaaccgtcagatcgcctgg
agacgccatccacgctgttttgacctccatagaagacaccgggaccgatccagcctccgcggccgggaacggtgcattggaacgcggat
tccccgtgccaagagtgactcaccgtccggatctcagcaagcaggtatgtactctccagggtgggcctggcttccccagtcaagactccag
ggatttgagggacgctgtgggctcttctcttacatgtaccttttgcttgcctcaaccctgactatcttccaggtcaggatcccagagtcaggggt
ctgtattttcctgctggtggctccagttcaggaacagtaaaccctgctccgaatattgcctctcacatctcgtcaatctccgcgaggactgggg
accctgtgacgaacatggctagcacccctggaacccagagccccttcttccttctgctgctgctgaccgtgctgactgtcgtgacaggctct
ggccacgccagctctacacctggcggcgagaaagagacaagcgccacccagagaagcagcgtgccaagcagcaccgagaagaacg
ccgtgtccatgaccagctccgtgctgagcagccactctcctggcagcggcagcagcacaacacagggccaggatgtgacactggcccct
gccacagaacctgcctctggatctgccgccacctggggacaggacgtgacaagcgtgccagtgaccagacctgccctgggctctacaac
accccctgcccacgatgtgaccagcgcccctgataacaagcctgcccctggaagcacagcccctccagctcatggcgtgacctctgcccc
agataccagaccagccccaggatctacagccccacccgcacacggcgtgacaagtgcccctgacacaagacccgctccaggctctact
gctcctcctgcccatggcgtgacaagcgctcccgatacaaggccagctcctggctccacagcaccaccagcacatggcgtgacatcagct
cccgacactagacctgctcccggatcaaccgctccaccagctcacggcgtgaccagcgcacctgataccagacctgctctgggaagcac
cgcccctcccgtgcacaatgtgacatctgcttccggcagcgccagcggctctgcctctacactggtgcacaacggcaccagcgccagag
ccacaacaaccccagccagcaagagcacccccttcagcatccctagccaccacagcgacacccctaccacactggccagccactccac
caagaccgatgcctctagcacccaccactccagcgtgccccctctgaccagcagcaaccacagcacaagcccccagctgtctaccggcg
tctcattcttctttctgtccttccacatcagcaacctgcagttcaacagcagcctggaagatcccagcaccgactactaccaggaactgcagc
gggatatcagcgagatgttcctgcaaatctacaagcagggcggcttcctgggcctgagcaacatcaagttcagacccggcagcgtggtgg
tgcagctgaccctggctttccgggaaggcaccatcaacgtgcacgacgtggaaacccagttcaaccagtacaagaccgaggccgccagc
cggtacaacctgaccatctccgatgtgtccgtgtccgacgtgcccttcccattctctgcccagtctggcgcaggcgtgccaggatggggaat
tgctctgctggtgctcgtgtgcgtgctggtggccctggccatcgtgtatctgattgccctggccgtgtgccagtgccggcggaagaattacg
gccagctggacatcttccccgccagagacacctaccaccccatgagcgagtaccccacataccacacccacggcagatacgtgccaccc
agctccaccgacagatccccctacgagaaagtgtctgccggcaacggcggcagctccctgagctacacaaatcctgccgtggccgctgc
ctccgccaacctgggatccggcagaatcttcaacgcccactacgccggctacttcgccgacctgctgatccacgacatcgagacaaaccc
tggccccaagctgaccattgagagcactcccttcaacgtggctgaggggaaggaggtgctgctcctggtgcacaatctgccccagcacct
gttcgggtactcctggtacaagggagaacgcgtggacgggaaccggcagatcataggctacgtcatcggaacccagcaggccacaccc
ggtccagcgtacagcggccgggagattatctacccgaacgcctccctgctgatccaaaacatcatccagaacgacaccggtttctacactc
tgcacgtgattaagtcagatctggtcaacgaagaggccaccggccaattcagggtgtaccccgaactccctaagccgttcatcacctcgaa
caacagcaacccggtcgaggatgaagatgcggtggccttgacgtgcgaacctgagatccagaacaccacctacttgtggtgggtgaaca
atcagagcctgccagtctccccacgactccagctgtcgaacgacaacaggaccctgactttgctgtccgtgactcggaacgacgtgggcc
cttatgaatgcggtatccagaacaagctgtccgtggaccacagcgaccctgtgatcctgaacgtcctttacgggccggacgaccccaccat
ttccccgtcgtacacttactaccggccgggcgtgaacctgtccctgtcgtgccacgctgcctccaatccgccggcccagtactcctggctca
tcgacggaaacatccagcagcacacccaagaactgttcatctccaacattaccgagaaaaactcgggactttacacctgtcaagccaacaa
ttccgccagcggccactcccgcaccactgtcaaaactatcactgtgtccgccgaactcccgaagcccagcatcagctccaacaactcgaa
gcccgtggaggataaggacgctgtcgcgttcacctgtgaaccagaggcacagaataccacctacctttggtgggtcaacggacagtccct
gcctgtctcaccgagactgcagctgtcaaacgggaataggactctgaccttgtttaacgtcacccggaacgacgcccgggcctacgtgtgc
ggcatccagaactccgtgagcgcaaaccggtctgacccagtgaccctggatgtgctgtacggccccgacactccgatcatttcacccccc
gattcatcctacctgtccggcgctaacctcaacctctcatgccactccgcatccaaccccagcccgcaatattcgtggcgcattaacggaatt
cctcagcaacatacccaggtcctgttcattgcgaagatcacccctaacaacaacggaacctacgcctgctttgtgtcaaacctggccactgg
tagaaacaactccatcgtgaagtccattaccgtgtcggcgtccggatccggcgagggcagaggcagcctgctgacatgtggcgacgtgg
aagagaaccctggccccggagctgccccggagccggagaggacccccgttggccagggatcgtgggcccatccgggacgcaccagg
ggaccatccgacaggggattctgtgtggtgtcaccggccaggccagcagaagaggcaaccagcctcgagggagcgttgtctggaacca
gacattcccacccgtcggtgggccggcagcaccacgcgggaccaccgtccacttccagaccgccacggccatgggacaccccttgccc
gcctgtgtatgccgagactaaacacttcctgtactcatccggagacaaggaacagcttcggccgtccttcctcctgtcgtcgctcagaccga
gcctgaccggagcacgcagattggtggaaactatcttccttgggtcacgtccgtggatgccaggtaccccacggcgcctcccgcgcctcc
cacagagatactggcagatgcggcctctgttcctggaattgctgggaaaccacgctcagtgcccgtacggagtcctgctcaagactcactg
ccctctgagggcggcggtcactccggcggccggagtgtgcgcacgggagaagccccagggaagcgtggcagctccggaagaggag
gacaccgatccgcgccgcctcgtgcaacttctgcgccagcactcctcgccctggcaagtctacgggttcgtccgcgcctgcctgcgccgc
ctggtgccgcctgggctctggggttcccggcataacgagcgccgcttcctgagaaatactaagaagtttatctcacttggaaaacatgccaa
gttgtcgctgcaagaactcacgtggaagatgtcagtccgcgattgcgcctggctgcgccgctcgccgggcgtcgggtgtgttccagctgca
gaacaccgcctgagagaagaaattctggccaaatttctgcattggctgatgtcagtgtacgtggtcgagctgctgcgctcctttttctacgtca
ctgagactacctttcaaaagaaccgcctgttcttctaccgcaaatctgtgtggagcaagctgcagtcaatcggcattcgccagcatctgaaga
gggtgcagctgcgggaactttccgaggcagaagtccgccagcaccgggaggcccggccggcgcttctcacgtcgcgtctgagattcatc
ccaaagcccgacgggctgaggcctatcgtcaacatggattacgtcgtgggcgctcgcacctttcgccgtgaaaagcgggccgaacgcttg
acctcacgggtgaaggccctcttctccgtgctgaactacgagagagcaagacggcctggcctgctgggagcttcggtgctgggactggac
gatatccaccgggcttggcggacctttgttctccgggtgagagcccaagaccctccgccggaactgtacttcgtgaaggtggcgatcaccg
gagcctatgatactattccgcaagatcgactcaccgaagtcatcgcctcgatcatcaaaccgcagaacacttactgcgtcaggcggtacgc
cgtggtccagaaggccgcgcatggccacgtgagaaaggcgttcaagtcgcacgtgtccactctcaccgacctccagccttacatgaggca
attcgttgcgcatttgcaagagacttcgcccctgagagatgcggtggtcatcgagcagagctccagcctgaacgaagcgagcagcggtct
gtttgacgtgttcctccgcttcatgtgtcatcacgcggtgcgaatcaggggaaaatcatacgtgcagtgccagggaatcccacaaggcagc
attctgtcgactctcttgtgttccctttgctacggcgatatggaaaacaagctgttcgctgggatcagacgggacgggttgctgctcagactgg
tggacgacttcctgctggtgactccgcacctcactcacgccaaaacctttctccgcactctggtgaggggagtgccagaatacggctgtgtg
gtcaatctccggaaaactgtggtgaatttccctgtcgaggatgaggcactcggaggaaccgcatttgtccaaatgccagcacatggcctgtt
cccatggtgcggtctgctgctggacacccgaactcttgaagtgcagtccgactactccagctatgcccggacgagcatccgcgccagcct
cactttcaatcgcggctttaaggccggacgaaacatgcgcagaaagcttttcggagtcctccggcttaaatgccattcgctctttctcgatctc
caagtcaattcgctgcagaccgtgtgcacgaacatctacaagatcctgctgctccaagcctaccggttccacgcttgcgtgcttcagctgcc
gtttcaccaacaggtgtggaagaacccgaccttctttctgcgggtcattagcgatactgcctccctgtgttactcaatcctcaaggcaaagaac
gccggaatgtcgctgggtgcgaaaggagccgcgggacctcttcctagcgaagcggtgcagtggctctgccaccaggctttcctcctgaag
ctgaccaggcacagagtgacctacgtcccgctgctgggctcgctgcgcactgcacagacccagctgtctagaaaactccccggcaccac
cctgaccgctctggaagccgccgccaacccagcattgccgtcagatttcaagaccatcttggactgaagatctgggccctaacaaaacaaa
aagatggggttattccctaaacttcatgggttacgtaattggaagttgggggacattgccacaagatcatattgtacaaaagatcaaacactgt
tttagaaaacttcctgtaaacaggcctattgattggaaagtatgtcaaaggattgtgggtcttttgggctttgctgctccatttacacaatgtggat
atcctgccttaatgcctttgtatgcatgtatacaagctaaacaggctttcactttctcgccaacttacaaggcctttctaagtaaacagtacatgaa
cctttaccccgttgctcggcaacggcctggtctgtgccaagtgtttgctgacgcaacccccactggctggggcttggccataggccatcagc
gcatgcgtggaacctttgtggctcctctgccgatccatactgcggaactcctagccgcttgttttgctcgcagccggtctggagcaaagctca
taggaactgacaattctgtcgtcctctcgcggaaatatacatcgtttcgatctacgtatgatctttttccctctgccaaaaattatggggacatcat
gaagccccttgagcatctgacttctggctaataaaggaaatttattttcattgcaatagtgtgttggaattttttgtgtctctcactcggaaggaatt
ctgcattaatgaatcggccaacgcgcggggagaggcggtttgcgtattgggcgctcttccgcttcctcgctcactgactcgctgcgctcggt
cgttcggctgcggcgagcggtatcagctcactcaaaggcggtaatacggttatccacagaatcaggggataacgcaggaaagaacatgtg
agcaaaaggccagcaaaaggccaggaaccgtaaaaaggccgcgttgctggcgtttttccataggctccgcccccctgacgagcatcaca
aaaatcgacgctcaagtcagaggtggcgaaacccgacaggactataaagataccaggcgtttccccctggaagctccctcgtgcgctctc
ctgttccgaccctgccgcttaccggatacctgtccgcctttctcccttcgggaagcgtggcgctttctcatagctcacgctgtaggtatctcagt
tcggtgtaggtcgttcgctccaagctgggctgtgtgcacgaaccccccgttcagcccgaccgctgcgccttatccggtaactatcgtcttga
gtccaacccggtaagacacgacttatcgccactggcagcagccactggtaacaggattagcagagcgaggtatgtaggcggtgctacag
agttcttgaagtggtggcctaactacggctacactagaagaacagtatttggtatctgcgctctgctgaagccagttaccttcggaaaaagag
ttggtagctcttgatccggcaaacaaaccaccgctggtagcggtggtttttttgtttgcaagcagcagattacgcgcagaaaaaaaggatctc
aagaagatcctttgatcttttctacggggtctgacgctcagtggaacgaaaactcacgttaagggattttggtcatgagattatcaaaaaggat
cttcacctagatccttttaaattaaaaatgaagttttaaatcaatctaaagtatatatgagtaaacttggtctgacagttaccaatgcttaatcagtg
aggcacctatctcagcgatctgtctatttcgttcatccatagttgcctgactc.
AdC68Y 1428 complete vector (nucleotide sequence)

SEQ ID NO: 66

ccatcttcaataatatacctcaaactttttgtgcgcgttaatatgcaaatgaggcgtttgaatttggggaggaagggcggtgattggtcgaggg
atgagcgaccgttaggggcggggcgagtgacgttttgatgacgtggttgcgaggaggagccagtttgcaagttctcgtgggaaaagtgac
gtcaaacgaggtgtggtttgaacacggaaatactcaattttcccgcgctctctgacaggaaatgaggtgtttctgggcggatgcaagtgaaa
acgggccattttcgcgcgaaaactgaatgaggaagtgaaaatctgagtaatttcgcgtttatggcagggaggagtatttgccgagggccga
gtagactttgaccgattacgtgggggtttcgattaccgtgtttttcacctaaatttccgcgtacggtgtcaaagtccggtgtttttactactgtaata
gtaatcaattacggggtcattagttcatagcccatatatggagttccgcgttacataacttacggtaaatggcccgcctggctgaccgcccaa
cgacccccgcccattgacgtcaataatgacgtatgttcccatagtaacgccaatagggactttccattgacgtcaatgggtggagtatttacg
gtaaactgcccacttggcagtacatcaagtgtatcatatgccaagtacgccccctattgacgtcaatgacggtaaatggcccgcctggcatta
tgcccagtacatgaccttatgggactttcctacttggcagtacatctacgtattagtcatcgctattaccatggtgatgcggttttggcagtacat
caatgggcgtggatagcggtttgactcacggggatttccaagtctccaccccattgacgtcaatgggagtttgttttggcaccaaaatcaacg
ggactttccaaaatgtcgtaacaactccgccccattgacgcaaatgggcggtaggcgtgtacggtgggaggtctatataagcagagctgtc
cctatcagtgatagagatctccctatcagtgatagagagtttagtgaaccgtcagatccgctagggtaccgcgatCACCatggctagcac
ccctggaacccagagccccttcttccttctgctgctgctgaccgtgctgactgtcgtgacaggctctggccacgccagctctacacctggcg
gcgagaaagagacaagcgccacccagagaagcagcgtgccaagcagcaccgagaagaacgccgtgtccatgaccagctccgtgctg
agcagccactctcctggcagcggcagcagcacaacacagggccaggatgtgacactggcccctgccacagaacctgcctctggatctgc
cgccacctggggacaggacgtgacaagcgtgccagtgaccagacctgccctgggctctacaacaccccctgcccacgatgtgaccagc
gcccctgataacaagcctgcccctggaagcacagcccctccagctcatggcgtgacctctgccccagataccagaccagccccaggatct
acagccccacccgcacacggcgtgacaagtgcccctgacacaagacccgctccaggctctactgctcctcctgcccatggcgtgacaag
cgctcccgatacaaggccagctcctggctccacagcaccaccagcacatggcgtgacatcagctcccgacactagacctgctcccggatc
aaccgctccaccagctcacggcgtgaccagcgcacctgataccagacctgctctgggaagcaccgcccctcccgtgcacaatgtgacat
ctgcttccggcagcgccagcggctctgcctctacactggtgcacaacggcaccagcgccagagccacaacaaccccagccagcaagag
cacccccttcagcatccctagccaccacagcgacacccctaccacactggccagccactccaccaagaccgatgcctctagcacccacc
actccagcgtgccccctctgaccagcagcaaccacagcacaagcccccagctgtctaccggcgtctcattcttctttctgtccttccacatca
gcaacctgcagttcaacagcagcctggaagatcccagcaccgactactaccaggaactgcagcgggatatcagcgagatgttcctgcaaa
tctacaagcagggcggcttcctgggcctgagcaacatcaagttcagacccggcagcgtggtggtgcagctgaccctggctttccgggaag
gcaccatcaacgtgcacgacgtggaaacccagttcaaccagtacaagaccgaggccgccagccggtacaacctgaccatctccgatgtg
tccgtgtccgacgtgcccttcccattctctgcccagtctggcgcaggcgtgccaggatggggaattgctctgctggtgctcgtgtgcgtgctg
gtggccctggccatcgtgtatctgattgccctggccgtgtgccagtgccggcggaagaattacggccagctggacatcttccccgccagag
acacctaccaccccatgagcgagtaccccacataccacacccacggcagatacgtgccacccagctccaccgacagatccccctacgag
aaagtgtctgccggcaacggcggcagctccctgagctacacaaatcctgccgtggccgctgcctccgccaacctgggatccggcagaat
cttcaacgcccactacgccggctacttcgccgacctgctgatccacgacatcgagacaaaccctggccccaagctgaccattgagagcac
tcccttcaacgtggctgaggggaaggaggtgctgctcctggtgcacaatctgccccagcacctgttcgggtactcctggtacaagggagaa
cgcgtggacgggaaccggcagatcataggctacgtcatcggaacccagcaggccacacccggtccagcgtacagcggccgggagatt
atctacccgaacgcctccctgctgatccaaaacatcatccagaacgacaccggtttctacactctgcacgtgattaagtcagatctggtcaac
gaagaggccaccggccaattcagggtgtaccccgaactccctaagccgttcatcacctcgaacaacagcaacccggtcgaggatgaaga
tgcggtggccttgacgtgcgaacctgagatccagaacaccacctacttgtggtgggtgaacaatcagagcctgccagtctccccacgactc
cagctgtcgaacgacaacaggaccctgactttgctgtccgtgactcggaacgacgtgggcccttatgaatgcggtatccagaacaagctgt
ccgtggaccacagcgaccctgtgatcctgaacgtcctttacgggccggacgaccccaccatttccccgtcgtacacttactaccggccggg
cgtgaacctgtccctgtcgtgccacgctgcctccaatccgccggcccagtactcctggctcatcgacggaaacatccagcagcacaccca
agaactgttcatctccaacattaccgagaaaaactcgggactttacacctgtcaagccaacaattccgccagcggccactcccgcaccactg
tcaaaactatcactgtgtccgccgaactcccgaagcccagcatcagctccaacaactcgaagcccgtggaggataaggacgctgtcgcgt
tcacctgtgaaccagaggcacagaataccacctacctttggtgggtcaacggacagtccctgcctgtctcaccgagactgcagctgtcaaa
cgggaataggactctgaccttgtttaacgtcacccggaacgacgcccgggcctacgtgtgcggcatccagaactccgtgagcgcaaacc
ggtctgacccagtgaccctggatgtgctgtacggccccgacactccgatcatttcaccccccgattcatcctacctgtccggcgctaacctca
acctctcatgccactccgcatccaaccccagcccgcaatattcgtggcgcattaacggaattcctcagcaacatacccaggtcctgttcattg
cgaagatcacccctaacaacaacggaacctacgcctgctttgtgtcaaacctggccactggtagaaacaactccatcgtgaagtccattacc
gtgtcggcgtccggatccggcgagggcagaggcagcctgctgacatgtggcgacgtggaagagaaccctggccccggagctgccccg
gagccggagaggacccccgttggccagggatcgtgggcccatccgggacgcaccaggggaccatccgacaggggattctgtgtggtgt
caccggccaggccagcagaagaggcaaccagcctcgagggagcgttgtctggaaccagacattcccacccgtcggtgggccggcagc
accacgcgggaccaccgtccacttccagaccgccacggccatgggacaccccttgcccgcctgtgtatgccgagactaaacacttcctgt
actcatccggagacaaggaacagcttcggccgtccttcctcctgtcgtcgctcagaccgagcctgaccggagcacgcagattggtggaaa
ctatcttccttgggtcacgtccgtggatgccaggtaccccacggcgcctcccgcgcctcccacagagatactggcagatgcggcctctgttc
ctggaattgctgggaaaccacgctcagtgcccgtacggagtcctgctcaagactcactgccctctgagggcggcggtcactccggcggcc
ggagtgtgcgcacgggagaagccccagggaagcgtggcagctccggaagaggaggacaccgatccgcgccgcctcgtgcaacttctg
cgccagcactcctcgccctggcaagtctacgggttcgtccgcgcctgcctgcgccgcctggtgccgcctgggctctggggttcccggcat
aacgagcgccgcttcctgagaaatactaagaagtttatctcacttggaaaacatgccaagttgtcgctgcaagaactcacgtggaagatgtc
agtccgcgattgcgcctggctgcgccgctcgccgggcgtcgggtgtgttccagctgcagaacaccgcctgagagaagaaattctggcca
aatttctgcattggctgatgtcagtgtacgtggtcgagctgctgcgctcctttttctacgtcactgagactacctttcaaaagaaccgcctgttctt
ctaccgcaaatctgtgtggagcaagctgcagtcaatcggcattcgccagcatctgaagagggtgcagctgcgggaactttccgaggcaga
agtccgccagcaccgggaggcccggccggcgcttctcacgtcgcgtctgagattcatcccaaagcccgacgggctgaggcctatcgtca
acatggattacgtcgtgggcgctcgcacctttcgccgtgaaaagcgggccgaacgcttgacctcacgggtgaaggccctcttctccgtgct
gaactacgagagagcaagacggcctggcctgctgggagcttcggtgctgggactggacgatatccaccgggcttggcggacctttgttct
ccgggtgagagcccaagaccctccgccggaactgtacttcgtgaaggtggcgatcaccggagcctatgatactattccgcaagatcgact
caccgaagtcatcgcctcgatcatcaaaccgcagaacacttactgcgtcaggcggtacgccgtggtccagaaggccgcgcatggccacg
tgagaaaggcgttcaagtcgcacgtgtccactctcaccgacctccagccttacatgaggcaattcgttgcgcatttgcaagagacttcgccc
ctgagagatgcggtggtcatcgagcagagctccagcctgaacgaagcgagcagcggtctgtttgacgtgttcctccgcttcatgtgtcatca
cgcggtgcgaatcaggggaaaatcatacgtgcagtgccagggaatcccacaaggcagcattctgtcgactctcttgtgttccctttgctacg
gcgatatggaaaacaagctgttcgctgggatcagacgggacgggttgctgctcagactggtggacgacttcctgctggtgactccgcacct
cactcacgccaaaacctttctccgcactctggtgaggggagtgccagaatacggctgtgtggtcaatctccggaaaactgtggtgaatttcc
ctgtcgaggatgaggcactcggaggaaccgcatttgtccaaatgccagcacatggcctgttcccatggtgcggtctgctgctggacacccg
aactcttgaagtgcagtccgactactccagctatgcccggacgagcatccgcgccagcctcactttcaatcgcggctttaaggccggacga
aacatgcgcagaaagcttttcggagtcctccggcttaaatgccattcgctctttctcgatctccaagtcaattcgctgcagaccgtgtgcacga
acatctacaagatcctgctgctccaagcctaccggttccacgcttgcgtgcttcagctgccgtttcaccaacaggtgtggaagaacccgacc
ttctttctgcgggtcattagcgatactgcctccctgtgttactcaatcctcaaggcaaagaacgccggaatgtcgctgggtgcgaaaggagcc
gcgggacctcttcctagcgaagcggtgcagtggctctgccaccaggctttcctcctgaagctgaccaggcacagagtgacctacgtcccg
ctgctgggctcgctgcgcactgcacagacccagctgtctagaaaactccccggcaccaccctgaccgctctggaagccgccgccaaccc
agcattgccgtcagatttcaagaccatcttggacTGAcgcaCctcgagctgatcataatcagccataccacatttgtagaggttttacttgct
ttaaaaaacctcccacacctccccctgaacctgaaacataaaatgaatgcaattgttgttgttaacttgtttattgcagcttataatggttacaaat
aaagcaatagcatcacaaatttcacaaataaagcatttttttcactgcattctagttgtggtttgtccaaactcatcaatgtatcttaccaggtgcc
gagcctgcgagtgcggagggaagcatgccaggttccagcccgtgtgtgtggatgtgacggaggacctgcgacccgatcatttggtgttgc
cctgcaccgggacggagttcggttccagcggggaagaatctgactagagtgagtagtgttctggggcgggggaggacctgcatgaggg
ccagaataactgaaatctgtgcttttctgtgtgttgcagcagcatgagcggaagcggctcctttgagggaggggtattcagcccttatctgac
ggggcgtctcccctcctgggcgggagtgcgtcagaatgtgatgggatccacggtggacggccggcccgtgcagcccgcgaactcttcaa
ccctgacctatgcaaccctgagctcttcgtcgttggacgcagctgccgccgcagctgctgcatctgccgccagcgccgtgcgcggaatgg
ccatgggcgccggctactacggcactctggtggccaactcgagttccaccaataatcccgccagcctgaacgaggagaagctgttgctgc
tgatggcccagctcgaggccttgacccagcgcctgggcgagctgacccagcaggtggctcagctgcaggagcagacgcgggccgcgg
ttgccacggtgaaatccaaataaaaaatgaatcaataaataaacggagacggttgttgattttaacacagagtctgaatctttatttgatttttcgc
gcgcggtaggccctggaccaccggtctcgatcattgagcacccggtggatcttttccaggacccggtagaggtgggcttggatgttgaggt
acatgggcatgagcccgtcccgggggtggaggtagctccattgcagggcctcgtgctcgggggtggtgttgtaaatcacccagtcatagc
aggggcgcagggcatggtgttgcacaatatctttgaggaggagactgatggccacgggcagccctttggtgtaggtgtttacaaatctgttg
agctgggagggatgcatgcggggggagatgaggtgcatcttggcctggatcttgagattggcgatgttaccgcccagatcccgcctgggg
ttcatgttgtgcaggaccaccagcacggtgtatccggtgcacttggggaatttatcatgcaacttggaagggaaggcgtgaaagaatttggc
gacgcctttgtgcccgcccaggttttccatgcactcatccatgatgatggcgatgggcccgtgggcggcggcctgggcaaagacgtttcgg
gggtcggacacatcatagttgtggtcctgggtgaggtcatcataggccattttaatgaatttggggcggagggtgccggactgggggacaa
aggtaccctcgatcccgggggcgtagttcccctcacagatctgcatctcccaggctttgagctcggagggggggatcatgtccacctgcgg
ggcgataaagaacacggtttccggggcgggggagatgagctgggccgaaagcaagttccggagcagctgggacttgccgcagccggt
ggggccgtagatgaccccgatgaccggctgcaggtggtagttgagggagagacagctgccgtcctcccggaggaggggggccacctc
gttcatcatctcgcgcacgtgcatgttctcgcgcaccagttccgccaggaggcgctctccccccagggataggagctcctggagcgaggc
gaagtttttcagcggcttgagtccgtcggccatgggcattttggagagggtttgttgcaagagttccaggcggtcccagagctcggtgatgtg
ctctacggcatctcgatccagcagacctcctcgtttcgcgggttgggacggctgcgggagtagggcaccagacgatgggcgtccagcgc
agccagggtccggtccttccagggtcgcagcgtccgcgtcagggtggtctccgtcacggtgaaggggtgcgcgccgggctgggcgctt
gcgagggtgcgcttcaggctcatccggctggtcgaaaaccgctcccgatcggcgccctgcgcgtcggccaggtagcaattgaccatgag
ttcgtagttgagcgcctcggccgcgtggcctttggcgcggagcttacctttggaagtctgcccgcaggcgggacagaggagggacttgag
ggcgtagagcttgggggcgaggaagacggactcgggggcgtaggcgtccgcgccgcagtgggcgcagacggtctcgcactccacga
gccaggtgaggtcgggctggtcggggtcaaaaaccagtttcccgccgttctttttgatgcgtttcttacctttggtctccatgagctcgtgtccc
cgctgggtgacaaagaggctgtccgtgtccccgtagaccgactttatgggccggtcctcgagcggtgtgccgcggtcctcctcgtagagg
aaccccgcccactccgagacgaaagcccgggtccaggccagcacgaaggaggccacgtgggacgggtagcggtcgttgtccaccag
cgggtccaccttttccagggtatgcaaacacatgtccccctcgtccacatccaggaaggtgattggcttgtaagtgtaggccacgtgaccgg
gggtcccggccgggggggtataaaagggtgcgggtccctgctcgtcctcactgtcttccggatcgctgtccaggagcgccagctgttggg
gtaggtattccctctcgaaggcgggcatgacctcggcactcaggttgtcagtttctagaaacgaggaggatttgatattgacggtgccggcg
gagatgcctttcaagagcccctcgtccatctggtcagaaaagacgatctttttgttgtcgagcttggtggcgaaggagccgtagagggcgtt
ggagaggagcttggcgatggagcgcatggtctggtttttttccttgtcggcgcgctccttggcggcgatgttgagctgcacgtactcgcgcg
ccacgcacttccattcggggaagacggtggtcagctcgtcgggcacgattctgacctgccagccccgattatgcagggtgatgaggtcca
cactggtggccacctcgccgcgcaggggctcattagtccagcagaggcgtccgcccttgcgcgagcagaaggggggcagggggtcca
gcatgacctcgtcgggggggtcggcatcgatggtgaagatgccgggcaggaggtcggggtcaaagtagctgatggaagtggccagatc
gtccagggcagcttgccattcgcgcacggccagcgcgcgctcgtagggactgaggggcgtgccccagggcatgggatgggtaagcgc
ggaggcgtacatgccgcagatgtcgtagacgtagaggggctcctcgaggatgccgatgtaggtggggtagcagcgccccccgcggatg
ctggcgcgcacgtagtcatacagctcgtgcgagggggcgaggagccccgggcccaggttggtgcgactgggcttttcggcgcggtaga
cgatctggcggaaaatggcatgcgagttggaggagatggtgggcctttggaagatgttgaagtgggcgtggggcagtccgaccgagtcg
cggatgaagtgggcgtaggagtcttgcagcttggcgacgagctcggcggtgactaggacgtccagagcgcagtagtcgagggtctcctg
gatgatgtcatacttgagctgtcccttttgtttccacagctcgcggttgagaaggaactcttcgcggtccttccagtactcttcgagggggaac
ccgtcctgatctgcacggtaagagcctagcatgtagaactggttgacggccttgtaggcgcagcagcccttctccacggggagggcgtag
gcctgggcggccttgcgcagggaggtgtgcgtgagggcgaaagtgtccctgaccatgaccttgaggaactggtgcttgaagtcgatatcg
tcgcagcccccctgctcccagagctggaagtccgtgcgcttcttgtaggcggggttgggcaaagcgaaagtaacatcgttgaagaggatct
tgcccgcgcggggcataaagttgcgagtgatgcggaaaggttggggcacctcggcccggttgttgatgacctgggcggcgagcacgatc
tcgtcgaagccgttgatgttgtggcccacgatgtagagttccacgaatcgcggacggcccttgacgtggggcagtttcttgagctcctcgta
ggtgagctcgtcggggtcgctgagcccgtgctgctcgagcgcccagtcggcgagatgggggttggcgcggaggaaggaagtccagag
atccacggccagggcggtttgcagacggtcccggtactgacggaactgctgcccgacggccattttttcgggggtgacgcagtagaaggt
gcgggggtccccgtgccagcgatcccatttgagctggagggcgagatcgagggcgagctcgacgagccggtcgtccccggagagtttc
atgaccagcatgaaggggacgagctgcttgccgaaggaccccatccaggtgtaggtttccacatcgtaggtgaggaagagcctttcggtg
cgaggatgcgagccgatggggaagaactggatctcctgccaccaattggaggaatggctgttgatgtgatggaagtagaaatgccgacg
gcgcgccgaacactcgtgcttgtgtttatacaagcggccacagtgctcgcaacgctgcacgggatgcacgtgctgcacgagctgtacctg
agttcctttgacgaggaatttcagtgggaagtggagtcgtggcgcctgcatctcgtgctgtactacgtcgtggtggtcggcctggccctcttct
gcctcgatggtggtcatgctgacgagcccgcgcgggaggcaggtccagacctcggcgcgagcgggtcggagagcgaggacgagggc
gcgcaggccggagctgtccagggtcctgagacgctgcggagtcaggtcagtgggcagcggcggcgcgcggttgacttgcaggagttttt
ccagggcgcgcgggaggtccagatggtacttgatctccaccgcgccattggtggcgacgtcgatggcttgcagggtcccgtgcccctgg
ggtgtgaccaccgtcccccgtttcttcttgggcggctggggcgacgggggcggtgcctcttccatggttagaagcggcggcgaggacgc
gcgccgggcggcaggggcggctcggggcccggaggcaggggcggcaggggcacgtcggcgccgcgcgcgggtaggttctggtac
tgcgcccggagaagactggcgtgagcgacgacgcgacggttgacgtcctggatctgacgcctctgggtgaaggccacgggacccgtga
gtttgaacctgaaagagagttcgacagaatcaatctcggtatcgttgacggcggcctgccgcaggatctcttgcacgtcgcccgagttgtcc
tggtaggcgatctcggtcatgaactgctcgatctcctcctcttgaaggtctccgcggccggcgcgctccacggtggccgcgaggtcgttgg
agatgcggcccatgagctgcgagaaggcgttcatgcccgcctcgttccagacgcggctgtagaccacgacgccctcgggatcgcGggc
gcgcatgaccacctgggcgaggttgagctccacgtggcgcgtgaagaccgcgtagttgcagaggcgctggtagaggtagttgagcgtg
gtggcgatgtgctcggtgacgaagaaatacatgatccagcggcggagcggcatctcgctgacgtcgcccagcgcctccaaacgttccatg
gcctcgtaaaagtccacggcgaagttgaaaaactgggagttgcgcgccgagacggtcaactcctcctccagaagacggatgagctcggc
gatggtggcgcgcacctcgcgctcgaaggcccccgggagttcctccacttcctcttcttcctcctccactaacatctcttctacttcctcctcag
gcggcagtggtggcgggggagggggcctgcgtcgccggcggcgcacgggcagacggtcgatgaagcgctcgatggtctcgccgcgc
cggcgtcgcatggtctcggtgacggcgcgcccgtcctcgcggggccgcagcgtgaagacgccgccgcgcatctccaggtggccgggg
gggtccccgttgggcagggagagggcgctgacgatgcatcttatcaattgccccgtagggactccgcgcaaggacctgagcgtctcgag
atccacgggatctgaaaaccgctgaacgaaggcttcgagccagtcgcagtcgcaaggtaggctgagcacggtttcttctggcgggtcatgt
tggttgggagcggggcgggcgatgctgctggtgatgaagttgaaataggcggttctgagacggcggatggtggcgaggagcaccaggt
ctttgggcccggcttgctggatgcgcagacggtcggccatgccccaggcgtggtcctgacacctggccaggtccttgtagtagtcctgcat
gagccgctccacgggcacctcctcctcgcccgcgcggccgtgcatgcgcgtgagcccgaagccgcgctggggctggacgagcgcca
ggtcggcgacgacgcgctcggcgaggatggcttgctggatctgggtgagggtggtctggaagtcatcaaagtcgacgaagcggtggtag
gctccggtgttgatggtgtaggagcagttggccatgacggaccagttgacggtctggtggcccggacgcacgagctcgtggtacttgagg
cgcgagtaggcgcgcgtgtcgaagatgtagtcgttgcaggtgcgcaccaggtactggtagccgatgaggaagtgcggcggcggctggc
ggtagagcggccatcgctcggtggcgggggcgccgggcgcgaggtcctcgagcatggtgcggtggtagccgtagatgtacctggacat
ccaggtgatgccggcggcggtggtggaggcgcgcgggaactcgcggacgcggttccagatgttgcgcagcggcaggaagtagttcat
ggtgggcacggtctggcccgtgaggcgcgcgcagtcgtggatgctctatacgggcaaaaacgaaagcggtcagcggctcgactccgtg
gcctggaggctaagcgaacgggttgggctgcgcgtgtaccccggttcgaatctcgaatcaggctggagccgcagctaacgtggtattggc
actcccgtctcgacccaagcctgcaccaaccctccaggatacggaggcgggtcgttttgcaacttttttttggaggccggatgagactagta
agcgcggaaagcggccgaccgcgatggctcgctgccgtagtctggagaagaatcgccagggttgcgttgcggtgtgccccggttcgag
gccggccggattccgcggctaacgagggcgtggctgccccgtcgtttccaagaccccatagccagccgacttctccagttacggagcga
gcccctcttttgttttgtttgtttttgccagatgcatcccgtactgcggcagatgcgcccccaccaccctccaccgcaacaacagccccctcca
cagccggcgcttctgcccccgccccagcagcaacttccagccacgaccgccgcggccgccgtgagcggggctggacagagttatgatc
accagctggccttggaagagggcgaggggctggcgcgcctgggggcgtcgtcgccggagcggcacccgcgcgtgcagatgaaaagg
gacgctcgcgaggcctacgtgcccaagcagaacctgttcagagacaggagcggcgaggagcccgaggagatgcgcgcggcccggtt
ccacgcggggcgggagctgcggcgcggcctggaccgaaagagggtgctgagggacgaggatttcgaggcggacgagctgacgggg
atcagccccgcgcgcgcgcacgtggccgcggccaacctggtcacggcgtacgagcagaccgtgaaggaggagagcaacttccaaaaa
tccttcaacaaccacgtgcgcaccctgatcgcgcgcgaggaggtgaccctgggcctgatgcacctgtgggacctgctggaggccatcgt
gcagaaccccaccagcaagccgctgacggcgcagctgttcctggtggtgcagcatagtcgggacaacgaagcgttcagggaggcgctg
ctgaatatcaccgagcccgagggccgctggctcctggacctggtgaacattctgcagagcatcgtggtgcaggagcgcgggctgccgct
gtccgagaagctggcggccatcaacttctcggtgctgagtttgggcaagtactacgctaggaagatctacaagaccccgtacgtgcccata
gacaaggaggtgaagatcgacgggttttacatgcgcatgaccctgaaagtgctgaccctgagcgacgatctgggggtgtaccgcaacga
caggatgcaccgtgcggtgagcgccagcaggcggcgcgagctgagcgaccaggagctgatgcatagtctgcagcgggccctgaccg
gggccgggaccgagggggagagctactttgacatgggcgcggacctgcactggcagcccagccgccgggccttggaggcggcggca
ggaccctacgtagaagaggtggacgatgaggtggacgaggagggcgagtacctggaagactgatggcgcgaccgtatttttgctagatg
caacaacaacagccacctcctgatcccgcgatgcgggcggcgctgcagagccagccgtccggcattaactcctcggacgattggaccca
ggccatgcaacgcatcatggcgctgacgacccgcaaccccgaagcctttagacagcagccccaggccaaccggctctcggccatcctg
gaggccgtggtgccctcgcgctccaaccccacgcacgagaaggtcctggccatcgtgaacgcgctggtggagaacaaggccatccgcg
gcgacgaggccggcctggtgtacaacgcgctgctggagcgcgtggcccgctacaacagcaccaacgtgcagaccaacctggaccgca
tggtgaccgacgtgcgcgaggccgtggcccagcgcgagcggttccaccgcgagtccaacctgggatccatggtggcgctgaacgcctt
cctcagcacccagcccgccaacgtgccccggggccaggaggactacaccaacttcatcagcgccctgcgcctgatggtgaccgaggtg
ccccagagcgaggtgtaccagtccgggccggactacttcttccagaccagtcgccagggcttgcagaccgtgaacctgagccaggctttc
aagaacttgcagggcctgtggggcgtgcaggccccggtcggggaccgcgcgacggtgtcgagcctgctgacgccgaactcgcgcctg
ctgctgctgctggtggcccccttcacggacagcggcagcatcaaccgcaactcgtacctgggctacctgattaacctgtaccgcgaggcc
atcggccaggcgcacgtggacgagcagacctaccaggagatcacccacgtgagccgcgccctgggccaggacgacccgggcaacct
ggaagccaccctgaactttttgctgaccaaccggtcgcagaagatcccgccccagtacgcgctcagcaccgaggaggagcgcatcctgc
gttacgtgcagcagagcgtgggcctgttcctgatgcaggagggggccacccccagcgccgcgctcgacatgaccgcgcgcaacatgga
gcccagcatgtacgccagcaaccgcccgttcatcaataaactgatggactacttgcatcgggcggccgccatgaactctgactatttcacca
acgccatcctgaatccccactggctcccgccgccggggttctacacgggcgagtacgacatgcccgaccccaatgacgggttcctgtgg
gacgatgtggacagcagcgtgttctccccccgaccgggtgctaacgagcgccccttgtggaagaaggaaggcagcgaccgacgcccgt
cctcggcgctgtccggccgcgagggtgctgccgcggcggtgcccgaggccgccagtcctttcccgagcttgcccttctcgctgaacagta
tccgcagcagcgagctgggcaggatcacgcgcccgcgcttgctgggcgaagaggagtacttgaatgactcgctgttgagacccgagcg
ggagaagaacttccccaataacgggatagaaagcctggtggacaagatgagccgctggaagacgtatgcgcaggagcacagggacga
tccccgggcgtcgcagggggccacgagccggggcagcgccgcccgtaaacgccggtggcacgacaggcagcggggacagatgtgg
gacgatgaggactccgccgacgacagcagcgtgttggacttgggtgggagtggtaacccgttcgctcacctgcgcccccgtatcgggcg
catgatgtaagagaaaccgaaaataaatgatactcaccaaggccatggcgaccagcgtgcgttcgtttcttctctgttgttgttgtatctagtat
gatgaggcgtgcgtacccggagggtcctcctccctcgtacgagagcgtgatgcagcaggcgatggcggcggcggcgatgcagccccc
gctggaggctccttacgtgcccccgcggtacctggcgcctacggaggggcggaacagcattcgttactcggagctggcacccttgtacga
taccacccggttgtacctggtggacaacaagtcggcggacatcgcctcgctgaactaccagaacgaccacagcaacttcctgaccaccgt
ggtgcagaacaatgacttcacccccacggaggccagcacccagaccatcaactttgacgagcgctcgcggtggggcggccagctgaaa
accatcatgcacaccaacatgcccaacgtgaacgagttcatgtacagcaacaagttcaaggcgcgggtgatggtctcccgcaagaccccc
aatggggtgacagtgacagaggattatgatggtagtcaggatgagctgaagtatgaatgggtggaatttgagctgcccgaaggcaacttct
cggtgaccatgaccatcgacctgatgaacaacgccatcatcgacaattacttggcggtggggcggcagaacggggtgctggagagcgac
atcggcgtgaagttcgacactaggaacttcaggctgggctgggaccccgtgaccgagctggtcatgcccggggtgtacaccaacgaggc
tttccatcccgatattgtcttgctgcccggctgcggggtggacttcaccgagagccgcctcagcaacctgctgggcattcgcaagaggcag
cccttccaggaaggcttccagatcatgtacgaggatctggaggggggcaacatccccgcgctcctggatgtcgacgcctatgagaaaagc
aaggaggatgcagcagctgaagcaactgcagccgtagctaccgcctctaccgaggtcaggggcgataattttgcaagcgccgcagcagt
ggcagcggccgaggcggctgaaaccgaaagtaagatagtcattcagccggtggagaaggatagcaagaacaggagctacaacgtacta
ccggacaagataaacaccgcctaccgcagctggtacctagcctacaactatggcgaccccgagaagggcgtgcgctcctggacgctgct
caccacctcggacgtcacctgcggcgtggagcaagtctactggtcgctgcccgacatgatgcaagacccggtcaccttccgctccacgcg
tcaagttagcaactacccggtggtgggcgccgagctcctgcccgtctactccaagagcttcttcaacgagcaggccgtctactcgcagcag
ctgcgcgccttcacctcgcttacgcacgtcttcaaccgcttccccgagaaccagatcctcgtccgcccgcccgcgcccaccattaccaccg
tcagtgaaaacgttcctgctctcacagatcacgggaccctgccgctgcgcagcagtatccggggagtccagcgcgtgaccgttactgacg
ccagacgccgcacctgcccctacgtctacaaggccctgggcatagtcgcgccgcgcgtcctctcgagccgcaccttctaaatgtccattct
catctcgcccagtaataacaccggttggggcctgcgcgcgcccagcaagatgtacggaggcgctcgccaacgctccacgcaacacccc
gtgcgcgtgcgcgggcacttccgcgctccctggggcgccctcaagggccgcgtgcggtcgcgcaccaccgtcgacgacgtgatcgacc
aggtggtggccgacgcgcgcaactacacccccgccgccgcgcccgtctccaccgtggacgccgtcatcgacagcgtggtggcCgacg
cgcgccggtacgcccgcgccaagagccggcggcggcgcatcgcccggcggcaccggagcacccccgccatgcgcgcggcgcgag
ccttgctgcgcagggccaggcgcacgggacgcagggccatgctcagggcggccagacgcgcggcttcaggcgccagcgccggcag
gacccggagacgcgcggccacggcggcggcagcggccatcgccagcatgtcccgcccgcggcgagggaacgtgtactgggtgcgc
gacgccgccaccggtgtgcgcgtgcccgtgcgcacccgcccccctcgcacttgaagatgttcacttcgcgatgttgatgtgtcccagcgg
cgaggaggatgtccaagcgcaaattcaaggaagagatgctccaggtcatcgcgcctgagatctacggccctgcggtggtgaaggagga
aagaaagccccgcaaaatcaagcgggtcaaaaaggacaaaaaggaagaagaaagtgatgtggacggattggtggagtttgtgcgcgag
ttcgccccccggcggcgcgtgcagtggcgcgggcggaaggtgcaaccggtgctgagacccggcaccaccgtggtcttcacgcccggc
gagcgctccggcaccgcttccaagcgctcctacgacgaggtgtacggggatgatgatattctggagcaggcggccgagcgcctgggcg
agtttgcttacggcaagcgcagccgttccgcaccgaaggaagaggcggtgtccatcccgctggaccacggcaaccccacgccgagcct
caagcccgtgaccttgcagcaggtgctgccgaccgcggcgccgcgccgggggttcaagcgcgagggcgaggatctgtaccccaccat
gcagctgatggtgcccaagcgccagaagctggaagacgtgctggagaccatgaaggtggacccggacgtgcagcccgaggtcaaggt
gcggcccatcaagcaggtggccccgggcctgggcgtgcagaccgtggacatcaagattcccacggagcccatggaaacgcagaccga
gcccatgatcaagcccagcaccagcaccatggaggtgcagacggatccctggatgccatcggctcctagtcgaagaccccggcgcaag
tacggcgcggccagcctgctgatgcccaactacgcgctgcatccttccatcatccccacgccgggctaccgcggcacgcgcttctaccgc
ggtcataccagcagccgccgccgcaagaccaccactcgccgccgccgtcgccgcaccgccgctgcaaccacccctgccgccctggtg
cggagagtgtaccgccgcggccgcgcacctctgaccctgccgcgcgcgcgctaccacccgagcatcgccatttaaactttcgccTgcttt
gcagatcaatggccctcacatgccgccttcgcgttcccattacgggctaccgaggaagaaaaccgcgccgtagaaggctggcggggaac
gggatgcgtcgccaccaccaccggcggcggcgcgccatcagcaagcggttggggggaggcttcctgcccgcgctgatccccatcatcg
ccgcggcgatcggggcgatccccggcattgcttccgtggcggtgcaggcctctcagcgccactgagacacacttggaaacatcttgtaat
aaaccAatggactctgacgctcctggtcctgtgatgtgttttcgtagacagatggaagacatcaatttttcgtccctggctccgcgacacggc
acgcggccgttcatgggcacctggagcgacatcggcaccagccaactgaacgggggcgccttcaattggagcagtctctggagcgggc
ttaagaatttcgggtccacgcttaaaacctatggcagcaaggcgtggaacagcaccacagggcaggcgctgagggataagctgaaagag
cagaacttccagcagaaggtggtcgatgggctcgcctcgggcatcaacggggtggtggacctggccaaccaggccgtgcagcggcag
atcaacagccgcctggacccggtgccgcccgccggctccgtggagatgccgcaggtggaggaggagctgcctcccctggacaagcgg
ggcgagaagcgaccccgccccgatgcggaggagacgctgctgacgcacacggacgagccgcccccgtacgaggaggcggtgaaac
tgggtctgcccaccacgcggcccatcgcgcccctggccaccggggtgctgaaacccgaaaagcccgcgaccctggacttgcctcctccc
cagccttcccgcccctctacagtggctaagcccctgccgccggtggccgtggcccgcgcgcgacccgggggcaccgcccgccctcatg
cgaactggcagagcactctgaacagcatcgtgggtctgggagtgcagagtgtgaagcgccgccgctgctattaaacctaccgtagcgctt
aacttgcttgtctgtgtgtgtatgtattatgtcgccgccgccgctgtccaccagaaggaggagtgaagaggcgcgtcgccgagttgcaagat
ggccaccccatcgatgctgccccagtgggcgtacatgcacatcgccggacaggacgcttcggagtacctgagtccgggtctggtgcagtt
tgcccgcgccacagacacctacttcagtctggggaacaagtttaggaaccccacggtggcgcccacgcacgatgtgaccaccgaccgca
gccagcggctgacgctgcgcttcgtgcccgtggaccgcgaggacaacacctactcgtacaaagtgcgctacacgctggccgtgggcga
caaccgcgtgctggacatggccagcacctactttgacatccgcggcgtgctggatcggggccctagcttcaaaccctactccggcaccgc
ctacaacagtctggcccccaagggagcacccaacacttgtcagtggacatataaagccgatggtgaaactgccacagaaaaaacctatac
atatggaaatgcacccgtgcagggcattaacatcacaaaagatggtattcaacttggaactgacaccgatgatcagccaatctacgcagata
aaacctatcagcctgaacctcaagtgggtgatgctgaatggcatgacatcactggtactgatgaaaagtatggaggcagagctcttaagcct
gataccaaaatgaagccttgttatggttcttttgccaagcctactaataaagaaggaggtcaggcaaatgtgaaaacaggaacaggcactac
taaagaatatgacatagacatggctttctttgacaacagaagtgcggctgctgctggcctagctccagaaattgttttgtatactgaaaatgtgg
atttggaaactccagatacccatattgtatacaaagcaggcacagatgacagcagctcttctattaatttgggtcagcaagccatgcccaaca
gacctaactacattggtttcagagacaactttatcgggctcatgtactacaacagcactggcaatatgggggtgctggccggtcaggcttctc
agctgaatgctgtggttgacttgcaagacagaaacaccgagctgtcctaccagctcttgcttgactctctgggtgacagaacccggtatttca
gtatgtggaatcaggcggtggacagctatgatcctgatgtgcgcattattgaaaatcatggtgtggaggatgaacttcccaactattgtttccct
ctggatgctgttggcagaacagatacttatcagggaattaaggctaatggaactgatcaaaccacatggaccaaagatgacagtgtcaatga
tgctaatgagataggcaagggtaatccattcgccatggaaatcaacatccaagccaacctgtggaggaacttcctctacgccaacgtggcc
ctgtacctgcccgactcttacaagtacacgccggccaatgttaccctgcccaccaacaccaacacctacgattacatgaacggccgggtgg
tggcgccctcgctggtggactcctacatcaacatcggggcgcgctggtcgctggatcccatggacaacgtgaaccccttcaaccaccacc
gcaatgcggggctgcgctaccgctccatgctcctgggcaacgggcgctacgtgcccttccacatccaggtgccccagaaatttttcgccat
caagagcctcctgctcctgcccgggtcctacacctacgagtggaacttccgcaaggacgtcaacatgatcctgcagagctccctcggcaa
cgacctgcgcacggacggggcctccatctccttcaccagcatcaacctctacgccaccttcttccccatggcgcacaacacggcctccacg
ctcgaggccatgctgcgcaacgacaccaacgaccagtccttcaacgactacctctcggcggccaacatgctctaccccatcccggccaac
gccaccaacgtgcccatctccatcccctcgcgcaactgggccgccttccgcggctggtccttcacgcgtctcaagaccaaggagacgccc
tcgctgggctccgggttcgacccctacttcgtctactcgggctccatcccctacctcgacggcaccttctacctcaaccacaccttcaagaag
gtctccatcaccttcgactcctccgtcagctggcccggcaacgaccggctcctgacgcccaacgagttcgaaatcaagcgcaccgtcgac
ggcgagggctacaacgtggcccagtgcaacatgaccaaggactggttcctggtccagatgctggcccactacaacatcggctaccaggg
cttctacgtgcccgagggctacaaggaccgcatgtactccttcttccgcaacttccagcccatgagccgccaggtggtggacgaggtcaac
tacaaggactaccaggccgtcaccctggcctaccagcacaacaactcgggcttcgtcggctacctcgcgcccaccatgcgccagggcca
gccctaccccgccaactacccctacccgctcatcggcaagagcgccgtcaccagcgtcacccagaaaaagttcctctgcgacagggtcat
gtggcgcatccccttctccagcaacttcatgtccatgggcgcgctcaccgacctcggccagaacatgctctatgccaactccgcccacgcg
ctagacatgaatttcgaagtcgaccccatggatgagtccacccttctctatgttgtcttcgaagtcttcgacgtcgtccgagtgcaccagcccc
accgcggcgtcatcgaggccgtctacctgcgcacccccttctcggccggtaacgccaccacctaagctcttgcttcttgcaagccatggcc
gcgggctccggcgagcaggagctcagggccatcatccgcgacctgggctgcgggccctacttcctgggcaccttcgataagcgcttccc
gggattcatggccccgcacaagctggcctgcgccatcgtcaacacggccggccgcgagaccgggggcgagcactggctggccttcgc
ctggaacccgcgctcgaacacctgctacctcttcgaccccttcgggttctcggacgagcgcctcaagcagatctaccagttcgagtacgag
ggcctgctgcgccgcagcgccctggccaccgaggaccgctgcgtcaccctggaaaagtccacccagaccgtgcagggtccgcgctcg
gccgcctgcgggctcttctgctgcatgttcctgcacgccttcgtgcactggcccgaccgccccatggacaagaaccccaccatgaacttgc
tgacgggggtgcccaacggcatgctccagtcgccccaggtggaacccaccctgcgccgcaaccaggaggcgctctaccgcttcctcaa
ctcccactccgcctactttcgctcccaccgcgcgcgcatcgagaaggccaccgccttcgaccgcatgaatcaagacatgtaaaccgtgtgt
gtatgttaaatgtctttaataaacagcactttcatgttacacatgcatctgagatgatttatttagaaatcgaaagggttctgccgggtctcggcat
ggcccgcgggcagggacacgttgcggaactggtacttggccagccacttgaactcggggatcagcagtttgggcagcggggtgtcggg
gaaggagtcggtccacagcttccgcgtcagttgcagggcgcccagcaggtcgggcgcggagatcttgaaatcgcagttgggacccgcgt
tctgcgcgcgggagttgcggtacacggggttgcagcactggaacaccatcagggccgggtgcttcacgctcgccagcaccgtcgcgtcg
gtgatgctctccacgtcgaggtcctcggcgttggccatcccgaagggggtcatcttgcaggtctgccttcccatggtgggcacgcacccgg
gcttgtggttgcaatcgcagtgcagggggatcagcatcatctgggcctggtcggcgttcatccccgggtacatggccttcatgaaagcctcc
aattgcctgaacgcctgctgggccttggctccctcggtgaagaagaccccgcaggacttgctagagaactggttggtggcgcacccggcg
tcgtgcacgcagcagcgcgcgtcgttgttggccagctgcaccacgctgcgcccccagcggttctgggtgatcttggcccggtcggggttct
ccttcagcgcgcgctgcccgttctcgctcgccacatccatctcgatcatgtgctccttctggatcatggtggtcccgtgcaggcaccgcagct
tgccctcggcctcggtgcacccgtgcagccacagcgcgcacccggtgcactcccagttcttgtgggcgatctgggaatgcgcgtgcacg
aagccctgcaggaagcggcccatcatggtggtcagggtcttgttgctagtgaaggtcagcggaatgccgcggtgctcctcgttgatgtaca
ggtggcagatgcggcggtacacctcgccctgctcgggcatcagctggaagttggctttcaggtcggtctccacgcggtagcggtccatca
gcatagtcatgatttccatacccttctcccaggccgagacgatgggcaggctcatagggttcttcaccatcatcttagcgctagcagccgcg
gccagggggtcgctctcgtccagggtctcaaagctccgcttgccgtccttctcggtgatccgcaccggggggtagctgaagcccacggcc
gccagctcctcctcggcctgtctttcgtcctcgctgtcctggctgacgtcctgcaggaccacatgcttggtcttgcggggtttcttcttgggcg
gcagcggcggcggagatgttggagatggcgagggggagcgcgagttctcgctcaccactactatctcttcctcttcttggtccgaggccac
gcggcggtaggtatgtctcttcgggggcagaggcggaggcgacgggctctcgccgccgcgacttggcggatggctggcagagcccctt
ccgcgttcgggggtgcgctcccggcggcgctctgactgacttcctccgcggccggccattgtgttctcctagggaggaacaacaagcatg
gagactcagccatcgccaacctcgccatctgcccccaccgccgacgagaagcagcagcagcagaatgaaagcttaaccgccccgccgc
ccagccccgccacctccgacgcggccgtcccagacatgcaagagatggaggaatccatcgagattgacctgggctatgtgacgcccgc
ggagcacgaggaggagctggcagtgcgcttttcacaagaagagatacaccaagaacagccagagcaggaagcagagaatgagcaga
gtcaggctgggctcgagcatgacggcgactacctccacctgagcgggggggaggacgcgctcatcaagcatctggcccggcaggcca
ccatcgtcaaggatgcgctgctcgaccgcaccgaggtgcccctcagcgtggaggagctcagccgcgcctacgagttgaacctcttctcgc
cgcgcgtgccccccaagcgccagcccaatggcacctgcgagcccaacccgcgcctcaacttctacccggtcttcgcggtgcccgaggc
cctggccacctaccacatctttttcaagaaccaaaagatccccgtctcctgccgcgccaaccgcacccgcgccgacgcccttttcaacctgg
gtcccggcgcccgcctacctgatatcgcctccttggaagaggttcccaagatcttcgagggtctgggcagcgacgagactcgggccgcg
aacgctctgcaaggagaaggaggagagcatgagcaccacagcgccctggtcgagttggaaggcgacaacgcgcggctggcggtgctc
aaacgcacggtcgagctgacccatttcgcctacccggctctgaacctgccccccaaagtcatgagcgcggtcatggaccaggtgctcatc
aagcgcgcgtcgcccatctccgaggacgagggcatgcaagactccgaggagggcaagcccgtggtcagcgacgagcagctggcccg
gtggctgggtcctaatgctagtccccagagtttggaagagcggcgcaaactcatgatggccgtggtcctggtgaccgtggagctggagtg
cctgcgccgcttcttcgccgacgcggagaccctgcgcaaggtcgaggagaacctgcactacctcttcaggcacgggttcgtgcgccagg
cctgcaagatctccaacgtggagctgaccaacctggtctcctacatgggcatcttgcacgagaaccgcctggggcagaacgtgctgcaca
ccaccctgcgcggggaggcccggcgcgactacatccgcgactgcgtctacctctacctctgccacacctggcagacgggcatgggcgt
gtggcagcagtgtctggaggagcagaacctgaaagagctctgcaagctcctgcagaagaacctcaagggtctgtggaccgggttcgacg
agcgcaccaccgcctcggacctggccgacctcattttccccgagcgcctcaggctgacgctgcgcaacggcctgcccgactttatgagcc
aaagcatgttgcaaaactttcgctctttcatcctcgaacgctccggaatcctgcccgccacctgctccgcgctgccctcggacttcgtgccgc
tgaccttccgcgagtgccccccgccgctgtggagccactgctacctgctgcgcctggccaactacctggcctaccactcggacgtgatcg
aggacgtcagcggcgagggcctgctcgagtgccactgccgctgcaacctctgcacgccgcaccgctccctggcctgcaacccccagct
gctgagcgagacccagatcatcggcaccttcgagttgcaagggcccagcgaaggcgagggttcagccgccaaggggggtctgaaactc
accccggggctgtggacctcggcctacttgcgcaagttcgtgcccgaggactaccatcccttcgagatcaggttctacgaggaccaatccc
atccgcccaaggccgagctgtcggcctgcgtcatcacccagggggcgatcctggcccaattgcaagccatccagaaatcccgccaagaa
ttcttgctgaaaaagggccgcggggtctacctcgacccccagaccggtgaggagctcaaccccggcttcccccaggatgccccgaggaa
acaagaagctgaaagtggagctgccgcccgtggaggatttggaggaagactgggagaacagcagtcaggcagaggaggaggagatg
gaggaagactgggacagcactcaggcagaggaggacagcctgcaagacagtctggaggaagacgaggaggaggcagaggaggag
gtggaagaagcagccgccgccagaccgtcgtcctcggcgggggagaaagcaagcagcacggataccatctccgctccgggtcggggt
cccgctcgaccacacagtagatgggacgagaccggacgattcccgaaccccaccacccagaccggtaagaaggagcggcagggatac
aagtcctggcgggggcacaaaaacgccatcgtctcctgcttgcaggcctgcgggggcaacatctccttcacccggcgctacctgctcttcc
accgcggggtgaactttccccgcaacatcttgcattactaccgtcacctccacagcccctactacttccaagaagaggcagcagcagcaga
aaaagaccagcagaaaaccagcagctagaaaatccacagcggcggcagcaggtggactgaggatcgcggcgaacgagccggcgca
aacccgggagctgaggaaccggatctttcccaccctctatgccatcttccagcagagtcgggggcaggagcaggaactgaaagtcaaga
accgttctctgcgctcgctcacccgcagttgtctgtatcacaagagcgaagaccaacttcagcgcactctcgaggacgccgaggctctcttc
aacaagtactgcgcgctcactcttaaagagtagcccgcgcccgcccagtcgcagaaaaaggcgggaattacgtcacctgtgcccttcgcc
ctagccgcctccacccatcatcatgagcaaagagattcccacgccttacatgtggagctaccagccccagatgggcctggccgccggtgc
cgcccaggactactccacccgcatgaattggctcagcgccgggcccgcgatgatctcacgggtgaatgacatccgcgcccaccgaaacc
agatactcctagaacagtcagcgctcaccgccacgccccgcaatcacctcaatccgcgtaattggcccgccgccctggtgtaccaggaaa
ttccccagcccacgaccgtactacttccgcgagacgcccaggccgaagtccagctgactaactcaggtgtccagctggcgggcggcgcc
accctgtgtcgtcaccgccccgctcagggtataaagcggctggtgatccggggcagaggcacacagctcaacgacgaggtggtgagctc
ttcgctgggtctgcgacctgacggagtcttccaactcgccggatcggggagatcttccttcacgcctcgtcaggccgtcctgactttggaga
gttcgtcctcgcagccccgctcgggtggcatcggcactctccagttcgtggaggagttcactccctcggtctacttcaaccccttctccggct
cccccggccactacccggacgagttcatcccgaacttcgacgccatcagcgagtcggtggacggctacgattgaatgtcccatggtggcg
cagctgacctagctcggcttcgacacctggaccactgccgccgcttccgctgcttcgctcgggatctcgccgagtttgcctactttgagctgc
ccgaggagcaccctcagggcccggcccacggagtgcggatcgtcgtcgaagggggcctcgactcccacctgcttcggatcttcagcca
gcgtccgatcctggtcgagcgcgagcaaggacagacccttctgactctgtactgcatctgcaaccaccccggcctgcatgaaagtctttgtt
gtctgctgtgtactgagtataataaaagctgagatcagcgactactccggacttccgtgtgtTTAAACtcacccccttatccagtgaaata
aagatcatattgatgatgattttacagaaataaaaaataatcatttgatttgaaataaagatacaatcatattgatgatttgagtttaacaaaaaaat
aaagaatcacttacttgaaatctgataccaggtctctgtccatgttttctgccaacaccacttcactcccctcttcccagctctggtactgcaggc
cccggcgggctgcaaacttcctccacacgctgaaggggatgtcaaattcctcctgtccctcaatcttcattttatcttctatcagatgtccaaaa
agcgcgtccgggtggatgatgacttcgaccccgtctacccctacgatgcagacaacgcaccgaccgtgcccttcatcaacccccccttcgt
ctcttcagatggattccaagagaagcccctgggggtgttgtccctgcgactggccgaccccgtcaccaccaagaacggggaaatcaccct
caagctgggagagggggtggacctcgattcctcgggaaaactcatctccaacacggccaccaaggccgccgcccctctcagtttttccaa
caacaccatttcccttaacatggatcaccccttttacactaaagatggaaaattatccttacaagtttctccaccattaaatatactgagaacaag
cattctaaacacactagctttaggttttggatcaggtttaggactccgtggctctgccttggcagtacagttagtctctccacttacatttgatact
gatggaaacataaagcttaccttagacagaggtttgcatgttacaacaggagatgcaattgaaagcaacataagctgggctaaaggtttaaa
atttgaagatggagccatagcaaccaacattggaaatgggttagagtttggaagcagtagtacagaaacaggtgttgatgatgcttacccaat
ccaagttaaacttggatctggccttagctttgacagtacaggagccataatggctggtaacaaagaagacgataaactcactttgtggacaac
acctgatccatcaccaaactgtcaaatactcgcagaaaatgatgcaaaactaacactttgcttgactaaatgtggtagtcaaatactggccact
gtgtcagtcttagttgtaggaagtggaaacctaaaccccattactggcaccgtaagcagtgctcaggtgtttctacgttttgatgcaaacggtg
ttcttttaacagaacattctacactaaaaaaatactgggggtataggcagggagatagcatagatggcactccatataccaatgctgtaggatt
catgcccaatttaaaagcttatccaaagtcacaaagttctactactaaaaataatatagtagggcaagtatacatgaatggagatgtttcaaaac
ctatgcttctcactataaccctcaatggtactgatgacagcaacagtacatattcaatgtcattttcatacacctggactaatggaagctatgttg
gagcaacatttggggctaactcttataccttctcatacatcgcccaagaatgaacactgtatcccaccctgcatgccaacccttcccacccca
ctctgtggaacaaactctgaaacacaaaataaaataaagttcaagtgttttattgattcaacagttttacaggattcgagcagttatttttcctcca
ccctcccaggacatggaatacaccaccctctccccccgcacagccttgaacatctgaatgccattggtgatggacatgcttttggtctccacg
ttccacacagtttcagagcgagccagtctcgggtcggtcagggagatgaaaccctccgggcactcccgcatctgcacctcacagctcaac
agctgaggattgtcctcggtggtcgggatcacggttatctggaagaagcagaagagcggcggtgggaatcatagtccgcgaacgggatc
ggccggtggtgtcgcatcaggccccgcagcagtcgctgccgccgccgctccgtcaagctgctgctcagggggtccgggtccagggact
ccctcagcatgatgcccacggccctcagcatcagtcgtctggtgcggcgggcgcagcagcgcatgcggatctcgctcaggtcgctgcag
tacgtgcaacacagaaccaccaggttgttcaacagtccatagttcaacacgctccagccgaaactcatcgcgggaaggatgctacccacgt
ggccgtcgtaccagatcctcaggtaaatcaagtggtgccccctccagaacacgctgcccacgtacatgatctccttgggcatgtggcggttc
accacctcccggtaccacatcaccctctggttgaacatgcagccccggatgatcctgcggaaccacagggccagcaccgccccgcccgc
catgcagcgaagagaccccgggtcccggcaatggcaatggaggacccaccgctcgtacccgtggatcatctgggagctgaacaagtcta
tgttggcacagcacaggcatatgctcatgcatctcttcagcactctcaactcctcgggggtcaaaaccatatcccagggcacggggaactct
tgcaggacagcgaaccccgcagaacagggcaatcctcgcacagaacttacattgtgcatggacagggtatcgcaatcaggcagcaccg
ggtgatcctccaccagagaagcgcgggtctcggtctcctcacagcgtggtaagggggccggccgatacgggtgatggcgggacgcgg
ctgatcgtgttcgcgaccgtgtcatgatgcagttgctttcggacattttcgtacttgctgtagcagaacctggtccgggcgctgcacaccgatc
gccggcggcggtctcggcgcttggaacgctcggtgttgaaattgtaaaacagccactctctcagaccgtgcagcagatctagggcctcag
gagtgatgaagatcccatcatgcctgatggctctgatcacatcgaccaccgtggaatgggccagacccagccagatgatgcaattttgttgg
gtttcggtgacggcgggggagggaagaacaggaagaaccatgattaacttttaatccaaacggtctcggagtacttcaaaatgaagatcgc
ggagatggcacctctcgcccccgctgtgttggtggaaaataacagccaggtcaaaggtgatacggttctcgagatgttccacggtggcttc
cagcaaagcctccacgcgcacatccagaaacaagacaatagcgaaagcgggagggttctctaattcctcaatcatcatgttacactcctgc
accatccccagataattttcatttttccagccttgaatgattcgaactagttcCtgaggtaaatccaagccagccatgataaagagctcgcgca
gagcgccctccaccggcattcttaagcacaccctcataattccaagatattctgctcctggttcacctgcagcagattgacaagcggaatatc
aaaatctctgccgcgatccctgagctcctccctcagcaataactgtaagtactctttcatatcctctccgaaatttttagccataggaccaccag
gaataagattagggcaagccacagtacagataaaccgaagtcctccccagtgagcattgccaaatgcaagactgctataagcatgctggct
agacccggtgatatcttccagataactggacagaaaatcgcccaggcaatttttaagaaaatcaacaaaagaaaaatcctccaggtggacgt
ttagagcctcgggaacaacgatgaagtaaatgcaagcggtgcgttccagcatggttagttagctgatctgtagaaaaaacaaaaatgaacat
taaaccatgctagcctggcgaacaggtgggtaaatcgttctctccagcaccaggcaggccacggggtctccggcgcgaccctcgtaaaaa
ttgtcgctatgattgaaaaccatcacagagagacgttcccggtggccggcgtgaatgattcgacaagatgaatacacccccggaacattgg
cgtccgcgagtgaaaaaaagcgcccgaggaagcaataaggcactacaatgctcagtctcaagtccagcaaagcgatgccatgcggatga
agcacaaaattctcaggtgcgtacaaaatgtaattactcccctcctgcacaggcagcaaagcccccgatccctccaggtacacatacaaag
cctcagcgtccatagcttaccgagcagcagcacacaacaggcgcaagagtcagagaaaggctgagctctaacctgtccacccgctctctg
ctcaatatatagcccagatctacactgacgtaaaggccaaagtctaaaaatacccgccaaataatcacacacgcccagcacacgcccaga
aaccggtgacacactcaaaaaaatacgcgcacttcctcaaacgcccaaaactgccgtcatttccgggttcccacgctacgtcatcaaaaca
cgactttcaaattccgtcgaccgttaaaaacgtcacccgccccgcccctaacggtcgcccgtctctcagccaatcagcgccccgcatcccc
aaattcaaacacctcatttgcatattaacgcgcacaaaaagtttgaggtatattattgatgatgg.
Plasmid 1424 ORF (RNA)

SEQ ID NO: 87

auggcuagcaccccuggaacccagagccccuucuuccuucugcugcugcugaccgugcugacugucgugacaggcucuggcc
acgccagcucuacaccuggcggcgagaaagagacaagcgccacccagagaagcagcgugccaagcagcaccgagaagaacgcc
guguccaugaccagcuccgugcugagcagccacucuccuggcagcggcagcagcacaacacagggccaggaugugacacugg
ccccugccacagaaccugccucuggaucugccgccaccuggggacaggacgugacaagcgugccagugaccagaccugcccug
ggcucuacaacacccccugcccacgaugugaccagcgccccugauaacaagccugccccuggaagcacagccccuccagcucau
ggcgugaccucugccccagauaccagaccagccccaggaucuacagccccacccgcacacggcgugacaagugccccugacaca
agacccgcuccaggcucuacugcuccuccugcccauggcgugacaagcgcucccgauacaaggccagcuccuggcuccacagc
accaccagcacauggcgugacaucagcucccgacacuagaccugcucccggaucaaccgcuccaccagcucacggcgugacca
gcgcaccugauaccagaccugcucugggaagcaccgccccucccgugcacaaugugacaucugcuuccggcagcgccagcggc
ucugccucuacacuggugcacaacggcaccagcgccagagccacaacaaccccagccagcaagagcacccccuucagcaucccu
agccaccacagcgacaccccuaccacacuggccagccacuccaccaagaccgaugccucuagcacccaccacuccagcgugccc
ccucugaccagcagcaaccacagcacaagcccccagcugucuaccggcgucucauucuucuuucuguccuuccacaucagcaa
ccugcaguucaacagcagccuggaagaucccagcaccgacuacuaccaggaacugcagcgggauaucagcgagauguuccugc
aaaucuacaagcagggcggcuuccugggccugagcaacaucaaguucagacccggcagcgugguggugcagcugacccuggc
uuuccgggaaggcaccaucaacgugcacgacguggaaacccaguucaaccaguacaagaccgaggccgccagccgguacaacc
ugaccaucuccgauguguccguguccgacgugcccuucccauucucugcccagucuggcgcaggcgugccaggauggggaau
ugcucugcuggugcucgugugcgugcugguggcccuggccaucguguaucugauugcccuggccgugugccagugccggc
ggaagaauuacggccagcuggacaucuuccccgccagagacaccuaccaccccaugagcgaguaccccacauaccacacccacg
gcagauacgugccacccagcuccaccgacagaucccccuacgagaaagugucugccggcaacggcggcagcucccugagcuac
acaaauccugccguggccgcugccuccgccaaccugggauccggcacaauccugucugagggcgccaccaacuucagccugcu
gaaacuggccggcgacguggaacugaacccuggcccuggagcugccccggagccggagaggacccccguuggccagggaucg
ugggcccauccgggacgcaccaggggaccauccgacaggggauucuguguggugucaccggccaggccagcagaagaggcaa
ccagccucgagggagcguugucuggaaccagacauucccacccgucggugggccggcagcaccacgcgggaccaccguccacu
uccagaccgccacggccaugggacaccccuugcccgccuguguaugccgagacuaaacacuuccuguacucauccggagacaa
ggaacagcuucggccguccuuccuccugucgucgcucagaccgagccugaccggagcacgcagauugguggaaacuaucuuc
cuugggucacguccguggaugccagguaccccacggcgccucccgcgccucccacagagauacuggcagaugcggccucugu
uccuggaauugcugggaaaccacgcucagugcccguacggaguccugcucaagacucacugcccucugagggcggcggucac
uccggcggccggagugugcgcacgggagaagccccagggaagcguggcagcuccggaagaggaggacaccgauccgcgccgc
cucgugcaacuucugcgccagcacuccucgcccuggcaagucuacggguucguccgcgccugccugcgccgccuggugccgc
cugggcucugggguucccggcauaacgagcgccgcuuccugagaaauacuaagaaguuuaucucacuuggaaaacaugccaa
guugucgcugcaagaacucacguggaagaugucaguccgcgauugcgccuggcugcgccgcucgccgggcgucgggugugu
uccagcugcagaacaccgccugagagaagaaauucuggccaaauuucugcauuggcugaugucaguguacguggucgagcug
cugcgcuccuuuuucuacgucacugagacuaccuuucaaaagaaccgccuguucuucuaccgcaaaucuguguggagcaagc
ugcagucaaucggcauucgccagcaucugaagagggugcagcugcgggaacuuuccgaggcagaaguccgccagcaccggga
ggcccggccggcgcuucucacgucgcgucugagauucaucccaaagcccgacgggcugaggccuaucgucaacauggauuac
gucgugggcgcucgcaccuuucgccgugaaaagcgggccgaacgcuugaccucacgggugaaggcccucuucuccgugcuga
acuacgagagagcaagacggccuggccugcugggagcuucggugcugggacuggacgauauccaccgggcuuggcggaccu
uuguucuccgggugagagcccaagacccuccgccggaacuguacuucgugaagguggcgaucaccggagccuaugauacuau
uccgcaagaucgacucaccgaagucaucgccucgaucaucaaaccgcagaacacuuacugcgucaggcgguacgccguggucc
agaaggccgcgcauggccacgugagaaaggcguucaagucgcacguguccacucucaccgaccuccagccuuacaugaggcaa
uucguugcgcauuugcaagagacuucgccccugagagaugcgguggucaucgagcagagcuccagccugaacgaagcgagca
gcggucuguuugacguguuccuccgcuucaugugucaucacgcggugcgaaucaggggaaaaucauacgugcagugccagg
gaaucccacaaggcagcauucugucgacucucuuguguucccuuugcuacggcgauauggaaaacaagcuguucgcugggau
cagacgggacggguugcugcucagacugguggacgacuuccugcuggugacuccgcaccucacucacgccaaaaccuuucuc
cgcacucuggugaggggagugccagaauacggcuguguggucaaucuccggaaaacuguggugaauuucccugucgaggau
gaggcacucggaggaaccgcauuuguccaaaugccagcacauggccuguucccauggugcggucugcugcuggacacccgaa
cucuugaagugcaguccgacuacuccagcuaugcccggacgagcauccgcgccagccucacuuucaaucgcggcuuuaaggc
cggacgaaacaugcgcagaaagcuuuucggaguccuccggcuuaaaugccauucgcucuuucucgaucuccaagucaauucg
cugcagaccgugugcacgaacaucuacaagauccugcugcuccaagccuaccgguuccacgcuugcgugcuucagcugccgu
uucaccaacagguguggaagaacccgaccuucuuucugcgggucauuagcgauacugccucccuguguuacucaauccucaa
ggcaaagaacgccggaaugucgcugggugcgaaaggagccgcgggaccucuuccuagcgaagcggugcaguggcucugccac
caggcuuuccuccugaagcugaccaggcacagagugaccuacgucccgcugcugggcucgcugcgcacugcacagacccagc
ugucuagaaaacuccccggcaccacccugaccgcucuggaagccgccgccaacccagcauugccgucagauuucaagaccauc
uuggacggauccggccagugcaccaauuacgcccugcugaagcuggccggcgacguggaaucuaacccuggcccugaaucgc
caagcgcacccccucaucgguggugcaucccuuggcaacgccuccuccugaccgccucacugcugacuuucuggaacccgccg
accaccgcaaagcugaccauugagagcacucccuucaacguggcugaggggaaggaggugcugcuccuggugcacaaucugc
cccagcaccuguucggguacuccugguacaagggagaacgcguggacgggaaccggcagaucauaggcuacgucaucggaac
ccagcaggccacacccgguccagcguacagcggccgggagauuaucuacccgaacgccucccugcugauccaaaacaucaucc
agaacgacaccgguuucuacacucugcacgugauuaagucagaucuggucaacgaagaggccaccggccaauucagggugua
ccccgaacucccuaagccguucaucaccucgaacaacagcaacccggucgaggaugaagaugcgguggccuugacgugcgaac
cugagauccagaacaccaccuacuuguggugggugaacaaucagagccugccagucuccccacgacuccagcugucgaacgac
aacaggacccugacuuugcuguccgugacucggaacgacgugggcccuuaugaaugcgguauccagaacaagcuguccgugg
accacagcgacccugugauccugaacguccuuuacgggccggacgaccccaccauuuccccgucguacacuuacuaccggccg
ggcgugaaccugucccugucgugccacgcugccuccaauccgccggcccaguacuccuggcucaucgacggaaacauccagca
gcacacccaagaacuguucaucuccaacauuaccgagaaaaacucgggacuuuacaccugucaagccaacaauuccgccagcg
gccacucccgcaccacugucaaaacuaucacuguguccgccgaacucccgaagcccagcaucagcuccaacaacucgaagcccg
uggaggauaaggacgcugucgcguucaccugugaaccagaggcacagaauaccaccuaccuuuggugggucaacggacaguc
ccugccugucucaccgagacugcagcugucaaacgggaauaggacucugaccuuguuuaacgucacccggaacgacgcccgg
gccuacgugugcggcauccagaacuccgugagcgcaaaccggucugacccagugacccuggaugugcuguacggccccgaca
cuccgaucauuucaccccccgauucauccuaccuguccggcgcuaaccucaaccucucaugccacuccgcauccaaccccagcc
cgcaauauucguggcgcauuaacggaauuccucagcaacauacccagguccuguucauugcgaagaucaccccuaacaacaac
ggaaccuacgccugcuuugugucaaaccuggccacugguagaaacaacuccaucgugaaguccauuaccgugucggcguccg
gaacuuccccgggccugagcgccggcgccaccgugggaauuaugaucggcgugcucgugggaguggcccugauc.
Plasmid 1425 ORF (RNA)

SEQ ID NO: 88

auggcuagcgaaucgccaagcgcacccccucaucgguggugcaucccuuggcaacgccuccuccugaccgccucacugcugac
uuucuggaacccgccgaccaccgcaaagcugaccauugagagcacucccuucaacguggcugaggggaaggaggugcugcuc
cuggugcacaaucugccccagcaccuguucggguacuccugguacaagggagaacgcguggacgggaaccggcagaucauag
gcuacgucaucggaacccagcaggccacacccgguccagcguacagcggccgggagauuaucuacccgaacgccucccugcug
auccaaaacaucauccagaacgacaccgguuucuacacucugcacgugauuaagucagaucuggucaacgaagaggccaccgg
ccaauucaggguguaccccgaacucccuaagccguucaucaccucgaacaacagcaacccggucgaggaugaagaugcggugg
ccuugacgugcgaaccugagauccagaacaccaccuacuuguggugggugaacaaucagagccugccagucuccccacgacuc
cagcugucgaacgacaacaggacccugacuuugcuguccgugacucggaacgacgugggcccuuaugaaugcgguauccaga
acaagcuguccguggaccacagcgacccugugauccugaacguccuuuacgggccggacgaccccaccauuuccccgucguac
acuuacuaccggccgggcgugaaccugucccugucgugccacgcugccuccaauccgccggcccaguacuccuggcucaucg
acggaaacauccagcagcacacccaagaacuguucaucuccaacauuaccgagaaaaacucgggacuuuacaccugucaagcca
acaauuccgccagcggccacucccgcaccacugucaaaacuaucacuguguccgccgaacucccgaagcccagcaucagcucca
acaacucgaagcccguggaggauaaggacgcugucgcguucaccugugaaccagaggcacagaauaccaccuaccuuuggug
ggucaacggacagucccugccugucucaccgagacugcagcugucaaacgggaauaggacucugaccuuguuuaacgucacc
cggaacgacgcccgggccuacgugugcggcauccagaacuccgugagcgcaaaccggucugacccagugacccuggaugugc
uguacggccccgacacuccgaucauuucaccccccgauucauccuaccuguccggcgcuaaccucaaccucucaugccacucc
gcauccaaccccagcccgcaauauucguggcgcauuaacggaauuccucagcaacauacccagguccuguucauugcgaagau
caccccuaacaacaacggaaccuacgccugcuuugugucaaaccuggccacugguagaaacaacuccaucgugaaguccauua
ccgugucggcguccggaacuuccccgggccugagcgccggcgccaccgugggaauuaugaucggcgugcucgugggagugg
cccugaucggauccggcgagggcagaggcagccugcugacauguggcgacguggaagagaacccuggccccaccccuggaac
ccagagccccuucuuccuucugcugcugcugaccgugcugacugucgugacaggcucuggccacgccagcucuacaccuggc
ggcgagaaagagacaagcgccacccagagaagcagcgugccaagcagcaccgagaagaacgccguguccaugaccagcuccgu
gcugagcagccacucuccuggcagcggcagcagcacaacacagggccaggaugugacacuggccccugccacagaaccugccu
cuggaucugccgccaccuggggacaggacgugacaagcgugccagugaccagaccugcccugggcucuacaacacccccugcc
cacgaugugaccagcgccccugauaacaagccugccccuggaagcacagccccuccagcucauggcgugaccucugccccaga
uaccagaccagccccaggaucuacagccccacccgcacacggcgugacaagugccccugacacaagacccgcuccaggcucuac
ugcuccuccugcccauggcgugacaagcgcucccgauacaaggccagcuccuggcuccacagcaccaccagcacauggcguga
caucagcucccgacacuagaccugcucccggaucaaccgcuccaccagcucacggcgugaccagcgcaccugauaccagaccu
gcucugggaagcaccgccccucccgugcacaaugugacaucugcuuccggcagcgccagcggcucugccucuacacuggugc
acaacggcaccagcgccagagccacaacaaccccagccagcaagagcacccccuucagcaucccuagccaccacagcgacacccc
uaccacacuggccagccacuccaccaagaccgaugccucuagcacccaccacuccagcgugcccccucugaccagcagcaacca
cagcacaagcccccagcugucuaccggcgucucauucuucuuucuguccuuccacaucagcaaccugcaguucaacagcagcc
uggaagaucccagcaccgacuacuaccaggaacugcagcgggauaucagcgagauguuccugcaaaucuacaagcagggcggc
uuccugggccugagcaacaucaaguucagacccggcagcgugguggugcagcugacccuggcuuuccgggaaggcaccauca
acgugcacgacguggaaacccaguucaaccaguacaagaccgaggccgccagccgguacaaccugaccaucuccgaugugucc
guguccgacgugcccuucccauucucugcccagucuggcgcaggcgugccaggauggggaauugcucugcuggugcucgug
ugcgugcugguggcccuggccaucguguaucugauugcccuggccgugugccagugccggcggaagaauuacggccagcug
gacaucuuccccgccagagacaccuaccaccccaugagcgaguaccccacauaccacacccacggcagauacgugccacccagc
uccaccgacagaucccccuacgagaaagugucugccggcaacggcggcagcucccugagcuacacaaauccugccguggccgc
ugccuccgccaaccugggauccggcacaauccugucugagggcgccaccaacuucagccugcugaaacuggccggcgacgug
gaacugaacccuggcccuggagcugccccggagccggagaggacccccguuggccagggaucgugggcccauccgggacgca
ccaggggaccauccgacaggggauucuguguggugucaccggccaggccagcagaagaggcaaccagccucgagggagcguu
gucuggaaccagacauucccacccgucggugggccggcagcaccacgcgggaccaccguccacuuccagaccgccacggccau
gggacaccccuugcccgccuguguaugccgagacuaaacacuuccuguacucauccggagacaaggaacagcuucggccgucc
uuccuccugucgucgcucagaccgagccugaccggagcacgcagauugguggaaacuaucuuccuugggucacguccgugga
ugccagguaccccacggcgccucccgcgccucccacagagauacuggcagaugcggccucuguuccuggaauugcugggaaa
ccacgcucagugcccguacggaguccugcucaagacucacugcccucugagggcggcggucacuccggcggccggagugugc
gcacgggagaagccccagggaagcguggcagcuccggaagaggaggacaccgauccgcgccgccucgugcaacuucugcgcc
agcacuccucgcccuggcaagucuacggguucguccgcgccugccugcgccgccuggugccgccugggcucugggguucccg
gcauaacgagcgccgcuuccugagaaauacuaagaaguuuaucucacuuggaaaacaugccaaguugucgcugcaagaacuca
cguggaagaugucaguccgcgauugcgccuggcugcgccgcucgccgggcgucggguguguuccagcugcagaacaccgcc
ugagagaagaaauucuggccaaauuucugcauuggcugaugucaguguacguggucgagcugcugcgcuccuuuuucuacg
ucacugagacuaccuuucaaaagaaccgccuguucuucuaccgcaaaucuguguggagcaagcugcagucaaucggcauucg
ccagcaucugaagagggugcagcugcgggaacuuuccgaggcagaaguccgccagcaccgggaggcccggccggcgcuucuc
acgucgcgucugagauucaucccaaagcccgacgggcugaggccuaucgucaacauggauuacgucgugggcgcucgcaccu
uucgccgugaaaagcgggccgaacgcuugaccucacgggugaaggcccucuucuccgugcugaacuacgagagagcaagacg
gccuggccugcugggagcuucggugcugggacuggacgauauccaccgggcuuggcggaccuuuguucuccgggugagagc
ccaagacccuccgccggaacuguacuucgugaagguggcgaucaccggagccuaugauacuauuccgcaagaucgacucaccg
aagucaucgccucgaucaucaaaccgcagaacacuuacugcgucaggcgguacgccgugguccagaaggccgcgcauggccac
gugagaaaggcguucaagucgcacguguccacucucaccgaccuccagccuuacaugaggcaauucguugcgcauuugcaag
agacuucgccccugagagaugcgguggucaucgagcagagcuccagccugaacgaagcgagcagcggucuguuugacguguu
ccuccgcuucaugugucaucacgcggugcgaaucaggggaaaaucauacgugcagugccagggaaucccacaaggcagcauu
cugucgacucucuuguguucccuuugcuacggcgauauggaaaacaagcuguucgcugggaucagacgggacggguugcug
cucagacugguggacgacuuccugcuggugacuccgcaccucacucacgccaaaaccuuucuccgcacucuggugaggggag
ugccagaauacggcuguguggucaaucuccggaaaacuguggugaauuucccugucgaggaugaggcacucggaggaaccgc
auuuguccaaaugccagcacauggccuguucccauggugcggucugcugcuggacacccgaacucuugaagugcaguccgac
uacuccagcuaugcccggacgagcauccgcgccagccucacuuucaaucgcggcuuuaaggccggacgaaacaugcgcagaaa
gcuuuucggaguccuccggcuuaaaugccauucgcucuuucucgaucuccaagucaauucgcugcagaccgugugcacgaac
aucuacaagauccugcugcuccaagccuaccgguuccacgcuugcgugcuucagcugccguuucaccaacagguguggaaga
acccgaccuucuuucugcgggucauuagcgauacugccucccuguguuacucaauccucaaggcaaagaacgccggaauguc
gcugggugcgaaaggagccgcgggaccucuuccuagcgaagcggugcaguggcucugccaccaggcuuuccuccugaagcug
accaggcacagagugaccuacgucccgcugcugggcucgcugcgcacugcacagacccagcugucuagaaaacuccccggcac
cacccugaccgcucuggaagccgccgccaacccagcauugccgucagauuucaagaccaucuuggac.
Plasmid 1426 ORF (RNA)

SEQ ID NO: 89

auggcuagcggagcugccccggagccggagaggacccccguuggccagggaucgugggcccauccgggacgcaccaggggac
cauccgacaggggauucuguguggugucaccggccaggccagcagaagaggcaaccagccucgagggagcguugucuggaac
cagacauucccacccgucggugggccggcagcaccacgcgggaccaccguccacuuccagaccgccacggccaugggacaccc
cuugcccgccuguguaugccgagacuaaacacuuccuguacucauccggagacaaggaacagcuucggccguccuuccuccu
gucgucgcucagaccgagccugaccggagcacgcagauugguggaaacuaucuuccuugggucacguccguggaugccaggu
accccacggcgccucccgcgccucccacagagauacuggcagaugcggccucuguuccuggaauugcugggaaaccacgcuca
gugcccguacggaguccugcucaagacucacugcccucugagggcggcggucacuccggcggccggagugugcgcacgggag
aagccccagggaagcguggcagcuccggaagaggaggacaccgauccgcgccgccucgugcaacuucugcgccagcacuccuc
gcccuggcaagucuacggguucguccgcgccugccugcgccgccuggugccgccugggcucugggguucccggcauaacga
gcgccgcuuccugagaaauacuaagaaguuuaucucacuuggaaaacaugccaaguugucgcugcaagaacucacguggaag
augucaguccgcgauugcgccuggcugcgccgcucgccgggcgucggguguguuccagcugcagaacaccgccugagagaag
aaauucuggccaaauuucugcauuggcugaugucaguguacguggucgagcugcugcgcuccuuuuucuacgucacugaga
cuaccuuucaaaagaaccgccuguucuucuaccgcaaaucuguguggagcaagcugcagucaaucggcauucgccagcaucu
gaagagggugcagcugcgggaacuuuccgaggcagaaguccgccagcaccgggaggcccggccggcgcuucucacgucgcgu
cugagauucaucccaaagcccgacgggcugaggccuaucgucaacauggauuacgucgugggcgcucgcaccuuucgccgug
aaaagcgggccgaacgcuugaccucacgggugaaggcccucuucuccgugcugaacuacgagagagcaagacggccuggccu
gcugggagcuucggugcugggacuggacgauauccaccgggcuuggcggaccuuuguucuccgggugagagcccaagaccc
uccgccggaacuguacuucgugaagguggcgaucaccggagccuaugauacuauuccgcaagaucgacucaccgaagucauc
gccucgaucaucaaaccgcagaacacuuacugcgucaggcgguacgccgugguccagaaggccgcgcauggccacgugagaaa
ggcguucaagucgcacguguccacucucaccgaccuccagccuuacaugaggcaauucguugcgcauuugcaagagacuucg
ccccugagagaugcgguggucaucgagcagagcuccagccugaacgaagcgagcagcggucuguuugacguguuccuccgcu
ucaugugucaucacgcggugcgaaucaggggaaaaucauacgugcagugccagggaaucccacaaggcagcauucugucgac
ucucuuguguucccuuugcuacggcgauauggaaaacaagcuguucgcugggaucagacgggacggguugcugcucagacu
gguggacgacuuccugcuggugacuccgcaccucacucacgccaaaaccuuucuccgcacucuggugaggggagugccagaa
uacggcuguguggucaaucuccggaaaacuguggugaauuucccugucgaggaugaggcacucggaggaaccgcauuuguc
caaaugccagcacauggccuguucccauggugcggucugcugcuggacacccgaacucuugaagugcaguccgacuacucca
gcuaugcccggacgagcauccgcgccagccucacuuucaaucgcggcuuuaaggccggacgaaacaugcgcagaaagcuuuu
cggaguccuccggcuuaaaugccauucgcucuuucucgaucuccaagucaauucgcugcagaccgugugcacgaacaucuac
aagauccugcugcuccaagccuaccgguuccacgcuugcgugcuucagcugccguuucaccaacagguguggaagaacccga
ccuucuuucugcgggucauuagcgauacugccucccuguguuacucaauccucaaggcaaagaacgccggaaugucgcuggg
ugcgaaaggagccgcgggaccucuuccuagcgaagcggugcaguggcucugccaccaggcuuuccuccugaagcugaccagg
cacagagugaccuacgucccgcugcugggcucgcugcgcacugcacagacccagcugucuagaaaacuccccggcaccacccu
gaccgcucuggaagccgccgccaacccagcauugccgucagauuucaagaccaucuuggacggauccggcacaauccugucug
agggcgccaccaacuucagccugcugaaacuggccggcgacguggaacugaacccuggcccuaccccuggaacccagagcccc
uucuuccuucugcugcugcugaccgugcugacugucgugacaggcucuggccacgccagcucuacaccuggcggcgagaaag
agacaagcgccacccagagaagcagcgugccaagcagcaccgagaagaacgccguguccaugaccagcuccgugcugagcagc
cacucuccuggcagcggcagcagcacaacacagggccaggaugugacacuggccccugccacagaaccugccucuggaucugc
cgccaccuggggacaggacgugacaagcgugccagugaccagaccugcccugggcucuacaacacccccugcccacgauguga
ccagcgccccugauaacaagccugccccuggaagcacagccccuccagcucauggcgugaccucugccccagauaccagacca
gccccaggaucuacagccccacccgcacacggcgugacaagugccccugacacaagacccgcuccaggcucuacugcuccucc
ugcccauggcgugacaagcgcucccgauacaaggccagcuccuggcuccacagcaccaccagcacauggcgugacaucagcuc
ccgacacuagaccugcucccggaucaaccgcuccaccagcucacggcgugaccagcgcaccugauaccagaccugcucuggga
agcaccgccccucccgugcacaaugugacaucugcuuccggcagcgccagcggcucugccucuacacuggugcacaacggcac
cagcgccagagccacaacaaccccagccagcaagagcacccccuucagcaucccuagccaccacagcgacaccccuaccacacug
gccagccacuccaccaagaccgaugccucuagcacccaccacuccagcgugcccccucugaccagcagcaaccacagcacaagc
ccccagcugucuaccggcgucucauucuucuuucuguccuuccacaucagcaaccugcaguucaacagcagccuggaagaucc
cagcaccgacuacuaccaggaacugcagcgggauaucagcgagauguuccugcaaaucuacaagcagggcggcuuccugggc
cugagcaacaucaaguucagacccggcagcgugguggugcagcugacccuggcuuuccgggaaggcaccaucaacgugcacg
acguggaaacccaguucaaccaguacaagaccgaggccgccagccgguacaaccugaccaucuccgauguguccguguccgac
gugcccuucccauucucugcccagucuggcgcaggcgugccaggauggggaauugcucugcuggugcucgugugcgugcug
guggcccuggccaucguguaucugauugcccuggccgugugccagugccggcggaagaauuacggccagcuggacaucuuc
cccgccagagacaccuaccaccccaugagcgaguaccccacauaccacacccacggcagauacgugccacccagcuccaccgac
agaucccccuacgagaaagugucugccggcaacggcggcagcucccugagcuacacaaauccugccguggccgcugccuccgc
caaccugggauccggcagaaucuucaacgcccacuacgccggcuacuucgccgaccugcugauccacgacaucgagacaaacc
cuggccccgaaucgccaagcgcacccccucaucgguggugcaucccuuggcaacgccuccuccugaccgccucacugcugacu
uucuggaacccgccgaccaccgcaaagcugaccauugagagcacucccuucaacguggcugaggggaaggaggugcugcucc
uggugcacaaucugccccagcaccuguucggguacuccugguacaagggagaacgcguggacgggaaccggcagaucauagg
cuacgucaucggaacccagcaggccacacccgguccagcguacagcggccgggagauuaucuacccgaacgccucccugcuga
uccaaaacaucauccagaacgacaccgguuucuacacucugcacgugauuaagucagaucuggucaacgaagaggccaccggc
caauucaggguguaccccgaacucccuaagccguucaucaccucgaacaacagcaacccggucgaggaugaagaugcgguggc
cuugacgugcgaaccugagauccagaacaccaccuacuuguggugggugaacaaucagagccugccagucuccccacgacucc
agcugucgaacgacaacaggacccugacuuugcuguccgugacucggaacgacgugggcccuuaugaaugcgguauccagaa
caagcuguccguggaccacagcgacccugugauccugaacguccuuuacgggccggacgaccccaccauuuccccgucguaca
cuuacuaccggccgggcgugaaccugucccugucgugccacgcugccuccaauccgccggcccaguacuccuggcucaucga
cggaaacauccagcagcacacccaagaacuguucaucuccaacauuaccgagaaaaacucgggacuuuacaccugucaagccaa
caauuccgccagcggccacucccgcaccacugucaaaacuaucacuguguccgccgaacucccgaagcccagcaucagcuccaa
caacucgaagcccguggaggauaaggacgcugucgcguucaccugugaaccagaggcacagaauaccaccuaccuuuggugg
gucaacggacagucccugccugucucaccgagacugcagcugucaaacgggaauaggacucugaccuuguuuaacgucaccc
ggaacgacgcccgggccuacgugugcggcauccagaacuccgugagcgcaaaccggucugacccagugacccuggaugugcu
guacggccccgacacuccgaucauuucaccccccgauucauccuaccuguccggcgcuaaccucaaccucucaugccacuccg
cauccaaccccagcccgcaauauucguggcgcauuaacggaauuccucagcaacauacccagguccuguucauugcgaagauc
accccuaacaacaacggaaccuacgccugcuuugugucaaaccuggccacugguagaaacaacuccaucgugaaguccauuac
cgugucggcguccggaacuuccccgggccugagcgccggcgccaccgugggaauuaugaucggcgugcucgugggaguggc
ccugauc.
Plasmid 1427 ORF (RNA)

SEQ ID NO: 90

auggcuagcggagcugccccggagccggagaggacccccguuggccagggaucgugggcccauccgggacgcaccaggggac
cauccgacaggggauucuguguggugucaccggccaggccagcagaagaggcaaccagccucgagggagcguugucuggaac
cagacauucccacccgucggugggccggcagcaccacgcgggaccaccguccacuuccagaccgccacggccaugggacaccc
cuugcccgccuguguaugccgagacuaaacacuuccuguacucauccggagacaaggaacagcuucggccguccuuccuccu
gucgucgcucagaccgagccugaccggagcacgcagauugguggaaacuaucuuccuugggucacguccguggaugccaggu
accccacggcgccucccgcgccucccacagagauacuggcagaugcggccucuguuccuggaauugcugggaaaccacgcuca
gugcccguacggaguccugcucaagacucacugcccucugagggcggcggucacuccggcggccggagugugcgcacgggag
aagccccagggaagcguggcagcuccggaagaggaggacaccgauccgcgccgccucgugcaacuucugcgccagcacuccuc
gcccuggcaagucuacggguucguccgcgccugccugcgccgccuggugccgccugggcucugggguucccggcauaacga
gcgccgcuuccugagaaauacuaagaaguuuaucucacuuggaaaacaugccaaguugucgcugcaagaacucacguggaag
augucaguccgcgauugcgccuggcugcgccgcucgccgggcgucggguguguuccagcugcagaacaccgccugagagaag
aaauucuggccaaauuucugcauuggcugaugucaguguacguggucgagcugcugcgcuccuuuuucuacgucacugaga
cuaccuuucaaaagaaccgccuguucuucuaccgcaaaucuguguggagcaagcugcagucaaucggcauucgccagcaucu
gaagagggugcagcugcgggaacuuuccgaggcagaaguccgccagcaccgggaggcccggccggcgcuucucacgucgcgu
cugagauucaucccaaagcccgacgggcugaggccuaucgucaacauggauuacgucgugggcgcucgcaccuuucgccgug
aaaagcgggccgaacgcuugaccucacgggugaaggcccucuucuccgugcugaacuacgagagagcaagacggccuggccu
gcugggagcuucggugcugggacuggacgauauccaccgggcuuggcggaccuuuguucuccgggugagagcccaagaccc
uccgccggaacuguacuucgugaagguggcgaucaccggagccuaugauacuauuccgcaagaucgacucaccgaagucauc
gccucgaucaucaaaccgcagaacacuuacugcgucaggcgguacgccgugguccagaaggccgcgcauggccacgugagaaa
ggcguucaagucgcacguguccacucucaccgaccuccagccuuacaugaggcaauucguugcgcauuugcaagagacuucg
ccccugagagaugcgguggucaucgagcagagcuccagccugaacgaagcgagcagcggucuguuugacguguuccuccgcu
ucaugugucaucacgcggugcgaaucaggggaaaaucauacgugcagugccagggaaucccacaaggcagcauucugucgac
ucucuuguguucccuuugcuacggcgauauggaaaacaagcuguucgcugggaucagacgggacggguugcugcucagacu
gguggacgacuuccugcuggugacuccgcaccucacucacgccaaaaccuuucuccgcacucuggugaggggagugccagaa
uacggcuguguggucaaucuccggaaaacuguggugaauuucccugucgaggaugaggcacucggaggaaccgcauuuguc
caaaugccagcacauggccuguucccauggugcggucugcugcuggacacccgaacucuugaagugcaguccgacuacucca
gcuaugcccggacgagcauccgcgccagccucacuuucaaucgcggcuuuaaggccggacgaaacaugcgcagaaagcuuuu
cggaguccuccggcuuaaaugccauucgcucuuucucgaucuccaagucaauucgcugcagaccgugugcacgaacaucuac
aagauccugcugcuccaagccuaccgguuccacgcuugcgugcuucagcugccguuucaccaacagguguggaagaacccga
ccuucuuucugcgggucauuagcgauacugccucccuguguuacucaauccucaaggcaaagaacgccggaaugucgcuggg
ugcgaaaggagccgcgggaccucuuccuagcgaagcggugcaguggcucugccaccaggcuuuccuccugaagcugaccagg
cacagagugaccuacgucccgcugcugggcucgcugcgcacugcacagacccagcugucuagaaaacuccccggcaccacccu
gaccgcucuggaagccgccgccaacccagcauugccgucagauuucaagaccaucuuggacggauccggccagugcaccaauu
acgcccugcugaagcuggccggcgacguggaaucuaacccuggcccugaaucgccaagcgcacccccucaucgguggugcau
cccuuggcaacgccuccuccugaccgccucacugcugacuuucuggaacccgccgaccaccgcaaagcugaccauugagagca
cucccuucaacguggcugaggggaaggaggugcugcuccuggugcacaaucugccccagcaccuguucggguacuccuggua
caagggagaacgcguggacgggaaccggcagaucauaggcuacgucaucggaacccagcaggccacacccgguccagcguaca
gcggccgggagauuaucuacccgaacgccucccugcugauccaaaacaucauccagaacgacaccgguuucuacacucugcac
gugauuaagucagaucuggucaacgaagaggccaccggccaauucaggguguaccccgaacucccuaagccguucaucaccuc
gaacaacagcaacccggucgaggaugaagaugcgguggccuugacgugcgaaccugagauccagaacaccaccuacuugugg
ugggugaacaaucagagccugccagucuccccacgacuccagcugucgaacgacaacaggacccugacuuugcuguccgugac
ucggaacgacgugggcccuuaugaaugcgguauccagaacaagcuguccguggaccacagcgacccugugauccugaacguc
cuuuacgggccggacgaccccaccauuuccccgucguacacuuacuaccggccgggcgugaaccugucccugucgugccacg
cugccuccaauccgccggcccaguacuccuggcucaucgacggaaacauccagcagcacacccaagaacuguucaucuccaaca
uuaccgagaaaaacucgggacuuuacaccugucaagccaacaauuccgccagcggccacucccgcaccacugucaaaacuauca
cuguguccgccgaacucccgaagcccagcaucagcuccaacaacucgaagcccguggaggauaaggacgcugucgcguucacc
ugugaaccagaggcacagaauaccaccuaccuuuggugggucaacggacagucccugccugucucaccgagacugcagcugu
caaacgggaauaggacucugaccuuguuuaacgucacccggaacgacgcccgggccuacgugugcggcauccagaacuccgu
gagcgcaaaccggucugacccagugacccuggaugugcuguacggccccgacacuccgaucauuucaccccccgauucauccu
accuguccggcgcuaaccucaaccucucaugccacuccgcauccaaccccagcccgcaauauucguggcgcauuaacggaauu
ccucagcaacauacccagguccuguucauugcgaagaucaccccuaacaacaacggaaccuacgccugcuuugugucaaaccu
ggccacugguagaaacaacuccaucgugaaguccauuaccgugucggcguccggaacuuccccgggccugagcgccggcgcc
accgugggaauuaugaucggcgugcucgugggaguggcccugaucggauccggcgagggcagaggcagccugcugacaugu
ggcgacguggaagagaacccuggccccaccccuggaacccagagccccuucuuccuucugcugcugcugaccgugcugacug
ucgugacaggcucuggccacgccagcucuacaccuggcggcgagaaagagacaagcgccacccagagaagcagcgugccaagc
agcaccgagaagaacgccguguccaugaccagcuccgugcugagcagccacucuccuggcagcggcagcagcacaacacaggg
ccaggaugugacacuggccccugccacagaaccugccucuggaucugccgccaccuggggacaggacgugacaagcgugcca
gugaccagaccugcccugggcucuacaacacccccugcccacgaugugaccagcgccccugauaacaagccugccccuggaag
cacagccccuccagcucauggcgugaccucugccccagauaccagaccagccccaggaucuacagccccacccgcacacggcgu
gacaagugccccugacacaagacccgcuccaggcucuacugcuccuccugcccauggcgugacaagcgcucccgauacaaggc
cagcuccuggcuccacagcaccaccagcacauggcgugacaucagcucccgacacuagaccugcucccggaucaaccgcuccac
cagcucacggcgugaccagcgcaccugauaccagaccugcucugggaagcaccgccccucccgugcacaaugugacaucugcu
uccggcagcgccagcggcucugccucuacacuggugcacaacggcaccagcgccagagccacaacaaccccagccagcaagagc
acccccuucagcaucccuagccaccacagcgacaccccuaccacacuggccagccacuccaccaagaccgaugccucuagcacc
caccacuccagcgugcccccucugaccagcagcaaccacagcacaagcccccagcugucuaccggcgucucauucuucuuucu
guccuuccacaucagcaaccugcaguucaacagcagccuggaagaucccagcaccgacuacuaccaggaacugcagcgggaua
ucagcgagauguuccugcaaaucuacaagcagggcggcuuccugggccugagcaacaucaaguucagacccggcagcguggu
ggugcagcugacccuggcuuuccgggaaggcaccaucaacgugcacgacguggaaacccaguucaaccaguacaagaccgagg
ccgccagccgguacaaccugaccaucuccgauguguccguguccgacgugcccuucccauucucugcccagucuggcgcagg
cgugccaggauggggaauugcucugcuggugcucgugugcgugcugguggcccuggccaucguguaucugauugcccugg
ccgugugccagugccggcggaagaauuacggccagcuggacaucuuccccgccagagacaccuaccaccccaugagcgaguac
cccacauaccacacccacggcagauacgugccacccagcuccaccgacagaucccccuacgagaaagugucugccggcaacggc
ggcagcucccugagcuacacaaauccugccguggccgcugccuccgccaaccug.
Plasmid 1428 ORF (RNA)

SEQ ID NO: 91

auggcuagcaccccuggaacccagagccccuucuuccuucugcugcugcugaccgugcugacugucgugacaggcucuggcc
acgccagcucuacaccuggcggcgagaaagagacaagcgccacccagagaagcagcgugccaagcagcaccgagaagaacgcc
guguccaugaccagcuccgugcugagcagccacucuccuggcagcggcagcagcacaacacagggccaggaugugacacugg
ccccugccacagaaccugccucuggaucugccgccaccuggggacaggacgugacaagcgugccagugaccagaccugcccug
ggcucuacaacacccccugcccacgaugugaccagcgccccugauaacaagccugccccuggaagcacagccccuccagcucau
ggcgugaccucugccccagauaccagaccagccccaggaucuacagccccacccgcacacggcgugacaagugccccugacaca
agacccgcuccaggcucuacugcuccuccugcccauggcgugacaagcgcucccgauacaaggccagcuccuggcuccacagc
accaccagcacauggcgugacaucagcucccgacacuagaccugcucccggaucaaccgcuccaccagcucacggcgugacca
gcgcaccugauaccagaccugcucugggaagcaccgccccucccgugcacaaugugacaucugcuuccggcagcgccagcggc
ucugccucuacacuggugcacaacggcaccagcgccagagccacaacaaccccagccagcaagagcacccccuucagcaucccu
agccaccacagcgacaccccuaccacacuggccagccacuccaccaagaccgaugccucuagcacccaccacuccagcgugccc
ccucugaccagcagcaaccacagcacaagcccccagcugucuaccggcgucucauucuucuuucuguccuuccacaucagcaa
ccugcaguucaacagcagccuggaagaucccagcaccgacuacuaccaggaacugcagcgggauaucagcgagauguuccugc
aaaucuacaagcagggcggcuuccugggccugagcaacaucaaguucagacccggcagcgugguggugcagcugacccuggc
uuuccgggaaggcaccaucaacgugcacgacguggaaacccaguucaaccaguacaagaccgaggccgccagccgguacaacc
ugaccaucuccgauguguccguguccgacgugcccuucccauucucugcccagucuggcgcaggcgugccaggauggggaau
ugcucugcuggugcucgugugcgugcugguggcccuggccaucguguaucugauugcccuggccgugugccagugccggc
ggaagaauuacggccagcuggacaucuuccccgccagagacaccuaccaccccaugagcgaguaccccacauaccacacccacg
gcagauacgugccacccagcuccaccgacagaucccccuacgagaaagugucugccggcaacggcggcagcucccugagcuac
acaaauccugccguggccgcugccuccgccaaccugggauccggcagaaucuucaacgcccacuacgccggcuacuucgccga
ccugcugauccacgacaucgagacaaacccuggccccaagcugaccauugagagcacucccuucaacguggcugaggggaagg
aggugcugcuccuggugcacaaucugccccagcaccuguucggguacuccugguacaagggagaacgcguggacgggaaccg
gcagaucauaggcuacgucaucggaacccagcaggccacacccgguccagcguacagcggccgggagauuaucuacccgaacg
ccucccugcugauccaaaacaucauccagaacgacaccgguuucuacacucugcacgugauuaagucagaucuggucaacgaa
gaggccaccggccaauucaggguguaccccgaacucccuaagccguucaucaccucgaacaacagcaacccggucgaggauga
agaugcgguggccuugacgugcgaaccugagauccagaacaccaccuacuuguggugggugaacaaucagagccugccaguc
uccccacgacuccagcugucgaacgacaacaggacccugacuuugcuguccgugacucggaacgacgugggcccuuaugaau
gcgguauccagaacaagcuguccguggaccacagcgacccugugauccugaacguccuuuacgggccggacgaccccaccauu
uccccgucguacacuuacuaccggccgggcgugaaccugucccugucgugccacgcugccuccaauccgccggcccaguacuc
cuggcucaucgacggaaacauccagcagcacacccaagaacuguucaucuccaacauuaccgagaaaaacucgggacuuuaca
ccugucaagccaacaauuccgccagcggccacucccgcaccacugucaaaacuaucacuguguccgccgaacucccgaagccca
gcaucagcuccaacaacucgaagcccguggaggauaaggacgcugucgcguucaccugugaaccagaggcacagaauaccacc
uaccuuuggugggucaacggacagucccugccugucucaccgagacugcagcugucaaacgggaauaggacucugaccuugu
uuaacgucacccggaacgacgcccgggccuacgugugcggcauccagaacuccgugagcgcaaaccggucugacccagugacc
cuggaugugcuguacggccccgacacuccgaucauuucaccccccgauucauccuaccuguccggcgcuaaccucaaccucuc
augccacuccgcauccaaccccagcccgcaauauucguggcgcauuaacggaauuccucagcaacauacccagguccuguuca
uugcgaagaucaccccuaacaacaacggaaccuacgccugcuuugugucaaaccuggccacugguagaaacaacuccaucgug
aaguccauuaccgugucggcguccggauccggcgagggcagaggcagccugcugacauguggcgacguggaagagaacccug
gccccggagcugccccggagccggagaggacccccguuggccagggaucgugggcccauccgggacgcaccaggggaccauc
cgacaggggauucuguguggugucaccggccaggccagcagaagaggcaaccagccucgagggagcguugucuggaaccaga
cauucccacccgucggugggccggcagcaccacgcgggaccaccguccacuuccagaccgccacggccaugggacaccccuug
cccgccuguguaugccgagacuaaacacuuccuguacucauccggagacaaggaacagcuucggccguccuuccuccugucg
ucgcucagaccgagccugaccggagcacgcagauugguggaaacuaucuuccuugggucacguccguggaugccagguaccc
cacggcgccucccgcgccucccacagagauacuggcagaugcggccucuguuccuggaauugcugggaaaccacgcucagug
cccguacggaguccugcucaagacucacugcccucugagggcggcggucacuccggcggccggagugugcgcacgggagaag
ccccagggaagcguggcagcuccggaagaggaggacaccgauccgcgccgccucgugcaacuucugcgccagcacuccucgcc
cuggcaagucuacggguucguccgcgccugccugcgccgccuggugccgccugggcucugggguucccggcauaacgagcg
ccgcuuccugagaaauacuaagaaguuuaucucacuuggaaaacaugccaaguugucgcugcaagaacucacguggaagaug
ucaguccgcgauugcgccuggcugcgccgcucgccgggcgucggguguguuccagcugcagaacaccgccugagagaagaaa
uucuggccaaauuucugcauuggcugaugucaguguacguggucgagcugcugcgcuccuuuuucuacgucacugagacua
ccuuucaaaagaaccgccuguucuucuaccgcaaaucuguguggagcaagcugcagucaaucggcauucgccagcaucugaa
gagggugcagcugcgggaacuuuccgaggcagaaguccgccagcaccgggaggcccggccggcgcuucucacgucgcgucug
agauucaucccaaagcccgacgggcugaggccuaucgucaacauggauuacgucgugggcgcucgcaccuuucgccgugaaa
agcgggccgaacgcuugaccucacgggugaaggcccucuucuccgugcugaacuacgagagagcaagacggccuggccugcu
gggagcuucggugcugggacuggacgauauccaccgggcuuggcggaccuuuguucuccgggugagagcccaagacccucc
gccggaacuguacuucgugaagguggcgaucaccggagccuaugauacuauuccgcaagaucgacucaccgaagucaucgcc
ucgaucaucaaaccgcagaacacuuacugcgucaggcgguacgccgugguccagaaggccgcgcauggccacgugagaaagg
cguucaagucgcacguguccacucucaccgaccuccagccuuacaugaggcaauucguugcgcauuugcaagagacuucgcc
ccugagagaugcgguggucaucgagcagagcuccagccugaacgaagcgagcagcggucuguuugacguguuccuccgcuuc
augugucaucacgcggugcgaaucaggggaaaaucauacgugcagugccagggaaucccacaaggcagcauucugucgacuc
ucuuguguucccuuugcuacggcgauauggaaaacaagcuguucgcugggaucagacgggacggguugcugcucagacugg
uggacgacuuccugcuggugacuccgcaccucacucacgccaaaaccuuucuccgcacucuggugaggggagugccagaaua
cggcuguguggucaaucuccggaaaacuguggugaauuucccugucgaggaugaggcacucggaggaaccgcauuugucca
aaugccagcacauggccuguucccauggugcggucugcugcuggacacccgaacucuugaagugcaguccgacuacuccagc
uaugcccggacgagcauccgcgccagccucacuuucaaucgcggcuuuaaggccggacgaaacaugcgcagaaagcuuuucg
gaguccuccggcuuaaaugccauucgcucuuucucgaucuccaagucaauucgcugcagaccgugugcacgaacaucuacaa
gauccugcugcuccaagccuaccgguuccacgcuugcgugcuucagcugccguuucaccaacagguguggaagaacccgacc
uucuuucugcgggucauuagcgauacugccucccuguguuacucaauccucaaggcaaagaacgccggaaugucgcugggug
cgaaaggagccgcgggaccucuuccuagcgaagcggugcaguggcucugccaccaggcuuuccuccugaagcugaccaggca
cagagugaccuacgucccgcugcugggcucgcugcgcacugcacagacccagcugucuagaaaacuccccggcaccacccuga
ccgcucuggaagccgccgccaacccagcauugccgucagauuucaagaccaucuuggac.
Plasmid 1429 ORF (RNA)

SEQ ID NO: 92

auggcuagcaagcugaccauugagagcacucccuucaacguggcugaggggaaggaggugcugcuccuggugcacaaucugc
cccagcaccuguucggguacuccugguacaagggagaacgcguggacgggaaccggcagaucauaggcuacgucaucggaac
ccagcaggccacacccgguccagcguacagcggccgggagauuaucuacccgaacgccucccugcugauccaaaacaucaucc
agaacgacaccgguuucuacacucugcacgugauuaagucagaucuggucaacgaagaggccaccggccaauucagggugua
ccccgaacucccuaagccguucaucaccucgaacaacagcaacccggucgaggaugaagaugcgguggccuugacgugcgaac
cugagauccagaacaccaccuacuuguggugggugaacaaucagagccugccagucuccccacgacuccagcugucgaacgac
aacaggacccugacuuugcuguccgugacucggaacgacgugggcccuuaugaaugcgguauccagaacaagcuguccgugg
accacagcgacccugugauccugaacguccuuuacgggccggacgaccccaccauuuccccgucguacacuuacuaccggccg
ggcgugaaccugucccugucgugccacgcugccuccaauccgccggcccaguacuccuggcucaucgacggaaacauccagca
gcacacccaagaacuguucaucuccaacauuaccgagaaaaacucgggacuuuacaccugucaagccaacaauuccgccagcg
gccacucccgcaccacugucaaaacuaucacuguguccgccgaacucccgaagcccagcaucagcuccaacaacucgaagcccg
uggaggauaaggacgcugucgcguucaccugugaaccagaggcacagaauaccaccuaccuuuggugggucaacggacaguc
ccugccugucucaccgagacugcagcugucaaacgggaauaggacucugaccuuguuuaacgucacccggaacgacgcccgg
gccuacgugugcggcauccagaacuccgugagcgcaaaccggucugacccagugacccuggaugugcuguacggccccgaca
cuccgaucauuucaccccccgauucauccuaccuguccggcgcuaaccucaaccucucaugccacuccgcauccaaccccagcc
cgcaauauucguggcgcauuaacggaauuccucagcaacauacccagguccuguucauugcgaagaucaccccuaacaacaac
ggaaccuacgccugcuuugugucaaaccuggccacugguagaaacaacuccaucgugaaguccauuaccgugucggcguccg
gauccggcgagggcagaggcagccugcugacauguggcgacguggaagagaacccuggccccggagcugccccggagccgga
gaggacccccguuggccagggaucgugggcccauccgggacgcaccaggggaccauccgacaggggauucugugugguguca
ccggccaggccagcagaagaggcaaccagccucgagggagcguugucuggaaccagacauucccacccgucggugggccggc
agcaccacgcgggaccaccguccacuuccagaccgccacggccaugggacaccccuugcccgccuguguaugccgagacuaaa
cacuuccuguacucauccggagacaaggaacagcuucggccguccuuccuccugucgucgcucagaccgagccugaccggag
cacgcagauugguggaaacuaucuuccuugggucacguccguggaugccagguaccccacggcgccucccgcgccucccaca
gagauacuggcagaugcggccucuguuccuggaauugcugggaaaccacgcucagugcccguacggaguccugcucaagacu
cacugcccucugagggcggcggucacuccggcggccggagugugcgcacgggagaagccccagggaagcguggcagcuccgg
aagaggaggacaccgauccgcgccgccucgugcaacuucugcgccagcacuccucgcccuggcaagucuacggguucguccg
cgccugccugcgccgccuggugccgccugggcucugggguucccggcauaacgagcgccgcuuccugagaaauacuaagaag
uuuaucucacuuggaaaacaugccaaguugucgcugcaagaacucacguggaagaugucaguccgcgauugcgccuggcugc
gccgcucgccgggcgucggguguguuccagcugcagaacaccgccugagagaagaaauucuggccaaauuucugcauuggcu
gaugucaguguacguggucgagcugcugcgcuccuuuuucuacgucacugagacuaccuuucaaaagaaccgccuguucuuc
uaccgcaaaucuguguggagcaagcugcagucaaucggcauucgccagcaucugaagagggugcagcugcgggaacuuuccg
aggcagaaguccgccagcaccgggaggcccggccggcgcuucucacgucgcgucugagauucaucccaaagcccgacgggcu
gaggccuaucgucaacauggauuacgucgugggcgcucgcaccuuucgccgugaaaagcgggccgaacgcuugaccucacgg
gugaaggcccucuucuccgugcugaacuacgagagagcaagacggccuggccugcugggagcuucggugcugggacuggac
gauauccaccgggcuuggcggaccuuuguucuccgggugagagcccaagacccuccgccggaacuguacuucgugaaggugg
cgaucaccggagccuaugauacuauuccgcaagaucgacucaccgaagucaucgccucgaucaucaaaccgcagaacacuuac
ugcgucaggcgguacgccgugguccagaaggccgcgcauggccacgugagaaaggcguucaagucgcacguguccacucuca
ccgaccuccagccuuacaugaggcaauucguugcgcauuugcaagagacuucgccccugagagaugcgguggucaucgagca
gagcuccagccugaacgaagcgagcagcggucuguuugacguguuccuccgcuucaugugucaucacgcggugcgaaucagg
ggaaaaucauacgugcagugccagggaaucccacaaggcagcauucugucgacucucuuguguucccuuugcuacggcgaua
uggaaaacaagcuguucgcugggaucagacgggacggguugcugcucagacugguggacgacuuccugcuggugacuccgc
accucacucacgccaaaaccuuucuccgcacucuggugaggggagugccagaauacggcuguguggucaaucuccggaaaac
uguggugaauuucccugucgaggaugaggcacucggaggaaccgcauuuguccaaaugccagcacauggccuguucccaugg
ugcggucugcugcuggacacccgaacucuugaagugcaguccgacuacuccagcuaugcccggacgagcauccgcgccagcc
ucacuuucaaucgcggcuuuaaggccggacgaaacaugcgcagaaagcuuuucggaguccuccggcuuaaaugccauucgcu
cuuucucgaucuccaagucaauucgcugcagaccgugugcacgaacaucuacaagauccugcugcuccaagccuaccgguucc
acgcuugcgugcuucagcugccguuucaccaacagguguggaagaacccgaccuucuuucugcgggucauuagcgauacugc
cucccuguguuacucaauccucaaggcaaagaacgccggaaugucgcugggugcgaaaggagccgcgggaccucuuccuagc
gaagcggugcaguggcucugccaccaggcuuuccuccugaagcugaccaggcacagagugaccuacgucccgcugcugggcu
cgcugcgcacugcacagacccagcugucuagaaaacuccccggcaccacccugaccgcucuggaagccgccgccaacccagcau
ugccgucagauuucaagaccaucuuggacggauccggcacaauccugucugagggcgccaccaacuucagccugcugaaacu
ggccggcgacguggaacugaacccuggcccuaccccuggaacccagagccccuucuuccuucugcugcugcugaccgugcug
acugucgugacaggcucuggccacgccagcucuacaccuggcggcgagaaagagacaagcgccacccagagaagcagcgugcc
aagcagcaccgagaagaacgccguguccaugaccagcuccgugcugagcagccacucuccuggcagcggcagcagcacaacac
agggccaggaugugacacuggccccugccacagaaccugccucuggaucugccgccaccuggggacaggacgugacaagcgu
gccagugaccagaccugcccugggcucuacaacacccccugcccacgaugugaccagcgccccugauaacaagccugccccug
gaagcacagccccuccagcucauggcgugaccucugccccagauaccagaccagccccaggaucuacagccccacccgcacacg
gcgugacaagugccccugacacaagacccgcuccaggcucuacugcuccuccugcccauggcgugacaagcgcucccgauaca
aggccagcuccuggcuccacagcaccaccagcacauggcgugacaucagcucccgacacuagaccugcucccggaucaaccgc
uccaccagcucacggcgugaccagcgcaccugauaccagaccugcucugggaagcaccgccccucccgugcacaaugugacau
cugcuuccggcagcgccagcggcucugccucuacacuggugcacaacggcaccagcgccagagccacaacaaccccagccagc
aagagcacccccuucagcaucccuagccaccacagcgacaccccuaccacacuggccagccacuccaccaagaccgaugccucu
agcacccaccacuccagcgugcccccucugaccagcagcaaccacagcacaagcccccagcugucuaccggcgucucauucuuc
uuucuguccuuccacaucagcaaccugcaguucaacagcagccuggaagaucccagcaccgacuacuaccaggaacugcagcg
ggauaucagcgagauguuccugcaaaucuacaagcagggcggcuuccugggccugagcaacaucaaguucagacccggcagc
gugguggugcagcugacccuggcuuuccgggaaggcaccaucaacgugcacgacguggaaacccaguucaaccaguacaaga
ccgaggccgccagccgguacaaccugaccaucuccgauguguccguguccgacgugcccuucccauucucugcccagucugg
cgcaggcgugccaggauggggaauugcucugcuggugcucgugugcgugcugguggcccuggccaucguguaucugauug
cccuggccgugugccagugccggcggaagaauuacggccagcuggacaucuuccccgccagagacaccuaccaccccaugagc
gaguaccccacauaccacacccacggcagauacgugccacccagcuccaccgacagaucccccuacgagaaagugucugccggc
aacggcggcagcucccugagcuacacaaauccugccguggccgcugccuccgccaaccug.

1. An antigen construct, comprising a nucleotide sequence encoding an immunogenic CEA polypeptide.

2. The antigen construct according to claim 1, further comprising: (1) a nucleotide sequence encoding an immunogenic MUC1 polypeptide; or (2) a nucleotide sequence encoding an immunogenic TERT polypeptide.

3. The antigen construct according to claim 2, wherein the immunogenic CEA polypeptide is selected from the group consisting of:
(1) a polypeptide comprising amino acids 2-702 of SEQ ID NO:2, amino acids 323-702 of SEQ ID NO:2, or amino acids 323-677 of SEQ ID NO:2;
(2) a polypeptide comprising amino acid sequence of SEQ ID NO:15 or amino acids 4-704 of SEQ ID NO:15;
(3) a polypeptide comprising the amino acid sequence of SEQ ID NO:17 or amino acids 4-526 of SEQ ID NO:17;
(4) a polypeptide comprising the sequence of SEQ ID NO:19 or amino acids 4-468 of SEQ ID NO:19; and
(5) a polypeptide that is a functional variant of any of the polypeptides (1)-(4) above.

4. The antigen construct according to claim 1, further comprising a nucleotide sequence encoding an immunogenic MUC1 polypeptide and a nucleotide sequence encoding an immunogenic TERT polypeptide.

5. The antigen construct according to claim 4, further comprising a spacer nucleotide sequence.

6. The antigen construct according to claim 5, wherein the spacer nucleotide sequence encodes a 2A peptide.

7. The antigen construct according to claim 6, wherein the 2A peptide is selected from the group consisting of EMC2A, ERA2A, ERB2A, and T2A.

8. The antigen construct according to claim 4, wherein the immunogenic CEA polypeptide is selected from the group consisting of:
(1) a polypeptide comprising amino acids 2-702 of SEQ ID NO:2, amino acids 323-702 of SEQ ID NO:2, or amino acids 323-677 of SEQ ID NO:2;
(2) a polypeptide comprising amino acid sequence of SEQ ID NO:15 or amino acids 4-704 of SEQ ID NO:15;
(3) a polypeptide comprising the amino acid sequence of SEQ ID NO:17 or amino acids 4-526 of SEQ ID NO:17;
(4) a polypeptide comprising the sequence of SEQ ID NO:19 or amino acids 4-468 of SEQ ID NO:19; and
(5) a polypeptide that is a functional variant of any of the polypeptides (1)-(4) above.

9. The antigen construct according to claim 4, wherein the nucleotide sequence that encodes the immunogenic CEA polypeptide is selected from the group consisting of:
(1) the nucleotide sequence of SEQ ID NO:14 or a nucleotide sequence comprising nucleotides 10-2112 of SEQ ID NO:14;
(2) the nucleotide sequence of SEQ ID NO:16 or a nucleotide sequence comprising nucleotides 10-1578 of SEQ ID NO:16;
(3) the nucleotide sequence of SEQ ID NO:18 or a nucleotide sequence comprising nucleotides 10-1404 of SEQ ID NO:18; and
(4) a nucleotide sequence that is a degenerate variant of any of the nucleotide sequences (1)-(3) above.

10. The antigen construct according to claim 4, wherein the immunogenic TERT polypeptide is selected from the group consisting of:
(1) a polypeptide comprising the amino acid sequence of SEQ ID NO:9 or amino acids 2-893 of SEQ ID NO:9;
(2) a polypeptide comprising the amino acid sequence of SEQ ID NO:11 or amino acids 3-791 of SEQ ID NO:11;
(3) a polypeptide comprising the amino acid sequence of SEQ ID NO:13 or amino acids 4-594 of SEQ ID NO:13; and
(4) a polypeptide that is a functional variant of any of the polypeptides (1)-(3) above.

11. The antigen construct according to claim 4, wherein the nucleotide sequence that encodes the immunogenic TERT polypeptide is selected from the group consisting of:
(1) the nucleotide sequence of SEQ ID NO:8 or a nucleotide sequence comprising nucleotides 4-2673 of SEQ ID NO:8;
(2) the nucleotide sequence of SEQ ID NO:10 or a nucleotide sequence comprising nucleotides 10-2373 of SEQ ID NO:10;
(3) the nucleotide sequence of SEQ ID NO:12 or a nucleotide sequence comprising nucleotides 10-1782 of SEQ ID NO:12; and
(4) a degenerate variant of any of the nucleotide sequences (1)-(3) above.

12. The antigen construct according to claim 4, wherein the immunogenic MUC1 polypeptide is selected from the group consisting of:
(1) a polypeptide comprising the amino acid sequence of SEQ ID NO:5 or amino acids 4-537 of SEQ ID NO:5;
(2) a polypeptide comprising the amino acid sequence of SEQ ID NO:7 or amino acids 4-517 of SEQ ID NO:7; and
(3) a functional variant of any of the polypeptides (1) or (2) above.

13. The antigen construct according to claim 4, wherein the nucleotide sequence that encodes the immunogenic MUC1 polypeptide is selected from the group consisting of:
(1) the nucleotide sequence of SEQ ID NO:4 or a nucleotide sequence comprising nucleotides 10-1611 of SEQ ID NO:4;
(2) the nucleotide sequence of SEQ ID NO:6 or a nucleotide sequence comprising nucleotides 10-1551 of SEQ ID NO:6; and
(3) a degenerate variant of the nucleotide sequence of SEQ ID NO:4 or SEQ ID NO 6.

14. The antigen construct according to claim 8, wherein the immunogenic TERT polypeptide is selected from the group consisting of:
(1) a polypeptide comprising the amino acid sequence of SEQ ID NO:9 or amino acids 2-893 of SEQ ID NO:9;
(2) a polypeptide comprising the amino acid sequence of SEQ ID NO:11 or amino acids 3-791 of SEQ ID NO:11;
(3) a polypeptide comprising the amino acid sequence of SEQ ID NO:13 or amino acids 4-594 of SEQ ID NO:13; and
(4) a polypeptide that is a functional variant of any of the polypeptides (1)-(3) above.

15. The antigen construct according to claim 8, wherein the immunogenic MUC1 polypeptide is selected from the group consisting of:
(1) a polypeptide comprising the amino acid sequence of SEQ ID NO:5 or amino acids 4-537 of SEQ ID NO:5;
(2) a polypeptide comprising the amino acid sequence of SEQ ID NO:7 or amino acids 4-517 of SEQ ID NO:7; and
(3) a functional variant of the polypeptide (1) or (2) above.

16. The antigen construct according to claim 14, wherein the immunogenic MUC1 polypeptide is selected from the group consisting of:
(1) a polypeptide comprising the amino acid sequence of SEQ ID NO:5 or amino acids 4-537 of SEQ ID NO:5;
(2) a polypeptide comprising the amino acid sequence of SEQ ID NO:7 or amino acids 4-517 of SEQ ID NO:7; and
(3) a functional variant of the polypeptide (1) or (2) above.

17. The antigen construct according to claim 4, wherein:
(1) the amino acid sequence of the immunogenic MUC1 polypeptide comprises amino acids 4-537 of SEQ ID NO:5;
(2) the amino acid sequence of the immunogenic TERT polypeptide comprises amino acids 2-893 of SEQ ID NO:9; and
(3) the amino acid sequence of the immunogenic CEA polypeptide comprises amino acids 4-468 of SEQ ID NO:19.

18. The antigen construct according to claim 4, wherein:
(1) the nucleotide sequence encoding the immunogenic MUC1 polypeptide comprises nucleotides 10-1611 of SEQ ID NO:4;
(2) the nucleotide sequence encoding the immunogenic TERT polypeptide comprises nucleotides 4-2673 of SEQ ID NO:8; and
(3) the nucleotide sequence encoding the immunogenic CEA polypeptide comprises nucleotides 10-1404 of SEQ ID NO:18.

19. The antigen construct according to claim 1, which comprises a nucleotide sequence encoding an amino acid sequence selected from the group consisting of:
(1) the amino acid sequence of SEQ ID NO:31 or an amino acid sequence comprising amino acids 4-1088 of SEQ ID NO:31;
(2) the amino acid sequence of SEQ ID NO:33 or an amino acid sequence comprising amino acids 4-1081 of SEQ ID NO:33;
(3) the amino acid sequence of SEQ ID NO:35 or an amino acid sequence comprising amino acids 4-1085 of SEQ ID NO:35;
(4) the amino acid sequence of SEQ ID NO:37 or an amino acid sequence comprising an amino acid sequence comprising amino acids 4-1030 of SEQ ID NO:37;
(5) the amino acid sequence of SEQ ID NO:39 or an amino acid sequence comprising amino acids 4-1381 of SEQ ID NO:39; and
(6) the amino acid sequence of SEQ ID NO:41 or an amino acid sequence comprising amino acids 4-1441 of SEQ ID NO:41.

20. The antigen construct according to claim 1, which comprises a nucleotide sequence selected from the group consisting of:
(1) the nucleotide sequence of SEQ ID NO:30 or a nucleotide sequence comprising nucleotides 10-3264 of SEQ ID NO:30;
(2) the nucleotide sequence of SEQ ID NO:32 or a nucleotide sequence comprising nucleotides 10-3243 of SEQ ID NO:32;
(3) the nucleotide sequence of SEQ ID NO:34 or a nucleotide sequence comprising nucleotides 10-3255 of SEQ ID NO:34;
(4) the nucleotide sequence of SEQ ID NO:36 or a nucleotide sequence comprising nucleotides 10-3090 of SEQ ID NO:36;
(5) the nucleotide sequence of SEQ ID NO:38 or a nucleotide sequence comprising nucleotides 10-4143 of SEQ ID NO:38;
(6) the nucleotide sequence of SEQ ID NO:40 or a nucleotide sequence comprising nucleotides 10-4323 of SEQ ID NO:40; and
(7) a nucleotide sequences that is a degenerate variant of any of the nucleotide sequences of (1)-(6) above.

21. The antigen construct according to claim 1, which comprises a nucleotide sequence encoding an amino acid sequence selected from the group consisting of:
(1) the amino acid sequence of SEQ ID NO:43 or an amino acid sequence comprising amino acids 4-2003 of SEQ ID NO:43;
(2) the amino acid sequence of SEQ ID NO:45 or an amino acid sequence comprising amino acids 4-2001 of SEQ ID NO:45;
(3) the amino acid sequence of SEQ ID NO:47 or an amino acid sequence comprising amino acids 4-2008 of SEQ ID NO:47;
(4) the amino acid sequence of SEQ ID NO:49 or an amino acid sequence comprising amino acids 4-1996 of SEQ ID NO: 49;
(5) the amino acid sequence of SEQ ID NO:51 or an amino acid sequence comprising amino acids 4-1943 of SEQ ID NO:51; and
(6) the amino acid sequence of SEQ ID NO:53 or an amino acid sequence comprising amino acids 4-1943 of SEQ ID NO:53.

22. The antigen construct according to claim 1, which comprises a nucleotide sequence selected from the group consisting of:
(1) the nucleotide sequence of SEQ ID NO:42 or a nucleotide sequence comprising nucleotides 10-6009 of SEQ ID NO:42;
(2) the nucleotide sequence of SEQ ID NO:44 or a nucleotide sequence comprising nucleotides 10-6003 of SEQ ID NO:44;
(3) the nucleotide sequence of SEQ ID NO:46 or a nucleotide sequence comprising nucleotides 10-6024 of SEQ ID NO:46;
(4) the nucleotide sequence of SEQ ID NO:48 or a nucleotide sequence comprising nucleotides 10-5988 of SEQ ID NO:48;
(5) the nucleotide sequence of SEQ ID NO:50 or a nucleotide sequence comprising nucleotides 10-5829 of SEQ ID NO:50;
(6) the nucleotide sequence of SEQ ID NO:52 or a nucleotide sequence comprising nucleotides 10-5829 of SEQ ID NO:52; and
(7) a nucleotide sequence that is a degenerate variant of any of the nucleotide sequences of (1)-(6) above.

23. The antigen construct according to claim 21, which comprises a nucleotide sequence encoding the amino acid sequence of SEQ ID NO:51 or an amino acid sequence comprising amino acids 4-1943 of SEQ ID NO:51.

24. The antigen construct according to claim 1, which comprises the nucleotide sequence of SEQ ID NO:50 or a nucleotide sequence comprising nucleotides 10-5829 of SEQ ID NO:50.

25. The antigen construct according to claim 1, which comprises:
(1) a nucleotide sequence of any of SEQ ID NOs: 87, 88, 89, 90, 91, and 92; or
(2) a degenerate variant of any of the nucleotide sequence of SEQ ID NOS: 87, 88, 89, 90, 91, or 92.

26. A vector, comprising an antigen construct according to claim 4.

27. The vector according to claim 26, wherein:
(1) the amino acid sequence of the immunogenic MUC1 polypeptide comprises amino acids 4-537 of SEQ ID NO:5;
(2) the amino acid sequence of the immunogenic TERT polypeptide comprises amino acids 2-893 of SEQ ID NO:9; and
(3) the amino acid sequence of the immunogenic CEA polypeptide comprises amino acids 4-468 of SEQ ID NO:19.

28. The vector according to claim 26, wherein:
(1) the nucleotide sequence encoding the immunogenic MUC1 polypeptide comprises nucleotides 10-1611 of SEQ ID NO:4;
(2) the nucleotide sequence encoding the immunogenic TERT polypeptide comprises nucleotides 4-2673 of SEQ ID NO:8; and
(3) the nucleotide sequence encoding the immunogenic CEA polypeptide comprises nucleotides 10-1404 of SEQ ID NO:18.

29. The vector according to claim 26, which is a plasmid vector.

30. The vector according to claim 26, which is a viral vector.

31. The vector according to claim 26, which comprises a nucleotide sequence encoding an amino acid sequence of SEQ ID NO:43, 45, 47 49, 51, or 53.

32. The vector according to claim 26, which comprises: (1) a nucleotide sequence SEQ ID NO:42, 44, 46, 48, 50, or 52; or (2) a degenerate variant of the nucleotide sequence SEQ ID NO:42, 44, 46, 48, 50, or 52.

33. The vector according to claim 26, which comprises a nucleotide sequence of SEQ ID NO: 57, 59, 61, 63, 65, 67, 69, 70, 71, 72, 73, or 74.

34. The vector according to claim 26, which comprises a nucleotide sequence of SEQ ID NO: 58, 60, 62, 64, 66, or 68.

35. A pharmaceutical composition comprising: (i) a vector according to claim 26; and (ii) a pharmaceutically acceptable carrier.

36. A method of treating cancer in a human in need of treatment, comprising administering to the human an effective amount of the pharmaceutical composition according to claim 35.
